SEQUENCE LISTING (336 sequences)

SEQ ID NO: 1 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 2 - protein, 98 aa, Canis lupus
  source 1..98: note = Canis lupus familiaris
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCAS

SEQ ID NO: 3 - protein, 98 aa, Canis lupus
  source 1..98: note = Canis lupus familiaris
EVQLVESGGDLVKPGGSLRLSCVASGFTFSSNYMSWIRQAPGKGLQWVSQISSDGSSTSYADAVKGRFTISRDNAKNTLYLQMNSLRDEDTAVYYCAR

SEQ ID NO: 4 - protein, 97 aa, Canis lupus
  source 1..97: note = Canis lupus familiaris
EVQLVESGGDMVKPGGSLRLSCVASGFTFSSYYMYWARQAPGKGLQWVSHINKDGSSTSYADAVKGRFTISRDNAKNTLYLQMNSLRAEDTAVYYCA

SEQ ID NO: 5 - protein, 98 aa, Canis lupus
  source 1..98: note = Canis lupus familiaris
EVQLVESGGDLVKPGGSLRLSCVASGFTFSSYYMYWVRQAPGKGLQWVARISSDGSSTYYADAVKGRFTISRDNAKNTLYLQMNSLRAEDTAMYYCAK

SEQ ID NO: 6 - protein, 98 aa, Canis lupus
  source 1..98: note = Canis lupus familiaris
EVQLVESGGDLVKPGGSLRLSCVASGFTFSSSDMSWIRQAPGKGLQWVAYISNDGSSTSYADAVKGRFTISRDNAKNTLYLQMNSLRAEDTAVYYCAD

SEQ ID NO: 7 - protein, 98 aa, Canis lupus
  source 1..98: note = Canis lupus familiaris
EVQLVESGGDLVKPGGSLRLSCVASGFTFSSNDMDWVRQAPGKGLQWLTRISNDGRSTGYADAVKGRFTISRDNAKNTLYLQMNSLRAEDTAVYYCAK

SEQ ID NO: 8 - protein, 98 aa, Canis lupus
  source 1..98: note = Canis lupus familiaris
EVQLVESGGDLVKPGGSLRLSCVASGFTFSSYYMEWVRQAPGKGLQWVAQISSDGSSTYYPDAVKGQFTISRDNAKNTLYLQMNSLGAEDTAVYYCAK

SEQ ID NO: 9 - protein, 98 aa, Canis lupus
  source 1..98: note = Canis lupus familiaris
EVQLVESGGDLVKPGGTLRLSCVASGFTFSSYDMSWVRQSPGKGLQWVAVIWNDGSSTYYADAVKGRFTISRDNAKNTLYLQMNSLRAEDTAVYYCAK

SEQ ID NO: 10 - protein, 98 aa, Canis lupus
  source 1..98: note = Canis lupus familiaris
EVQLVESGGDLEKPGGSLRLSCVASGFTFSSYGMSWVRQAPGKGLQGVSLIRYDGSSTRYADAVKGRFTISRDNAKNTLYLQMNSLRAEDTAVYSCAK

SEQ ID NO: 11 - protein, 98 aa, Canis lupus
  source 1..98: note = Canis lupus familiaris
EVQLVESGGDLMKPGGSLRLSCVASGFTISSNYMNWVRQAPGKGLQWVGYISSDGSSTSYADAVKGRFTISRDNAKNTLYLQMNSLRAEDTAVYYCVK

SEQ ID NO: 12 - protein, 98 aa, Canis lupus
  source 1..98: note = Canis lupus familiaris
EVQLVESGGDLVKPGGSLRLSCVASGFTFSSYAMSWVRQAPGKGLQWVAYINSGGSSTYYADAVKGRFTISRDNARNTLYLQMNSLRSEDTAVYYCPK

SEQ ID NO: 13 - protein, 98 aa, Canis lupus
  source 1..98: note = Canis lupus familiaris
EVQLVESGGDLVKPGGSLRLSCVTSGFTFSSYWMSCVRQAPGKELQWVAYINSGGSSTWYTDAVKGRFTISRDNAKNTLYLQMNNLRAEDTAVYYCAR

SEQ ID NO: 14 - protein, 98 aa, Canis lupus
  source 1..98: note = Canis lupus familiaris
EVQLVESGGNLVKPGGSLRLSCVASGFTFSSYYMDWVRQAPGKRLQWVAGISSDGSSTYYPQAVKGRFTISRDNAKNTLYLQMNSLRAEDSAVYYCAM

SEQ ID NO: 15 - protein, 98 aa, Canis lupus
  source 1..98: note = Canis lupus familiaris
EVQLVESGGDLVKPEGSLRLSCVVSGFTFSSYDMSWVRQAPGKGLQWVAYISSDGRSTSYTDAVKGRFTISRDNAKNTLYLQMNSLRTEDTAVYYCAK

SEQ ID NO: 16 - protein, 98 aa, Canis lupus
  source 1..98: note = Canis lupus familiaris
EVQLVESGGDLVKPGGSLRLSCVASGFTFSSYYMFWIRQAPGKGNQWVGYINKDGSSTYYPDAVKGRFTISRDNAKNTLYLQMNSLTVEDTALYYCAR

SEQ ID NO: 17 - protein, 96 aa, Canis lupus
  source 1..96: note = Canis lupus familiaris
ELTLQESGPGLVKPSQTLSLTCVVSGGSVTSSYYWNWIRQRPGRGLEWMGYWTGSTNYNPAFQGRISITADTAKNQFSLQLSSMTTEDTAVYYCAR

SEQ ID NO: 18 - protein, 98 aa, Canis lupus
  source 1..98: note = Canis lupus familiaris
EVQLVESGEDLVKPGGSLRLSCVASGFTFSSSEMSWVHQAPGQGLQWVSWIRYDGSSSRYADTVKGRFTISRDNAKNTLYLQMNSLRAEDTAIYYCAD

SEQ ID NO: 19 - protein, 98 aa, Canis lupus
  source 1..98: note = Canis lupus familiaris
EVPLVESGGELVKPEGSLRFSCVASGFTFSSYWISWVRQAPGKGLHWVSVINKDGSTTYHADAVKGRFTISRDNAKNTLYLQMNSLRAEGTTVYYCAH

SEQ ID NO: 20 - protein, 98 aa, Canis lupus
  source 1..98: note = Canis lupus familiaris
EVQLVESGGDLVKPAGSLRLSCVASGFTFSSYSMSWVRQAPEKGLQLVAGINSGGSSTYYTDAVKGRFTISRDNAKNTVYLQMNSLRAEDTAMYYCAK

SEQ ID NO: 21 - protein, 98 aa, Canis lupus
  source 1..98: note = Canis lupus familiaris
EVQLVESGGDLVKPEGSLRLSCVASGFTFSSFYMSWFCQAPRKGLQWVAEISSSGSSTSYADIVKGRFTISRDNAKNMLYLQMNSLRAEDMAVYYCAR

SEQ ID NO: 22 - protein, 98 aa, Canis lupus
  source 1..98: note = Canis lupus familiaris
EVQLVESGGDLVKSGGSLRLSCVASGFTFSSYYMHWVRQATGKGLQWVTRISNDGSSTRYADAMKGQFTISRDNSKNTLYLQMNSQRAEDMALYYCAR

SEQ ID NO: 23 - protein, 98 aa, Canis lupus
  source 1..98: note = Canis lupus familiaris
EMQLVEAGGDLVKLGGSLRLFCVASGFTFSSYWMSWVGQAPGKGLQWVAYINSGGSSTYYADAVKGRFTISRDNAKNTLYLQMNCLRAEDTAVYYCVG

SEQ ID NO: 24 - protein, 98 aa, Canis lupus
  source 1..98: note = Canis lupus familiaris
EVQLVESGGDLAKPGGSLRLSCVASGLTFSSYSMSWVRQAPGKGLQWVTAISYDGSSTYYTDAVKGRFTISRDNARNTVYLQMNSLRAEDTAVYYCVD

SEQ ID NO: 25 - protein, 98 aa, Canis lupus
  source 1..98: note = Canis lupus familiaris
ELQLVESGGDLVKPGGSLRLSCVSSGFTFSSYWMHWVLQAPGKGLEWVAIINSGGGSIYYADTVKGRFTISRENAKNTLYLQMNSLRAEDRAMHYCAK

SEQ ID NO: 26 - protein, 98 aa, Canis lupus
  source 1..98: note = Canis lupus familiaris
GVQLVESGGDLVKPGGSLTLSCVAYGFTFSSYSMQWVCQAPGKGVQWVAYINSGGSSTSSADAVKGRFIISRDNVKNTLYLQMNSLRAEDTAVYYCAG

SEQ ID NO: 27 - protein, 98 aa, Canis lupus
  source 1..98: note = Canis lupus familiaris
EVQLVESGEDLVKPGGSLRLSCVASGFTFSSSEMSWVHQAPGQGLQWVSWIRYDGSISRYADTVKGRFTISRDNVKNTLYLQMNSLRAEDTAIYYCAD

SEQ ID NO: 28 - protein, 98 aa, Canis lupus
  source 1..98: note = Canis lupus familiaris
EVQLVESGGNLVKPGGSLRLSCVASGLTFYSYAIYWVHEAPGKGLQWVAAITTDGSSTYYTDAVKGRFTISRDNAKNTLYLQMNSLRAEDMPVYYCAR

SEQ ID NO: 29 - protein, 98 aa, Canis lupus
  source 1..98: note = Canis lupus familiaris
EVQLVESGGDLVKPERSLRLSCVASGFTFSSFYMSWFCQAPGKGLQCVAEISSSGNSTSYADAVKGRFTISRDNAKNTLYLRMHSLRAEDTAVYYCAR

SEQ ID NO: 30 - protein, 97 aa, Canis lupus
  source 1..97: note = Canis lupus familiaris
GAAGGVWGDLVKPGGSLRLSCVASGFTFSSYYMSWVRQAPGKGLQWVGYINSGGSSTYYADAVKGRFTISRDNAKNTLYLQMNSLRAEDTAVYYCGK

SEQ ID NO: 31 - protein, 98 aa, Canis lupus
  source 1..98: note = Canis lupus familiaris
EVQLVETEGDLVKPGGSLRLSCVASGFTFSSYDMDWVYQAPGKGLQWVTYISNGGSSTRYADAVKGQFTISRDNARNTLYLQMNSLRDKDMAVYYCVS

SEQ ID NO: 32 - protein, 98 aa, Canis lupus
  source 1..98: note = Canis lupus familiaris
EEQLVEFGGHMVNPGGSLGLSCQASGFTFSSYGMSWVRQAQKKGLQWVGHISYDGSSTYYADTLRDRFTISRDNTKNMLYLQMNSLRAEDTAVYYCMR

SEQ ID NO: 33 - protein, 98 aa, Canis lupus
  source 1..98: note = Canis lupus familiaris
EVQLVEPGGELVKPGASLRLSCVVPGFTFSSYNMGWAHQPPGKGMQWVAGFNSGGSSTSYTDAVKGEFTISRDNVKNTLYLQMNSLRSEDTAVYYCVK

SEQ ID NO: 34 - protein, 87 aa, Canis lupus
  source 1..87: note = Canis lupus familiaris
GEAWGSLRLSCVTSGFTFSSYAMHWVRQAPGKGLQWVAVISRDGSSTNYADAVKGRFTISRDNAKNMLYLQMNSLRAEDTAMYYCAK

SEQ ID NO: 35 - protein, 90 aa, Canis lupus
  source 1..90: note = Canis lupus familiaris
GGLVKPGGSLRLSCVDPGFTFSSYWMYWVHQAPGMGLQWLAEISSTGSSTNYADAVRGPFTISRDNAKNTLYLQVNSLRAEDTAVYYCVS

SEQ ID NO: 36 - protein, 96 aa, Canis lupus
  source 1..96: note = Canis lupus familiaris
AAGGVWGNLVKPGESLRLSCVASGFTFSSYWMHWVCQAPGKGLGWVAIINSGGGSTYYADTVKGQFTIFRDNAKNMLYLQMNSLRAQDMTAYYCVS

SEQ ID NO: 37 - protein, 86 aa, Canis lupus
  source 1..86: note = Canis lupus familiaris
SLGGSLRLSCVASEFIFSGYWKYWIHQAPGKGLQWVTWISNDGSSKSYADAVKGQFTISKDNAKYTLYLQMNSLRAEDMAVYYCMM

SEQ ID NO: 38 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 39 - protein, 98 aa, Canis lupus
  source 1..98: note = Canis lupus familiaris
EVQLVESGGELVKPGGSLRLSCVASGFTFSSYYMSWIRQAPGKGLQWVADISDSGGSTYYTDAVKGRFTISRDNVKNSLYLQMNSLRAEDTAVYYCAK

SEQ ID NO: 40 - protein, 98 aa, Canis lupus
  source 1..98: note = Canis lupus familiaris
EVQLVESGGDLVKPGGSLRLSCVASGFTFSSHAKSWVRQAPGKGLKWVAVISSSGSSTGSADTVKGRFTISRDNAKNTLYLQMNSLRAEDTAVYYCAK

SEQ ID NO: 41 - protein, 98 aa, Canis lupus
  source 1..98: note = Canis lupus familiaris
EVQLVESGGDLVKPGGSLRLSCVASGFTFSSYYMSWIRQAPGKGLQWVADISDSGGSTGYADAVKGRFTISRENAKNKLYLQMNSLRAEDTAVYYCAK

SEQ ID NO: 42 - protein, 98 aa, Canis lupus
  source 1..98: note = Canis lupus familiaris
EVQLVESGGDLVKPGGSLRLSCVASGFTFSNYDMSWVRQAPGKGLQWVAAISYDGSSTYYTDAVKGRFTISRDNARNTVYLQMNSLRAEDTAVYYCAK

SEQ ID NO: 43 - protein, 98 aa, Canis lupus
  source 1..98: note = Canis lupus familiaris
ELQLVELGGDLVKPGGSLRLSCVASGFTFSSYAMSWVCQAPGKGLQWVAAISSSGSSTYHVDAVKGRFTISRDNAKNTVYLQMNSLRAEDTAVYYCAD

SEQ ID NO: 44 - protein, 95 aa, Canis lupus
  source 1..95: note = Canis lupus familiaris
LVESGGDLMKPGGSLRLSCVASGFTFRSYGMSWVCQASGKGLQWVAAISYDGRSTYYTDTVKGRFTISRDNGKNTLYLQMNSLRAEDTAVYYCAS

SEQ ID NO: 45 - protein, 96 aa, Canis lupus
  source 1..96: note = Canis lupus familiaris
SWWSLGEDLVKPGGSLRLSCVTSGFTFSRYAMSWVGQAPGKGLQWVAAISSSGSSTYYVDAVKGRFTISIDNAKNMVYLQMNSLRAEDIAVYYCGK

SEQ ID NO: 46 - protein, 98 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 47 - protein, 98 aa, Canis lupus
  source 1..98: note = Canis lupus familiaris
EVQLVESGGDLVKPGGSLRLSCVASGFTFSSYDMDWVRQAPGKGLQWLSEISSSGSSTYYADAVKGRFTISRDNAKNTLYLQMNSLRAEDTAVYYCAR

SEQ ID NO: 48 - protein, 98 aa, Canis lupus
  source 1..98: note = Canis lupus familiaris
EVQLVESGGDLVKPVGSLRLSCVASGFTFSSYDMNWVRQAPGKGLQWVAYISSGGSSTYYADAVKGRFTISRDNAKNTLYLQMNSLRAEDTAMYYCAG

SEQ ID NO: 49 - protein, 98 aa, Canis lupus
  source 1..98: note = Canis lupus familiaris
EVQLVESGGDLVKPGGSLRLSCVASGFTFSNYEMYWVRQAPGKGLEWVARIYESGSTTYYAEAVKGRFTISRDNAKNMAYLQMNSLRAEDTAVYYCAS

SEQ ID NO: 50 - protein, 98 aa, Canis lupus
  source 1..98: note = Canis lupus familiaris
EVQLLESGGDRVKPGGSQRLSCVASRFTFSSYSMHCLRQSPGMGLQWVTYISSNGSSTYYADAVKGRFTISRDKAKNMLYLQMNSLRAQDIALYYCAD

SEQ ID NO: 51 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 52 - protein, 98 aa, Canis lupus
  source 1..98: note = Canis lupus familiaris
EEQLVEFGGHMVNPGGSLGLSCQASGFTFSSYGMSWVRQAQKKGLQWVGHISYDGSSTYYTDTVRDRFTISRDNTKNMLYLQMNSLRAEDTAVYYCMR

SEQ ID NO: 53 - protein, 98 aa, Homo sapiens
EVQLVESGGGLVQPGGSLRLSCSASGFTFSSYAMHWVRQAPGKGLEYVSAISSNGGSTYYADSVKGRFTISRDNSKNTLYLQMSSLRAEDTAVYYCVK

SEQ ID NO: 54 - protein, 81 aa, Canis lupus
  source 1..81: note = Canis lupus familiaris
SPVSCVASGFTFSNYSMIWVRQAPRKGLQWITTISNSGSSTNHADTVKGRFTISRDNTKNTLYLQMSSLGADDTALYYCVR

SEQ ID NO: 55 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 56 - protein, 98 aa, Canis lupus
  source 1..98: note = Canis lupus familiaris
EVQLVQSGAEVKKPGASVKVSCKTSGYTFINYYMIWVRQAPGAGLDWMGQIDPEDGATSYAQKFQGRVTLTADTSTSTAYMELSSLRAGDIAVYYCAR

SEQ ID NO: 57 - protein, 93 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYC

SEQ ID NO: 58 - protein, 93 aa, Canis lupus
  source 1..93: note = Canis lupus familiaris
DIVMTQTPLSLSVSPGEPASISCKASQSLLHSNGNTYLYWFRQKPGQSPQRLIYKVSNRDPGVPDRFSGSGSGTDFTLRISRVEADDAGVYYC

SEQ ID NO: 59 - protein, 93 aa, Canis lupus
  source 1..93: note = Canis lupus familiaris
DIVMTQTPLSLSVSPGEPASISCRASQSLLHSNGNTYLYWFRQKPGQSPQGLIYLVSNRFSWVPDRFSGSGSGTDFTLRISRVEADDAGVYYC

SEQ ID NO: 60 - protein, 93 aa, Canis lupus
  source 1..93: note = Canis lupus familiaris
DIVMTQTPPSLSVSPRETASISCKASQSLLHSDGNTYLDWYLQKPGQSPQLLIYLVSNRFTGVSDRFSGSGSGTDFTLRISRVEANDTGVYYC

SEQ ID NO: 61 - protein, 93 aa, Canis lupus
  source 1..93: note = Canis lupus familiaris
DIVMTQTPLSLSVSPGETASISCKASQSLLHSDGNTYLNWFRQKPGQSPQRLIYKVSNRDPGVPDRFSGSGSGTDFTLRISRVEADDTGVYYC

SEQ ID NO: 62 - protein, 93 aa, Canis lupus
  source 1..93: note = Canis lupus familiaris
EVVMIQTPLSLSVSPGEPASISCRASQSLRHSNGNTYLYWYLQKPGQSPQLLIDLVSNHFTGVSDRFSGSGSGTDFTLRISRVEAEDVGVYYC

SEQ ID NO: 63 - protein, 93 aa, Canis lupus
  source 1..93: note = Canis lupus familiaris
EAVMTQTPLSLAVTPGELATISCRASQSLLRSDGKSYLNWYLQKPGQTPRPLIYEASKRFSGVSDRFSGSGSGTDFTLKISRVEAEDVGVYYC

SEQ ID NO: 64 - protein, 93 aa, Canis lupus
  source 1..93: note = Canis lupus familiaris
DIVMTQTPPSLSVSPGEPASISCKASQSLLHSNGNTYLNWFRQKPGQSPQGLIYRVSNRSTGVSDRFSGSGSGTDFTLRISRVEADDAGVYYC

SEQ ID NO: 65 - protein, 93 aa, Canis lupus
  source 1..93: note = Canis lupus familiaris
DIVMTQNPLSLSVSPGETASISCKASQSLLHSNGNTYLNWFRQKPGQSPQGLIYKVSNRDPGVPDRFSGSGSGTDFTLRISRVEADDAGVYYC

SEQ ID NO: 66 - protein, 93 aa, Canis lupus
  source 1..93: note = Canis lupus familiaris
DIVMTQTPLSVSVSPGETASISCRASQSLLHSDGNTYLDWYLQKPGQIPKDLIYRVSNCFTGVSDRFSGSGSGTDFTLRISRVEADNAGVYYC

SEQ ID NO: 67 - protein, 93 aa, Canis lupus
  source 1..93: note = Canis lupus familiaris
DIVMTQTPLSLSVSPGETASISCKASQSLLHSNGNTYLFWFRQKPGQSPQRLINLVSNRDPGVPHRFSGSGSGTDFTLRISRVEADDAGVYYC

SEQ ID NO: 68 - protein, 93 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYC

SEQ ID NO: 69 - protein, 93 aa, Canis lupus
  source 1..93: note = Canis lupus familiaris
DIVMTQTPLSLSVSPGETASISCRANQSLLHSNGNTYLDWYMQKPGQSPQGLIYRVSNHFTGVSDRFSGSGSGTDFTLKISRVEADDAGVYYC

SEQ ID NO: 70 - protein, 94 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYC

SEQ ID NO: 71 - protein, 94 aa, Canis lupus
  source 1..94: note = Canis lupus familiaris
EIVMTQSPGSLAGSAGESVSINCKSSQSLLYSFNQKNYLAWYQQKPGERPKLLIYLASSWASGVPARFSSSGSGTDFTLTINNLQAEDVGDYYC

SEQ ID NO: 72 - protein, 88 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYC

SEQ ID NO: 73 - protein, 88 aa, Canis lupus
  source 1..88: note = Canis lupus familiaris
EIVMTQSPASLSLSQEEKVTITCRASQSVSSYLAWYQQKPGQAPKLLIYGTSNRATGVPSRFSGSGSGTDFSFTISSLEPEDVAVYYC

SEQ ID NO: 74 - protein, 89 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYC

SEQ ID NO: 75 - protein, 89 aa, Canis lupus
  source 1..89: note = Canis lupus familiaris
QSVLTQPASVSGSLGQRVTISCTGSSSNIGRGYVGWYQQLPGTGPRTLIYDNSNRPSGVPDRFSGSKSGSTATLTISGLQAEDEADYYC

SEQ ID NO: 76 - protein, 89 aa, Canis lupus
  source 1..89: note = Canis lupus familiaris
QSVLTQPASVSGSLGQRVTISCTGSSSNIGRDYVGWYQQLPGTRPRTLIYGNSNRPSGVPDRFSGSKSGSTATLTISGLQAEDEADYYC

SEQ ID NO: 77 - protein, 89 aa, Canis lupus
  source 1..89: note = Canis lupus familiaris
QSVLTQPPSVSGFLGQRVTISCTGSSSNIGRGYVHWYQQLPGTGPRTLIYGISNRPSGVPDRFSGSRSGSTATLTISGLQAEDEADYYC

SEQ ID NO: 78 - protein, 89 aa, Canis lupus
  source 1..89: note = Canis lupus familiaris
QSVLTQPASVSGSLGQRVTISCTGSSSNIGRGYVGWYQQLPGTGPRTLIYGNSNRPSGVPDRFSGSRSGSTATLTISGLQAEDEADYYC

SEQ ID NO: 79 - protein, 89 aa, Canis lupus
  source 1..89: note = Canis lupus familiaris
QSVLTQPASVSGSLGQRITISCTGSSSNIGGNNVGWYQQLPGRGPRTVIYSTNSRPSGVPDRFSGSKSGSTATLTISGLQAEDEADYYC

SEQ ID NO: 80 - protein, 89 aa, Canis lupus
  source 1..89: note = Canis lupus familiaris
QSVLTQPPSVSGSLGQRVTISCSGSTNNISIVGASWYQQLPGKAPKLLVDSDGDRPSGVPDRFSGSKSGKSATLTITGLQAEDEADYYC

SEQ ID NO: 81 - protein, 89 aa, Canis lupus
  source 1..89: note = Canis lupus familiaris
QSVLTQPTSVSGSLGQRVTISCSGSTNNIGIVGASWYQQLPGKAPKLLVYSVGDRPSGVPDRFSGSNSGNSATLTITGLQAEDEADYYC

SEQ ID NO: 82 - protein, 89 aa, Canis lupus
  source 1..89: note = Canis lupus familiaris
QSVLTQPTSVSGSLGQRVTISCSGSTNNIGIVGASWYQQLPGKAPKLLVDSDGDRPSGVPDRFSGSKSGNSATLTITGLQAEDEADYYC

SEQ ID NO: 83 - protein, 89 aa, Canis lupus
  source 1..89: note = Canis lupus familiaris
QSMLTQPASVSGSLGQKVTISCTGSSSNIGGNYVGWYQQLPGIGPRTVIYGNNYRPSGVPDRFSGSKSGSSATLTISGLQAEDEAEYYC

SEQ ID NO: 84 - protein, 89 aa, Canis lupus
  source 1..89: note = Canis lupus familiaris
QSVLTQPASVSGSLGQRVTISCTGSSSNIGRGYVGWYQQLPGTGPRTLIYDSSSRPSGVPDRFSGSRSGSTATLTISGLQAEDEADYYC

SEQ ID NO: 85 - protein, 89 aa, Canis lupus
  source 1..89: note = Canis lupus familiaris
QSVLTQPTSVSGSLGQRVTISCSGSTNNIGIVGASWYQQLPGKAPKLLVYSDGDRPSGVPDRFSGSNSGNSDTLTITGLQAEDEADYYC

SEQ ID NO: 86 - protein, 89 aa, Canis lupus
  source 1..89: note = Canis lupus familiaris
QSVLTQPASVSGSLGQRVTISCTGSSSNIGRYSVGWFQQLPGKGPRTVIYSSSNRPSGVPDRFSGSKSGSTATLTISGLQAEDEADYYC

SEQ ID NO: 87 - protein, 89 aa, Canis lupus
  source 1..89: note = Canis lupus familiaris
QPVLTQPPSVSGFLGQRVTISCTGSSSNILGNSVNWYQQLTGRGPRTVIYYDNNRPSGVPDQFSGSKSGNSATLTISGLQAEDETDYYC

SEQ ID NO: 88 - protein, 89 aa, Canis lupus
  source 1..89: note = Canis lupus familiaris
QSVLTQPPSVSGSVGQRITISCSGSTNSIGILGVNWYQLLSGKAPKLLVDGTGNRPSGVPDRFSGSKSGNSGTLTITGLQPEDEADYYC

SEQ ID NO: 89 - protein, 89 aa, Canis lupus
  source 1..89: note = Canis lupus familiaris
QSVLNQPPSVSGSLGQRITISCSGSTNDIGMLGVNWYQQLPGNAPKLLVDGTGNRPSGVPDQFSGSKSGNSGTLTITGLQAEDEADYYC

SEQ ID NO: 90 - protein, 89 aa, Canis lupus
  source 1..89: note = Canis lupus familiaris
QSVLTQPASASGSLGQRVTVSCSSSTNNIGIIGVKWYQQIPGKAHKLLIYDNEKRPSGVPNRFSGSKSGDLSTLTINGLQGEDEADYYC

SEQ ID NO: 91 - protein, 89 aa, Canis lupus
  source 1..89: note = Canis lupus familiaris
QSVLTQPASVTGSLGQRVTISCTGSSSNIGGYNVGWFQQLPGTGPRTVIYSSSNRPSGVPDRFSGSRSGSTATLTISGLQAEDEAEYYC

SEQ ID NO: 92 - protein, 89 aa, Canis lupus
  source 1..89: note = Canis lupus familiaris
QSVLTQPASVSGVLGQRVTISCTGSSSNIGGNYVSWHQQVPETGPRNIIYADNYRASGVPDRFSGSKSGSTATLTISVLQAEDEADYYC

SEQ ID NO: 93 - protein, 89 aa, Canis lupus
  source 1..89: note = Canis lupus familiaris
QSVLTQPTSVSWSLGQRVTISCSRSTNNIGIVGASWYQQLPGKAPKLLVDSDGDQLSGVPDRFSGSKSGNSANLTITGLQAEDKADYYC

SEQ ID NO: 94 - protein, 89 aa, Canis lupus
  source 1..89: note = Canis lupus familiaris
QSVLNQLPSVLGSLGQRITISCSGSTNDIGMLGVNWYQELPGKAPKLLVDGTGNRPSGVPDRFSGSKSGNSGTLTITGLQAEDEADYYC

SEQ ID NO: 95 - protein, 89 aa, Canis lupus
  source 1..89: note = Canis lupus familiaris
QSVLTQPASVSGSLGQRVIISCTGSSSSIGRGYVGWYQQLPGTGPRTLIYGISNLPPGVPNRFSGSRSGSTATLTIAELQAEDEADYYC

SEQ ID NO: 96 - protein, 89 aa, Canis lupus
  source 1..89: note = Canis lupus familiaris
QSVLTQPPSVFRSLGQRVTISCTGSSCNVGRGYVIWYQQLLGTRPRTLIYGSSNQPSGVPNRFSGSRSGSTATLTISGFQAEDEADYYC

SEQ ID NO: 97 - protein, 89 aa, Canis lupus
  source 1..89: note = Canis lupus familiaris
QSVLTQPPSVFRSLGQRVTISCTGSSSNVGRGYVIWYQQLLGTRPRTLIYGSSNQPSGVPNQFSGSRSGSTDTLTISGFQAEDEADYYC

SEQ ID NO: 98 - protein, 76 aa, Canis lupus
  source 1..76: note = Canis lupus familiaris
PGPEVTVSCTGSCFNIGRYSVSWLQQLPGTGPRTIIYYDRSRPSGVPDRFSGSKSGSTATLTISGLQAEDEADYYC

SEQ ID NO: 99 - protein, 90 aa, Homo sapiens
QTVVTQEPSFSVSPGGTVTLTCGLSSGSVSTSYYPSWYQQTPGQAPRTLIYSTNTRSSGVPDRFSGSILGNKAALTITGAQADDESDYYC

SEQ ID NO: 100 - protein, 90 aa, Canis lupus
  source 1..90: note = Canis lupus familiaris
QTVVTQEPSLSVSPGGTVTLTCGLSSGSVSTSNYPGWYQQTLGRAPRTIIYRTSSRPSGVPNRFSGSISGNKAALTITGAQPEDEADYYC

SEQ ID NO: 101 - protein, 90 aa, Canis lupus
  source 1..90: note = Canis lupus familiaris
QTVVTQEPSLSVSPGGTVTLTCGLSSGSVSTSNYPGWYQQTQGRAPRTIIYNTSSRPSGVPNRFSGSISGNKAALTITGAQPEDEADYYC

SEQ ID NO: 102 - protein, 90 aa, Canis lupus
  source 1..90: note = Canis lupus familiaris
QTVVTQEPSLSVSLGGTVTLTCGLSSGSVSTSNYPNWSQQTPGQAPRTIIYNTNSRPSGVPNRFTGSISGNKAALTITGAQPEDEADYYC

SEQ ID NO: 103 - protein, 90 aa, Canis lupus
  source 1..90: note = Canis lupus familiaris
QTVVTQEPSLSVSPGGTVTLTCGLSSGSVSTSNHPSWYQQTQGKAPRMLIYNTNNRPSGIPNCFSGSISGNKASLTITGAQPEDETDYYC

SEQ ID NO: 104 - protein, 90 aa, Canis lupus
  source 1..90: note = Canis lupus familiaris
QTVVTQKPSLSVSPGGTVTLICGLSSGSVSTSNYPGWYQQTQGRASRTIIYSTSSRPSGVPNRFTGSISGNKAALTITGAQPEDEADYYC

SEQ ID NO: 105 - protein, 90 aa, Canis lupus
  source 1..90: note = Canis lupus familiaris
QTVVTQKPSLSVSPGGTVTLICGFSSGSVSTSNYPGWYQQTQGRASRTIIYSTSSRPSGVPNRFPGSISGNKAALTITGAQPEDEADYYC

SEQ ID NO: 106 - protein, 90 aa, Canis lupus
  source 1..90: note = Canis lupus familiaris
QTVVTQKPSLSVSPGGTVTLICGLSSGSVSTSNYPGWYQQTQGRASRTIIYSTSSRPSGVPNRFPGSISGNKAALIITGAQPEDEADYYC

SEQ ID NO: 107 - protein, 90 aa, Canis lupus
  source 1..90: note = Canis lupus familiaris
QIVVTQEPSLSVSPGGTVTLTCGLSSGSVTISNYPDWYQQTPGRSPRMLIYNTNNRPSGVPNHFSGSISGNKAALTITGAQPEDEAYYYC

SEQ ID NO: 108 - protein, 90 aa, Canis lupus
  source 1..90: note = Canis lupus familiaris
QTVVTQEPSLSVSPGGTVTLTCGLSSGSVSTGNKPGWYQHTPGQAPRRIIYDTSSRPSGVPDRFSGSISENKAALTITEAQPEDEAAYHC

SEQ ID NO: 109 - protein, 90 aa, Canis lupus
  source 1..90: note = Canis lupus familiaris
QTVVTQESSVSVSPVGTVTLTCGLSSGSLTTSNYTSWYQQTQGQSPRMLVYDTSSCPSEVPDHFSGSISGNKAALTITGAQPEDEADYYC

SEQ ID NO: 110 - protein, 83 aa, Canis lupus
  source 1..83: note = Canis lupus familiaris
QEPSLSPGGTVLLTCGLSSGSVTTSNYSSWYQQTPGRAPRTIIYNTNSHPSGVPDRFSGSISGNKAALTITGAQPEDEADYYC

SEQ ID NO: 111 - protein, 90 aa, Canis lupus
  source 1..90: note = Canis lupus familiaris
QTVVTQDPSLSVSPGGTVTLTCGLSSGSVSTSNYPGWYQQTQVKAPCMLIYSTNSYPSGVPNCFTGSISGKKAALTITGDQPEDETIIVH

SEQ ID NO: 112 - protein, 90 aa, Canis lupus
  source 1..90: note = Canis lupus familiaris
QTVITQDTSLSVSPGGTVTLTCGLSSGSVSTSNYPSWYQQTQGRDPRMLIYSTNSHPSGVPNCFTRSISGKKAALTITGAQPEDETIIVH

SEQ ID NO: 113 - protein, 75 aa, Canis lupus
  source 1..75: note = Canis lupus familiaris
GTVTLTCGLSSGSVSTSNYPNWYQQTQGRAPHRVIYNTNNRPSGVPDRFSGSISGNKAALTITAAQPEDEADYYC

SEQ ID NO: 114 - protein, 87 aa, Canis lupus
  source 1..87: note = Canis lupus familiaris
GGQEALLSVSPGGRVTLTCGLSSGSVTTSNYPNWFQQTPGRAPGTIIYSTKDCPSGVPDCFSRSISGNKAALTITGAQSEDEAITVL

SEQ ID NO: 115 - protein, 86 aa, Canis lupus
  source 1..86: note = Canis lupus familiaris
GQEALLSVSSGGRVTLTCGLSSGSVTTSNYPNWFQQTPGRAPGTIMYSTKDCPSGVPDCFSRSISGNKAALTITGAQSEDEVITVL

SEQ ID NO: 116 - protein, 90 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYC

SEQ ID NO: 117 - protein, 90 aa, Canis lupus
  source 1..90: note = Canis lupus familiaris
QAVLTQPPSVSAVLGQRVTISCTGSSTNIGSGYDVQWYQQLPGKSPKTIIYGNSNRPSGVPDRFSGSKSGSTASLTITGLQAEDEADYYC

SEQ ID NO: 118 - protein, 90 aa, Canis lupus
  source 1..90: note = Canis lupus familiaris
QAVLTQPPSVSAALGQRVTISCTGSNTNIGSGYDVQWYQQLPGKSPKTIIYGNSNRPSGVPVRFSGSKSGSTATLTITGIQAEDEADYYC

SEQ ID NO: 119 - protein, 90 aa, Canis lupus
  source 1..90: note = Canis lupus familiaris
QSVLTQPASVSGSLGQRVTISCTGSTNNIGGDNYVHWYQQLPGKAPSLLIYGDDNRESGVPERFSGSKSGSSATLTITGLQAEDEADYYC

SEQ ID NO: 120 - protein, 90 aa, Canis lupus
  source 1..90: note = Canis lupus familiaris
QAVLTQPASVSGSLGQRVTISCTGSSSNVGYGNYVGWYQQLPGTGPRTLIYGSSYRPSGVPDRFSGSSSGSSATLTISGLQAEDEADYYC

SEQ ID NO: 121 - protein, 90 aa, Canis lupus
  source 1..90: note = Canis lupus familiaris
QSVLTQPASVSGSLGQRVTISCTGSSSNVGYGNYVGWYQQLPGTGPRTLIYRSSSRPSGVPDRFSGSRSGSTATLTISGLQAEDEADYYC

SEQ ID NO: 122 - protein, 90 aa, Canis lupus
  source 1..90: note = Canis lupus familiaris
QSVLTQPASVSGSLGQRVTISCTGSSSNVGYGNYVGWYQQLPGTSPRTLIYDSSSRPSGVPDRFSGSRSGSTATLTISGLQAEDEADYYC

SEQ ID NO: 123 - protein, 90 aa, Canis lupus
  source 1..90: note = Canis lupus familiaris
QSVLTQPASVSGSLGQRVTISCTRSSSNVGYGNDVGWYQQLPGTGPRTIIYNTNTRPSGVPDRFSGSKSGSTATLTISGLQAEDEADYYC

SEQ ID NO: 124 - protein, 90 aa, Canis lupus
  source 1..90: note = Canis lupus familiaris
QSVLTQPASVSQSLGQRVTISCTGSSSNVGYNSYVSWYQQLPGTVPRTIIYYTNTRPYGVPDRFSGSKSGNSATLTIAGLQAEDEADYYC

SEQ ID NO: 125 - protein, 90 aa, Canis lupus
  source 1..90: note = Canis lupus familiaris
QAVLTPLPSVSAALGQTVTISCTGNSTQIGSGYAVQWYQQLPGKSPETIIYGDSNRPSGVPDRFSGFSSGNSATLAITGLQDEDEADYYC

SEQ ID NO: 126 - protein, 90 aa, Canis lupus
  source 1..90: note = Canis lupus familiaris
QAVLTPLPSVSAALGQTVTISCTGNSTQISSGYAVQWYQQLPGKSPETIIYGDSNRPSGVPDRFSGFSSGNSATLAITGLQDEDEADYYC

SEQ ID NO: 127 - protein, 90 aa, Homo sapiens
QSALTQPPSVSGSPGQSVTISCTGTSSDVGSYNRVSWYQQPPGTAPKLMIYEVSNRPSGVPDRFSGSKSGNTASLTISGLQAEDEADYYC

SEQ ID NO: 128 - protein, 90 aa, Canis lupus
  source 1..90: note = Canis lupus familiaris
QSALTQPSSVSGTLGQTVTISCDGSSSNIGSSNYIEWYQQFPGTSPKLLIYYTNNRPSGIPARFSGSKSGNTASLTISGLQAEDEADYYC

SEQ ID NO: 129 - protein, 90 aa, Canis lupus
  source 1..90: note = Canis lupus familiaris
QSVLTQPASVSGSLGQRVTISCTGSSSNVGYSSSVGWYQQFPGTGPRTIIYYDSSRPSGVPDRFSGSKSGSTATLTISGLQAEDEADYYC

SEQ ID NO: 130 - protein, 90 aa, Canis lupus
  source 1..90: note = Canis lupus familiaris
QSVLTQPASVSGSLGQRVTISCTGSSSNVGYGNYVGWYQQLPGTGPRTIICYTNTRPSGVPDRYSGSKSGSTATLTISGLQAEDETDYYC

SEQ ID NO: 131 - protein, 90 aa, Canis lupus
  source 1..90: note = Canis lupus familiaris
QSALTQTASMSGSLGQRVTVSCTGSSSNVGYRSYVGWYQQLPGTGPRTIIYNTNTRPSGVPDRFSGSISGSTATLTIAGLQAEDEADYYC

SEQ ID NO: 132 - protein, 90 aa, Canis lupus
  source 1..90: note = Canis lupus familiaris
QSALTQPPSMSGTLGQTIIISCTGSGSDIGRYSYVSWYQELPSTSPTLLIYGTNNRPLEIPARFSGSKSGNTAPMTISGLQAEDEANYYC

SEQ ID NO: 133 - protein, 78 aa, Canis lupus
  source 1..78: note = Canis lupus familiaris
GPGPEVTISCTRSSSNVGYSSYVGWYQQLPGTGPKTIIYNTNTRPSGVPDRFSGSKSGRTATLTIAGLQAEDEADYYC

SEQ ID NO: 134 - protein, 78 aa, Canis lupus
  source 1..78: note = Canis lupus familiaris
GPGPEVTISCTRSSSNVGYSSYVGWYQQLPGTGPKTIIYNTNTRPSGVPDRFSGSKSGSTATLTIAGLQAEDEADYYC

SEQ ID NO: 135 - protein, 87 aa, Homo sapiens
SYELTQPLSVSVALGQTARITCGGNNIGSKNVHWYQQKPGQAPVLVIYRDSNRPSGIPERFSGSNSGNTATLTISRAQAGDEADYYC

SEQ ID NO: 136 - protein, 87 aa, Canis lupus
  source 1..87: note = Canis lupus familiaris
SYVLTQLPSVSVTLRQTARITCGGDSIGSKSVYWYQQKLGQAPVLIIYRDSNRPTGIPERFSGANSGNTATLTISGALAEDEADYYC

SEQ ID NO: 137 - protein, 87 aa, Canis lupus
  source 1..87: note = Canis lupus familiaris
SYVLTQLPSVSVTLRQTARITCGGDSIGSKNVYWYQQKLGQAPVLIIYDDSSRPSGIPERFSGANSGNTATLTISGALAEDEADYYC

SEQ ID NO: 138 - protein, 87 aa, Canis lupus
  source 1..87: note = Canis lupus familiaris
SYVLTQLPSVNVTLRQPAHITCGGDSIGSKSVHWYQQKLGQAPVLIIYGDSNRPSGIPERFSGDNSGNTATLTISGALAEDEAYYYC

SEQ ID NO: 139 - protein, 87 aa, Canis lupus
  source 1..87: note = Canis lupus familiaris
SYVLTQSPSVSVTLGQTASITCRGNSIGRKDVHWYQQKPGQAPLLIIYNDNSQPSGIPERFSGTNSGSTATLTISEAQTNDEADYYC

SEQ ID NO: 140 - protein, 87 aa, Canis lupus
  source 1..87: note = Canis lupus familiaris
SYVLTQLPSMSVTLRQTARITCEGDSIGSKRVYWYQQKLGQVPVLIIYDDSSRPSGIPERFSGANSGNTATLTISGALAEDEADYYC

SEQ ID NO: 141 - protein, 84 aa, Canis lupus
  source 1..84: note = Canis lupus familiaris
LSQPPSATVTLRQTARLTCGGDSIGSKSVEWYQQKPGQPPVLIIYGDSSRPSGIPERFSGANSGNTATLTISGALAEDEADYYC

SEQ ID NO: 142 - protein, 74 aa, Canis lupus
  source 1..74: note = Canis lupus familiaris
LKQPAHITCGGDNIGSKSVHWYQQKLGQAPVLIIYYDSSRPTGIPERFSGANSGNTATLTISGALAEDEADYYC

SEQ ID NO: 143 - protein, 87 aa, Canis lupus
  source 1..87: note = Canis lupus familiaris
ANKLTQSLFMSVALGQMARITCGRDNSGRKSAHWYQQKPSQAPVMLIDDDCFQPSGFSEQFSGTNSGNTATLTISGPPARTRLLLCH

SEQ ID NO: 144 - protein, 87 aa, Homo sapiens
SYELMQPPSVSVSPGQTARITCSGDALPKQYAYWYQQKPGQAPVLVIYKDSERPSGIPERFSGSSSGTTVTLTISGVQAEDEADYYC

SEQ ID NO: 145 - protein, 87 aa, Canis lupus
  source 1..87: note = Canis lupus familiaris
SSVLTQPPSVSVSLGQTATITCSGESLSRYYAQWYQQKPGQAPMTVIYGDRERPSGIPDRFSSSSSENTHTLTISGAQAEDEAEYYC

SEQ ID NO: 146 - protein, 87 aa, Canis lupus
  source 1..87: note = Canis lupus familiaris
SPGLNQPPSVLVALGQMATNTCSGDVLGKRYAYWYQHKPSQAPVLLINKNNELGSGIPDRFSGSNSGNTATLTISGARAEDEADYYC

SEQ ID NO: 147 - protein, 84 aa, Canis lupus
  source 1..84: note = Canis lupus familiaris
LNQAPSVLVALGQMETITCSRDVLGKRYAYRYQHKPSQAPVLLINKNNEQDSGIPDRFSGSNSGNTATLTISGARAEDEAEYYC

SEQ ID NO: 148 - protein, 87 aa, Homo sapiens
SYELTQPSSVSVSPGQTARITCSGDVLAKKYARWFQQKPGQAPVLVIYKDSERPSGIPERFSGSSSGTTVTLTISGAQVEDEADYYC

SEQ ID NO: 149 - protein, 87 aa, Canis lupus
  source 1..87: note = Canis lupus familiaris
SSVLTQPPSVSVSLGQTATISCSGESLSKYYAQWFQQKAGQVPVLVIYKDTERPSGIPDRFSGSSSGNTHTLTISGARAEDEADYYC

SEQ ID NO: 150 - protein, 80 aa, Canis lupus
  source 1..80: note = Canis lupus familiaris
SSVLVALGQMETITCSRDVLGKRYAYRYQHKPSQAPVLLINKNNEQDSGIPDRFSGSNSGNTATLTISGARAEDEAEYYC

SEQ ID NO: 151 - protein, 94 aa, Homo sapiens
QPVLTQPPSSSASPGESARLTCTLPSDINVGSYNIYWYQQKPGSPPRYLLYYYSDSDKGQGSGVPSRFSGSKDASANTGILLISGLQSEDEADY

SEQ ID NO: 152 - protein, 96 aa, Canis lupus
  source 1..96: note = Canis lupus familiaris
QPVLTQPPSLSASLGTTARLTCTLSSGFSVGDYDMYWYQQKPGSPPRDLLYYYSDSYKNQGSGVSKSFSGSKDTSANAGLLLISGLQPEDEADYYC

SEQ ID NO: 153 - protein, 96 aa, Canis lupus
  source 1..96: note = Canis lupus familiaris
QLVVTQPPSLSASLGSSARLTCTLSSGFSVGSYSVTWFQQKPGSPLWYLLYYHSDSDKHQGSRVPSRFSGSKDTSANAGLLLISGLQPEDEADYYC

SEQ ID NO: 154 - protein, 87 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYC

SEQ ID NO: 155 - protein, 87 aa, Canis lupus
  source 1..87: note = Canis lupus familiaris
SYVLTQLPSVSVNLGKTASITCEGNNIGDKYAYWYQQKPGQAPVLIIYEDSKRPSGIPERFSGSNSGNTATLTISGARAEDEADYYC

SEQ ID NO: 156 - protein, 87 aa, Homo sapiens
SYELTQPPSVSVSLGQMARITCSGEALPKKYAYWYQQKPGQFPVLVIYKDSERPSGIPERFSGSSSGTIVTLTISGVQAEDEADYYC

SEQ ID NO: 157 - protein, 80 aa, Canis lupus
  source 1..80: note = Canis lupus familiaris
PSMLVALGQMETITCSGDILGKRYAYWYQHKPSQAPVLLINKNNERASGIPHWFSGSNSGNMATLTISGARAEDEADYYC

SEQ ID NO: 158 - protein, 89 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYC

SEQ ID NO: 159 - protein, 89 aa, Canis lupus
  source 1..89: note = Canis lupus familiaris
QAVLNQPASVSGALGQKVTISCSGSTNDIDIFGVSWYQQLPGKAPKLLVDSDGDRPSGVPDRFSGSSSGNSGTLTITGLQAEDEADYYC

SEQ ID NO: 160 - protein, 94 aa, Homo sapiens
QPVLTQPTSLSASPGASARFTCTLRSGINVGTYRIYWYQQKPGSLPRYLLRYKSDSDKQQGSGVPSRFSGSKDASTNAGLLLISGLQSEDEADY

SEQ ID NO: 161 - protein, 96 aa, Canis lupus
  source 1..96: note = Canis lupus familiaris
QPVLTQPPSLSASLGSTARPTCTLSSGFSVGSYHILWFQQKSESPPRYLLRFYSDSNEHQGPGVPSRFSGSKDTSTYAGLLLISGLQPEDEADYYC

SEQ ID NO: 162 - protein, 98 aa, Canis lupus
  source 1..98: note = Canis lupus dingo
EVQLVESGGDLVKPAGSLRLSCVASGFTFSSYGMSWVRQAPGKGLQWVAGINSGGSSTSYADAVKGRFTISRDNAKNTLYLQMNSLRAEDTAVYYCAK

SEQ ID NO: 163 - protein, 98 aa, Canis lupus
  source 1..98: note = Canis lupus dingo
EVQLVESGGDMVKPGGSLRLSCVASGFTFSSYYMYWARQAPGKGLQWVSHINKDGSSTSYADAVKGRFTISRDNAKNTLYLQMNSLRAEDTAVYYCAK

SEQ ID NO: 164 - protein, 98 aa, Canis lupus
  source 1..98: note = Canis lupus dingo
EVQLAESGGDLVKPGGSLRLSCVASGFTFSSNYMSWIRQAPGKGLQWVSQISSDGSSTSYADAVKGRFTISRDNAKNTLYLQMNSLRDEDTAVYYCAR

SEQ ID NO: 165 - protein, 98 aa, Canis lupus
  source 1..98: note = Canis lupus dingo
EVQLVESGGDLVKPGGSLRLSCVTSGFTFSSYWMSCVRQAPGKELQWVAYINSGGSSTWYTDAVKGRFTISRDNAKNTLYLQMNSLRAEDTAVYYCAR

SEQ ID NO: 166 - protein, 98 aa, Canis lupus
  source 1..98: note = Canis lupus dingo
EVQLVESGGDLVKPGGSLRLSCVASGFTFSSSDMSWIRQAPEKGLQWVAYISNDGSSTSYADAVKGRFTISRDNAKNTLYLQMSSLRAEDTAVYYCAD

SEQ ID NO: 167 - protein, 98 aa, Canis lupus; identical to SEQ ID NO: 16
  source 1..98: note = Canis lupus dingo

SEQ ID NO: 168 - protein, 98 aa, Canis lupus
  source 1..98: note = Canis lupus dingo
EVQLVESRGDLVKPGGSLRLSCVAYGFTFSSYSMQCVRQAPGKGVQWVAYINSGGSSTSSADAVKGRFIISRDNVKNTLYLQMNSLRAEDTAVYYCAG

SEQ ID NO: 169 - protein, 98 aa, Canis lupus
  source 1..98: note = Canis lupus dingo
EVQLLESGGDLVKPGGSLRLSCVASEFIFSGYWKYWIHQAPGKGLQWVTWISNDGSSKSYADAVKGRFTISKDNAKNTLYLQMNSLRAEDMAVYYCMM

SEQ ID NO: 170 - protein, 98 aa, Canis lupus
  source 1..98: note = Canis lupus dingo
EVQLVETEGDLVKPGGSLRLSCVASGFIFSSYDMDWVCQAPGKGLQWVTYISNGGSSTRYADAVKGQFTISRDNARNTLYLQMNNLRDEDMAVYYCVS

SEQ ID NO: 171 - protein, 98 aa, Canis lupus; identical to SEQ ID NO: 33
  source 1..98: note = Canis lupus dingo

SEQ ID NO: 172 - protein, 98 aa, Canis lupus
  source 1..98: note = Canis lupus dingo
EVQLVESGGDLVKPGGSLRLSCVASGFTFSSYDMSWVRQSPGKGLQWVAAIMRSGSSTYYADAVKGRFTISRDNAKNTLYLQMNSLRAEDTAVYYCAK

SEQ ID NO: 173 - protein, 98 aa, Canis lupus
  source 1..98: note = Canis lupus dingo
EVQLVESGGDLVKPGGSLRLSCVASGFTFSSYYMSWIRQAPGKGLQWVADISDSGGSTGYADAVKGRFTISRENAKNTLYLQMNSLRAEDTAVYYCAK

SEQ ID NO: 174 - protein, 98 aa, Canis lupus; identical to SEQ ID NO: 39
  source 1..98: note = Canis lupus dingo

SEQ ID NO: 175 - protein, 98 aa, Canis lupus; identical to SEQ ID NO: 42
  source 1..98: note = Canis lupus dingo

SEQ ID NO: 176 - protein, 98 aa, Canis lupus
  source 1..98: note = Canis lupus dingo
EVQLVESGGDLVKPVASLRLSCVASGFTFSNYAMSWVHQAPGKGLQWVAEISHSGRSTGYTDAMKGRFTITRDNAKNMPYLKMNSLTAKDMAMYYCAR

SEQ ID NO: 177 - protein, 98 aa, Canis lupus; identical to SEQ ID NO: 47
  source 1..98: note = Canis lupus dingo

SEQ ID NO: 178 - protein, 98 aa, Canis lupus; identical to SEQ ID NO: 48
  source 1..98: note = Canis lupus dingo

SEQ ID NO: 179 - protein, 98 aa, Canis lupus
  source 1..98: note = Canis lupus dingo
EVQLVESGGDLVKPGGSLRLSCVASGFTFSNYEMYWVRQAPGKGLEWVARIYESGSTTYYAEAVKGRFTISRDNAKNMVYLQMNSLRAEDTVRGPQCE

SEQ ID NO: 180 - protein, 98 aa, Canis lupus
  source 1..98: note = Canis lupus dingo
EVQLLESGGDRVKPGGSQRLSCVASRFTFSSYSMHWLRQSPGMGLQWVTYISSSGSSTYYADAVKGRFTISRDKAKNMLYLQMNSLRAQDIALYYCAD

SEQ ID NO: 181 - protein, 98 aa, Canis lupus; identical to SEQ ID NO: 52
  source 1..98: note = Canis lupus dingo

SEQ ID NO: 182 - protein, 98 aa, Vulpes vulpes
  source 1..98: note = canis vulpes
EVQLVESGGALVKPGGSLRLSCVASGFTFSSYAMSWVRQAPGKGLQWVGYISSDGSSTYYADAVKGRFTISRDNAKNTLYLQMNSLRVEDTAVYYCAR

SEQ ID NO: 183 - protein, 98 aa, Vulpes vulpes
  source 1..98: note = canis vulpes
EVQLVESGGDLVKPGGSLRLSCVASGFTFSSYDMHWVRQAPGKGLQWVAAISYGGSSTNYADAVKGRFTISRDNAKNTLYLQMNSLRAEDTAVYYCAR

SEQ ID NO: 184 - protein, 98 aa, Vulpes vulpes
  source 1..98: note = canis vulpes
EVQLVESGGDLVKPGGSLRLSCVASGFTFSSYYMSWIRQAPGKGLQWVAYISSDGSSTRYADAVKGRFTISRDNAKNTLYLQMNSLRAEDTAVYYCAR

SEQ ID NO: 185 - protein, 98 aa, Vulpes vulpes
  source 1..98: note = canis vulpes
EVQLVESGGDLVKPGGSLRLSCVASGFTFSSYYMHWVRQAPGKGLKWVARISSDGSSTRYADALKGQFTISRDNPKNTLYLQMNSLRAEDTALYYCAR

SEQ ID NO: 186 - protein, 98 aa, Vulpes vulpes
  source 1..98: note = Canis vulpes
EVQLVESGGDLVKPGGSLRLSCVASGFTFSSDNMQWVRQAPGKGLQWVAYISSGGSSTNYADAVKGRFTISRDNAKNTLYLQMNSLRAEDTAVYYCAR

SEQ ID NO: 187 - protein, 98 aa, Vulpes vulpes
  source 1..98: note = Canis vulpes
EVQLVESGGDLVKPGGSLRLSCVASGFTFSSYYMSWIRQAPGKGLQWVAYINSGGSSTNYADAVKGRFTISRDNAKNTLYLQMNSLRAEDTAVYYCAG

SEQ ID NO: 188 - protein, 98 aa, Vulpes vulpes
  source 1..98: note = Canis vulpes
EVQLVESGGDLVKPGGSLRLSCVASGFTFSDYAMDWVRQAPGKGLQWVAYINSGGSSTSYADTVKGRFTISRDNAKNTLYLQMNSLKAEDTAVYYCAK

SEQ ID NO: 189 - protein, 98 aa, Vulpes vulpes
  source 1..98: note = Canis vulpes
EVQLVESGGDLVKPGGSLRLSCVASGFTFSSYYMHWVRQAPGKGLQWVAQISNDGSSTGYADAVKGRFTISRDNSKNTLYLQMNSLRAEDTALYYCAR

SEQ ID NO: 190 - protein, 98 aa, Vulpes vulpes
  source 1..98: note = Canis vulpes
EVQLVESGGDLVKPGGSLRLSCVGSGFTFSSYWMYWVRQAPGKGLQWVAEISGSGSSTNYADAVKGRFTLSRDNAKNTLYLQMNSLRAEDTAVYYCAR

SEQ ID NO: 191 - protein, 98 aa, Vulpes vulpes
  source 1..98: note = Canis vulpes
EVQLVESGGDLVKPGGSLRLSCVVSGFTFSSYNMQWVPQAPGKGLQWVAYISTGGSSTSYADAVKGRFTISRDNAKNKLYLQMNSLRAEDTAVYYCAR

SEQ ID NO: 192 - protein, 98 aa, Vulpes vulpes
  source 1..98: note = Canis vulpes
EVQLVESGGDLVKPGGSLKLSCVASGFTFSDYYMSWVCQAPGKGLQWVASIIYDGSSTSYTDAVKGRFTISRDNAKNTLYLQMNSLRAEDTAVYYCAS

SEQ ID NO: 193 - protein, 98 aa, Vulpes vulpes
  source 1..98: note = Canis vulpes
EVQLVESGGDLVKPGGSLRLSCVASGVTFSSYYMDWVFQAPGKRLQWVAGISSDGSSTYYPDAVKGRFTISRDNAKNTLYLQMNSLRAEDSAVYYCAM

SEQ ID NO: 194 - protein, 98 aa, Vulpes vulpes
  source 1..98: note = Canis vulpes
QVQLVESGGDLVKLGGSLRLSCVASGFTFSSCWMHWVRQAPGKGLQWVAEISGSGSSTNYADAVKGQFTISRDNAKNTLYLQMNSLRAKDTAVYYCVR

SEQ ID NO: 195 - protein, 98 aa, Vulpes vulpes
  source 1..98: note = Canis vulpes
EVQLGESGGDLVKPGRSLRLSCVASGFTFSSYHMSWVHQAPGKGLQWVSRIISDGSSTYYADAVKGQFTISRDNAKKTLYLQMNSLRTEDTAVYYCGK

SEQ ID NO: 196 - protein, 98 aa, Vulpes vulpes
  source 1..98: note = Canis vulpes
EVQLVESGGDLVKPGGSLRLSCVASGFTFSNYKIYWVRQAPGKGLEWVARIYESGSTTNYAEAVKGRFTISRDNAKNTLYLQMNSLRAEDTAVYYCAS

SEQ ID NO: 197 - protein, 98 aa, Vulpes vulpes
  source 1..98: note = Canis vulpes
EVQLVETGGDLVKPGGSLRLSCVASGFTFNSYYMDWVCQAPGKGLQWVAYISSDGSSTSYTDAVKGQFTISRDNAKNTLYLQMNSLRAEDTAVYYCTS

SEQ ID NO: 198 - protein, 98 aa, Vulpes vulpes
  source 1..98: note = Canis vulpes
EVQLLESGGNLVKPRGSLRLSCVASGFTFSSYTMQWVPQAPGKGLQWVTYINSGGSSTSSADTVKGPFIISRDNAKNPLYLQMNSLRAEDTAVYYCAD

SEQ ID NO: 199 - protein, 98 aa, Vulpes vulpes
  source 1..98: note = Canis vulpes
EVQLVESGGDLVKPGGSLGLSCVASGFTFSSYDMHWVRQAPGKGLQWVAWIYASGSSPSYSDAVKGRFTISRDNAKYMVHLQMNSLRAEDTAVYYCAS

SEQ ID NO: 200 - protein, 98 aa, Vulpes vulpes
  source 1..98: note = Canis vulpes
EVQLVESGGDLVKPEGSLRLSCVASGFTFSSSYMNWVRQDPRKGLQWVAAISNDGRSTYYADAVKGRFTISRDNAKNTVYLQMNSLRAEDTAMYYCAR

SEQ ID NO: 201 - protein, 98 aa, Vulpes vulpes
  source 1..98: note = Canis vulpes
EEQLVEFGGHMVNPGGSLGLSCQASGFTFSSYGMSWVRQAQKKGLQWVGHISYDGSSTYYADTVRDRFTISRDNTKNMLYLQMNSLRAEDTAVYYCMR

SEQ ID NO: 202 - protein, 98 aa, Vulpes vulpes
  source 1..98: note = Canis vulpes
CAAGGVWGRPGDAWGVSLRLSCVASGFTFSRYAMSWVLQAPGKELQWVAYISSDGSSTSYTDAVKGQFAILRANAKNTLYLQMNSLRAEDTAVYYCAR

SEQ ID NO: 203 - protein, 98 aa, Vulpes vulpes
  source 1..98: note = Canis vulpes
EVQLVESGGDLVKPGGSLRLSCVASGFTFSSYGMSWVRQAPGKGLQWVAYISYDGSSTYYTDAVKGRFTISRDNAKNTLYLQMNSLRAEDTAVYYCAK

SEQ ID NO: 204 - protein, 98 aa, Vulpes vulpes
  source 1..98: note = Canis vulpes
EVQLVESGGDLVKPGGSLRLSCVVSGFAFSSNEMSWVRQAPGKGLQWVAAISYDGSSTYYVDAVKGRFTTSRDNAKNTLYLQMNSLRAEDTAVYYCAD

SEQ ID NO: 205 - protein, 98 aa, Vulpes vulpes
  source 1..98: note = Canis vulpes
EVQLVESGGDLVKPGGSLRLSCVASGFTFSSYGVSWVRQAPGKGLQWVAAIWYDGTETYYADAVKGRFTISSDNAKNTLYLQMNSLRAEDTAVYYCVK

SEQ ID NO: 206 - protein, 98 aa, Vulpes vulpes
  source 1..98: note = Canis vulpes
EVQLVESGGDLVKPGGSLRLSCVASGFTFSSYGMSWVRQAPGKGLQLVAAIWYDGSRTYYADAVKGRFTISRDNAKNTLYLQMNSLKAEDTAMYFCAK

SEQ ID NO: 207 - protein, 98 aa, Vulpes vulpes
  source 1..98: note = Canis vulpes
EVQLVESGRDLVKPGGSLRLSCVASEFTFSSYSMSCVCQAPGKGLQWVAEISSSGSSTYYTDAVKGRFTISRDNAKNTLYLQMNSLRAEDMAVYYCAD

SEQ ID NO: 208 - protein, 98 aa, Vulpes vulpes
  source 1..98: note = Canis vulpes
EVQQLESGGDRVKPRGSQRLSCVASRFTFSSYSMHWLHQSPGKGLQWITYISSSGSSTYYADAVKGRFTISRDKDKNMLYLQMNSLRAQDIALYYCAD

SEQ ID NO: 209 - protein, 98 aa, Vulpes vulpes
  source 1..98: note = Canis vulpes
EVQQLESGGDRVKPRGSQRLSCVASRFTFSSYSMHWLHQSPGKGLQWITYISSSESSTYYVDAVKGRFAISRDKDKNMLYLQMNSLKAQDIALYYCAD

SEQ ID NO: 210 - protein, 98 aa, Vulpes vulpes
  source 1..98: note = Canis vulpes
EVQLVESGGDLVKPGGSLRLSCVASGFTFSSYAMSWVRQAPGKGLQWVAGISNSGSSTGYADAVKGRFTISRDNAKNTLYLQMNSLKAEDTAVYYCAK

SEQ ID NO: 211 - protein, 98 aa, Vulpes vulpes
  source 1..98: note = Canis vulpes
EVQLVESGGDLVKPGGSLRLSCVASGFTFSSNWMSWVRQAPGKGLQWVAGISNSGSSTYYADAVKGRFTISRDNAKNTLYLQMNSLRTEDTAMYYCAK

SEQ ID NO: 212 - protein, 98 aa, Vulpes vulpes
  source 1..98: note = Canis vulpes
EVQLVESGGDLVKPGGFLRLSCVASGFTFSSYAMSWVHKAPVKGLQWVTRIYASGSRTSYADAVKGRFTISRDNVKNTLYLQMNSLRAEDTAVHYCLK

SEQ ID NO: 213 - protein, 98 aa, Vulpes vulpes
  source 1..98: note = Canis vulpes
EVQLVQSGAEVKKPGASVKVSCKTSGYTFINYYMHWVRQAPGAGLDWMGRIDPEDGATNYAQKFQGRVTLTADTSTSTAYMELRSLRAEDTAVYYCAR

SEQ ID NO: 214 - protein, 98 aa, Vulpes vulpes
  source 1..98: note = Canis vulpes
EVQLVQSGAEVKKPGASVKVSCKTSGYTFINYYMYWVRQAPGAGFDWMGLIDPEDGATRYAQKIQGRVTLTADTSTSTAYMELNSLRAEDTAVYYCAR

SEQ ID NO: 215 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 216 - protein, 98 aa, Vulpes vulpes
  source 1..98: note = Canis vulpes
EVQLVESGGDLVKPGGSLRLSCVASGFTFSSYGMNWVRQAPGKGLQWVAVIWYDGSRTYYNDAVKGRFTISRDNAKNTLYLQMNSLRAEDTAVYYCAK

SEQ ID NO: 217 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 218 - protein, 97 aa, Vulpes vulpes
  source 1..97: note = Canis vulpes
EVQLVESGGDLVKPGGSLRLSCVASGFTFSSYAMSWVRQAPGKGLQWVAAISYGGSTYYTDAVKGRFTISRDNAKNTLYLQMNSLRAEDTAVYYCAK

SEQ ID NO: 219 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 220 - protein, 98 aa, Vulpes vulpes
  source 1..98: note = Canis vulpes
EVQLVESGGDLVKPGVSLRLSCVASGFTFSDYGMSWVRQAPGKGLQWVGEISSSGSSTYYADAVKGRFTISRDNAKNTLYLQMNSLRAEDMAVYYCAT

SEQ ID NO: 221 - protein, 98 aa, Canis lupus; identical to SEQ ID NO: 3

SEQ ID NO: 222 - protein, 98 aa, Canis lupus
EVQLVESGGDLVKPGGSLRLSCVASGFTFSSYWMYWVRQAPGKGLQWVAGINSGGSSTSYADAVKGRFTISRDNAKNTVYLQMNSLRAEDTAMYYCAK

SEQ ID NO: 223 - protein, 98 aa, Canis lupus
EVQLVESGGDLVKPAGSLRLSCVASGFTFSSYSMSWVRQAPGKGLQWVAGINSGGSSTSYADAVKGRFTISRDNAKNTLYLQMNSLRAEDTAVYYCAD

SEQ ID NO: 224 - protein, 98 aa, Canis lupus
EVQLVESGGDLVKPGGSLRLSCVASGFTFSSYHMSWVRQAPGKGLQWVAYINSGGSSTYYADAVKGRFTISRDNAKNTLYLQMNSLRAEDTAVYYCAD

SEQ ID NO: 225 - protein, 98 aa, Canis lupus
EVQLVESGGDVVKPGGSLRLSCVASGFTFSSYYMYWARQAPGKGLQWVSHINKDGSSTSYADAVKGRFTISRDNAKNTLYLQMNSLRAEDTAVYYCAK

SEQ ID NO: 226 - protein, 98 aa, Canis lupus; identical to SEQ ID NO: 6

SEQ ID NO: 227 - protein, 98 aa, Canis lupus; identical to SEQ ID NO: 165

SEQ ID NO: 228 - protein, 98 aa, Canis lupus
EVQLVESGGELVKPAGSLRLSCVTSGFTFSSYGMSWVRQAPGKGLQWVAGINSGGSSTSYADAVKGRFTISRDNAKNTVYLQMNSLRAEDTAVYYCAK

SEQ ID NO: 229 - protein, 98 aa, Canis lupus
EVLLVESGGDLVKPGGSLRLSCVASGFTFSSYDMGWVRQAPGKGLQWVAYISNGGSSTRYADAVKGRFTISRDNAKNTLYLQMNSLRAEDTAVYYCAS

SEQ ID NO: 230 - protein, 98 aa, Canis lupus; identical to SEQ ID NO: 11

SEQ ID NO: 231 - protein, 98 aa, Canis lupus; identical to SEQ ID NO: 7

SEQ ID NO: 232 - protein, 98 aa, Canis lupus; identical to SEQ ID NO: 15

SEQ ID NO: 233 - protein, 98 aa, Canis lupus
EVQLVESGGALVKSGGSLRLSCVGSGFTFSSYWMYWDRQAPGKGLQWVAWIRYDGSSTSYAEAVKGRFTVSRDNAKNTLYLQMNSLRAEDTAVYYCVR

SEQ ID NO: 234 - protein, 98 aa, Canis lupus
EEQLVESGEDPVKPGGSLRLSCVASGFTFSSYAVSWVRQAPGKGLQWVRYISSDGSSTYYADAVKGRFTISRDNAKNTLYLPMNSLRAEDTAVYYCAR

SEQ ID NO: 235 - protein, 98 aa, Canis lupus
EEQLMESEGDLVKPGGSLRLSCVASGFTFSSYDMSWVRQAPGKGLQWVGYINSGGSSTYYADAVKGRFTISRDNAKNTLYLQMNSLRAEDTAVYYCVK

SEQ ID NO: 236 - protein, 98 aa, Canis lupus
EEQLMESEGDLVKPGGSLRLFCVASGFTFSSYDMSWVRQAPGKGLQWVGYINSGGSSTYYADAVKGRFTISRDNAKNTLYLQMNSLRAEDTAVYYCVK

SEQ ID NO: 237 - protein, 98 aa, Canis lupus; identical to SEQ ID NO: 16

SEQ ID NO: 238 - protein, 98 aa, Canis lupus; identical to SEQ ID NO: 234

SEQ ID NO: 239 - protein, 98 aa, Canis lupus
EMQLVEAWGDMVEPGGSLRISCVASGFTFSSYWMSWVCQAPGKGLQWVAYINSGGSSTYYADAVKGRFTISRDNAKNTLYLQMNSLRAEDMAVYYCVR

SEQ ID NO: 240 - protein, 98 aa, Canis lupus
EVQLVESGGDLAKPGGSLRLSCVASGFTFSSYEMNWVCQAPGKGLQWVAYINCGESSTYYADAVKGRFTISRDNARNTLYLQMNSLRTEDTAMYYCAK

SEQ ID NO: 241 - protein, 98 aa, Canis lupus; identical to SEQ ID NO: 24

SEQ ID NO: 242 - protein, 98 aa, Canis lupus
EVQLVESGGDLVKPGLPLRLSCVASGFTFSSYWISWVHQAPGKGLQWVSVINKYGSTTYHADAVKGQFTIFRDNAKNTLYLQMNSLRAEDTAVYYCAH

SEQ ID NO: 243 - protein, 98 aa, Canis lupus
EVQLVESGGDLVKPGGSLRLSCVASGFTFNSYDMSWVHQVRGKRLQWVAYFSSDGNSTSSADAVKGRFTISRDNAKNTLYLQMNSLRAEDTAVYYCAK

SEQ ID NO: 244 - protein, 98 aa, Canis lupus
EVQLVESGGDLAKPGGSRRLSCVASGFTFSSYEINWVCQAPGKGLQWVAYINCGGSSTYYADAVKGWFTISRDNARNTLYLQMNSLRAEDTVVYYCAK

SEQ ID NO: 245 - protein, 98 aa, Canis lupus
EVQLVETEGDLVKPGGSLRLSCVASGFTFSSYDMDWVCQAPGKGLQWVTYISNGGSSTRYADAVKGPFTISRDNARNTLYLQMNSLRAEDTAVYYCVS

SEQ ID NO: 246 - protein, 98 aa, Canis lupus
EVQLVESGGDLVKPGLPLRLSCVASGFTFSSYWISWVHQTPGKGLQWVSVMNKYGSTTYHADAVKGQFTIFRDNAKNTLYLQMNSLRSEDMAVYYCAH

SEQ ID NO: 247 - protein, 98 aa, Canis lupus
EVQLVESGGDLVKPGLPLRLSCVASGFTFSSYWISWVHQAPGKGLQWVSVMNKYGSTTYHADAVKGQFTIFRDNAKNTLYLQMNSLRAEDMDVYYCVH

SEQ ID NO: 248 - protein, 98 aa, Canis lupus; identical to SEQ ID NO: 32

SEQ ID NO: 249 - protein, 98 aa, Canis lupus; identical to SEQ ID NO: 47

SEQ ID NO: 250 - protein, 98 aa, Canis lupus
EVQLVESGGDLVKPGGSLRLSCVASGFTFSSYDMNWVRQAPGKGLQWVAYISSGGSSTYYADAVKGRFTISRDNAKNTLYLQMNSLRAEDTAMYYCAG

SEQ ID NO: 251 - protein, 98 aa, Canis lupus
EVQLVESGGDLVKPGRSLRLSCVASGFTFSSNEMNWVRQAPGKGLQWVAAISSNGSSTYYADAVKGRFTISRDNAKNTLYLQMNSLRAEDTAVYYCAD

SEQ ID NO: 252 - protein, 98 aa, Canis lupus
EVQLVESGGDLVKPGGSLRLSCVASGFTFSNYEMYWVRQAPGKGLEWVARIYESGSTTYYAEAVKGRFTISRDNAKNTLYLQMNSLRAEDTAVYYCAS

SEQ ID NO: 253 - protein, 98 aa, Canis lupus
EVQLVESGGDLVKPGGSLRLSCVTSGFTFSSNEMNWVRQDPGKGLQWVAAISSSGSSTYYADAVKGRFTISRDNAKNTLYLQMNSLRAEDMAVYYCAD

SEQ ID NO: 254 - protein, 98 aa, Canis lupus
EVQLLESGGDRVKPGGSQRLSCVASRFTFSSYSMHWLRQSPGMGLQWVTYISSSGSSTYFADAVKGRFTISRDKAKNMLYLQMNSLRAQDIALYYCAD

SEQ ID NO: 255 - protein, 98 aa, Canis lupus
EVQLVESGGDLVKPGGSLRLSCVASGFTFSSYGMSWVRQSPGKGLQWVAAISSSGSSTYYADAVKGRFTISRDNAKNTLYLQMNSLRAEDTAVYYCAK

SEQ ID NO: 256 - protein, 98 aa, Canis lupus
EVQLVESGGDLVKPGGSLRLSCVASGFTFSSYYMSWIRQAPGKGLKWVADISDSGGSTYYTDAVKGRFTISRDNAKNTLYLQMNSLRAEDTAVYYCAK

SEQ ID NO: 257 - protein, 98 aa, Canis lupus
KVQLVKSGGDLVKPAGSLRLSCVASGFTFSSYYMSWIRQAPEKGLQWVADINDSGGSTGYADAVKGRFTISRDNAKNTLYLQMNSLRAEDTAVYYCAK

SEQ ID NO: 258 - protein, 98 aa, Canis lupus
EVQLVESGGDLVKPVASLRLSCVASGFTFSNYAMSWVRQAPGKGLQWVAEISHSGRSTGYTDAVKGRFTITRDNAKNMLYLKMNSLTAKDMAMYYCAR

SEQ ID NO: 259 - protein, 98 aa, Canis lupus
EVQLVESGGALVKPGASLRLSCVASGFTFSSYDMSWVRQAPGKGLQWVAFISYDGSSTYYADAVRGRFTISRDNAKNSLYLQMNSLRAEDTAVYYCGK

SEQ ID NO: 260 - protein, 98 aa, Canis lupus; identical to SEQ ID NO: 52

SEQ ID NO: 261 - protein, 97 aa, Canis lupus
EVQLVESGGDLVKPGGSLRLSCVASGFTFSSYGMSWVRQSPGKGLQWVADISSSGSTYYADAVKGRFTISRDNAKNTLYLQMNSLRAEDTAVYYCAK

SEQ ID NO: 262 - protein, 97 aa, Canis lupus
EVQLVESGGDLVKPAGSLRLSCVASGFTFSSYGMSWVRQAPGKGLQWVAGINSGGNTGYADAVKGRFTISRDNAKNTVYLQMNSLRAEDTAVYYCAK

SEQ ID NO: 263 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 264 - protein, 98 aa, Canis lupus
EVQLVQSAAEVKKPGASVKVSCKTSGYTFTDYYMHWVQQAPGAGLDWMGWIDPEDGATSYAQKFQGRVTLTADTSTSTAYMELSSLRAEDTAVYYCAR

SEQ ID NO: 265 - protein, 98 aa, Homo sapiens; identical to SEQ ID NO: 47

SEQ ID NO: 266 - protein, 127 aa, Canis lupus
  source 1..127: note = Canis lupus familiaris
EVQLVESGGGLVKPGGSLRLSCAASGFTFSSYSMNWVRQAPGKGLEWVSCISSSSSFIYYADSVKGRFTISRDNAKNSLYLQMNSLRAEDTAVYYCARDPVWVDGELLSGGIPFDYWGQGTLVTVSS

SEQ ID NO: 267 - protein, 124 aa, Canis lupus
  source 1..124: note = Canis lupus familiaris
EVQLVESGGGLVKPGGSLRLSCAASGFTFSGYSMNWVRQAPGKGLEWVSSISSSSSYIYYADSVKGRFTISRDNAKNSLYLQMNSLRAEDTAVYYCARWLQLRSDYYYFGMDVWGQGTTVTVSS

SEQ ID NO: 268 - protein, 126 aa, Canis lupus
  source 1..126: note = Canis lupus familiaris
EVQLVESGGGLVQPGGSLRLSCAASGFTFSSYEMNWVRQAPGKGLEWVSYISSSGSAIYYADSVKGRFTISRDNAKNSLYLQMNSLRVEDTAVYYCAREARSRYFDWLPSYYFDYWGQGTLVTVSS

SEQ ID NO: 269 - protein, 128 aa, Canis lupus
  source 1..128: note = Canis lupus familiaris
EVQLVESGGGLVKPGGSLRLSCAASGFTFSSYSMNWVRQAPGKGLEWVSSISNSNSFIYYADSMKGRFTISRDNAKNSLYLQMNSLRAEDTAVYYCARVNGNSNWNFGSYYYYYMDVWGKGTTVTVSS

SEQ ID NO: 270 - protein, 124 aa, Canis lupus
  source 1..124: note = Canis lupus familiaris
EVQLVESGGGLVQPGGSLRLSCEASGFTFSSSEINWVRQAPGKGLEWVSHISSSGSIIYYADSVKGRFTISRDNAKNSLYLQMNSLRAEDTAVYYCARRSYRSSWYYYYGMDVWGQGTTVTVSS

SEQ ID NO: 271 - protein, 98 aa, Homo sapiens; identical to SEQ ID NO: 2

SEQ ID NO: 272 - protein, 120 aa, Canis lupus
  source 1..120: note = Canis lupus familiaris
EVQLVESGGGLVQPGGSLRLSCAVSGFTFSSYWMHWVRQAPGKGLVWVSRINSDGSSTSYADSVKGRFTISRDNAKNTLYLQMNSLRAEDTAVYYCAREVEQLAHMVDYWGQGTLVTVSS

SEQ ID NO: 273 - protein, 119 aa, Canis lupus
  source 1..119: note = Canis lupus familiaris
EVQLVESGGGLVQPGGSLRLSCAASGFTFSSYWMHWVRQVPGKGLVWVSRINSDGSSTSYADSVKGRFTISRDNAKNTLYLEMNSLRAQDTAVYYCAGSPWLRGDIDYWGQGTLVTVSS

SEQ ID NO: 274 - protein, 129 aa, Canis lupus
  source 1..129: note = Canis lupus familiaris
EVQLVESGGGLVQPGGSLRLSCAASGFTFSSYWMSWVRQAPGKGLEWVANIKEDGSEKYYVDSVKGRFTISRDNAKNSLYLQMNSLRAEDTAVYYCARDVGGYSGYDLGFDYYYYMDVWGKGTTVTVSS

SEQ ID NO: 275 - protein, 121 aa, Canis lupus
  source 1..121: note = Canis lupus familiaris
EVQLVESGGGLVQPGGSLRLSCVASEFTFSSYWMSWFRQAPGKGLEWVSGINWNGGSTGYADSVKGRFTISRDNAKNSLYLQMNSLKTEDTAVYYCASVITFGGVIVRSYWGQGTLVTVSS

SEQ ID NO: 276 - protein, 119 aa, Canis lupus
  source 1..119: note = Canis lupus familiaris
EVQLVESGGGLVQPGGSLRLSCAASGFTFSIYWMSWVRQAPGKGLQWVANIKQDASEKYYVDSVKGRFTISRDNAKNSLYLQMNSLRAEDTAVYYCARLGGSSWHFDYWGQGTLVTVSS

SEQ ID NO: 277 - protein, 98 aa, Homo sapiens; identical to SEQ ID NO: 56

SEQ ID NO: 278 - protein, 122 aa, Canis lupus
  source 1..122: note = Canis lupus familiaris
QVQLVQSGAEVKKPGASVKVSCKASGYTFTSYYMHWVRQAPGQGLEWMGIINPSAGSTSYAQKFQGRVTMTRDTSTSTVYMELSSLRSEDTAVYYCARGTLIPAHRGAFDIWGQGTMVTVSS

SEQ ID NO: 279 - protein, 124 aa, Canis lupus
  source 1..124: note = Canis lupus familiaris
QVQLVQSGAEVKKPGASVKVSCKASGYTFTSHYMHWVRQAPGQGLEWMGIINPSGGSTSYAQKFQGRVTMTGDTSTSTVYMELSSLRSEDTAVYYCARDLAGVPAALGCWFDPWGQGTLVTVSS

SEQ ID NO: 280 - protein, 121 aa, Canis lupus
  source 1..121: note = Canis lupus familiaris
QVQLVQAGAEVKKPGASVKVSCKASGYTFTSYYMHWVRQAPGQGLEWMGIINPSGGSTSYAQKFQGRVTMTRDTSTSTVYMELSSLRSEDTAVYYCARGAAVPAAGEFDYWGQGTLVTVSS

SEQ ID NO: 281 - protein, 122 aa, Canis lupus
  source 1..122: note = Canis lupus familiaris
QVQLVQSGAEVKKPGASVKVSCKASGYTFTSYYMHWVRQAPGQGLEWMGIINPGGGSTTYAQKFQGRVTMTSDTSTSTVYMELSSLRSEDTAMYYCARGAIPPNSRAEIDYWGQGTLVTVSS

SEQ ID NO: 282 - protein, 98 aa, Homo sapiens; identical to SEQ ID NO: 5

SEQ ID NO: 283 - protein, 118 aa, Canis lupus
  source 1..118: note = Canis lupus familiaris
QVQLVESGGGVVQPGRSLRLSCAASGFTFSTYGMHWVRQAPGKGLEWVAVISYDGSNKYYADSVKGRFTISRDNSKNTLYLQMNSLRAEDTAMYYCAKDGSIAAADYWGQGTLVTVSS

SEQ ID NO: 284 - protein, 124 aa, Canis lupus
  source 1..124: note = Canis lupus familiaris
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLTIVVIPAAPNFDYWGQGTLVTVSS

SEQ ID NO: 285 - protein, 125 aa, Canis lupus
  source 1..125: note = Canis lupus familiaris
QVQLVESGGGVVQPGRSLRLSCVASGFTFSSYGMHWVRQAPGKGLEWVAVISFDGSNKYYADSVKGRFTISRDNSKNTLYLQMNSLRAEDTAVFYCAKDLPPYASGWYEGGFDYWGRGTQVTVSS

SEQ ID NO: 286 - protein, 126 aa, Canis lupus
  source 1..126: note = Canis lupus familiaris
QVQLVESGGGVVQPGRSLRLSCAASGFTFSSYGMHWVRQAPGKGLEWVAVISSDGSNKYYAGSVKGRFTISRDNSKNTLYLQMNSLRAEDTAVYYCAKDMVEPLFSHYYYYGMDVWGQGTTVTVSS

SEQ ID NO: 287 - protein, 98 aa, Homo sapiens; identical to SEQ ID NO: 9

SEQ ID NO: 288 - protein, 128 aa, Canis lupus
  source 1..128: note = Canis lupus familiaris
QVQLVESGGGVVQPGRSLRLSCAASGFTFSSYGMHWVRQAPGKGLEWVAVIWYDGSNKYYAESVKGRFTISRDNSKNTLYLQMNSLRAEDTAVYYCARDLHQDWVVVVAANVYGMDVWGQGTTVTVSS

SEQ ID NO: 289 - protein, 124 aa, Canis lupus
  source 1..124: note = Canis lupus familiaris
QVQLVESGGGVVQPGRSLRLSCAASGFTFSSYGMHWVRQAPGKGLEWVAVIWFDGSNKHYADSVKGRFTISRDNSKNTLYLQMNSLRAEDTAVYYCAREGDFWSGYYTGWFDPWGQGTLVTVSS

SEQ ID NO: 290 - protein, 128 aa, Canis lupus
  source 1..128: note = Canis lupus familiaris
QVQLVESGGGVVQPGRSLRLSCAASGFTFSSYGMHWVRQAPGKGLEWVAVIWYDGSKKDYADSVKGRFTISRDNSKNTLYLQMNSLRAEDTAVYYCARDQSQGAYILTGYRGYGMDVWGQGTTVTVSS

SEQ ID NO: 291 - protein, 120 aa, Canis lupus
  source 1..120: note = Canis lupus familiaris
QVQLVESGGGVVQPGRSLRLSCAASGFTFSSYGMHWVRQAPDKGLEWVAVIWYDGSNKYYADSVKGRFTISRDNSKNTLYLQMNSLRAEDTAVYYCAREGQWPNQAFDIWGQGTMVTVSS

SEQ ID NO: 292 - protein, 98 aa, Canis lupus
  source 1..98: note = Canis lupus familiaris
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAR

SEQ ID NO: 293 - protein, 98 aa, Canis lupus
  source 1..98: note = Canis lupus familiaris
EVQLVESGGGLVQPGRSLRLSCAASGFTFDDYAMHWVRQAPGKGLEWVSGISWNSGSIGYADSVKGRFTISRDNAKNSLYLQMNSLRAEDTALYHCAK

SEQ ID NO: 294 - protein, 98 aa, Canis lupus
  source 1..98: note = Canis lupus familiaris
QVQLVQSGAEVKKPGSSVKVSCKASGGTFSSYAISWVRQAPGQGLEWMGRIIPIFGIANYAQKFQGRVTITADKSTSTAYMELSSLRSEDTAVYYCAR

SEQ ID NO: 295 - protein, 98 aa, Homo sapiens; identical to SEQ ID NO: 9

SEQ ID NO: 296 - protein, 98 aa, Canis lupus
  source 1..98: note = Canis lupus familiaris
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 297 - protein, 98 aa, Canis lupus
  source 1..98: note = Canis lupus familiaris
EVQLVESGGGLVKPGGSLRLSCAASGFTFSSYSMNWVRQAPGKGLEWVSSISSSGSTIYYADSVKGRFTISRDNAKNSLYLQMNSLRAEDTAVYYCAR

SEQ ID NO: 298 - protein, 98 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 299 - protein, 98 aa, Canis lupus; identical to SEQ ID NO: 48
  source 1..98: note = Canis lupus familiaris

SEQ ID NO: 300 - protein, 95 aa, Homo sapiens
QSVLTQPPSVSVSPGQSVTITCTGSSSDVGGSNYVSWYQQKPGQAPKLLIYEKSDGIDSNRPSGVPDRFSGSKSGNTASLTISGLQAEDEADYYC

SEQ ID NO: 301 - protein, 94 aa, Canis lupus
  source 1..94: note = Canis lupus familiaris
QSVLTQPPSVSGSLGQRVTISCTGSSSNVIGSNYVGWYQQLPGTAPRTLIYGYSDSTSNRPSGVPDRFSGSKSGNTATLTISGLQAEDEADYYC

SEQ ID NO: 302 - protein, 94 aa, Homo sapiens
DIVMTQSPSSLSASPGDRVTISCRASQSLLHSDGISSYLAWYQQKPGKAPKLLIYAASSLQSGVPSRFSGSGSGTDFTLTISSLQPEDFATYYC

SEQ ID NO: 303 - protein, 94 aa, Canis lupus
  source 1..94: note = Canis lupus familiaris
DIVMTQTPLSLSVSPGETASISCKASQSLLHSFNGNTYLNWFRQKPGQSPQRLIYKVSNRDPGVPDRFSGSGSGTDFTLRISRVEADDAGVYYC

SEQ ID NO: 304 - protein, 115 aa, Canis lupus
  source 1..115: note = Canis lupus familiaris
EVQLVESGGDLVKPAGSLRLSCVASGFPFSDSDMNWVRQAPGKGLQWVAGIDEGATDTGYADAVKGRFTISRDDAKNTLYLQMNSLRADDTAMYYCAAGGNFAYWGQGTLVTVSS

SEQ ID NO: 305 - protein, 112 aa, Canis lupus
  source 1..112: note = Canis lupus familiaris
DIVMTQTPLSLSVSPGEPASISCKASQSLLHSNGNTYLYWFRQKPGQSPQGLIYLVSKRFSWVPDRFSGSGSGTDFTLRISRVEADDAGLYYCGQGIQYPYTFSQGTKLEIK

SEQ ID NO: 306 - protein, 114 aa, Canis lupus
  source 1..114: note = Canis lupus familiaris
GVQLVESGGDLVKPAGSLRLSCVASGFTFSNYGMSWVRQAPGKGLQWVASIASGGSATAYADAVKGRFTLSRDDAKNTLYLQMNNLRTEDTSMYYCVAPGFQYWGQGTLVTVSS

SEQ ID NO: 307 - protein, 112 aa, Canis lupus
  source 1..112: note = Canis lupus familiaris
DIVMTQTPLSLSVSPGETASISCKASQSLLHSNGNTYLFWFRQKSGQSPQRLIYKVSNRDPGVPDRLSGSGSGTDFTLRISRVEADDAGVYYCGQGIQIPRTFGQGTKLEIK

SEQ ID NO: 308 - protein, 93 aa, Canis lupus; identical to SEQ ID NO: 61
  source 1..93: note = Canis lupus familiaris

SEQ ID NO: 309 - protein, 93 aa, Canis lupus; identical to SEQ ID NO: 61
  source 1..93: note = Canis lupus familiaris

SEQ ID NO: 310 - protein, 109 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARWGQGTLVTVSS

SEQ ID NO: 311 - protein, 109 aa, Mus musculus
EVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARWGQGTSVTVSS

SEQ ID NO: 312 - protein, 109 aa, Oryctolagus cuniculus
CQQLKESGGGLVKPGGSLKLCCKASGFTFSSYYMCWVRQAPGKGLEWIGCIYAGSGSTHYASWVNGRFTLSRDNAQSTVCLQLNSLTAADTATYFCARWGQGTLVTVSS

SEQ ID NO: 313 - protein, 109 aa, Rattus norvegicus
EVQLQESGPGLVKPSQSLSLTCSVTGYSITSSYRWNWIRKFPGNKLEWMGYINSAGSTNYNPSLKSRISITRDTSKNQFFLQVNSVTTEDTATYYCARWGQGTLVTVSS

SEQ ID NO: 314 - protein, 109 aa, Gallus gallus
AVTLDESGGGLQTPGGGLSLVCKASGFDFSSNAMGWVRQAPGKGLEWVAGIDDDGSFTLYAPAVKGRATISRDNGQSTVRLQLNNLRAEDTATYYCARWGHGTEVIVSS

SEQ ID NO: 315 - protein, 103 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCFGQGTKLEIK

SEQ ID NO: 316 - protein, 103 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCFGSGTKLEIK

SEQ ID NO: 317 - protein, 100 aa, Oryctolagus cuniculus
DVVMTQTPSPVSAAVGSTVTISCQASQSVYNNNNLAWFQQKPGQPPKLLIYAASNLASGVPSRFKGSGSGTQFTLTISGVQCDDAATYYCFGKGTKLEIK

SEQ ID NO: 318 - protein, 103 aa, Rattus norvegicus
DVVMTQTPVSLSVSLGGQTSISCRSSQSLVHSDGNTYLYWYLQKPGQSPQLLIYKVSNRFSGVPDRFSGSGSGTDFTLKISRVEPEDLGVYYCFGSGTKLEIK

SEQ ID NO: 319 - protein, 110 aa, Mus musculus
EVQLVESGGDLVKPGGSLKLSCAASGFTFSSYGMSWVRQATPGKGLEWIGCIYAGSGSTHYASWVNGRFTISRDNAQSTVCLQLNSLTAADTATYFCARWGQGTLVTVSS

SEQ ID NO: 320 - protein, 110 aa, Rattus norvegicus
QVTLKESGPGILQPSQTLSLTCTFSGFSLSTYGMGVGWIRQPSGKGLEWLANIWWDDDKYYNPSLKNRLTISKDTSNNQAFLKITNVDTADTATYYCARWGQGTLVTVSS

SEQ ID NO: 321 - protein, 109 aa, Gallus gallus
AVTLDESGGGLQTPRGALSLVCKASGFTFSSYGMGWVRQAPGKGLEWVAGIGSSGSGTAYGSAVKGRATISRDNGQSTVRLQLNNLRAEDTGTYYCAKWGHGTEVIVSS

SEQ ID NO: 322 - protein, 103 aa, Rattus norvegicus
DVVMTQTPVSLSVSLGGQVSISCRSSQSFVHSDGNTYLNWYLQKPGQSPQLLIYKVSNRLSGVPDRFSGSGSGTDFTLKISRVEHDDLGVYYCFGSGTKLEIK

SEQ ID NO: 323 - protein, 115 aa, Synthetic construct
EVQLVESGGGLVQPGGSLRLSCAASGFTFSDSDMNWVRQAPGKGLEWVAGIDEGATDTGYADAVKGRFTISRDNAKNSLYLQMNSLRAEDTAVYYCAAGGNFAYWGQGTLVTVSS

SEQ ID NO: 324 - protein, 115 aa, Synthetic construct
EVQLVESGGGLVQPGGSLRLSCAASGFTFSDSDMNWVRQAPGKGLEWVSGIDEGATDTGYADAVKGRFTISRDNAKNSLYLQMNSLRAEDTAVYYCAAGGNFAYWGQGTLVTVSS

SEQ ID NO: 325 - protein, 112 aa, Synthetic construct
DIVMTQTPLSLSVTPGQPASISCKASQSLLHSNGNTYLYWYLQKPGQPPQLLIYLVSKRFSGVPDRFSGSGSGTDFTLKISRVEAEDVGVYYCGQGIQYPYTFGQGTKLEIK

SEQ ID NO: 326 - protein, 112 aa, Synthetic construct
DIVMTQTPLSLSVTPGQPASISCKASQSLLHSNGNTYLYWYLQKPGQPPQLLIYLVSKRFSWVPDRFSGSGSGTDFTLKISRVEAEDVGVYYCGQGIQYPYTFGQGTKLEIK

SEQ ID NO: 327 - protein, 114 aa, Synthetic construct
EVQLVESGGGLVQPGGSLRLSCAASGFTFSNYGMSWVRQAPGKGLEWVASIASGGSATAYADAVKGRFTISRDNAKNSLYLQMNSLRAEDTAVYYCVAPGFQYWGQGTLVTVSS

SEQ ID NO: 328 - protein, 114 aa, Synthetic construct
EVQLVESGGGLVKPGGSLRLSCAASGFTFSNYGMSWVRQAPGKGLEWVASIASGGSATAYADAVKGRFTISRDNAKNSLYLQMNSLRAEDTAVYYCVAPGFQYWGQGTLVTVSS

SEQ ID NO: 329 - protein, 112 aa, Synthetic construct
DIVMTQTPLSLSVTPGQPASISCKASQSLLHSNGNTYLFWYLQKPGQPPQLLIYKVSNRDPGVPDRFSGSGSGTDFTLKISRVEAEDVGVYYCGQGIQIPRTFGQGTKLEIK

SEQ ID NO: 330 - protein, 109 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARWGQGTLVTVSS

SEQ ID NO: 331 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 332 - protein, 585 aa, Homo sapiens
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 333 - DNA, 345 bp, Canis lupus
  source 1..345: mol_type = genomic DNA; note = Canis lupus familiaris
gaggtacagctggtggaatctgggggagacctggtgaagcctgcagggtccctgagactgtcctgtgtggcctctggattccccttcagtgactctgacatgaactgggtccgccaggctcctgggaaggggctacagtgggtcgcaggtattgacgagggtgcaactgacacaggctacgcagatgcagtgaagggccgattcaccatctccagagacgacgccaagaacacactttatctacagatgaacagcctgagagccgacgacacggccatgtactattgtgccgcggggggtaatttcgcctactggggccagggaaccctggtcaccgtctcgagt

SEQ ID NO: 334 - DNA, 336 bp, Canis lupus
  source 1..336: mol_type = genomic DNA; note = Canis lupus familiaris
gatatcgtcatgacgcagaccccactgtccctgtccgtcagccctggagagccggcctccatctcctgcaaggccagtcagagcctcctgcacagtaacgggaacacctatttgtactggtttcgacagaagccaggccagtctccacagggcctgatctacttggtttccaaacgtttctcttgggtcccagacaggttcagtggcagcgggtcagggacggatttcaccctgagaatcagcagagtggaggctgatgatgctggactttattactgcgggcaaggtatacaatatccttatactttcagccagggaaccaagctggagataaaa

SEQ ID NO: 335 - DNA, 342 bp, Canis lupus
  source 1..342: mol_type = genomic DNA; note = Canis lupus familiaris
ggggtgcagctggtggagtctgggggagacctggtgaagcctgcggggtccctgagactgtcctgtgtggcctctggattcaccttcagtaactatggcatgagctgggtccgccaggctcctgggaaggggctgcagtgggtcgcgtctattgccagtggtggaagtgccactgcttacgcagacgctgtgaagggccgattcaccctctccagagacgacgccaagaacacactgtatctgcagatgaacaacctgagaaccgaggacacgtccatgtattactgtgtggccccgggatttcagtactggggccagggcaccctggtcactgtctcgagt

SEQ ID NO: 336 - DNA, 336 bp, Canis lupus
  source 1..336: mol_type = genomic DNA; note = Canis lupus familiaris
gatatcgtcatgacacagaccccactgtccctgtccgtcagccctggagagactgcctccatctcctgcaaggccagtcagagcctcctgcacagtaacgggaacacctatttgttttggtttcgacagaagtcaggccagtctccacagcgtttgatctataaggtctccaacagagaccctggggtcccagacaggctcagtggcagcgggtcagggacagatttcacgctgagaatcagcagagtggaggctgacgatgctggagtttattactgcgggcaaggtatacagattcctcgcacttttggccaagggaccaaactggagatcaaa

(19) United States
(12) Patent Application Publication
(10) Pub. No.: US 2026/0028399 A1
(43) Pub. Date: Jan. 29, 2026

(54) HUMAN-LIKE TARGET-BINDING PROTEINS

(71) Applicant: AbnomX BV, Lievegem (BE)

(72) Inventor: Khalil El Mazouari, Lievegem (BE)

(73) Assignee: AbnomX BV, Lievegem (BE)

(21) Appl. No.: 18/840,779

(22) PCT Filed: Feb. 24, 2023
(86) PCT No.: PCT/EP2023/054742

§ 371 Date: Aug. 22, 2024

(30) Foreign Application Priority Data
Feb. 24, 2022 (EP) 22158562.3

(51) Int. Cl. C07K 16/28; C07K 16/18

(52) U.S. Cl. CPC C07K 16/2803; C07K 16/18; C07K 2317/24; C07K 2317/622

(57) ABSTRACT

The application discloses antigen binding polypeptides comprising a VH domain and a VL domain wherein at least one hypervariable loop or complementary determining region (CDR) in the VHI domain or the VL domain is obtained from a VH or VL domain of a species in the family Canidae. Also provided are methods for producing such antigen binding polypeptides, expression vectors comprising said antigen binding polypeptides, methods for preparing a library of said expression vectors and methods for producing such expression vectors. The application also provides said antigen binding polypeptides for use in medicine.

### CROSS-REFERENCE TO RELATED APPLICATIONS

This patent application is a U.S. National Phase of International Patent Application No. PCT/EP2023/054742, filed Feb. 24, 2023, which claims priority to European Patent Application No. 22158562.3, filed Feb. 24, 2022, both of which are hereby incorporated by reference in their entireties herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 364,100 Byte XML (Extensible Markup Language) file named “771654-2nd-ReplacementSequenceListing.xml” created on Jun. 27, 2025.

### FIELD OF THE INVENTION

The invention relates to methods and processes for the production of antigen binding polypeptides, including antibodies that have a high degree of sequence and structural homology with the variable domains of human antibodies.

### BACKGROUND OF THE INVENTION

The use of monoclonal antibodies as therapeutic or diagnostic agents has increased over the years and multiple monoclonal antibodies received regulatory approval and are currently used in the treatment of several diseases, such as auto-immune diseases, inflammatory diseases, infectious diseases, asthma, cardiovascular diseases, transplant rejection, and cancer.

In order to minimize the human immune response against therapeutic antibodies, antibody technology has evolved from full mouse antibodies to chimeric antibodies, having mouse variable domains grafted on a human IgG backbone), to humanized antibodies, which have mouse CDRs grafted on a human IgG backbone. Even further, fully human antibodies derived from synthetic libraries or immunized transgenic mice expressing part of the human IgG repertoire have been developed as well.

For the production of fully human or “humanized” monoclonal antibodies against specific therapeutic target antigens, a number of antibody technology platforms have been developed, each with its own characteristics and potential shortcomings.

Humanization of mouse monoclonal antibodies was initially achieved by development of chimeric antibodies wherein mouse variable domains are combined with human constant domains, resulting in about 70% human content in these antibodies. Next, complementarity determining regions (CDRs) of mouse monoclonal antibodies were grafted onto human framework regions (FRs) of the variable antibody domains of human antibodies. In addition, several amino acid residues present in those framework regions were identified as interacting with the CDRs or antigen and were back mutated in the humanized antibody to improve binding. (Almagro et al. Frontiers in Bioscience. 13:1619-1633 (2008)). This technology resulted in a relatively high degree of primary sequence homology to human VH and VL domain sequences after humanization, but a drawback is that hypervariable loops might not have human-like structures since not all mouse encoded CDRs use canonical folds, and canonical fold combinations, which are not found in human antibodies (Almagro et al., Mol. Immunol. 34:1 199-1214 (1997); Almagro et al., Immunogen. 47:355-63 (1998)). Another disadvantage is the large number of mutations typically required to humanize such antibodies (the procedure for which is complex and time-consuming), with the consequent risk of losing affinity and potency as a result of the number of changes needed for humanization and, the fact that VKappa domains are mainly used in the murine repertoire, whereas approximately half of all human antibodies possess VLambda domains.

“Fully human” monoclonal antibodies can be produced by two very different approaches. The first approach is selection from a fully synthetic human combinatorial antibody library (for example HuCAL®, MorphoSys). The potential drawback of this approach is that the synthetic library only approximates the functional diversity naturally present in the human germline, thus the diversity is somewhat limited. Also, antibodies generated using this approach are not derived from in vivo selection of CDRs via active immunization, and typically affinity maturation has to be done in order to improve affinity for the target antigen. Affinity maturation is a lengthy process which may add considerable time to the antibody discovery process. Also, in the process of affinity maturation certain amino acid residues may be changed which may negatively affect the binding specificity or stability of the resulting antibody (Wu et al., J. Mol. Biol. 368:652-65 (2007)).

Alternative “fully human” platforms are based on transgenic mice which have been engineered to replace the murine immunoglobulin encoding region with antibody-encoding sequences from the human germline (for example HuMab, Medarex). These systems have the advantage that antibodies are raised by active immunization, with the target antigen, i.e. they have a high starting affinity for the antigen, and that no or only minimal antibody engineering of the original antibodies is required in order to make them more human-like. However, the transgenic mouse strains are by definition highly inbred and this has adverse consequences for the strength and diversity of the antibody response. Another drawback with this platform may be impaired B cell maturation due to human Fc/mouse Fc receptor interaction in some transgenic mouse systems.

A further platform is based on immunization of non-human primates, specifically cynomolgus monkeys. Due to the high degree of amino acid sequence identity between monkey and human immunoglobulins it is postulated that antibodies raised in monkeys will require little or no additional “humanization” in the variable domains in order to render them useful as human therapeutics (see WO 93/02108).

### SUMMARY

The present invention is at least in part based on the inventor's discovery of a high degree of sequence identity of both the VH and the VL domains of conventional antibodies from the family Canidae with the VH and VL domains of human antibodies over the framework regions. In particular, the inventors found that the degree of sequence identity between Canidae conventional VH domains and human VH domains, and between Canidae conventional VL domains and human VL domains can be considered to be highly similar to that observed between humans and other primate species, e.g. cynomolgus monkeys, notwithstanding the phylogenetic distance between humans and Canidae.

The high degree of primary amino acid sequence homology with the framework regions of human antibodies, plus the fact that Canidae conventional antibodies can be raised by active immunization of an outbred animal population, which are phylogenetically quite distant from humans, has led to the finding that conventional antibodies from the family Canidae are attractive for engineering monoclonal antibodies having potential utility for use in medicine, such as for example as human therapeutics.

A first aspect of the invention thus provides a method for generating a polynucleotide sequence encoding for an antigen binding polypeptide or an antigen binding polypeptide-based fragment immunoreactive with a target antigen, said method comprising:
a) determining one or more nucleotide sequences encoding at least one hypervariable loop or complementary determining region (CDR) of the VH and/or VL domain of a Canidae conventional antibody immunoreactive with said target antigen;
b) designing a polynucleotide sequence comprising:
   the one or more nucleotide sequences determined in step a),
   one or more nucleotide sequences encoding one or more framework regions (FR) of the VH and/or VL domain of a human antibody, and
   optionally, one or more nucleotide sequences encoding constant domains of a human antibody,
wherein said polynucleotide sequence encodes an antigen binding polypeptide or antigen binding polypeptide-based fragment comprising a VH domain and/or a VL domain that is immunoreactive with said target antigen.

In some embodiments, in step a) nucleotide sequences are determined that encode at least one hypervariable loop or CDR of the VH and the VL domain of a Canidae conventional antibody immunoreactive with said target antigen.

In some embodiments, the polynucleotide sequence designed in step b) comprises nucleotide sequences encoding one or more FRs of the VH and the VL domain of a human antibody. In some embodiments, the nucleotide sequences in step a) encode a CDR1, a CDR2 and a CDR3 of the VH and/or VL domain; preferably the VH and the VL domain, of a Canidae conventional antibody immunoreactive with the target antigen.

In some embodiments, the polynucleotide sequence designed in step b) comprises nucleotide sequences encoding one or more of the framework regions FR1, FR2, FR3 and FR4 of a human antibody.

In some embodiments, step a) further comprises expressing polypeptides comprising VH and VL domains and screening said polypeptides for immunoreactivity with said target antigen so as to identify antigen binding polypeptides comprising said VH and VL domains.

In some embodiments, prior to step a) a step aa) is performed, said step aa) comprising immunizing an animal of a species of the family Canidae, thereby raising said conventional Canidae antibodies against the target antigen, and collecting the antibodies reactive with the target antigen.

In some embodiments, the method further comprises a step c) wherein said step c) comprises generating an antigen binding polypeptide encoded by the polynucleotide sequence designed in step b). In some embodiments, the polypeptides are screened for homology with human antibodies in the VH and/or VL domain.

In some embodiments, the method further comprises generating amino acid substitutions in one or more of the framework regions of the VH and/or VL domain of the human antibody.

In some embodiments, the method for generating a polynucleotide sequence encoding for an antigen binding polypeptide or an antigen binding polypeptide-based fragment immunoreactive with a target antigen comprises:
a) determining one or more nucleotide sequences encoding at least one hypervariable loop or complementary determining region (CDR) of the VH and/or the VL domain of a Canidae conventional antibody immunoreactive with said target antigen, expressing polypeptides comprising said VH and VL domains and screening said polypeptides for immunoreactivity with said target antigen so as to identify antigen-binding polypeptides comprising said VH and VL domains;
b) designing a polynucleotide sequence comprising:
   the one or more nucleotide sequences determined in step a);
   one or more nucleotide sequences encoding one or more framework regions (FR) of the VH and VL domain of a human antibody, and
   optionally, one or more nucleotide sequences encoding constant domains of a human antibody;
wherein said polynucleotide sequence encodes an antigen binding polypeptide or antigen binding polypeptide-based fragment comprising a VH domain and a VL domain that is immunoreactive with said target antigen;
c) generating an antigen binding polypeptide encoded by the polynucleotide sequence designed in step b);
d) optionally, screening the antigen binding polypeptides for homology with human antibodies in the VH and VL domain;
e) optionally, increasing the affinity of said VH and VL domains for said target antigen by modification of one or more amino acid residues in one or more CDRs of the polypeptide.
thereby producing an antigen binding polypeptide or antigen-binding polypeptide-based fragment comprising a VH domain and a VL domain that is immunoreactive with said target antigen.

In some embodiments, the polynucleotide sequence designed in step b) comprises nucleotide sequences encoding one or more of the framework regions FR1, FR2, FR3, FR4 of a human antibody. In some embodiments, the polynucleotide sequence designed in step b) comprises the nucleotide sequences determined in step a) wherein said nucleotide sequences encode a CDR1, a CDR2, and a CDR3 of the VH and/or VL domain of the Canidae conventional antibody immunoreactive with the target antigen.

In some embodiments, the method as disclosed herein comprises the steps of:
I. preparing an expression vector encoding an antigen binding polypeptide immunoreactive with a target antigen by:
   aa) optionally, actively immunizing an animal of a species in the family Canidae, thereby raising conventional Canidae antibodies against said target antigen;
   a) determining one or more nucleotide sequences encoding at least one hypervariable loop or complementary determining region (CDR) of the VH and/or the VL domain of a Canidae conventional antibody immunoreactive with said target antigen, expressing polypeptides comprising VH and VL domains and screening said polypeptides for immunoreactivity with said target antigen so as to identify antigen-binding polypeptides comprising said VH and VL domains;
   b) designing a polynucleotide sequence comprising:
      the one or more nucleotide sequences determined in step a),
      one or more nucleotide sequences encoding one or more framework regions (FR) in the VH and/or VL domain of a human antibody; and
      optionally, one or more nucleotide sequences encoding constant domains of a human antibody;
   wherein said polynucleotide sequence encodes an antigen binding polypeptide or antigen binding polypeptide-based fragment comprising a VH domain and a VL domain that is immunoreactive with said target antigen;
   c) generating an antigen binding polypeptide encoded by the polynucleotide sequence designed in step b) by
      c1) preparing cDNA or genomic DNA from a sample comprising lymphoid tissue (e.g. circulating B cells) from said immunized animals of a species of the family Canidae;
      c2) amplifying regions of said cDNA or genomic DNA to obtain amplified gene segments, each gene segment comprising a sequence of nucleotides encoding a hypervariable loop or CDR of a VH domain and/or a sequence of nucleotides encoding a hypervariable loop or CDR of a VL domain of a Canidae conventional antibody.
      c3) cloning the gene segments obtained in c2) into expression vectors, such that each expression vector contains a gene segment encoding one or more hypervariable loops or CDRs of a VH domain and/or a gene segment encoding one or more hypervariable loops or CDRs of a VL domain and directs expression of an antigen binding polypeptide comprising said one or more hypervariable loops or CDRs of a VH domain and/or said one or more hypervariable loops or CDRs of a VL domain, thereby producing a library of expression vectors;
      c4) screening antigen binding polypeptides encoded by the library obtained in step c3) for immunoreactivity with said target antigen, and thereby selecting an expression vector encoding an antigen binding polypeptide immunoreactive with said target antigen;
      c5) cloning the gene segment encoding the one or more hypervariable loops or CDRs of a VH domain of the vector selected in part c4) and the gene segment encoding the one or more hypervariable loops or CDRs of a VL domain of the vector selected in part c4) into a further expression vector, in operably linkage with a sequence of nucleotides encoding at least one or more framework regions (FR) in the VH and/or VL domain of a human antibody, and optionally, one or more constant domains of a human antibody, thereby producing an expression vector encoding an antigen binding polypeptide;
II. introducing said expression vector into a host cell or a cell-free expression system under conditions which permit expression of the encoded antigen binding polypeptide; and
III. recovering the expressed antigen binding polypeptide.

In some preferred embodiments, the polynucleotide sequence designed in step b) comprises the nucleotide sequences determined in step a) wherein said nucleotide sequences encode a CDR1, a CDR2, and a CDR3 of the VH and/or VL domain of the Canidae conventional antibody immunoreactive with said target antigen.

In some embodiments, the polynucleotide sequence designed in step b) comprises nucleotide sequences encoding one or more of the framework regions FR1, FR2, FR3 and FR4 of a human antibody.

In some embodiments, the species in the family Canidae is selected from domestic dogs, wolves, coyotes, foxes, jackals and dingoes.

A further aspect of the invention provides a method of producing an antigen binding polypeptide or antigen binding polypeptide-based fragment immunoreactive with a target antigen, said method comprising:
a) immunizing an animal of a species of the family Canidae, thereby raising conventional Canidae antibodies against a target antigen;
b) identifying gene segments encoding VH and VL domains in the genome of a sample of lymphoid tissue obtained from said immunized animal and ensuring expression of polypeptides comprising said VH and VL domains;
c) screening said polypeptides for immunoreactivity with said target antigen so as to identify antigen binding polypeptides comprising said VH and VL domains;
d) generating an antigen binding polypeptide comprising a VH domain and a VL domain and optionally further comprising one or more constant domains, wherein
   at least one hypervariable loop or complementary determining region (CDR) in said VH domain or VL domain is obtained from a VH or VL domain identified in step c); and
   one or more of the framework regions in the VL and VL domain are from a human antibody.

In a related aspect, a method for producing a antigen binding polypeptide or antigen binding polypeptide-based fragments immunoreactive with a target antigen, said method comprising the steps of:
I. preparing an expression vector encoding an antigen binding polypeptide immunoreactive with a target antigen by:
a) actively immunizing a species in the family Canidae, thereby raising conventional Canidae antibodies against said target antigen;
b) preparing cDNA or genomic DNA from a sample comprising lymphoid tissue (e.g. circulating B cells) from said immunized species of the family Canidae;
c) amplifying regions of said cDNA or genomic DNA to obtain amplified gene segments, each gene segment comprising a sequence of nucleotides encoding a VH domain or a sequence of nucleotides encoding a VL domain of a Canidae conventional antibody.
d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, thereby producing a library of expression vectors;
e) screening antigen binding polypeptides encoded by the library obtained in step d) for immunoreactivity with said target antigen, and thereby selecting an expression vector encoding an antigen binding polypeptide immunoreactive with said target antigen;
f) cloning the gene segment encoding the VH domain of the vector selected in part e) and the gene segment encoding the VL domain of the vector selected in part e) or one or more hypervariable loop or complementary determining region (CDR) into a further expression vector, in operably linkage with a sequence of nucleotides encoding at least one or more framework regions (FR) in the VH and VL domain of a human antibody, and optionally, one or more constant domains of a human antibody, thereby producing an expression vector encoding an antigen binding polypeptide;
II. introducing said expression vector into a host cell or a cell-free expression system under conditions which permit expression of the encoded antigen binding polypeptide; and
III. recovering the expressed antigen binding polypeptide.

In some embodiments, the further expression vector comprises nucleotide sequences encoding a at least the CDR3 of the VH and/or VL domain, preferably the CDR1, CDR2 and CDR3 of the VH and/or VL domain, of the Canidae conventional antibody immunoreactive with said target antigen. In some embodiments, the nucleotide sequences encoding one or more CDRs are in operably linkage with a sequence of nucleotides encoding the framework regions FR1, FR2, FR3 and FR4 of a human antibody, and optionally, one or more constant domains of a human antibody.

A related aspect provides an antigen binding polypeptide comprising a VH domain and a VL domain and optionally further comprising one or more constant domains, wherein at least one hypervariable loop or complementary determining regions (CDRs) in the VH domain and the VL domain is obtained from a VH and VL domain of a species in the family Canidae and wherein one or more of the framework regions; preferably all framework regions FR1, FR2, FR3 and FR4, in the VH and VL domain are from a human antibody, and optionally wherein the one or more constant domains is human.

In some embodiments, the antigen binding polypeptide comprises a VH domain and a VL domain and optionally further one or more constant domains, wherein at least the CDR3 in the VH domain and the VL domain is obtained from a species in the family Canidae.

In some embodiments, the antigen binding polypeptide comprises a VH domain and a VL domain and optionally further one or more constant domains, wherein all of the CDRs (i.e., CDR1, CDR2 and CDR3) in the VH domain and the VL domain are obtained from a species in the family Canidae.

In some embodiments, the antigen binding polypeptide comprises a VH domain and a VL domain and optionally further one or more constant domains, wherein all of the CDRs (i.e., CDR1, CDR2 and CDR3) in the VH domain and the VL domain are obtained from a species in the family Canidae, and wherein all of the framework regions FR1, FR2, FR3 and FR4 in the VH and the VL domain, and optionally all of the constant domains, are human.

In some embodiments, the antigen binding polypeptide as disclosed herein further comprises one or more amino acid substitutions in one or more of the CDRs as compared to the amino acid sequence of the CDRs of the species in the family Canidae.

In some embodiments, he antigen binding polypeptide as disclosed herein further comprises one or more amino acid substitutions in one or more of the framework regions as compared to the amino acid sequence of the framework regions of the human antibody.

In some embodiments, the antigen binding polypeptide according to the invention comprises hypervariable loops or complementarity determining regions which have been obtained by active immunization of a species in the family Canidae.

In some embodiments, the antigen binding polypeptide of the invention is immunoreactive with a target antigen. In another embodiments the antigen binding polypeptide specifically binds to a target antigen.

In some embodiments, the antigen binding polypeptide of the invention is a recombinant polypeptide, a chimeric polypeptide, a monoclonal antibody and/or a recombinantly expressed chimeric monoclonal antibody.

In some embodiments, the antigen binding polypeptide of the invention is for use in medicine.

A related aspect provides a polynucleotide molecule encoding an antigen binding polypeptide or a fragment of said antigen binding polypeptide as disclosed herein. In some embodiments, a polynucleotide molecule encoding a fragment of an antigen binding polypeptide as disclosed herein is provided, wherein the fragment comprises at least one hypervariable loop or CDR obtained from a VH or VL domain of a species in the family Canidae.

A related aspect provides an expression vector comprising the polynucleotide defined above operably linked to regulatory sequences which permit expression of the antigen binding polypeptide in a host cell or cell-free expression system, a host cell or cell-free expression system containing the expression vector, and a method of producing a recombinant antigen binding polypeptide which comprises culturing the host cell or cell free expression system under conditions which permit expression of the antigen binding polypeptide and recovering the expressed antigen binding polypeptide.

These and further aspects and preferred embodiments of the invention are described in detailed in the following sections and in the appended claims. The subject-matter of the appended claims is hereby specifically incorporated in this specification.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As used herein, amino acid residues will be indicated either by their full name or according to the standard three-letter or one-letter amino acid code.

As used herein, the terms “polypeptide”, “protein”, “peptide”, and “amino acid sequence” are used interchangeably, and refer to a polymeric form of amino acids of any length, which can include coded and non-coded amino acids, chemically or biochemically modified or derivatized amino acids, and polypeptides having modified peptide backbones.

As used herein, the terms ‘nucleic acid molecule’, ‘polynucleotide’, ‘polynucleic acid’, ‘nucleic acid’ are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural similarity between two macromolecules, particularly between two polypeptides or polynucleotides, from same or different taxons, wherein said similarity is due to shared ancestry. Hence, the term “homologues” denotes so-related macromolecules having said secondary and optionally tertiary structural similarity. For comparing two or more nucleotide sequences, in some embodiments, the “(percentage of) sequence identity” between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence identity between two amino acid sequences, in some embodiments, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art. Amino acid sequences and nucleic acid sequences are said to be “exactly the same” if they have 100% sequence identity over their entire length.

The term “affinity”, as used herein, refers to the degree to which a polypeptide, in particular an antigen binding polypeptide, such as an antibody, binds to an antigen so as to shift the equilibrium of antigen and polypeptide toward the presence of a complex formed by their binding. Thus, for example, where an antigen and antibody (e.g., antibody fragment) are combined in relatively equal concentration, an antibody (e.g., antibody fragment) of high affinity will bind to the available antigen so as to shift the equilibrium toward a high concentration of the resulting complex. The dissociation constant is commonly used to describe the affinity between the protein binding domain and the antigenic target. Typically, the dissociation constant is lower than 10⁻⁵ M. In some embodiments, the dissociation constant is lower than 10⁻⁶ M, more preferably, lower than 10⁻⁷ M, most preferably, lower than 10⁻⁸ M, such as lower than 10⁻⁹ M.

The term “antigen binding polypeptide” refers to any polypeptide comprising a VH domain and a VL domain which is immunoreactive with, exhibits specific binding to, a target antigen. Exemplary antigen binding polypeptides include antibodies and immunoglobulins, and also antibody fragments, as discussed elsewhere herein.

As used herein, the term “antibody” or “conventional antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated VH) and a heavy chain constant region. The heavy chain constant region comprises three domains, CH1, CH2 and CH3. Each light chain comprises a light chain variable region (abbreviated VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). As used herein, the term “antibody” includes antibodies of any isotype, including IgA, IgG, IgD, IgE or IgM, and includes polyclonal antibodies, monoclonal antibodies, humanized antibodies, single-chain antibodies, and fragments thereof such as Fab, F (ab) 2, Fv, and other fragments or portions that retain the antigen binding function of the parent antibody. As such, an antibody may refer to an immunoglobulin, or a fragment or portion thereof, or to a construct comprising an antigen-binding portion comprised within a modified immunoglobulin-like framework, or to an antigen-binding portion comprised within a construct comprising a non-immunoglobulin-like framework or scaffold.

The term “antigen”, when referring to the “target antigen” against which the antigen binding polypeptide is immunoreactive, takes its normal meaning to a person of ordinary skill in the art, and includes, inter alia, polypeptide, peptide, polysaccharide, glycoprotein, polynucleotide (e.g. DNA), or synthetic chemical antigens.

The term “antigen” can also be used to describe the material employed in the immunization of animals (e.g. Canidae) during the manufacture of antigen binding polypeptides of the invention. In this context the term “antigen” may take a wider meaning, and could encompass purified forms of the antigen, and also crude or semi-purified preparations of the antigen, such as for example cells, cell lysates or supernatants, cell fractions, e.g. cell membranes, etc., plus haptens conjugated with an appropriate carrier protein. The “antigen” used in an immunization protocol is not necessarily structurally identical to the “target antigen” with which the resulting antigen binding polypeptide is to immune-react. Typically the “antigen” used for immunization may be a truncated form of the “target antigen”, e.g. a fragment containing an immunogenic epitope. Further characteristics of “antigens” used for active immunization are described elsewhere herein, and would be generally known to a person skilled in the art.

The terms “specifically bind” and “specific binding”, as used herein, generally refer to the ability of a polypeptide, in particular an antigen binding polypeptide, such as an antibody or immunoglobulin, to preferentially bind to a particular antigen that is present in a homogeneous mixture of different antigens. An antigen binding polypeptide binds “specifically” to its target antigen if it binds an epitope on the target antigen in preference to other epitopes. “Specific binding” does not exclude cross-reactivity with other antigens bearing similar antigenic epitopes. In certain embodiments, a specific binding interaction will discriminate between desirable and undesirable antigens in a sample, in some embodiments more than about 10 to 100-fold or more (e.g., more than about 1000- or 10,000-fold). Accordingly, an amino acid sequence, in particular an antigen binding polypeptide as disclosed herein is said to “specifically bind to” a particular target when that amino acid sequence has affinity for, specificity for and/or is specifically directed against that target (or for at least one part or fragment thereof).

Native or naturally occurring “conventional” Canidae antibodies are usually heterotetrameric glycoproteins, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end (N-terminal) a variable domain (VH) followed by a number of constant domains. Each light chain has a variable domain (VL) at one end (N-terminal) and a constant domain (CL) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “immunoglobulin”—whether used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as VHH domains or VH/VL domains, respectively).

The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain

The term “immunoglobulin domain” as used herein refers to a globular region of an antibody chain (such as e.g., a chain of a conventional 4-chain antibody or of a heavy chain antibody), or to a polypeptide that essentially consists of such a globular region. Immunoglobulin domains are characterized in that they retain the immunoglobulin fold characteristic of antibody molecules, which consists of a two-layer sandwich of about seven antiparallel beta-strands arranged in two beta-sheets, optionally stabilized by a conserved disulphide bond.

The term “variable domain” or “immunoglobulin variable domain” as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of a variable domain can be indicated as follows: FR1-CDR1-FR2-CDR2-FR3-CDR3-FR4. It is the variable domain(s) that confer specificity to an antibody for the antigen by carrying the antigen-binding site. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ). The first, second and third hypervariable loops of the VKappa light chain domain are referred to herein as L1(κ), L2(κ) and L3(κ). The first, second and third hypervariable loops of the VH domain are referred to herein as H1, H2 and H3.

Unless otherwise indicated, the terms L1, L2 and L3 respectively refer to the first, second and third hypervariable loops of a VL domain, and in the context of this application, encompass hypervariable loops obtained from both Vkappa and Vlambda isotypes from Canidae. The terms H1, H2 and H3 respectively refer to the first, second and third hypervariable loops of the VH domain, and in the context of this application, encompass hypervariable loops obtained from any of the known heavy chain isotypes from Canidae.

The hypervariable loops L1, L2, L3, H1, H2 and H3 may each comprise part of a “complementarity determining region” or “CDR”. The term CDR thus refers to variable regions of either the H (heavy) or L (light) chains (also abbreviated as VH and VL, respectively). These CDRs account for the basic specificity of the antibody for a particular antigenic determinant structure. The term “hypervariable loop” and “CDR” are not strict synonymous, since the hypervariable loops (HVs) are defined on the basis of the structure, whereas CDRs are defined based on sequence variability (Kabat et al., Sequences of Proteins of Immunological Interest, 5th Ed. Public Health Service, National Institutes of Health, Bethesda, MD., 1983) and the limits of the HVs and the CDRs may be different in some VH and VL domains.

The CDRs of the VL and VH domains can typically be defined as comprising the following amino acids: residues 24-34 (CDRL1), 50-56 (CDRL2) and 89-97 (CDRL3) in the light chain variable domain, and residues 31-35 or 31-35b (CDRH1), 50-65 (CDRH2) and 95-102 (CDRH3) in the heavy chain variable domain; Kabat et al., Sequences of Proteins of Immunological Interest, 5th Ed. Public Health Service, National Institutes of Health, Bethesda, MD. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR). The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et ai, J. Mol. Biol. 227:799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

Determination of CDR regions may also be done according to different methods. Generally, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

With the present invention, the inventors surprisingly found a high degree of sequence identity of both the VH and the VL domains of conventional antibodies from the family Canidae with the VH and VL domains of human antibodies over the framework regions. In particular, the degree of sequence identity between Canidae conventional VH domains and human VH domains, and between Canidae conventional VL domains and human VL domains can be considered to be highly similar to that observed between humans and other primate species, e.g. cynomolgus monkeys, notwithstanding the phylogenetic distance between humans and Canidae.

Furthermore, the present inventors observed that Canidae germline and somatically mutated DNA sequences encoding both the VH and the VL domains of conventional antibodies from species in the family Canidae exhibit a high degree of sequence identity/sequence homology with the human germline DNA sequences which encode VH and VL domains of human antibodies, over the framework regions. This high degree of primary amino acid sequence homology in addition to the fact that Canidae conventional antibodies can be raised by active immunization of an outbred animal population, which are phylogenetically quite distant from humans, has led the present inventors to conclude that conventional antibodies from the family Canidae are an attractive starting point for engineering monoclonal antibodies having potential utility for use in medicine, such as for example as human therapeutics.

Thus in a first aspect of the invention a method for generating a polynucleotide sequence encoding for an antigen binding polypeptide or an antigen binding polypeptide-based fragment immunoreactive with a target antigen is provided. Said method comprises the steps of:
a) determining one or more nucleotide sequences encoding at least one hypervariable loop or complementary determining region (CDR) of the VH and/or VL domain; preferably of the VH and VL domain, of a Canidae conventional antibody immunoreactive with said target antigen;
b) designing a polynucleotide sequence comprising:
   the one or more nucleotide sequences determined in step a),
   one or more nucleotide sequences encoding one or more framework regions (FR) of the VH and/or VL domain of a human antibody, and
   optionally, one or more nucleotide sequences encoding constant domains of a human antibody,
wherein said polynucleotide sequence encodes an antigen binding polypeptide or antigen binding polypeptide-based fragment comprising a VH domain and/or a VL domain; preferably a VH and VL domain, that is immunoreactive with said target antigen.

In some embodiments, step a) further comprises expressing polypeptides comprising VH and VL domains and screening said polypeptides for immunoreactivity with said target antigen so as to identify antigen binding polypeptides comprising said VH and VL domains.

In some embodiments, a step aa) is performed prior to step a), wherein said step aa) comprises immunizing an animal of a species of the family of Canidae, thereby raising said conventional Canidae antibodies against said target antigen, and collecting the antibodies reactive with the target antigen.

In some embodiments, the method for generating a polynucleotide sequence encoding for an antigen binding polypeptide or an antigen binding polypeptide-based fragment immunoreactive with a target antigen further comprises a step c) wherein step c) comprises generating an antigen binding polypeptide encoded by the polynucleotide sequence designed in step b).

In some embodiments, the method further comprises screening the polypeptides for homology with human antibodies in the VH and VL domain.

In some embodiments, the method further comprises increasing the affinity of said VH and VL domains for said target antigen by one or more amino acid substitutions or modification of one or more amino acid residues in one or more CDRs of the polypeptide.

In some embodiments, the method further comprises generating one or more amino acid substitutions in one or more of the framework regions of the VH and/or VL domain of the human antibody.

In some embodiments, a method for generating a polynucleotide sequence encoding for an antigen binding polypeptide or an antigen binding polypeptide-based fragment immunoreactive with a target antigen is thus provided, said method comprising:
a) determining one or more nucleotide sequences encoding at least one hypervariable loop or complementary determining region (CDR) of the VH and the VL domain of a Canidae conventional antibody immunoreactive with said target antigen, expressing polypeptides comprising said VH and VL domains and screening said polypeptides for immunoreactivity with said target antigen so as to identify antigen-binding polypeptides comprising said VH and VL domains;
b) designing a polynucleotide sequence comprising:
   the one or more nucleotide sequences determined in step a);
   nucleotide sequences encoding one or more of the framework regions; preferably all framework regions FR1, FR2, FR3 and FR4, in the VH and VL domain of a human antibody; and
   optionally, one or more nucleotide sequences encoding constant domains of a human antibody; and
wherein said polynucleotide sequence encodes an antigen binding polypeptide or antigen binding polypeptide-based fragment comprising a VH domain and a VL domain that is immunoreactive with said target antigen;
c) generating an antigen binding polypeptide encoded by the polynucleotide sequence designed in step b);
d) optionally, screening the antigen binding polypeptides for homology with human antibodies in the VH and VL domain;
e) optionally, increasing the affinity of said VH and VL domains for said target antigen by modification of one or more amino acid residues in one or more CDRs of the polypeptide.
thereby producing an antigen binding polypeptide or antigen-binding polypeptide-based fragment comprising a VH domain and a VL domain that is immunoreactive with said target antigen.

In some embodiments, the polynucleotide sequence designed in step b) comprises nucleotide sequences encoding a CDR1, a CDR2, and a CDR3 of the VH and/or VL domain of the Canidae conventional antibody immunoreactive with the target antigen.

In some embodiments, the polynucleotide sequence designed in step b) comprises nucleotide sequences encoding the framework regions FR1, FR2, FR3 and FR4 of the VH and VL domain of a human antibody, or of human origin.

Also provided is a method for generating a polynucleotide sequence encoding for an antigen binding polypeptide or an antigen binding polypeptide-based fragment immunoreactive with a target antigen, or a method of producing an antigen binding polypeptide or antigen binding polypeptide-based fragment immunoreactive with a target antigen. Said method comprises:
aa) optionally, actively immunizing an animal of a species in the family Canidae, thereby raising conventional Canidae antibodies against said target antigen;
a) determining one or more nucleotide sequences encoding at least one hypervariable loop or complementary determining region (CDR) of the VH and/or the VL domain of a Canidae conventional antibody immunoreactive with said target antigen, expressing polypeptides comprising VH and VL domains and screening said polypeptides for immunoreactivity with said target antigen so as to identify antigen-binding polypeptides comprising said VH and VL domains;
b) designing a polynucleotide sequence comprising:
   the one or more nucleotide sequences determined in step a),
   one or more nucleotide sequences encoding one or more framework regions (FR) in the VH and VL domain of a human antibody; and
   optionally, one or more nucleotide sequences encoding constant domains of a human antibody;
wherein said polynucleotide sequence encodes an antigen binding polypeptide or antigen binding polypeptide-based fragment comprising a VH domain and a VL domain that is immunoreactive with said target antigen;
c) generating an antigen binding polypeptide encoded by the polynucleotide sequence designed in step b) by
   c1) preparing cDNA or genomic DNA from a sample comprising lymphoid tissue (e.g. circulating B cells) from said immunized animals of a species of the family Canidae;
   c2) amplifying regions of said cDNA or genomic DNA to obtain amplified gene segments, each gene segment comprising a sequence of nucleotides encoding a hypervariable loop or CDR of a VH domain and/or a sequence of nucleotides encoding a hypervariable loop or CDR of a VL domain of a Canidae conventional antibody.
   c3) cloning the gene segments obtained in c2) into expression vectors, such that each expression vector contains a gene segment encoding one or more hypervariable loops or CDRs of a VH domain and/or a gene segment encoding one or more hypervariable loops or CDRs of a VL domain and directs expression of an antigen binding polypeptide comprising said one or more hypervariable loops or CDRs of a VH domain and/or said one or more hypervariable loops or CDRs of a VL domain, thereby producing a library of expression vectors;
   c4) screening antigen binding polypeptides encoded by the library obtained in step c3) for immunoreactivity with said target antigen, and thereby selecting an expression vector encoding an antigen binding polypeptide immunoreactive with said target antigen;
   c5) cloning the gene segment encoding the one or more hypervariable loops or CDRs of a VH domain of the vector selected in part c4) and the gene segment encoding the one or more hypervariable loops or CDRs of a VL domain of the vector selected in part c4) into a further expression vector, in operably linkage with a sequence of nucleotides encoding at least one or more framework regions (FR) in the VH and/or VL domain of a human antibody, and optionally, one or more constant domains of a human antibody, thereby producing an expression vector encoding an antigen binding polypeptide;
      II. introducing said expression vector into a host cell or a cell-free expression system under conditions which permit expression of the encoded antigen binding polypeptide; and
      III. recovering the expressed antigen binding polypeptide.

In some embodiments, the polynucleotide sequence designed in step b) comprises nucleotide sequences that encode a CDR1, a CDR2, and/or a CDR3 of the VH and/or VL domain of the Canidae conventional antibody immunoreactive with the target antigen.

In some embodiments, the polynucleotide sequence designed in step b) comprises nucleotide sequences encoding one or more of the framework regions FR1, FR2, FR3 and FR4 of the VH and VL domain of a human antibody, such as for example two, three or all four framework regions of the VH and VL domain of a human antibody. In some embodiments, the polynucleotide sequence designed in step b) comprises nucleotide sequences encoding the framework regions FR1, FR2, FR3 and FR4 of the VH and VL domain of a human antibody. In some further embodiments, additional amino acid substitutions in one or more of the FR1, FR2, FR3 or FR4 of the amino acid sequences of the human antibody can be applied, for example to increase the affinity with the target antigen. In some embodiments, the polynucleotide sequence designed in step b) comprises nucleotide sequences that encode at least the CDR3 of the VH and/or VL domain of the Canidae conventional antibody immunoreactive with said target antigen. In some embodiments, the polynucleotide sequence designed in step b) comprises nucleotide sequences that encode a CDR1, a CDR2 and a CDR3 of the VH and/or VL domain of the Canidae conventional antibody immunoreactive with said target antigen.

Further provided is a method of producing an antigen binding polypeptide or antigen binding polypeptide-based fragment immunoreactive with a target antigen is provided. Said method comprises the steps of:
a) immunizing an animal of a species of the family Canidae, thereby raising conventional Canidae antibodies against a target antigen;
b) identifying gene segments encoding VH and VL domains in the genome of a sample of lymphoid tissue obtained from said immunized animal and ensuring expression of polypeptides comprising said VH and VL domains;
c) screening said polypeptides for immunoreactivity with said target antigen so as to identify antigen binding polypeptides comprising said VH and VL domains;
d) generating an antigen binding polypeptide comprising a VH domain and a VL domain and optionally further comprising one or more constant domains, wherein
   at least one hypervariable loop or complementary determining region (CDR) in said VH domain or VL domain is obtained from a VH or VL domain identified in step c); and
   one or more of the framework regions are of a human antibody, and
   optionally one or more constant domains, is human.

In some embodiments, the antigen binding polypeptide further comprises one or more hypervariable loops or CDRs of human origin.

In some embodiments, the step d) of the method comprises fusing one or more antigen binding polypeptides comprising a VH and VL domain identified in step c) with one or more constant domains of a human antibody.

A related aspect of the invention provides a method of producing an antigen binding polypeptide or antigen binding polypeptide-based fragment, in particular a monoclonal antibody, or an antibody-based fragment thereof which is immunoreactive with a target antigen. Said method comprises the steps of:
I. preparing an expression vector encoding an antigen binding polypeptide immunoreactive with a target antigen by:
a) optionally, actively immunizing an animal of species in the family Canidae, thereby raising conventional Canidae antibodies against said target antigen;
b) preparing cDNA or genomic DNA from a sample comprising lymphoid tissue (e.g. circulating B cells) from said immunized species of the family Canidae;
c) amplifying regions of said cDNA or genomic DNA to obtain amplified gene segments, each gene segment comprising a sequence of nucleotides encoding a VH domain or a sequence of nucleotides encoding a VL domain of a Canidae conventional antibody.
d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, thereby producing a library of expression vectors;
e) screening antigen binding polypeptides encoded by the library obtained in step d) for immunoreactivity with said target antigen, and thereby selecting an expression vector encoding an antigen binding polypeptide immunoreactive with said target antigen;
f) cloning the gene segment encoding the VH domain of the vector selected in part e) and the gene segment encoding the VL domain of the vector selected in part e) or one or more hypervariable loop or complementary determining region (CDR) into a further expression vector, in operably linkage with a sequence of nucleotides encoding at least one or more framework regions in the VH and VL domain of a human antibody, and optionally one or more constant domains of a human antibody, thereby producing an expression vector encoding an antigen binding polypeptide, in particular a chimeric antigen binding polypeptide;
II. introducing said expression vector into a host cell or a cell-free expression system under conditions which permit expression of the encoded antigen binding polypeptide; and
III. recovering the expressed antigen binding polypeptide.

In some embodiments, the method for producing an antigen binding polypeptide or antigen binding polypeptide-based fragment, in particular a monoclonal antibody, or an antibody-based fragment thereof which is immunoreactive with a target antigen, comprises the steps of:
aa) optionally, actively immunizing an animal of a species in the family Canidae, thereby raising conventional Canidae antibodies against said target antigen;
a) determining one or more nucleotide sequences encoding at least one hypervariable loop or complementary determining region (CDR) of the VH and/or the VL domain of a Canidae conventional antibody immunoreactive with said target antigen, expressing polypeptides comprising VH and VL domains and screening said polypeptides for immunoreactivity with said target antigen so as to identify antigen-binding polypeptides comprising said VH and VL domains;
b) designing a polynucleotide sequence comprising:
   the one or more nucleotide sequences determined in step a),
   one or more nucleotide sequences encoding one or more framework regions (FR) in the VH and/or VL domain of a human antibody; and
   optionally, one or more nucleotide sequences encoding constant domains of a human antibody;
wherein said polynucleotide sequence encodes an antigen binding polypeptide or antigen binding polypeptide-based fragment comprising a VH domain and a VL domain that is immunoreactive with said target antigen;
c) generating an antigen binding polypeptide encoded by the polynucleotide sequence designed in step b) by
   c1) preparing cDNA or genomic DNA from a sample comprising lymphoid tissue (e.g. circulating B cells) from said immunized animals of a species of the family Canidae;
   c2) amplifying regions of said cDNA or genomic DNA to obtain amplified gene segments, each gene segment comprising a sequence of nucleotides encoding a hypervariable loop or CDR of a VH domain and/or a sequence of nucleotides encoding a hypervariable loop or CDR of a VL domain of a Canidae conventional antibody.
   c3) cloning the gene segments obtained in c2) into expression vectors, such that each expression vector contains a gene segment encoding one or more hypervariable loops or CDRs of a VH domain and/or a gene segment encoding one or more hypervariable loops or CDRs of a VL domain and directs expression of an antigen binding polypeptide comprising said one or more hypervariable loops or CDRs of a VH domain and/or said one or more hypervariable loops or CDRs of a VL domain, thereby producing a library of expression vectors;
   c4) screening antigen binding polypeptides encoded by the library obtained in step c3) for immunoreactivity with said target antigen, and thereby selecting an expression vector encoding an antigen binding polypeptide immunoreactive with said target antigen;
   c5) cloning the gene segment encoding the one or more hypervariable loops or CDRs of a VH domain of the vector selected in part c4) and the gene segment encoding the one or more hypervariable loops or CDRs of a VL domain of the vector selected in part c4) into a further expression vector, in operably linkage with a sequence of nucleotides encoding at least one or more framework regions (FR) in the VH and VL domain of a human antibody, and optionally, one or more constant domains of a human antibody, thereby producing an expression vector encoding an antigen binding polypeptide;
      II. introducing said expression vector into a host cell or a cell-free expression system under conditions which permit expression of the encoded antigen binding polypeptide; and
      III. recovering the expressed antigen binding polypeptide.

In some embodiments, the nucleotide sequences in step a) encode a CDR1, a CDR2 and a CDR3 of the VH and/or VL domain of a Canidae conventional antibody immunoreactive with said target antigen.

In some embodiments, the polynucleotide sequence designed in step b) comprises a CDR1, a CDR2 and/or a CDR3 of the VH and/or VL domain of a Canidae conventional antibody immunoreactive with said target antigen. In some embodiments, the polynucleotide sequence designed in step b) comprises nucleotide sequences that encode at least the CDR3 of the VH and/or VL domain of the Canidae conventional antibody immunoreactive with said target antigen. In some embodiments, the polynucleotide sequence designed in step b) comprises nucleotide sequences that encode a CDR1, a CDR2 and a CDR3 of the VH and/or VL domain of the Canidae conventional antibody immunoreactive with said target antigen.

In some embodiments, the polynucleotide sequence designed in step b) comprises nucleotide sequences encoding the framework regions FR1, FR2, FR3, and/or FR4 of the VH and/or VL domain of a human antibody. In some embodiments, the polynucleotide sequence designed in step b) comprises nucleotide sequences encoding the framework regions FR1, FR2, FR3 and FR4 of the VH and VL domain of a human antibody.

The antigen binding polypeptide produced in the methods of the invention thus comprises a VH domain and a VL domain, wherein at least one hypervariable loop or CDR in the VH domain or the VL domain is obtained from a VH or VL domain of a species in the family Canidae.

In some embodiments, the antigen binding polypeptide comprises a VH domain and/or a VL domain wherein the hypervariable loop H3 (also referred to as CDR3 or CDR-H3) in the VH domain, or hypervariable loop L3 (also referred to as CDR3 or CDR-L3) in the VL domain or both is/are obtained from a VH or VL domain of a species in the family Canidae.

In some embodiments, the antigen binding polypeptide comprises a VH domain and a VL domain wherein either hypervariable loop H1 (also referred to as CDR1 or CDR-H1), or hypervariable loop H2 (also referred to as CDR2 or CDR-H2), or both hypervariable loop H1 and hypervariable loop H2 are obtained from a VH domain of a species in the family Canidae, and/or wherein either hypervariable loop L1 (also referred to as CDR1 or CDR-L1), or hypervariable loop L2 (also referred to as CDR2 or CDR-L2), or both hypervariable loop L1 and hypervariable loop L2 are obtained from a VL domain of a species in the family Canidae. In some further embodiments, said antigen binding polypeptide comprises a VH domain and a VL domain wherein the hypervariable loop H3 (also referred to as CDR3) in the VH domain, or hypervariable loop L3 (also referred to as CDR3) in the VL domain or both is/are obtained from a VH or VL domain of a species in the family Canidae.

In some embodiments, the antigen binding polypeptide comprises a VH domain and a VL domain wherein either hypervariable loop L1 (CDR-L1) or hypervariable loop L2 (CDR-L2), or both hypervariable loop L1 (CDR-L1) and hypervariable loop L2 (CDR-L2) are obtained from VL domain of a species in the family Canidae. In some further embodiments, said antigen binding polypeptide comprises a VH domain and a VL domain wherein the hypervariable loop H3 (CDR-H3) in the VH domain, or hypervariable loop L3 (CDR-L3) in the VL domain or both is/are obtained from a VH or VL domain of a species in the family Canidae.

In some embodiments, the antigen binding polypeptide comprises a VH domain and a VL domain wherein either hypervariable loop H1 (CDR-H1) or hypervariable loop H2 (CDR-H2), or both hypervariable loop H1 (CDR-H1) and hypervariable loop H2 (CDR-H2) are obtained from a VH domain of a species in the family Canidae and either hypervariable loop L1 (CDR-L1) or hypervariable loop L2 (CDR-L2), or both hypervariable loop L1 (CDR-L1) and hypervariable loop L2 (CDR-L2) are obtained from a VL domain of a species in the family Canidae. In some specific embodiments, said antigen binding polypeptide comprises a VH domain and a VL domain wherein the hypervariable loop H3 (CDR-H3) in the VH domain, or hypervariable loop L3 (CDR-L3) in the VL domain or both is/are obtained from a VH or VL domain of a species in the family Canidae. In one specific embodiment, each of the hypervariable loops H1 (CDR-H1), H2 (CDR-H2), H3 (CDR-H3), L1 (CDR-L1), L2 (CDR-L2) and L3 (CDR-L3) in both the VH domain and the VL domain are obtained from a species in the family Canidae.

In some embodiments, the antigen binding polypeptide comprises a VH domain and a VL domain wherein each of the hypervariable loops or CDR in both the VH domain and the VL domain are obtained from a species in the family Canidae. In some embodiments, the entire VH domain and/or the entire VL domain may be obtained from a species in the family Canidae. The Canidae VH domain and/or the Canidae VL domain may then be subject to protein engineering, in which one or more amino acid mutations, such as substitutions, insertions or deletions are introduced into the Canidae species. These engineered changes preferably include amino acid substitutions relative to the Canidae sequence. Such changes include “humanization” or “germlining” wherein one or more amino residues in a Canidae-encoded VH or VL domain are replaced with equivalent residues from a homologous human-encoded VH or VL domain.

Thus, in some embodiments, the antigen binding polypeptide comprises a VH domain and a VL domain wherein the hypervariable loop(s) or CDR(s) obtained from a VH or VL domain of a species in the family of Canidae have an amino acid sequence that is encoded by a VH or VL gene of a species in the family Canidae. In some embodiments, the antigen binding polypeptide comprises a VH domain and a VL domain wherein the hypervariable loop(s) or CDR(s) obtained from a VH or VL domain of a species in the family Canidae have amino acid sequence(s) substantially identical to the amino acid sequence of hypervariable loops or CDRs of a conventional antibody obtained by active immunization of a species in the family Canidae. In some embodiments, the antigen binding polypeptide contains at least one amino acid substitution in at least one hypervariable loop or CDR of either the VH domain or the VL domain in comparison to a Canidae VH or VL domain obtained by active immunization of a species in the family Canidae, or in comparison to a VH or VL domain encoded by a VH or VL gene of a species in the family of Canidae.

By “hypervariable loop” or “complementarity determining region (CDR)” obtained from a VH domain or a VL domain of a species in the family Canidae is thus meant that that hypervariable loop (HV) or CDR has an amino acid sequence which is identical, or substantially identical, to the amino acid sequence of a hypervariable loop or CDR which is encoded by a Canidae immunoglobulin gene. In this context “immunoglobulin gene” includes germline genes, immunoglobulin genes which have undergone rearrangement, and also somatically mutated genes. Thus, the amino acid sequence of the HV or CDR obtained from a VH or VL domain of a Canidae species may be identical to the amino acid sequence of a hypervariable loop or CDR present in a mature Canidae antibody. The term “obtained from” in this context implies a structural relationship, in the sense that the hypervariable loops or CDRs of the antigen binding polypeptide of the invention embody an amino acid sequence (or minor variants thereof) which was originally encoded by a Canidae immunoglobulin gene. However, this does not necessarily imply a particular relationship in terms of the production process used to prepare the antigen binding polypeptide of the invention. As will be discussed below, there are several processes which may be used to prepare antigen binding polypeptides comprising hypervariable loops or CDRs with amino acid sequences identical to (or substantially identical to) sequences originally encoded by a Canidae immunoglobulin gene.

For the avoidance of doubt, the term “VH domain obtained from a species of Canidae” encompass VH domains which are the products of synthetic or engineered recombinant genes (including codon-optimized synthetic genes), which VH domains have an amino acid sequence identical to (or substantially identical to) the amino acid sequence of a VH domain encoded by a Canidae immunoglobulin gene (germline, rearranged or somatically mutated). Similarly, the terms “VL domain of a conventional antibody of a Canidae” and “VL domain obtained from a species of Canidae” are used synonymously and encompass VL domains which are the products of synthetic or engineered recombinant genes (including codon-optimized synthetic genes), which VL domains have an amino acid sequence identical to (or substantially identical to) the amino acid sequence of a VL domain encoded by a Canidae immunoglobulin gene (germline, rearranged or somatically mutated).

The VL domains in the polypeptide of the invention may be of the VLambda type or the Vkappa type. The term “VL domain” therefore refers to both VKappa and VLambda isotypes from Canidae, and engineered variants thereof which contain one or more amino acid substitutions, insertions or deletions relative to a Canidae VL domain.

In certain embodiments, Canidae hypervariable loops (or CDRs) may be thus obtained by active immunization of a species in the family Canidae with the desired target antigen. As discussed in detail herein, following immunization of Canidae with the target antigen, B cells producing antibodies having specificity for the desired antigen can be identified and a polynucleotide encoding the VH and VL domains of such antibodies can be isolated using known techniques.

Thus, in a specific embodiment, the invention provides a recombinant antigen-binding polypeptide immunoreactive with a target antigen, the polypeptide comprising a VH domain and a VL domain, wherein at least one hypervariable loop or complementarity determining region in the VH domain or the VL domain is obtained from a VH or VL domain of a species in the family Canidae, which antigen-binding polypeptide is obtainable by a process comprising the steps of:
(a) immunizing a species in the family Canidae with a target antigen or with a polynucleotide encoding said target antigen and raising an antibody to said target antigen;
(b) determining the nucleotide sequence encoding at least one hypervariable loop or complementarity determining region (CDR) of the VH and/or the VL domain of a Canidae antibody immunoreactive with said target antigen; and
(c) expressing an antigen-binding polypeptide immunoreactive with said target antigen, said antigen binding polypeptide comprising a VH and a VL domain, wherein at least one hypervariable loop or complementarity determining region (CDR) of the VH domain or the VL domain has an amino acid sequence encoded by the nucleotide sequence determined in part (b).

In a related aspect, the invention thus provides a method of producing a recombinant antigen-binding polypeptide immunoreactive with a target antigen, the polypeptide comprising a VH domain and a VL domain, wherein at least one hypervariable loop or complementarity determining region in the VH domain or the VL domain is obtained from a VH or VL domain of a species in the family Canidae. Said method comprises the steps of:
(a) immunizing a species in the family Canidae with a target antigen or with a polynucleotide encoding said target antigen and raising an antibody to said target antigen;
(b) determining the nucleotide sequence encoding at least one hypervariable loop or complementarity determining region (CDR) of the VH and/or the VL domain of a Canidae antibody immunoreactive with said target antigen; and
(c) expressing an antigen-binding polypeptide immunoreactive with said target antigen, said antigen binding polypeptide comprising a VH and a VL domain, wherein at least one hypervariable loop or complementarity determining region (CDR) of the VH domain or the VL domain has an amino acid sequence encoded by the nucleotide sequence determined in part (b).

Isolated Canidae VH and VL domains obtained by active immunization can be used as a basis for engineering antigen-binding polypeptides according to the invention. Starting from intact Canidae VH and VL domains or parts of said VH and VL domains, such as hypervariable loops or CDRs, it is possible to engineer one or more amino acid mutations, such as substitutions, insertions or deletions which depart from the starting Canidae sequence. In certain embodiments, such substitutions, insertions or deletions may be present in the framework regions of the VH domain and/or the VL domain. The purpose of such changes in primary amino acid sequence may be to reduce presumably unfavorable properties (e.g. immunogenicity in a human host (so-called humanization), sites of potential product heterogeneity and or instability (glycosylation, deamidation, isomerization, etc.) or to enhance some other favorable property of the molecule (e.g. solubility, stability, bioavailability, etc.). In other embodiments, changes in primary amino acid sequence can be engineered in one or more of the hypervariable loops (or CDRs) of a Canidae VH and/or VL domain obtained by active immunization. Such changes may be introduced in order to enhance antigen-binding affinity and/or specificity, or to reduce presumably unfavorable properties, e.g. immunogenicity in a human host (so-called humanization), sites of potential product heterogeneity and or instability, glycosylation, deamidation, isomerization, etc., or to enhance some other favorable property of the molecule, e.g. solubility, stability, bioavailability, etc.

Thus, in one embodiment, the recombinant antigen binding polypeptide contains at least one amino acid substitution in at least one framework or CDR region, preferably at least one framework region, of either the VH domain or the VL domain in comparison to a Canidae VH or VL domain obtained by active immunization of a species in the family Canidae with a target antigen. This particular embodiment excludes antigen-binding polypeptides containing native Canidae VH and VL domains produced by active immunization

As an alternative to “active immunization” with a target antigen (or a composition comprising the target antigen or a polynucleotide encoding it) it is also possible to make use of immune responses in diseased Canidae animals or naturally occurring immune responses within Canidae species as a source of VH and/or VL domains which can be used as components of antigen binding polypeptides with the desired antigen-binding properties. Such VH/VL domains may also be used as the starting point for engineering antigen-binding polypeptides in an analogous manner to VH/VL domains obtained by active immunization. The invention still further encompasses the use of non-immune libraries, and to antigen-binding polypeptides obtained/derived therefrom.

In other embodiments, the invention encompasses “chimeric” antibody molecules comprising VH and VL domains from Canidae (or engineered variants thereof) and one or more constant domains from a non-Canidae antibody, for example, human-encoded constant domains (or engineered variants thereof), and methods of producing said chimeric antibody molecules. The invention also extends to chimeric antigen-binding polypeptides (e.g. antibody molecules) wherein one of the VH or the VL domain is Canidae-encoded, and the other variable domain is non-Canidae (e.g. human). In such embodiments it is preferred that both the VH domain and the VL domain are obtained from the same species of Canidae. In such embodiments both the VH and the VL domain may be derived from a single animal, particularly a single animal which has been actively immunized.

In other embodiments, the invention encompasses “chimeric” antibody molecules comprising hypervariable loops or CDRs in the VH and VL domains from Canidae (or engineered variants thereof) and other regions of the VH and VL domains and optionally one or more constant domains from a non-Canidae antibody, for example, human-encoded framework regions and/or human-encoded constant domains (or engineered variants thereof), and methods of producing said chimeric antibody molecules. The invention also extends to chimeric antigen-binding polypeptides (e.g. antibody molecules) wherein one of the VH or the VL domain comprises hypervariable loops or CDRs that are Canidae-encoded, and the other hypervariable loop or CDR is non-Canidae (e.g. human), in combination with framework regions and/or constant domains that are non-Canidae (e.g. human). In such embodiments it is preferred that the CDRs of both the VH domain and the VL domain are obtained from the same species of Canidae. In such embodiments the CDRs of both the VH and the VL domain may be derived from a single animal, particularly a single animal which has been actively immunized.

As an alternative to engineering changes in the primary amino acid sequence of Canidae VH and/or VL domains, individual Canidae hypervariable loops or CDRs, or combinations thereof, can be isolated from Canidae VH/VL domains and transferred to an alternative (i.e. non-Canidae) framework, e.g. a human VH/VL framework, by CDR grafting.

As already indicated and further disclosed in the experimental section herein, the inventors found that Canidae germline and somatically mutated DNA sequences encoding both the VH and VL domains of conventional antibodies from species in the family of Canidae exhibit a high degree of sequence identity/homology with the human germline DNA sequences which encode VH and VL domains of human antibodies, over the framework regions.

The antigen binding polypeptides as disclosed herein are thus characterized by a high degree of amino acid sequence homology with VH and VL domains of human antibodies.

In some embodiments, the antigen binding polypeptides as disclosed herein or produced by any of the methods disclosed herein contain at least one amino acid substitution in at least one framework region (FR) of either the VH domain or the VL domain, in comparison to a Canidae VH or VL domain obtained by active immunization of a species in the family Canidae or in comparison to a VH or VL domain encoded by a VH or VL gene of a species in the family Canidae.

In some embodiments, the antigen binding polypeptide comprises a VH domain wherein the VH domain contains at least one amino acid sequence mismatch across the framework regions FR1, FR2, FR3 and FR4 in comparison with VH domains encoded by human germline or somatically mutated VH genes. In these embodiments, polypeptides comprising a VH domain, or both VH and VL domains, in which the framework region has an entirely human sequence are thus excluded.

In some embodiments the VH domain of the antigen-binding polypeptide according to the invention may exhibit an amino acid sequence identity or sequence homology of 80% or greater with one or more human VH domains across the framework regions FR1, FR2, FR3 and FR4. In other embodiments the amino acid sequence identity or sequence homology between the VH domain of the polypeptide of the invention and one or more human VH domains may be 85% or greater, 90% or greater, 95% or greater, 97% or greater, or up to 99% or even 100%, of course with the proviso that at least one hypervariable loop or CDR is obtained from Canidae, i.e. has an amino acid sequence which is identical (or substantially identical) to the amino acid sequence of a hypervariable loop or CDR encoded by a Canidae VH gene.

In some embodiments, the antigen biding polypeptide thus comprises a VH domain and a VL domain wherein one or more; preferably all, framework regions FR1, FR2 FR3 and FR4 in the VH and VL domain are from a human antibody. In some further embodiments, the antigen binding polypeptide contains at least one amino acid mutation, preferably at least one amino acid substitution, in at least one of the framework regions of either the VH domain or the VL domain or both in comparison to the human VH or VL domain of the human antibody.

In some embodiments, the antigen binding polypeptide comprises a VL domain wherein the VL domain contains at least one amino acid sequence mismatch across the framework regions FR1, FR2, FR3 and FR4 in comparison with VH domains encoded by human germline or somatically mutated VL genes. In these embodiments, polypeptides comprising a VL domain, or both VH and VL domains, in which the framework region has an entirely human sequence are thus excluded.

In some embodiments the VL domain of the antigen-binding polypeptide according to the invention may exhibit an amino acid sequence identity or sequence homology of 80% or greater with one or more human VL domains across the framework regions FR1, FR2, FR3 and FR4. In other embodiments the amino acid sequence identity or sequence homology between the VL domain of the polypeptide of the invention and one or more human VH domains may be 85% or greater, 90% or greater, 95% or greater, 97% or greater, or up to 99% or even 100%, of course with the proviso that at least one hypervariable loop or CDR is obtained from Canidae, i.e. has an amino acid sequence which is identical (or substantially identical) to the amino acid sequence of a hypervariable loop or CDR encoded by a Canidae VL gene.

The antigen binding polypeptide of the invention or the antigen binding polypeptide produced by any of the methods discloses herein may comprise a “fully human” VH or VL domain, provided that only one fully human variable domain is present, and then in combination with a variable domain comprising at least one hypervariable loop or CDR obtained from Canidae.

Representative alignments of Canidae (e.g. dog, wolf, fox, dingo) and human germline sequences included in the accompanying examples reveal that the VH and VL domains exhibit a remarkably high sequence homology to their human counterparts. From these examples it can be concluded that typically few amino acid residues present in the framework regions of a VH or VL domain differ in a given position from the closest human germline-encoded sequences. Given that there are no structural limitations associated with those positions, humanization by site directed mutagenesis is expected to be straightforward.

Therefore, in a particular embodiment, the antigen binding polypeptides of the invention or produced by any of the methods disclosed herein may comprise VH and/or VL domains of conventional Canidae antibodies, for example conventional Canidae antibodies obtained (obtainable) by active immunization of a species of the family Canidae with a target antigen (or polynucleotide encoding the target antigen), wherein said VH and VL domains have been (independently) engineered to introduce a total of between 1 and 10, i.e. 1, 2, 3, 4, 5, 6, 7, 8, 9 or 10 amino acid substitutions across the framework regions FR1, FR2, FR3 and FR4 in either one or both of the VH domain and the VL domain. Such amino acid substitutions may include (but are not limited to) substitutions which result in “humanization”, by replacing mis-matched amino acid residues in a starting Canidae VH or VL domain with the equivalent residue found in a human germline-encoded VH or VL domain. It is also possible to independently make amino acid substitutions in the hypervariable loops (CDRs) of said Canidae-derived VH and VL domains, and such variants may form part of the present invention. References herein to “amino acid substitutions” include substitutions in which a naturally occurring amino acid is replaced with a non-natural amino acid, or an amino acid subjected to post-translational modification.

Human germline best matching family member hit (HuGermHit) that has the highest degree of sequence identity with the Canidae variable region of interest is chosen for scoring the sequence identity. Canidae VGenes and HuGermHit may or may not share the same length of HCDR1 (also referred to as CDR-H1) and HCDR2 (also referred to as CDR-H2) for heavy chain and may or may not share the same length of LCDR1 (also referred to as CDR-L1) and LCDR2 (also referred to as CDR-L2) of the light chains.

Antibody-Extractor™ Germaligner alignment algorithm was used to compute the percentage frameworks sequence identity between Canidae VH and VL Frameworks 1, 2 and 3 amino acid sequences and the HuGermHit or the global identity between Canidae VH and VL including Frameworks 1,2 and 3 and CDR1 and CDR2 and the HuGermHit Frameworks 1,2 and 3 and CDR1 and CDR2.

Canidae VH and VL genes with respectively the same length of HCDR1 and HCDR2 for heavy chain and the same length of LCDR1 and LCDR2 for light chains are considered better human-like families than Canidae VH and VL with different HCDR1 and HCDR2, LCDR1 and LCDR2. However, these sequences are also considered as true human-like hits as part they can be used as template in Humanization processes.

Despite the high sequence homology between Canidae and human across the framework regions of the VH and VL domains, it is nevertheless possible to distinguish Canidae-encoded VL or VH from human-encoded VL or VH using the Antibody-Extractor™ platform or manual analysis based on Canidae conserved residues scoring.

It is well established in the art that although the primary amino acid sequences of hypervariable loops (CDRs) present in both VH domains and VL domains encoded by the human germline are, by definition, highly variable, all hypervariable loops, except CDR H3 of the VH domain, adopt only a few distinct structural conformations, termed canonical folds (Chothia et al., J. Mol. Biol. 196:901-917 (1987); Tramontano et al. Proteins 6:382-94 (1989)), which depend on both the length of the hypervariable loop and presence of the so-called canonical amino acid residues (Chothia et al., J. Mol. Biol. 196:901-917 (1987)). Actual canonical structures of the hypervariable loops in intact VH or VL domains can be determined by structural analysis (e.g. X-ray crystallography), but it is also possible to predict canonical structure on the basis of key amino acid residues which are characteristic of a particular structure (discussed further below). In essence, the specific pattern of residues that determines each canonical structure forms a “signature” which enables the canonical structure to be recognized in hypervariable loops of a VH or VL domain of unknown structure; canonical structures can therefore be predicted on the basis of primary amino acid sequence alone.

Based on the identity between human and Canidae heavy chain CDR1 (CDR-H1) and CDR2 (CDR-H2) and light chain CDR1 (CDR-L1) and CDR2 (CDR-L2) the present inventors predict that the hypervariable loops of Canidae VH and VL domains (with the exception of H3 in the VH domain and sometimes also L3 in the VL domain) also adopt canonical fold structures which are substantially identical or highly similar to canonical fold structures adopted by the hypervariable loops of human antibodies.

In the case of the VH domain or VL (Kappa or Lambda) domain derived from Canidae may be considered as having a canonical fold structure identical or highly similar to a canonical fold structure known to occur in human antibodies if at least the first, and preferable both, of the following criteria are fulfilled:
1. An identical length, determined by the number of residues, to the closest matching human heavy chain VGene.
2. At least 33% identity, preferably at least 50% identity with the corresponding human CDR1 and CDR2.

Thus, in some embodiments, the antigen binding polypeptide as disclosed herein or produced by any of the methods disclosed herein is characterized in that at least one hypervariable loop or CDR in either the VH domain or the VL domain is obtained from a VH or VL domain of a species in the family of Canidae and exhibits a predicted or actual canonical fold structure which is substantially identical to a canonical fold structure which occurs in human antibodies. In some embodiments, the hypervariable loop H1 (CDR-H1) and hypervariable loop H2 (CDR-H2) are each obtained from a VH domain of a species in the family Canidae and each exhibit a predicted or actual canonical fold structure which is substantially identical to a canonical fold structure which occurs in human antibodies and wherein hypervariable loop H1 and hypervariable loop H2 in the VH domain form a combination of predicted or actual canonical fold structures which is identical to a combination of canonical fold structures known to occur in human germline VH domain.

In some embodiments, the antigen binding polypeptide exhibits a sequence identity of 80% or greater, preferably of 85% or greater, more preferably of 90% or greater, even more preferably of 95% or greater with a human VH domain across the framework regions FR1, FR2, FR3 and FR4, and wherein hypervariable loop H1 and hypervariable loop H2 form a combination of predicted or actual canonical fold structures which is the same as a canonical fold combination known to occur naturally in the same human VH domain.

In some embodiments, the hypervariable loop L1 (CDR-L1) and hypervariable loop L2 (CDR-L2) in the VL domain are each obtained from a VH or VL domain of a species in the family Canidae and each exhibit a predicted or actual canonical fold structure which is substantially identical to a canonical fold structure which occurs in human antibodies and wherein hypervariable loop L1 and hypervariable loop L2 in the VL domain form a combination of predicted or actual canonical fold structures which is identical to a combination of canonical fold structures known to occur in human germline VL domain.

Thus, in a further embodiment H1 (HCDR1 or CDR-H1) and H2 (HCDR2 or CDR-H2), and L1 (LCDR1 or CDR-L1) and L2 (LCDR2 or CDR-L2) in the VH and VL domains of the antigen binding polypeptide of the invention are obtained from a VH and VL domain of a Canidae species, yet form a combination of predicted or actual canonical fold structures which is identical to a combination of canonical fold structures known to occur in a human germline or somatically mutated VH and VL domain.

It is preferred that the VH and VL domains of the antigen binding polypeptide of the invention exhibit both high sequence identity/sequence homology with human VH and VL, and also that the hypervariable loops in the VH and VL domain show high identity in HCDR1 and HCDR2, and LCDR1 and LCDR2, and structural homology with human VH and VL.

It may be advantageous for HCDR1 and HCDR2 in the VH domain and LCDR1 and LCDR2 of the antigen binding polypeptide according to the invention, and the combination thereof, to be “correct” for the human VH and VL germline sequence which represents the closest match with the VH and VL domain of the antigen binding polypeptide of the invention in terms of overall primary amino acid sequence identity.

Thus, in one embodiment the VH domain of the antigen binding polypeptide of the invention may exhibit a sequence identity or sequence homology of 75% or greater, 80% or greater, 85% or greater, 90% or greater, 95% or greater, 97% or greater, or up to 99% or even 100% with a human VH domain and human VL domain across the framework regions FR1, FR2, FR3 and FR4, and in addition HCDR1 and HCDR2, and LCDR1 and LCDR2, in the same antigen binding polypeptide are obtained from a VH domain and a VL domain of a Canidae species, but form a combination of predicted or actual canonical fold structures which is the same as a canonical fold combination known to occur naturally in the same human VH domain and human VL domain.

It is, of course, envisaged that VH domains exhibiting high sequence identity/sequence homology with human VH, and also structural homology with hypervariable loops of human VH will be combined with VL domains exhibiting high sequence identity/sequence homology with human VL, and also structural homology with hypervariable loops of human VL to provide antigen binding polypeptides containing (Canidae-derived) VH/VL pairings with maximal sequence and structural homology to human-encoded VH/VL pairings. A particular advantage of the Canidae platform provided by the invention is that both the VH domain and the VL domain exhibit high sequence homology with the variable domains of human antibodies.

The antigen binding polypeptides as disclosed herein can take various different embodiments, provided that both a VH domain and a VL domain are present. Thus, in non-limiting embodiments the antigen binding polypeptide may be an immunoglobulin, an antibody or antibody fragment. The term “antibody” herein is used in the broadest sense and encompasses, but is not limited to, monoclonal antibodies (including full length monoclonal antibodies), polyclonal antibodies, multi-specific antibodies (e.g., bispecific antibodies), so long as they exhibit the appropriate specificity for a target antigen.

In preferred embodiments, the antigen binding polypeptides are monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” comprise a portion of a full length antibody, generally the antigen binding or variable domain thereof. Examples of antibody fragments include Fab, Fab1, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1 126-36 (2005), the contents of which are incorporated herein by reference).

The antigen binding polypeptides of the invention or produced by any methods as disclosed herein are not naturally occurring human antibodies, specifically human autoantibodies, due to the requirement for at least one hypervariable loop (or CDR) from Canidae. By “naturally occurring” human antibody is meant an antibody which is naturally expressed within a human subject. Antigen binding polypeptides having an amino acid sequence which is 100% identical to the amino acid sequence of a naturally occurring human antibody, or a fragment thereof, which natural antibody or fragment is not chimeric and has not been subject to any engineered changes in amino acid sequence (excluding somatic mutations) are excluded from the scope of the invention.

In non-limiting embodiments, antibodies and antibody fragments according to the invention or produced by any methods as disclosed herein may comprise CH1 domains and/or CL domains, the amino acid sequence of which is fully or substantially human. Where the antigen binding polypeptide of the invention is an antibody intended for human therapeutic use, it is typical for the entire constant region of the antibody, or at least a part thereof, to have fully or substantially human amino acid sequence. Therefore, an antibody of the invention must comprise VH and VL domains, at least one of which includes at least one hypervariable loop derived from Canidae, but one or more or any combination of the CH1 domain, hinge region, CH2 domain, CH3 domain and CL domain (and CH4 domain if present) may be fully or substantially human with respect to its amino acid sequence.

Advantageously, the CH1 domain, hinge region, CH2 domain, CH3 domain and CL domain (and CH4 domain if present) may all have fully or substantially human amino acid sequence. In the context of the constant region of a humanized or chimeric antibody, or an antibody fragment, the term “substantially human” refers to an amino acid sequence identity of at least 90%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence.

Advantageously, the framework regions (FR1, FR2, FR3 and FR4) may all have fully or substantially human amino acid sequence. In the context of the framework region of a humanized or chimeric antibody, or an antibody fragment, the term “substantially human” refers to an amino acid sequence identity of at least 90%, or at least 95%, or at least 97%, or at least 99% with a human framework region. The term “human amino acid sequence” in this context refers to an amino acid sequence which is encoded by a human immunoglobulin gene, which includes germline, rearranged and somatically mutated genes. The invention also contemplates polypeptides comprising framework region of “human” sequence which have been altered, by one or more amino acid additions, deletions or substitutions with respect to the human sequence.

As discussed elsewhere herein, it is contemplated that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites). Depending on the intended use of the antibody, it may be desirable to modify the antibody of the invention with respect to its binding properties to Fc receptors, for example to modulate effector function. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp. Med. 176:1 191-1195 (1992) and Shopes, B. J. Immunol. 148:2918-2922 (1992). Alternatively, an antibody can be engineered which has dual Fc regions and may thereby have enhanced complement lysis and ADCC capabilities. See Stevenson et al., Anti-Cancer Drug Design 3:219-230 (1989). The invention also contemplates immunoconjugates comprising an antibody as described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate). Fc regions may also be engineered for half-life extension.

The antigen binding polypeptides disclosed herein are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. Canidae and human. Thus, the invention can, in certain embodiments, encompass chimeric antibodies, and in particular chimeric antibodies in which the hypervariable loops or CDRs in the VH and VL domains are of fully Canidae sequence and the remainder of the antibody is of fully human sequence. In preferred embodiments the invention also encompasses “humanized” or “germlined” Canidae antibodies, and Canidae/human chimeric antibodies, in which the VH and VL domains contain one or more amino acid substitutions in the framework regions in comparison to Canidae VH and VL domains obtained by active immunization. Such “humanization” increases the % sequence identity with human germline VH or VL domains by replacing mis-matched amino acid residues in a starting Canidae VH or VL domain with the equivalent residue found in a human germline-encoded VH or VL domain.

Also disclosed herein are CDR-grafted antibodies in which CDRs (or hypervariable loops) derived from a Canidae antibody, for example an Canidae antibody raised by active immunization with a target antigen, or otherwise encoded by a Canidae gene, are grafted onto a human VH and VL framework, with the remainder of the antibody also being of fully human origin. However, given the high degree of amino acid sequence homology and structural homology they have observed between Canidae and human immunoglobulins, the inventors anticipate that in the majority of cases it will be possible to achieve the levels of human homology required for in vivo therapeutic use via “humanization” of the framework regions of Canidae-derived VH and VL domains without the need for CDR grafting or via CDR grafting on to limited number of backbone sequences without the need for veneering (also see Almagro et al, Frontiers in Bioscience 13: 1619-1633 (2008), the contents of which are incorporated herein by reference).

Humanized, chimeric and CDR-grafted antibodies according to the invention or produced according to any of the methods disclosed herein, particularly antibodies comprising hypervariable loops derived from active immunization of Canidae with a target antigen, can be readily produced using conventional recombinant DNA manipulation and expression techniques, making use of prokaryotic and eukaryotic host cells engineered to produce the polypeptide of interest and including but not limited to bacterial cells, yeast cells, mammalian cells, insect cells, plant cells.

The invention also encompasses antigen binding polypeptides wherein either one or other of the VH or VL domain is obtained from Canidae, or contains at least one CDR or hypervariable region derived from Canidae, and the “other” variable domain has non-Canidae, e.g. human, amino acid sequence. Thus, it is contemplated to pair a Canidae VH domain with a human VL domain, or to pair a human VH domain with a Canidae VL domain. Such pairings may increase the available antigen-binding repertoire from which to select high affinity binders with the desired antigen binding properties.

As detailed herein, the invention provides chimeric monoclonal antigen binding polypeptides characterized in that they comprise a VH domain and a VL domain and optionally further comprising one or more constant domains, wherein at least one hypervariable loop or complementary determining region (CDR) in the VH domain and/or the VL domain is obtained from a VH or VL domain of a species in the family Canidae and wherein one or more of the framework regions in the VH and VL domains are from a human antibody, and optionally wherein one or more constant domains is human. In further embodiments, the FR1, FR2, FR3 and FR4 framework regions of the VH and VL domains are of human origin.

In particular embodiments, the chimeric monoclonal antibody of the invention comprises one or more underlined amino acids of the Dog C1 sequence (heavy chain, Table 1) at a corresponding position in a CDR or hypervariable loop of said VH domain.

In particular embodiments, the chimeric monoclonal antibody of the invention comprises at least 1, 2, 3, 4, 5, 6, 7, 8, 9, 10, 11 12, 13, 14, 15 or 16, or any one of 17 to 43, preferably at least 2, more preferably at least 5 or all of the underlined amino acids in the Dog C1 sequence of Table 1, at corresponding positions in a VH domain thereof.

In alternative embodiments, the chimeric monoclonal antibody of the invention comprises at least 1, 2, 3, 4, 5, 6, 7, 8, 9, 10, 11 12, 13, 14, 15 or 16, or any one of 17 to 43, preferably at least 2, more preferably at least 5 or all of the amino acids specific for dog based on the consensus sequence of the heavy chain in Table 2, i.e. the amino acids identified in the dog consensus sequence but not in the human consensus sequence of the heavy chain.

In particular embodiments, the chimeric monoclonal antibody of the invention comprises one or more underlined amino acids of the Dog C2 sequence (lambda chain, Table 1) at a corresponding position in a CDR or hypervariable loop of said VL domain.

In particular embodiments, the chimeric monoclonal antibody of the invention comprises 1, 2, 3, 4, 5, 6, 7, 8, 9, 10, 11 12, 13, 14, 15, 16, or 17, preferably at least 2, more preferably at least 5 or all of the underlined amino acids in the Dog C2 sequence of Table 1, at corresponding positions in a VL domain thereof.

In alternative embodiments, the chimeric monoclonal antibody of the invention comprises at least 1, 2, 3, 4, 5, 6, 7, 8, 9, 10, 11 12, 13, 14, 15, 16 or 17, preferably at least 2, more preferably at least 5 or all of the amino acids specific for dog based on the consensus sequence of the lambda chain in Table 2, i.e. the amino acids identified in the dog consensus sequence but not in the human consensus sequence of the lambda chain.

In particular embodiments, the chimeric monoclonal antibody of the invention comprises one or more underlined amino acids of the Dog C3 sequence (kappa chain, Table 1) at a corresponding position in a CDR or hypervariable loop of said VL domain.

In particular embodiments, the chimeric monoclonal antibody of the invention comprises 1, 2, 3, 4, 5, 6, 7, 8, 9, 10, 11 12, 13, 14, 15, 16, or any one of 17 to 35, preferably at least 2, more preferably at least 5 or all of the underlined amino acids in the Dog C3 sequence of Table 1, at corresponding positions in a VL domain thereof.

In alternative embodiments, the chimeric monoclonal antibody of the invention comprises the chimeric monoclonal antibody of the invention comprises 1, 2, 3, 4, 5, 6, 7, 8, 9, 10, 11 12, 13, 14, 15, 16, or any one of 17 to 35, preferably at least 2, more preferably at least 5 or all of the amino acids specific for dog based on the consensus sequence of the kappa chain in Table 2, i.e. the amino acids identified in the dog consensus sequence but not in the human consensus sequence of the kappa chain.

Some embodiments provide antigen binding polypeptides wherein the hypervariable loop(s) or CDR(s) of the VH domain and/or the VL domain are obtained from Canidae, but wherein at least one of said (Canidae-derived) hypervariable loops or CDRS has been engineered to include one or more amino acid substitutions, additions or deletions relative to the Canidae-encoded sequence. Such changes include “humanization” of the hypervariable loops/CDRs. Canidae-derived HVs/CDRs which have been engineered in this manner may still exhibit an amino acid sequence which is “substantially identical” to the amino acid sequence of a Canidae-encoded HV/CDR. In this context, “substantial identity” may permit no more than one, or no more than two amino acid sequence mis-matches with the Canidae-encoded HV/CDR.

Antibodies according to the invention or produced by any of the methods disclosed herein may be of any isotype. Antibodies intended for human therapeutic use will typically be of the IgA, IgD, IgE IgG, IgM type, often of the IgG type, in which case they can belong to any of the four sub-classes IgGI, IgG2a and b, IgG3 or IgG4. Within each of these sub-classes it is permitted to make one or more amino acid substitutions, insertions or deletions within the Fc portion, or to make other structural modifications, for example to enhance or reduce Fc-dependent functionalities.

In all aspects and embodiments disclosed herein, the species in the family Canidae is selected from the group consisting of domestic dogs, wolves, coyotes, foxes, jackals, dingoes and other dog-like mammals and any crossings thereof. In a preferred embodiment, the species of the family Canidae is a dog or dog-like mammal.

Antigen binding polypeptides according to the embodiments disclosed herein may be immunoreactive with a target antigen or specifically bind to a target antigen. Preferably the antigen is a non-Canidae antigen. In some embodiments the antigen is a human antigen. In some embodiments, the antigen is a viral antigen or a bacterial antigen.

Antigen binding polypeptides disclosed herein may be useful in a wide range of applications, both in research and in the diagnosis and/or treatment of diseases. In one aspect, the antigen binding polypeptides as disclosed herein are for use in medicine. In some embodiments, the antigen binding polypeptides as disclosed herein are for use in the diagnosis of a disease. In some embodiments, the antigen binding polypeptides as disclosed herein are for use in the treatment of a disease. In some embodiments, the antigen binding polypeptides as disclosed herein are for use in the diagnosis and treatment of a disease.

Because of the high degree of amino acid sequence identity with the VH and VL domains of natural human antibodies, and the high degree of structural homology (specifically the correct combinations of canonical folds as are found in human antibodies) the antigen binding polypeptides of the invention, particularly in the form of monoclonal antibodies, will thus find particular utility as human therapeutic agents.

The invention provides a platform for production of antigen binding polypeptides, and specifically monoclonal antibodies, against a wide range of antigens and in its broadest aspect the invention is not intended to be limited with respect to the exact identity of the target antigen, nor indeed the specificity or affinity of binding to the target antigen. However, in particular, non-limiting, embodiments the target antigen may be a non-Canidae antigen, a bacterial antigen, a viral antigen or a human antigen. In a preferred embodiment the target antigen may be an antigen of particular therapeutic importance. The term “target of therapeutic importance” refers to a target involved in formation, onset, progression, mediation of human or animal diseases or of the effects related to the respective disease. Included within this definition are targets wherein the expression levels and/or activity of the target are modulated by antibody binding (e.g. receptors whose activity may be modulated by binding of agonist or antagonist antibodies), and targets wherein the activity and/or expression of the target has a direct or indirect impact on a disease.

By way of example, “human antigens” may include naturally occurring human polypeptides (proteins) which function as receptors, receptor ligands, cell-signalling molecules, hormones, cytokines or cytokine receptors, neurotransmitters, etc. By “naturally occurring” is meant that the polypeptide is expressed within the human body, at any stage if its development, including polypeptides expressed by the human body during the course of a disease.

Non-limiting embodiments of the antigen binding polypeptide of the invention include the following:

A chimeric antigen binding polypeptide comprising a VH domain and a VL domain, wherein at least one hypervariable loop or complementarity determining region (CDR) in the VH domain or the VL domain is obtained from a VH or VL domain of a species in the family Canidae. In a particular embodiment at least one of the CDRs of the VH domain and at least one of the CDRs of the VL domain are obtained from a species in the family Canidae, preferably a dog. In a further embodiment, all three CDRs of the VH domain and all three CDRs of the VL domain are obtained from a species in the family Canidae, preferably a dog. The chimeric antigen binding polypeptide further comprises one or more framework regions in the VH and VL domain of a human antibody; and optionally one or more constant domains of a human antibody. In some embodiments, the chimeric antigen binding polypeptide comprises the framework region FR1, FR2, FR3 and FR4 derived from a human antibody.

A recombinant expressed antigen binding polypeptide is provided, said polypeptide comprising a VH domain and a VL domain, wherein at least one hypervariable loop or complementarity determining region (CDR) in the VH domain or the VL domain is obtained from a VH or VL domain of a species in the family Canidae. In a particular embodiment at least one of the CDRs of the VH domain and at least one of the CDRs of the VL domain are obtained from a species in the family Canidae, preferably from a dog. In a further embodiment, all three CDRs of the VH domain and all three CDRs of the VL domain are obtained from a species in the family Canidae, preferably a dog. The recombinant expressed antigen binding polypeptide further comprises one or more framework regions in the VH and VL domain of a human antibody; and optionally one or more constant domains of a human antibody. In some embodiments, the recombinant expressed antigen binding polypeptide comprises the framework regions FR1, FR2, FR3 and FR4 derived from a human antibody.

A monoclonal antibody is provided, said antibody comprising a VH domain and a VL domain, wherein at least one hypervariable loop or complementarity determining region (CDR) in the VH domain or the VL domain is obtained from a VH or VL domain of a species in the family Canidae. In a particular embodiment at least one of the CDRs of the VH domain and at least one of the CDRs of the VL domain are obtained from a species in the family Canidae, preferably from a dog. In a further embodiment, all three CDRs of the VH domain and all three CDRs of the VL domain are obtained from a species in the family Canidae, preferably a dog. The monoclonal antibody further comprises one or more framework regions in the VH and VL domain of a human antibody; and optionally one or more constant domains of a human antibody. In some embodiments, the monoclonal antibody comprises framework regions FR1, FR2, FR3 and FR4 derived from a human antibody.

An antigen binding polypeptide is provided comprising a VH domain and a VL domain, wherein at least one hypervariable loop or complementarity determining region (CDR) in the VH domain or the VL domain is obtained from a VH or VL domain of a species in the family Canidae and wherein said antigen binding polypeptide is immunoreactive with a target antigen of therapeutic or diagnostic importance. In a particular embodiment at least one of the CDRs of the VH domain and at least one of the CDRs of the VL domain are obtained from a species in the family Canidae, preferably from a dog. In a further embodiment, all three CDRs of the VH domain and all three CDRs of the VL domain are obtained from a species in the family Canidae, preferably a dog. The antigen binding polypeptide further comprises one or more framework regions in the VH and VL domain of a human antibody; and optionally one or more constant domains of a human antibody. In some embodiments, the antigen binding polypeptide comprises the framework regions FR1, FR2, FR3, and FR4 derived from a human antibody.

Also provided is a chimeric antigen binding polypeptide comprising a VH domain of a conventional antibody of a Canidae (in particular Dog), or a VL domain of a conventional antibody of a Canidae (in particular Dog) and one or more constant domains of a human antibody. In a particular embodiment all CDRs of the VH domain and all CDRs of the VL domain are obtained from Canidae, in particular from a dog. The chimeric antigen binding polypeptide further comprises one or more framework regions in the VH and VL domain of a human antibody; and optionally one or more constant domains of a human antibody. In some embodiments, the antigen binding polypeptide comprises the framework regions FR1, FR2, FR3, and FR4 derived from a human antibody. Also provided is a chimeric antigen binding polypeptide immunoreactive with a target antigen of therapeutic or diagnostic importance, which antigen binding polypeptide comprises a VH domain of a conventional antibody of a Canidae (in particular Dog), or a VL domain of a conventional antibody of a Canidae (in particular Dog), wherein the other VH or VL domain comprises one or more framework regions of a human antibody, and wherein the polypeptide further one or more constant domains of a human antibody.

Also provided is a chimeric antibody comprising a VH domain of a conventional antibody of a Canidae (in particular Dog), or a VL domain of a conventional antibody of a Canidae (in particular Dog) and the constant domains of a human antibody of an isotype selected from the group consisting of: IgG, IgM, IgD, IgE and IgA, wherein the other VH or VL domain comprises one or more framework regions of a human antibody.

Further provided is a chimeric antigen binding polypeptide immunoreactive with a target antigen of therapeutic or diagnostic importance, which antigen binding polypeptide comprises a VH domain of a conventional antibody of a Canidae (in particular Dog), or a VL domain of a conventional antibody of a Canidae (in particular Dog) and the constant domains of a human antibody of an isotype selected from the group consisting of: IgG, IgM, IgD, IgE, IgA, wherein the other VH or VL domain comprises one or more framework regions of human VH and VL domains.

In particular embodiments of the foregoing, at least the hypervariable loops of both the VH and the VL domain may be from the same species of Canidae, in particular a dog, and may even be from the same animal within this species, for example a single animal which has been actively immunized. In particular, both the VH domain and the VL domain may be obtained from a single actively immunized Canidae. However, it is not excluded that the VH and VL domain may be obtained from different animals, or non-immune libraries.

In the foregoing embodiments, the terms “VH domain of a conventional antibody of a Canidae” and “VL domain of a conventional antibody of a Canidae” are intended to encompass variants which have been engineered to introduce one or more changes in amino acid sequence, such as variants which have been “humanized” or “germlined” in one or more framework regions, as described elsewhere herein, and also encompass the products of synthetic (e.g. codon-optimized) genes, as described elsewhere herein.

The invention also provides a polynucleotide molecule encoding the antigen binding polypeptide disclosed herein, an expression vector containing a nucleotide sequence encoding the antigen binding polypeptide disclosed herein operably linked to regulatory sequences which permit expression of the antigen binding polypeptide in a host cell or cell-free expression system, and a host cell or cell-free expression system containing this expression vector.

Polynucleotide molecules encoding the antigen binding polypeptide of the invention include, for example, recombinant DNA molecules.

The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated.

For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). A n isolated polynucleotide (either DNA or RNA) may further represent a molecule produced directly by biological or synthetic means and separated from other components present during its production. For recombinant production of an antigen binding polypeptide according to the invention, a recombinant polynucleotide encoding it may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 851 10503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antigen binding polypeptide according to the invention has been introduced are explicitly excluded from the scope of the invention.

In an important aspect, the invention also provides a method of producing a recombinant antigen binding polypeptide which comprises culturing a host cell (or cell free expression system) containing polynucleotide (e.g. an expression vector) encoding the recombinant antigen binding polypeptide under conditions which permit expression of the antigen binding polypeptide, and recovering the expressed antigen binding polypeptide. This recombinant expression process can be used for large scale production of antigen binding polypeptides according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

Related aspects of the invention relate to test kits, including diagnostic kits etc. comprising an antigen binding polypeptide according to the invention, and also pharmaceutical formulations comprising an antigen binding polypeptide according to the invention.

Where the antigen binding polypeptide is intended for diagnostic use, for example where the antigen binding polypeptide is specific for an antigen which is a biomarker of a disease state or a disease susceptibility, then it may be convenient to supply the antigen binding polypeptide as a component of a test kit. Diagnostic tests typically take the form of standard immunoassays, such as ELISA, radioimmunoassay, Elispot, etc. The components of such a test kit may vary depending on the nature of the test or assay it is intended to carry out using the antigen binding polypeptide of the invention, but will typically include additional reagents required to carry out an immunoassay using the antigen binding polypeptide of the invention. Antigen binding polypeptides for use as diagnostic reagents may carry a revealing label, such as for example a fluorescent moiety, enzymatic label, or radiolabel.

Antigen binding polypeptides intended for in vivo therapeutic use are typically formulated into pharmaceutical dosage forms, together with one or more pharmaceutically acceptable diluents, carriers or excipients (Remington's Pharmaceutical Sciences, 16th edition., Osol, A. Ed. 1980). Antigen binding polypeptides according to the invention are typically formulated as sterile aqueous solutions, to be administered intravenously, or by intramuscular, intraperitoneal, intra-cerebrospinal, intratumoral, oral, peritumoral, subcutaneous, intra-synovial, intrathecal, topical, sublingual or inhalation routes, to a mammalian subject, typically a human patient, in need thereof. For the prevention or treatment of disease, the appropriate dosage of antigen binding polypeptide will depend on the type of disease to be treated, the severity and clinical course of the disease, plus the patient's age, weight and clinical history, and will be determined by the judgement of the attending physician.

A related aspect of the present application relates to methods or processes for the production of high affinity antigen binding polypeptides, and specifically monoclonal antibodies, against a target antigen of interest.

Accordingly, the invention provides a process for preparing an antigen binding polypeptide immunoreactive with a target antigen, preferably a chimeric monoclonal antibody, said process comprising:
(a) determining the nucleotide sequence encoding at least one hypervariable loop or complementarity determining region (CDR) of the VH and/or the VL domain of a Canidae conventional antibody immunoreactive with said target antigen; and
(b) expressing an antigen binding polypeptide immunoreactive with said target antigen, said antigen binding polypeptide comprising a VH and a VL domain, wherein at least one hypervariable loop or complementarity determining region (CDR) of the VH domain or the VL domain has an amino acid sequence encoded by the nucleotide sequence determined in part (a).

In some embodiments, the Canidae conventional antibody of part (a) is obtained after immunizing a species in the family Canidae, thereby raising a conventional antibody which is immunoreactive with said target antigen.

In some embodiments, step (a) comprises determining the nucleotide sequence encoding the VH and/or the VL domain of a Canidae conventional antibody immunoreactive with said target antigen; and step (b) comprises expressing an antigen binding polypeptide immunoreactive with said target antigen, said antigen binding polypeptide comprising a VH and a VL domain, wherein at least one of the VH domain or the VL domain has an amino acid sequence encoded by the nucleotide sequence determined in part (a).

In some embodiments, the antigen binding polypeptide expressed in step (b) comprises at least one constant domain of a non-Canidae antibody, preferably a human antibody.

In some embodiments, the antigen binding polypeptide expressed in part (b) is not identical to the Canidae conventional antibody of part (a).

In one non-limiting embodiment, the invention provides a process for preparing a recombinant antigen binding polypeptide, preferably a chimeric monoclonal antibody, that is immunoreactive with or specifically binds to a target antigen, said an antigen binding polypeptide comprising a VH domain and a VL domain, wherein at least one hypervariable loop or complementarity determining region (CDR) in the VH domain or the VL domain is obtained from a species in the family Canidae, said process comprising the steps of:
(a) isolating Canidae nucleic acid encoding at least one hypervariable loop or complementarity determining region (CDR) of the VH and/or the VL domain of a Canidae conventional antibody immunoreactive with said target antigen;
(b) preparing a recombinant polynucleotide comprising a nucleotide sequence encoding hypervariable loop(s) or complementarity determining region(s) having amino acid sequence identical to the hypervariable loop(s) or complementarity determining region(s) encoded by the nucleic acid isolated in step (a), which recombinant polynucleotide encodes an antigen binding polypeptide comprising a VH domain and a VL domain that is immunoreactive with or specifically binds to said target antigen; and
(c) expressing said antigen binding polypeptide from the recombinant polynucleotide of step (b). In one embodiment, the antigen binding polypeptide expressed in part (c) is not identical to the Canidae conventional antibody of part (a).

In some embodiments, the process for a recombinant antigen binding polypeptide is characterized in that step (a) comprises isolating Canidae nucleic acid encoding the VH domain and/or the VL domain of said antibody and altering the sequence of said nucleic acid such that it encodes a VH domain and/or a VL domain with one or more amino acid substitutions, deletions or additions.

In some embodiments, the recombinant polynucleotide prepared in step (b) additionally comprises a nucleotide sequence encoding one or more constant domains of a non-Canidae antibody, preferably a human antibody.

In some embodiments, the antigen binding polypeptide prepared any of the processes disclosed herein is not identical to the Canidae conventional antibody of part (a) of these processes.

In some embodiments, an antigen binding polypeptide is provided that is obtainable by a process according to any of the embodiments as disclosed herein.

The foregoing methods may be referred to herein as “general processes” for preparing antigen binding polypeptides.

The first step of either process may involve active immunization of a species in the family Canidae in order to elicit an immune response against the target antigen, thereby raising Canidae conventional antibodies immunoreactive with the target antigen. Protocols for immunization of Canidae are described in the accompanying examples. The antigen preparation used for immunization may be a purified form of the target antigen, for example recombinantly expressed polypeptide, or an immunogenic fragment thereof. However, it is also possible to immunize with crude preparations of the antigen, such as like isolated cells or tissue preparations expressing or encoding the target antigen, cell lysates, cell supernatants or fractions such as cell membranes, etc., or with a polynucleotide encoding said target antigen (a DNA immunization).

The process will typically involve immunization of animals of a Canidae species, such as animals selected from domestic dogs, wolves, coyotes, foxes, jackals, dingoes and other dog-like mammals and any crossings thereof, and advantageously these animals will belong to an outbred population. However, it is also contemplated to use transgenic animals (e.g. transgenic mice) containing the Canidae conventional Ig locus, or at least a portion thereof.

A topic of increasing interest seems to be the difference between the complementarity determining regions (CDRs) of in vivo and in vitro generated antibodies. The inventors surmise that the in vivo selection has a favorable impact on the immunogenicity, functionality, stability and therefore improved manufacturability of the resulting antibodies, whilst synthetic CDRs generated and selected in vitro may have a disadvantage from this point of view. This is important since a given therapeutic antibody risks to be neutralized by the so called anti-idiotypic antibody response from the patient (Lonberg, Nature Biotechnology, 23: 1117-1 125, (2005)).

A key advantage of processes according to the invention based on active immunization of species of Canidae in large outbred populations where the individual animals have a different genetic background. It is therefore possible to use active immunization to elicit a strong and diverse immune response against the antigen of interest from which a diverse pool of potential antigen binding molecules can be obtained.

The ability to use active immunization in an outbred population which is phylogenetically distant from human would not be particularly advantageous if the antibodies so-produced were to exhibit a low sequence and structural homology with human antibodies such that substantial “protein engineering” would be required to create a candidate antibody with therapeutic potential. It is therefore extremely important that the inventors have shown that the Canidae germline (and somatically mutated sequences) encodes both VH and VL domains with a very high degree of sequence and structural homology with human VH and VL domains (as explained above). This high degree of homology in combination with the availability of large outbred populations results in a very powerful platform for development of monoclonal antibodies for use as human therapeutics. Following active immunization with the target antigen, peripheral blood lymphocytes or biopsies such as lymph nodes or spleen biopsies may be isolated from the immunized animal and screened for production of conventional Canidae antibodies against the target antigen. Techniques such as enrichment using panning or FACS sorting may be used at this stage to reduce the complexity of the B cell repertoire to be screened, as illustrated in the examples. Antigen-specific B cells are then selected and used for total RNA extraction and subsequent cDNA synthesis. Nucleic acid encoding the native Canidae VH and VL domains (specific for the target antigen) can be isolated by PCR. It is not essential to use active immunization in order to identify Canidae convention antibodies immunoreactive with a target of interest. In other embodiments it may be possible to make use of the Canidae's own immune response, either the immunodiversity naturally present in the animal, or for example a diseased animal or animal which has been naturally exposed to a particular pathogen, e.g. by normal infection routes. In this regard, the invention encompasses the use of non-immune libraries. If “natural” immune responses within the Canidae already give rise to antibodies which bind the target antigen of interest, then it is possible to make use of the genetic engineering techniques described herein, and other standard techniques known in the art, in order to culture and isolate B cells producing such antibodies, or produce monoclonal cultures of such antibodies, and/or to determine the nucleotide sequence of the Canidae gene segments encoding the VH and/or VL domains of such antibodies. Armed with this sequence information, it is then possible to engineer recombinant DNA constructs encoding antigen binding polypeptides which embody the Canidae derived VH and/or VL, or the hypervariable loops (or CDRs) thereof. Nucleic acid encoding Canidae VH and VL domains (whether obtained by active immunization or by other means) may be cloned directly into an expression vector for the production of an antigen binding polypeptide according to the invention. In particular, these sequences could be cloned into an expression vector which also encodes a human antibody constant region, or a portion thereof, in order to produce a chimeric antibody. However, it is typical to carry out further manipulations on the isolated Canidae VH and VL sequences before cloning and expression with human constant region sequences.

As a first step, candidate Canidae VH and VL sequences (including sequences isolated following the active immunization) may be used to prepare a Canidae libraries (e.g. Fab libraries, as described in the accompanying examples). The library may then be screened (e.g. using phage display) for binding to the target antigen. Promising lead candidates can be further tested for target antigen binding, for example using Biacore or a suitable bioassay. Finally, the sequences encoding the VH and VL domains of the most promising leads can be cloned as an in-frame fusion with sequences encoding a human antibody constant region.

It is not essential that the polynucleotide sequence used to encode the (Canidae-derived) HVs/CDRs (e.g. for recombinant expression of the antigen binding polypeptide of the invention) is identical to the native polynucleotide sequence which naturally encodes the HVs/CDRs in the Canidae. Therefore, the invention encompasses/permits codon optimization, and other changes in polynucleotide sequence related to cloning and/or expression, which do not alter the encoded amino acid sequence.

In certain embodiments, “chain shuffling” may be performed in which a particular variable domain known to bind the antigen of interest is paired with each of a set of variable domains of the opposite type (i.e. VH paired with VL library or wee versa), to create libraries, and the resulting “promiscuous” combinations of VH/VL tested for antigen binding affinity and/or specificity. Alternatively, a library of VH domains could be paired with a library of VL domains, either randomly or in a hierarchical manner, and the resulting combinations tested (see Clackson et al., Nature., Vol. 352. pp624-638, 1991). In this process, the libraries may be libraries of rearranged VH and VL (VKappa or Vlambda) from Canidae which display immunity to the antigen of interest (including animals which have been actively immunized). The chain shuffling process can increase immunodiversity and produce pairings with significantly enhanced affinity.

The invention also contemplates performing epitope imprinted selection (so-called “guided selection”) starting from a Canidae VH or VL domain, wherein the other variable domain is taken from a non-Canidae species, e.g. human. Thus, in one embodiment a Canidae VH domain may be “shuffled” with a library of human-encoded VL domains, to replace the native Canidae-encoded VL domain, resulting in Canidae VH/human VL pairings. One or more of these pairings may then be subjected a second chain shuffling step in which the human VL domain is shuffled against a library of VH domains, which may be human-encoded. This second step may produce human-encoded VH/VL combinations which have the epitope imprint of the original Canidae-encoded VH/VL combination.

Also included within the scope of the invention is the reverse “chain shuffling” process, starting with non-Canidae (preferably human)-encoded VH/VL domain combination which binds to an antigen of interest. This could be, for example, a fully human therapeutic antibody against a validated disease target. Starting from this VH/VL combination, it is possible to carry out a first round of selection in which the VH domain is “shuffled” with a library of Canidae-encoded VL domains (or vice versa), and the pairings tested for antigen binding. Selected non-Canidae (e.g. human) VH/Canidae VL pairings may then be subjected to a second round of selection in which the Canidae-encoded VL is shuffled against a library of Canidae-encoded VH, and the resulting pairings tested for antigen binding. As a result, it may be possible to produce a Canidae VH/Canidae VL combination which carries the epitope imprint of the starting VH/VL combination. This Canidae VH/VL combination could be further engineered/modified and combined with human-encoded constant domains as required, using any of the processes described herein.

In the processes of the invention, “native” Canidae-derived VH and VL domains may be subject to protein engineering in which one or more selective amino acid substitutions are introduced, typically in the framework regions. The reasons for introducing such substitutions into the “wild type” Canidae sequence can be (i) humanization of the framework region, (ii) improvement in stability, bioavailability, product uniformity, tissue penetration, etc., or (iii) optimization of target antigen binding.

“Humanization” of Canidae-derived VH and VL domains by selective replacement of one or more amino acid residues in the framework regions may be carried out according to well-established principles (as illustrated in the accompanying examples, and reviewed by Almagro et al. Frontiers in Bioscience 13:1619-1633 (2008), the contents of which are specifically incorporated herein by reference). It will be appreciated that the precise identity of the amino acid changes made to achieve acceptable “humanization” of any given VH domain, VL domain or combination thereof will vary on a case-by-case basis, since this will depend upon the sequence of the framework regions derived from Canidae and the starting homology between these framework regions and the closest aligning human germline (or somatically mutated) framework region, and possible also on the sequence and conformation of the hypervariable loops which form the antigen binding site.

The overall aim of humanization is to produce a molecule in which the VH and VL domains exhibit minimal immunogenicity when introduced into a human subject, whilst retaining the specificity and affinity of the antigen binding site formed by the parental VH and VL domains encoded by Canidae and obtained by active immunization. There are a number of established approaches to humanization which can be used to achieve this aim. Techniques can be generally classified as either rational approaches or empirical approaches. Rational approaches include CDR-grafting, resurfacing or veneering, super humanization and human string content optimization. Empirical approaches include the FR library approach, guided selection, FR shuffling and humaneering. All of these techniques are reviewed in Almagro, Frontiers in Bioscience 2008, ibid and any of these techniques, or combinations or modifications thereof, can be used to prepare “humanized” antigen binding polypeptides according to the invention.

Another aspect of the application provides a method of producing a library of expression vectors encoding VH and/or VL domains of Canidae conventional antibodies, said method comprising the steps:
a) amplifying regions of nucleic acid molecules encoding VH and/or VL domains of Canidae conventional antibodies to obtain amplified gene segments, each gene segment containing a sequence of nucleotides encoding a VH domain or a sequence of nucleotides encoding a VL domain of a Canidae conventional antibody, and
b) cloning the gene segments obtained in a) into expression vectors, such that each expression vector contains at least a gene segment encoding a VH domain and/or a gene segment encoding a VL domain, whereby a library of expression vectors is obtained.

The above methods of “library construction” may also form part of the general process for production of antigen binding polypeptides of the invention, described above. Hence, any feature described as being preferred or advantageous in relation to this aspect of the invention may also be taken as preferred or advantageous in relation to the general process, and wee versa, unless otherwise stated.

In one embodiment, the nucleic acid amplified in step a) comprises cDNA or genomic DNA prepared from lymphoid tissue of a Canidae, said lymphoid tissue comprising one or more B cells, lymph nodes, spleen cells, bone marrow cells, or a combination thereof. Preferably, said lymphoid tissue is obtained from a Canidae which has been actively immunized. Circulating B cells are particularly preferred. Peripheral blood lymphocytes (PBLs) can be used as a source of nucleic acid encoding VH and VL domains of conventional Canidae antibodies, i.e. there is sufficient quantity of plasma cells (expressing antibodies) present in a sample of PBLs to enable direct amplification. This is advantageous because PBLs can be prepared from a whole blood sample taken from the animal (Canidae). This avoids the need to use invasive procedures to obtain tissue biopsies (e.g. from spleen or lymph node), and means that the sampling procedure can be repeated as often as necessary, with minimal impact on the animal. Preparing a sample containing PBLs from a Canidae, preparing cDNA or genomic DNA from the PBLs and using this cDNA or genomic DNA as a template for amplification of gene segments of interest encoding VH or VL domains of Canidae conventional antibodies.

In some embodiments, the lymphoid tissue comprises one or more B cells selected for the expression of Canidae conventional antibodies with desired antigen binding properties.

Conveniently, total RNA (or mRNA) can be prepared from the lymphoid tissue sample (e.g. peripheral blood cells or tissue biopsy) and converted to cDNA by standard techniques. It is also possible to use genomic DNA as a starting material.

Various conventional methods may be used to select Canidae B cells expressing antibodies with desired antigen-binding characteristics. For example, B cells can be stained for cell surface display of conventional IgG with fluorescently labelled monoclonal antibody (mAb, specifically recognizing conventional antibodies from Dog or other Canidae) and with target antigen labelled with another fluorescent dye. Individual double positive B cells may then be isolated by FACS, and total RNA (or genomic DNA) extracted from individual cells. Alternatively cells can be subjected to in vitro proliferation and culture supernatants with secreted IgG can be screened, and total RNA (or genomic DNA) extracted from positive cells. In a still further approach, individual B cells may be transformed with specific genes or fused with tumour cell lines to generate cell lines, which can be grown “at will”, and total RNA (or genomic DNA) subsequently prepared from these cells.

Instead of sorting by FACS, target specific B cells expressing conventional IgG can be “panned” on immobilized monoclonal antibodies (directed against Canidae conventional antibodies) and subsequently on immobilized target antigen. RNA (or genomic DNA) can be extracted from pools of antigen specific B cells or these pools can be transformed and individual cells cloned out by limited dilution or FACS.

B cell selection methods may involve positive selection, or negative selection.

Whether using a diverse library approach without any B cell selection, or a B cell selection approach, nucleic acid (cDNA or genomic DNA) prepared from the lymphoid tissue is subject to an amplification step in order to amplify gene segments of interest encoding individual VH domains or VL domains.

Total RNA extracted from the lymphoid tissue (e.g. peripheral B cells or tissue biopsy) may be converted into random primed cDNA or oligo dT primer can be used for cDNA synthesis, alternatively Ig specific oligonucleotide primers can be applied for cDNA synthesis, or mRNA (i.e. poly A RNA) can be purified from total RNA with oligo dT cellulose prior to cDNA synthesis. Genomic DNA isolated from B cells can be used for PCR.

PCR amplification of heavy chain and light chain (kappa and lambda) gene segments encoding at least VH or VL can be performed with FR1 primers annealing to the 5′ end of the variable region in combination with primers annealing to the 3′ end of CH1 or Ckappa/Clambda region with the advantage that for these constant region primers only one primer is needed for each type. This approach enables Canidae Fabs to be cloned. Alternatively sets of FR4 primers annealing to the 3′ end of the variable regions can be used, again for cloning as Fabs (fused to vector encoded constant regions) or as scFv (single chain Fv, in which the heavy and light chain variable regions are linked via a flexible linker sequence); alternatively the variable regions can be cloned in expression vectors allowing the production of full length IgG molecules displayed on mammalian cells.

In general the amplification is performed in two steps; in the first step with non-tagged primers using a large amount of cDNA (to maintain diversity) and in the second step the amplicons are re-amplified in only a few cycles with tagged primers, which are extended primers with restriction sites introduced at the 5′ for cloning. Amplicons produced in the first amplification step (non-tagged primers) may be gel-purified to remove excess primers, prior to the second amplification step. Alternatively, promoter sequences may be introduced, which allow transcription into RNA for ribosome display. Instead of restriction sites recombination sites can be introduced, like the Cre-Lox or TOPO sites, that permit the site directed insertion into appropriate vectors.

Amplified gene segments encoding Canidae conventional VH and VL domains may then be cloned into vectors suitable for expression of VH/VL combinations as functional antigen binding polypeptides. By way of example, amplified VHCH1/VKCK/VLCL gene segments from pools of B cells (or other lymphoid tissue not subject to any B cell selection) may be first cloned separately as individual libraries (primary libraries), then in a second step Fab or scFV libraries may be assembled by cutting out the light chain fragments and ligating these into vectors encoding the heavy chain fragments. The two step procedure supports the generation of large libraries, because the cloning of PCR products is relatively inefficient (due to suboptimal digestion with restriction enzymes). scFv encoding DNA fragments can be generated by splicing-by-overlap extension PCR (SOE) based on a small overlap in sequence in amplicons; by mixing VH and VL encoding amplicons with a small DNA fragment encoding the linker in a PCR a single DNA fragment is formed due to the overlapping sequences.

Amplicons comprising VH and VL-encoding gene segments can be cloned in phage or phagemid vectors, allowing selection of target specific antibody fragments by using phage display based selection methods. Alternatively amplicons can be cloned into expression vectors which permit display on yeast cells (as Fab, scFv or full length IgG) or mammalian cells (as IgG).

In other embodiments, cloning can be avoided by using the amplicons for ribosome display, in which a T7 (or other) promoter sequence and ribosome binding site is included in the primers for amplification. After selection for binding to target antigen, pools are cloned and individual clones are analyzed. In theory, larger immune repertoires can be sampled using this approach as opposed to a phage display library approach, because cloning of libraries and selection with phage is limited to 1010 to 1012 clones.

When applying B cell sorting, amplicons contain VH or VL-encoding gene segments of individual target specific B cells can be cloned directly into bacterial or mammalian expression vectors for the production of antibody fragments (scFVs or Fabs) or even full length IgG.

In a particular, non-limiting, embodiment of the “library construction” process, the invention provides a method of producing a library of expression vectors encoding VH and VL domains of Canidae conventional antibodies, said method comprising the steps: a) actively immunising a Canidae, thereby raising conventional Canidae antibodies against a target antigen;
b) preparing cDNA or genomic DNA from a sample comprising lymphoid tissue (e.g. circulating B cells) from said immunised Canidae;
c) amplifying regions of said cDNA or genomic DNA to obtain amplified gene segments, each gene segment comprising a sequence of nucleotides encoding a VH domain or a sequence of nucleotides encoding a VL domain of a Canidae conventional antibody; and
d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, whereby a library of expression vectors is obtained.

The foregoing methods may be used to prepare libraries of Canidae-encoded VH and VL domains, suitable for expression of VH/VL combinations as functional antigen-binding polypeptides, e.g. in the form of scFVs, Fabs or full-length antibodies.

Libraries of expression vectors prepared according to the foregoing process, and encoding Canidae VH and VL domains, also form part of the subject-matter of the present invention.

In a particular embodiment the invention provides a library of phage vectors encoding Fab or scFV molecules, wherein each Fab or scFV encoded in the library comprises a VH domain of a Canidae conventional antibody and a VL domain of a Canidae conventional antibody.

In one embodiment the library is a “diverse” library, in which the majority of clones in the library encode VH domains of unique amino acid sequence, and/or VL domains of unique amino acid sequence, including diverse libraries of Canidae VH domains and Canidae VL domains. Therefore, the majority of clones in a diverse library encode a VH/VL pairing which differs from any other VH/VL pairing encoded in the same library with respect to amino acid sequence of the VH domain and/or the VL domain.

The invention also encompasses expression vectors containing VH and VL-encoding gene segments isolated from a single selected B cell of a Canidae.

In a further aspect, the present invention also provides a method of selecting an expression vector encoding an antigen binding polypeptide, preferably a chimeric monoclonal antibody, immunoreactive with a target antigen, the method comprising steps of:
i) providing a library of expression vectors, wherein each vector in said library comprises a gene segment encoding a VH domain and a gene segment encoding a VL domain, wherein at least one of said VH domain or said VL domain is from a Canidae conventional antibody, and wherein each vector in said library directs expression of an antigen binding polypeptide comprising said VH domain and VL domain;
ii) screening antigen binding polypeptides encoded by said library for immunoreactivity with said target antigen, and thereby selecting an expression vector encoding an antigen binding polypeptide immunoreactive with said target antigen.

This method of the invention encompasses screening/selection of clones immunoreactive with target antigen, from a library of clones encoding VH/VL pairings. The method may also encompass library construction, which may be carried out using the library construction method described above. Optional downstream processing/optimisation steps may be carried out on selected clones, as described below. This method of selection and screening, may also form part of the general process for production of antigen binding polypeptides of the invention, described above. Hence, any feature described as being preferred or advantageous in relation to this aspect of the invention may also be taken as preferred or advantageous in relation to the general process, and vice versa, unless otherwise stated.

Screening/selection typically involves contacting expression products encoded by clones in the library (ie. VH/VL pairings in the form of antigen binding polypeptides, e.g. Fabs, scFVs or antibodies) with a target antigen, and selecting one or more clones which encode a VH/VL pairings exhibiting the desired antigen binding characteristics.

Phage display libraries may be selected on immobilized target antigen or on soluble (often biotinylated) target antigen. The Fab format allows affinity driven selection due to its monomeric appearance and its monovalent display on phage, which is not possible for scFv (as a consequence of aggregation and multivalent display on phage) and IgG (bivalent format). Two to three rounds of selections are typically needed to get sufficient enrichment of target specific binders.

Affinity driven selections can be performed by lowering the amount of target antigen in subsequent rounds of selection, whereas extended washes with non-biotinylated target enables the identification of binders with extremely good affinities.

The selection procedure allows the user to home in on certain epitopes; whereas the classical method for elution of phage clones from the immobilized target is based on a pH shock, which denatures the antibody fragment and/or target, competition with a reference mAb against the target antigen or soluble receptor or cytokine leads to the elution of phage displaying antibody fragments binding to the relevant epitope of the target (this is of course applicable to other display systems as well, including the B cells selection method).

Individual clones taken from the selection outputs may be used for small scale production of antigen-binding polypeptides (e.g. antibody fragments) using periplasmic fractions prepared from the cells or the culture supernatants, into which the fragments “leaked” from the cells. Expression may be driven by an inducible promoter (e.g. the lac promoter), meaning that upon addition of the inducer (IPTG) production of the fragment is initiated. A leader sequence ensures the transport of the fragment into the periplasm, where it is properly folded and the intramolecular disulphide bridges are formed.

The resulting crude protein fractions may be used in target binding assays, such as ELISA. For binding studies, phage prepared from individual clones can be used to circumvent the low expression yields of Fabs, which in general give very low binding signals. These protein fractions can also be screened using in vitro receptor-ligand binding assays to identify antagonistic antibodies; ELISA based receptor-ligand binding assays can be used, also high throughput assays like Alphascreen are possible.

Screening may be performed in radiolabeled ligand binding assays, in which membrane fractions of receptor overexpressing cell lines are immobilized; the latter assay is extremely sensitive, since only picomolar amounts of radioactive cytokine are needed, meaning that minute amounts of antagonistic Fabs present in the crude protein fraction will give a positive read-out. Alternatively, FACS can be applied to screen for antibodies, which inhibit binding of a fluorescently labelled cytokine to its receptor as expressed on cells, while FMAT is the high throughput variant of this. Fabs present in periplasmic fractions or partially purified by IMAC on its hexa-histidine tag or by protein G (known to bind to the CH1 domain of Fabs) can be directly used in bioassays using cells, which are not sensitive to bacterial impurities; alternatively, Fabs from individual E. coli cells can be re-cloned in mammalian systems for the expression of Fabs or IgG and subsequently screened in bioassays.

Following identification of positive expression vector clones, i.e. clones encoding a functional VH/VL combination which binds to the desired target antigen, it is a matter of routine to determine the nucleotide sequences of the variable regions, and hence deduce the amino acid sequences of the encoded VH and VL domains.

If desired, the Fab (or scFV) encoding region may be re-cloned into an alternative expression platform, e.g. a bacterial expression vector (identical to the phagemid vector, but without the gene 3 necessary for display on phage), which allows larger amounts of the encoded fragment to be produced and purified.

The affinity of target binding may be determined for the purified Fab (or scFV) by surface plasmon resonance (e.g. Biacore) or via other methods, and the neutralizing potency tested using in vitro receptor—ligand binding assays and cell based assays.

Families of antigen-binding, and especially antagonistic Fabs (or scFVs) may be identified on the basis of sequence analysis (mainly of VH, in particular the length and amino acid sequence of CDR3 of the VH domain).

Clones identified by screening/selection as encoding a VH/VL combination with affinity for the desired target antigen may, if desired, be subject to downstream steps in which the affinity and/or neutralizing potency is optimized.

Potency optimization of the best performing member of each VH family can be achieved via light chain shuffling, heavy chain shuffling or a combination thereof, thereby selecting the affinity variants naturally occurring in the animal. This is particularly advantageous in embodiments where the original Canidae VH/VL domains were selected from an actively immunized Canidae, since it is possible to perform chain shuffling using the original library prepared from the same immunized animal, thereby screening affinity variants arising in the same immunized animal.

For light chain shuffling the gene segment encoding the VH region (or VHCH1) of VH/VL pairing with desirable antigen binding characteristics (e.g. an antagonistic Fab) may be used to construct a library in which this single VH-encoding gene segment is combined with the light chain repertoire of the library from which the clone was originally selected. For example, if the VH-encoding segment was selected from a library (e.g. Fab library) prepared from a Canidae animal actively immunized to elicit an immune response against a target antigen, then the “chain shuffling” library may be constructed by combining this VH-encoding segment with the light chain (VL) repertoire of the same immunized Canidae. The resulting library may then be subject to selection of the target antigen, but under stringent conditions (low concentrations of target, extensive washing with non-biotinylated target in solution) to ensure the isolation of the best affinity variant. Off-rate screening of periplasmic fractions may also assist in the identification of improved clones. After sequence analysis and re-cloning into a bacterial production vector, purified selected Fabs may be tested for affinity (e.g. by surface plasmon resonance) and potency (e.g. by bioassay).

Heavy chain shuffling can be performed by cloning back the gene segment encoding the light chain (VL) of a clone selected after light-chain shuffling into the original heavy chain library from the same animal (from which the original VH/VL-encoding clone was selected). Alternatively a CDR3 specific oligonucleotide primer can be used for the amplification of the family of VH regions, which can be cloned as a repertoire in combination with the light chain of the antagonistic Fab. Affinity driven selections and off-rate screening then allow the identification of the best performing VH within the family.

It will be appreciated that the light chain shuffling and heavy chain shuffling steps may, in practice, be performed in either order, i.e. light chain shuffling may be performed first and followed by heavy chain shuffling, or heavy chain shuffling may be performed first and followed by light chain shuffling. Both possibilities are encompassed within the scope of the invention.

From light chains or heavy chains of VH/VL pairings (e.g. Fabs) with improved affinity and potency the sequences of, in particular, the CDRs can be used to generate engineered variants in which mutations of the individual Fabs are combined. It is known that often mutations can be additive, meaning that combining these mutations may lead to an even more increased affinity.

The VH and VL-encoding gene segments of selected expression clones encoding VH/VL pairings exhibiting desirable antigen-binding characteristics (e.g. phage clones encoding scFVs or Fabs) may be subjected to downstream processing steps and re-cloned into alternative expression platforms, such as vectors encoding antigen binding polypeptide formats suitable for human therapeutic use (e.g. full length antibodies with fully human constant domains).

Promising “lead” selected clones may be engineered to introduce one or more changes in the nucleotide sequence encoding the VH domain and/or the VL domain, which changes may or may not alter the encoded amino acid sequence of the VH domain and/or the VL domain. Such changes in sequence of the VH or VL domain may be engineered for any of the purposes described elsewhere herein, including germlining or humanization, codon optimization, enhanced stability, optimal affinity etc.

The general principles germlining or humanization described herein apply equally in this embodiment of the invention. By way of example, lead selected clones containing Canidae-encoded VH and VL domains may be germlined/humanized in their framework regions (FRs) by applying a library approach. After alignment against the closest human germline (for VH and VL) and other human germlines with the identical canonical folds of CDR1 and CDR2, the residues to be changed in the FRs are identified and the preferred human residue selected, as described elsewhere herein in detail. Whilst germlining may involve replacement of Canidae-encoded residues with an equivalent residue from the closest matching human germline this is not essential, and residues from other human germlines could also be used.

Once the amino acid sequences of the lead VH and VL domains (following potency optimization, as appropriate) are known, synthetic genes of VH and VL can be designed, in which residues deviating from the human germline are replaced with the preferred human residue (from the closest matching human germline, or with residues occurring in other human germlines, or even the Canidae wild type residue). At this stage the gene segments encoding the variable domains may be re-cloned into expression vectors in which they are fused to human constant regions of the Fab, either during gene synthesis or by cloning in an appropriate display vector.

The resulting VH and VL synthetic genes can be recombined into a Fab library or the germlined VH can be recombined with the wild type VL (and vice versa, referred to as “hybrid” libraries). Affinity-driven selections will allow the isolation of the best performing germlined version, in case of the “hybrid” libraries, the best performing germlined VH can be recombined with the best performing germlined VL.

Amino acid and nucleotide sequence information for the germlined Fabs can be used to generate codon-optimized synthetic genes for the production of full length human IgG of the preferred isotype (IgGI for ADCC and CDC, IgG2 for limited effector functions, IgG4 as for IgG2, but when monovalent binding is required). For non-chronic applications and acute indications bacterially or mammalian cell produced human Fab can produced as well.

Combining steps of the above-described processes, in a particular non-limiting embodiment the present invention provides a method of producing an expression vector encoding a chimeric antigen binding polypeptide, preferably a chimeric monoclonal antibody, immunoreactive with a target antigen, said method comprising the steps of:
a) actively immunizing a species of the family Canidae thereby raising conventional Canidae antibodies against a target antigen;
b) preparing cDNA or genomic DNA from a sample from said immunized species of the family Canidae, said sample comprising lymphoid tissue (e.g. circulating B cells);
c) amplifying regions of said cDNA or genomic DNA to obtain amplified gene segments, each gene segment comprising a sequence of nucleotides encoding a VH domain or a sequence of nucleotides encoding a VL domain of a Canidae conventional antibody;
d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, thereby producing a library of expression vectors;
e) screening antigen binding polypeptides encoded by the library obtained in step d) for immunoreactivity with said target antigen, and thereby selecting an expression vector encoding an antigen binding polypeptide immunoreactive with said target antigen;
f) optionally performing a light chain shuffling step and/or a heavy chain shuffling step to select an expression vector encoding a potency-optimized antigen binding polypeptide immunoreactive with said target antigen;
g) optionally subjecting the gene segment encoding the VH domain of the vector selected in step e) or step f) and/or the gene segment encoding the VL domain of the vector selected in step e) or step f) to germlining and/or codon optimization; and
h) cloning the gene segment encoding the VH domain of the vector selected in part e) or f) or the germlined and/or codon optimized VH gene segment produced in step g) and the gene segment encoding the VL domain of the vector selected in part e) or f) or the germlined and/or codon optimized VL gene segment produced in step g) into a further expression vector, in operable linkage with a sequence of nucleotides encoding one or more constant domains of a human antibody, thereby producing an expression vector encoding a chimeric antigen binding polypeptide comprising the VH and VL domains fused to one or more constant domains of a human antibody.

The invention also extends to expression vectors prepared according to the above-described processes, and to a method of producing an antigen binding polypeptide immunoreactive with a target antigen, the method comprising steps of:
I. preparing expression vector encoding an antigen binding polypeptide immunoreactive with a target antigen by:
a) actively immunizing a species of the family Canidae, thereby raising conventional Canidae antibodies against a target antigen;
b) preparing cDNA or genomic DNA from a sample comprising lymphoid tissue (e.g. circulating B cells) from said immunized species of the family Canidae;
c) amplifying regions of said cDNA or genomic DNA to obtain amplified gene segments, each gene segment comprising a sequence of nucleotides encoding a VH domain or a sequence of nucleotides encoding a VL domain of a Canidae conventional antibody;
d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, thereby producing a library of expression vectors;
e) screening antigen binding polypeptides encoded by the library obtained in step d) for immunoreactivity with said target antigen, and thereby selecting an expression vector encoding an antigen binding polypeptide immunoreactive with said target antigen;
f) cloning the gene segment encoding the VH domain of the vector selected in part e) and the gene segment encoding the VL domain of the vector selected in part e) or one or more hypervariable loop or complementary determining region (CDR) into a further expression vector, in operable linkage with a sequence of nucleotides encoding at least one other hypervariable loop or complementary determining region (CDR) of a human antibody or one or more constant domains of a human antibody, thereby producing an expression vector encoding a chimeric antigen binding polypeptide;
II. introducing said expression vector into host cell or cell-free expression system under conditions which permit expression of the encoded antigen binding polypeptide; and
III. recovering the expressed antigen binding polypeptide.

In some embodiments, the method further comprises between step e) and f), the step of performing a light chain shuffling step and/or a heavy chain shuffling step to select an expression vector encoding a potency-optimized antigen binding polypeptide immunoreactive with said target antigen.

In some embodiments, the method further comprises, after step f) the step f) of subjecting the gene segment encoding the VH domain of the vector to germlining and/or codon optimization.

In some embodiments, the processes enclosed herein encompass bulk production and large scale manufacture of the antigen binding polypeptides as disclosed herein. In particular, bulk production and large scale manufacture of antibodies for use in medicine, in particular for use in the prevention and/or treatment of a disease, are intended. In some embodiments, the antigen binding polypeptide as disclosed herein are thus for use as pharmaceutically active agents, such as immunotherapeutic agents. In such embodiments, the expression vector and the host cell or expression system are selected to be suitable for large-scale production of recombinant antigen binding polypeptides, in particular antibodies, intended for administration to subjects, in particular human subjects. The general characteristics of suitable vectors and expression systems for this purpose are well-known in the art.

It is apparent that there have been provided in accordance with the invention products, methods, and uses, that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Example 1: Generation of Antigen Binding Polypeptide According to the Invention

Preparation of an expression vector encoding an antigen binding polypeptide immunoreactive with a target antigen

A species of the family Canidae is immunized with two, three or more intramuscular injections of a target (e.g., human TIGIT, amino acid sequence represented in SEQ ID NO: 331; or human CD96; amino acid sequence represented in SEQ ID NO: 332) with or without (Freund) adjuvant. After an average of four to six weeks, blood samples are taken from immunized Canidae, and the immune response is confirmed in-vitro. In case the immune response is judged too low by a skilled person, extra injection(s) of the antigen (target) can be done before proceeding to the next step.

cDNA coding for heavy chain (VH) and the light chain (VL) variable domains is prepared from circulating B cells of the immunized animal and the cDNA is PCR-amplified using specific primers and cloned into a phagemid vector using the standardized approaches for phage display or any other expression system for producing and screening single-chain variable fragment (ScFv) at the surface of the phage. Best ScFv clones are investigated and characterized further.

### Next-Generation Sequencing for Antibody Discovery

Screening of the antigen binding polypeptides encoded by the library can be performed using NGS of B-cell receptor repertoire or phage-displayed antibody library and provides a global overview of VH and VL under in-vivo selective pressure and library diversity. Antibody-Extractor™ proprietary platform extracts VH and, or VL from the immune repertoire. The sequences are then synthesized and tested for further characterization.

Combining Expression Systems with Immune Repertoire Next-Generation Sequencing

The gene segments are thus cloned into expression systems, such as expression vectors. Expression Systems enable the selection of specific binders (VH). Using in-vitro selected VH for in-silico screening B-cell receptor immune repertoire helps find binders variants missed in the Expression Systems. Such naturally occurring variants are then investigated and further characterized. They also serve as a template for Antibody-Engineering operations.

The expression vector thus contains a gene segment encoding a VH domain and a gene segment encoding a VL domain and directs expression of antigen binding polypeptide comprising said VH domain and said VL domain, thereby producing a library of expression vectors. Screening of the library of expression vectors allows for selecting an expression vector encoding an antigen binding polypeptide immunoreactive with a specific target antigen.

The gene segment encoding the VH domain and the gene segment encoding the VL domain and one or more hypervariable loop or CDRs is then cloned in a further expression vector, in operably linkage with a sequence of nucleotides encoding at least one hypervariable loop or CDR of a human antibody or one or more constant domains of a human antibody, thereby producing an expression vector encoding a chimeric antigen binding polypeptide.

The so obtained expression vector is then introduced into a host cell or cell-free expression system under conditions which permit expression of the encoded antigen binding polypeptide, whereafter the expressed antigen biding polypeptide is recovered.

### Antibody-Engineering Rationalization

Finding variants in the immune repertoire helps determine antibody signature and conserved/variable residues and position. It rationalizes the humanization and in-vitro affinity maturation processes.

### Example 2. Sequence Alignment Analysis for VH and VL Domains of Different Origins Between Human and Several Species of the Canidae Family (Dog, Fox, Wolf, Dingo)

Human germline best matching family member hit (HuGermHit) that has the highest degree of sequence identity with the Canidae variable region of interest is chosen for scoring the sequence identity. Canidae VGenes and HuGermHit may or may not share the same length of HCDR1 and HCDR2 for heavy chain and may or may not share the same length of LCDR1 and LCDR2 of the light chains.

Antibody-Extractor™ Germaligner alignment algorithm was used to compute the percentage frameworks sequence identity between Canidae VH and VL Frameworks 1, 2 and 3 amino acid sequences of dog, wolf, dingo and fox, and the HuGermHit or the global identity between Canidae VH and VL including Frameworks 1,2 and 3 and CDR1 and CDR2 and the HuGermHit Frameworks 1,2 and 3 and CDR1 and CDR2. Results of the alignment analyses are shown in Tables 3-8. As evidenced in these sequence alignments, sequence similarity with the human Vgene varied between 58% and 88% (dog), between 72% and 87% (dingo), between 69% and 87% (fox) and between 75% and 87% (wolf) for the complete VH domain; whereas sequence similarity with only the human framework regions varied between 70% and 93% (dog), between 75% and 93% (dingo), between 69% and 92% (fox) and between 77% and 93% (wolf). Sequence alignments for the VL (kappa) domain between the human Vgene and the dog Vgene varied between 76% and 83%, whereas sequence alignment for only the FRs in the VL (kappa) domain between human and dog varied between 78% and 87%. Sequence alignments for the VL (lambda) domain between the human Vgene and the dog Vgene varied between 58% and 83%, whereas sequence alignment for only the FRs in the VL (lambda) domain between human and dog varied between 62% and 86%.

Table 9 provides an alignment analysis between VH domains of human and dog anti-SARS antibodies demonstrating that the sequence similarity also occurs in matured antibodies and not only in germline sequences.

Table 10 provides an alignment analysis between VH domains of human and dog naïve antibodies demonstrating that there is a similarity of between 76% to 88% between the human and dog VGene, whereas sequence similarity between human and dog sequences within the framework regions only varied between 84% and 93%.

### Example 3. Identification of Functional Canine Antibodies with Human Identity

To isolate functional target specific canine antibodies, the inventors used the naïve canine scFv-phage display library from Proteogenix (https://www.proteogenix.science/antibody-production/phage-display-services/dog.library/). Shortly, this library was created by collecting the PBMCs from healthy animals of 6 different breeds (Beagle, German Shephard, Labrador, English Coonhound, Great Dane, Chinese Rural Dog). After total RNA extraction and cDNA synthesis, the VH and VKappa (not the VLambda) were amplified and cloned into an optimized phagemid in scFv format.

Target specific antibodies against CD96 and TIGIT were isolated using standard phage display methods. After up to eight rounds of phage display selections using the Proteogenix canine naïve library, the binding of the selected antibodies was tested in phage ELISA. Shortly, the targets (CD96 and TIGIT) were coated on a 96-well plate and the binding of phage expressing single scFv clones was tested. The scFv of the phages binding to the target but not to either uncoated wells or albumin-coated wells were sequenced. For each target, one antibody fragment that specifically binds to its target was identified and sequenced. Clone G1 binds to the extracellular domain of CD96 (target #1). Clone C2 binds to the extracellular domain of TIGIT (target #2). The sequence of each V domain (VH and VKappa) of these clones is indicated in Table 11.

The binding strength and specificity of the identified antibodies (anti-CD96 clone G1 and anti-TIGIT clone C2) were further confirmed in a phage ELISA against their specific coated human target (CD96 for clone G1 and TIGIT for clone C2) and compared to their binding to BSA or binding to the target of an irrelevant phage (Table 12). Shortly, 4 μg/ml of target was coated, and after washing a titration of phage (from 1011 to 9×10⁷ pfu/ml was added. After washing, bound phages were revealed using anti-M13-HRP antibodies and TMB according to the manufacturer recommendation.

### Example 4. Sequence Analysis of the CD96 Clone G1 Antibody and TIGIT Clone C2 Antibody and Sequence Alignment with Canine Germline Sequences

The ten closest canine germline sequences from which the antibodies were derived were identified. Clone G1 had 94% framework identity to dog IGHV3-35 (and dog IGHV3-67) and 97% framework identity to dog IGKV2S13 (Table 13). Clone C2 had 89% framework identity to dog IGHV3-35 (and dog IGHV3-67) and 96% framework identity to dog IGJV2S13 (Table 14). Interestingly, some unexpected mutations (non conserved in any germline) were found in the framework of the functional antibodies compared to the closest germline (3 in VH and 2 in VK of clone G1 and C2; underlined in Tables 13 and 14). These mutations could either be due to a difference in canine species, somatic mutations due to intrinsic sequence instability of the phage display method during library construction, or due to the high number of rounds of phage display selection.

Furthermore, the closest human germline was identified and the human identity of the framework regions (based on Kabat numbering) was calculated using each VH and VKappa domain (Table 15). The sequence identity of the VH and VKappa was confirmed to be relatively high and ranged from 82-87% and 85%, respectively (see Table 18).

Amino acid and corresponding DNA sequences of the anti-CD96 G1 clone (VH and VL domains) are represented in SEQ ID NOs: 304 and 305 and SEQ ID NOs: 333 and 334, respectively (Table 22). Amino acid and corresponding DNA sequences of the anti-TIGIT C2 clone (VH and VL domains) are represented in SEQ ID NOs: 306 and 307, and SEQ ID NOs: 335 and 336, respectively.

### Example 5. Comparison of Functional Canine Antibodies to Germline Sequences of Other Species

Heavy and light chain sequences from the dog naïve repertoire were used to screen germline genes from different animals commonly used in human immunotherapeutic antibody discovery. The highest germline gene sequence identity for each organism is computed in order to determine the nearest V and J germline genes of different organisms to the anti-CD96 G1 and anti-TIGIT C2 antibodies. CDR3 is not included in percentage identity computing. For all V genes tested (VH or VK), the closest germline was the human germline, followed by the mouse germline (Table 16, 17 and 18).

These data confirm the close identity between the canine and human V genes making the canine V genes an ideal candidate to humanize.

### Example 6. Humanization of Canine Antibodies

Although the sequence identity of the canine antibodies to the human germline was high, the inventors maximized the human identity by grafting the human framework into the canine scFv antibody. For this, one or two germlines were tested depending on the existing diversity of human germline: IGHV3-7, IGHV3-48 and IGKV2D-29 for clone C1 (CD96) and IGHV3-7, IGHV3-21 and IGKV2D-29 for clone C2 (TIGIT). The residues in the canine antibody whose position is known to affect the CDR loop conformations were conserved in some variants. The sequences of the humanized VH and VK domains (comprising the CDR regions of clones C1 and G2) are indicated in Tables 19 and 20. Sequence alignments between VH and VL domains of the humanized antigen binding polypeptides and sequences of human VH and VL domains are shown in Table 21. VHhum and VKhum can be combined together to form up to 6 scFv antibodies and cloned into phagemid. After production of the scFv antibodies, their target binding will be compared with the original canine antibodies of clone G1 and C2 by phage ELISA. The ease by which functional fully antibodies can be generated is dependent on the high starting human identity of the canine sequences.

In a next step, the humanized V domains (VH and VKappa) can be recloned fused to human constant domains to generate purified antibodies (for example as full IgG or any other fusion protein), and production, affinity and stability can be tested.

TABLE 1
Germline V Genes consensus sequences for human
and dog Heavy, Kappa and Lambda chains. CDRs
are indicated in bold; signatory canine amino
acids are underlined.
Heavy Chain

| | |
|---|---|
| Human | EVQLVQSGAEVKKPGESLRISCKGSGYSFTSYWIS |
| | WVRQMPGKGLEWMGRIDPSDSYTNYSPSFQG |
| | HVTISADKSISTAYLQWSSLKASDTAMYYCAR |
| Dog | EVQLVESGGDLVKPVGSLRLSCVASGFTFSSYDMN |
| C1 | WVRQAPGKGLQWVAYISSGGSSTYYADAVKG |
| | RFTISRDNAKNTLYLQMNSLRAEDTAMYYCAG |

Lambda V Gene

| | |
|---|---|
| Human | QSVLTQPPSVSVSPGQSVTITCTGSSSDVGG |
| | SNYVSWYQQKPGQAPKLLIYEKSDGIDSNRPS |
| | GVPDRFSGSKSGNTASLTISGLQAEDEADYYC |
| Dog | QSVLTQPPSVSGSLGQRVTISCTGSSSNVIGSN |
| C2 | YVGWYQQLPGTAPRTLIYGYSDSTSNRPS |
| | GVPDRFSGSKSGNTATLTISGLQAEDEADYYC |

Kappa V gene

| | |
|---|---|
| Human | DIVMTQSPSSLSASPGDRVTISCRASQSLLHS |
| | DGISSYLAWYQQKPGKAPKLLIYAASSLQS |
| | GVPSRFSGSGSGTDFTLTISSLQPEDFATYYC |
| Dog | DIVMTQTPLSLSVSPGETASISCKASQSLLHS |
| C3 | FNGNTYLNWFRQKPGQSPQRLIYKVSNRDP |
| | GVPDRFSGSGSGTDFTLRISRVEADDAGVYYC |

TABLE 2
Sequence alignments for heavy, kappa and lambda chains for the Germline V Genes consensus
sequences for human and dog.

>Dog_Heavy_Consensus
EVQLVESGGDLVKPVGSLRLSCVASGFTFSSYDMNWVRQAPGKGLQWVAYISSGGSSTYYADAVKGRFTISRDNAKNTLYLQMNSLRAEDTAMYYCAG
>Human_Heavy_Consensus
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

| | -------------- | | ------ | ------CDR2 | ----------------FW3 |
|---|---|---|---|---|---|
| | FW1------------- | CDR1- | FW2----- | ------- | -------------- |
| Dog_ | \|EVQLVESGGDLVKP | \|SYDMN\| | \|WVRQAPG | \|YISSGGSST | \|RFTISRDNAKNTLYLQMNS |
| Heavy_ | VGSLRLSCVASGFTFS\| | | KGLQWVA\| | YYADAVKG\| | LRAEDTAMYYCAG\| |
| Consensus | | | | | |
| Human_ | \|.....Q..AEVK..GE | \|..WIS\| | \|....M.. | \|R.DPSD.Y. | \|HV...A.KSIS.A...KS. |
| Heavy_ | ...I..KG..YS.T\| | ----- | ...E.MG\| | N.SPSFQ.\| | .K.S........R\| |
| Consensus | ---------------- | | ------- | --------- | ------------------- |
| | -------------- | | ------- | -------- | ------------- |

>Dog_Lambda_Consensus
QSVLTQPPSVSGSLGQRVTISCTGSSSNVIGSNYVGWYQQLPGTAPRTLIYGYSDSTSNRPSGVPQRFSGSKSGNTATLTISGLQAEDEADYYC
>Human_Lambda_Consensus
QSVLTQPPSVSVSPGQSVTITCTGSSSDVGGSNYVSWYQQKPGQAPKLLIYEKSDGIDSNRPSGVPDRFSGSKSGNTASLTISGLQAEDEADYYC

| | ----------FW1--- | ----- | ------ | ----CDR2 | ---------------FW3 |
|---|---|---|---|---|---|
| | ------ | CDR1----- | FW2------ | ---- | -------------- |
| Dog_ | \|QSVLTQPPSVSGSLGQ | \|TGSSSNVI | \|WYQQLPGT | \|GYSDS- | \|GVPDRFSGSKSGNTATLT |
| Lambda_ | RVTISC\| | GSNYVG\| | APRTLIY\| | TSNRPS\| | ISGLQAEDEADVYC\| |
| Consensus | | | | | |
| Human_ | \|...........V.P.. | \|.....D.G. | \|....K..Q. | \|EK..GID | \|...............S.. |
| Lambda_ | S...T.\| | ....S\| | .KL...\| | .....\| | ..............\| |
| Consensus | ---------------- | --------- | --------- | ------------ | ------------------ |
| | ------ | ----- | ------ | | -------------- |

>Dog_Kappa_Consensus
DIVMTQTPLSLSVSPGETASISCKASQSLLHSFNGNTYLNWFRQKPGQSPQRLIYKVSNRDPGVPDRFSGSGSGTDFTLRISRVEADDAGVYYC
>Human_Kappa_Consensus
DIVMTQSPSSLSASPGDRVTISCRASQSLLHSDGTSSYLAWYQQKPGKAPKLLIYAASSLQSGVPSRFSGSGSGTDFTLTISSLQPEDFATYYC

| | ----------FW1- | ------CDR1 | ------ | | --------------- |
|---|---|---|---|---|---|
| | --------- | ------- | FW2------ | -CDR2-- | FW3-------------- |
| >Dog_ | \|DIVMTQTPLSLSVS | \|KASQSLLHSF | \|WFRQKPGQ | \|KVSNRDP\| | \|GVPDRFSLSLSLTDFTL |
| Kappa_ | PGETASISC\| | NGNTYLN\| | SPQRLIY\| | | RISRVEADDAGVYVC\| |
| Consensus | | | | | |
| >Human_ | \|......S.S...A.. | \|R........D | \|.YQ....KA. | \|AA.SLQS\| | \|...S............. |
| Kappa_ | .DRVT...\| | GISS..A\| | KL...\| | ------- | T..SLQPE.FAT...\| |
| Consensus | --------------- | ---------- | ---------- | | ------FR--------- |
| | --------- | -------- | ------ | | --------------- |

TABLE 3
Sequence alignments for VH domains between human VGene and dog (canis familiaris)
VGene
--- IGHV3-74- (36) ---

| ACCES- | | % | % | -------------- | | ------ | ------CDR2 | --------------- |
|---|---|---|---|---|---|---|---|---|
| SIONS | Count | VGENE | FWs | FW1------------ | CDR1- | FW2----- | ------- | FW3-------------- |
| IGHV3- | 1 | | | \|EVQLVESGGGLVQPG | \|SYWMH\| | \|WVRQAPG | \|RINSDGSSTS | \|RFTISRDNAKNTLYLQ |
| 74 | | | | GSLRLSCAASGFTFS\| | | KGLVWVS\| | YADSVKG\| | MNSLRAEDTAVYYCAR\| |
| Dog_ | 1 | 88.0 | 92.0 | \|.........D..K.. | \|..R.S\| | \|....... | \|Y...G..... | \|................ |
| IGHV3- | | | | .......V.......\| | | ...Q..A\| | ...A...\| | ...............S\| |
| 5*01 | | | | | | | | |
| Dog_ | 1 | 87.0 | 92.0 | \|.........D..K.. | \|.NY.S\| | \|.I..... | \|Q.S....... | \|................ |
| IGHV3- | | | | .......V.......\| | | ...Q...\| | ...A...\| | .....D..........\| |
| 2*01 | | | | | | | | |
| Dog_ | 1 | 87.0 | 90.0 | \|.........DM.K.. | \|..Y.Y\| | \|.A..... | \|H..K...... | \|................ |
| IGHV3- | | | | .......V.......\| | | ...Q...\| | ...A...\| | ...............K\| |
| 3*01 | | | | | | | | |
| Dog_ | 1 | 87.0 | 90.0 | \|.........D..K.. | \|..Y.Y\| | \|....... | \|..S......Y | \|................ |
| IGHV3- | | | | .......V.......\| | | ...Q..A\| | ...A...\| | ..........M....K\| |
| 67*01 | | | | | | | | |
| Dog_ | 1 | 85.0 | 90.0 | \|.........D..K.. | \|.SD.S\| | \|.I..... | \|Y.SN...... | \|................ |
| IGHV3- | | | | .......V.......\| | | ...Q..A\| | ...A...\| | ...............D\| |
| 6*01 | | | | | | | | |
| Dog_ | 1 | 84.0 | 90.0 | \|.........D..K.. | \|.MD.D\| | \|....... | \|..SN..R..G | \|................ |
| IGHV3- | | | | .......V.......\| | | ...Q.LT\| | ...A...\| | ...............K\| |
| 50*01 | | | | | | | | |
| Dog_ | 1 | 84.0 | 89.0 | \|.........D..K.. | \|..Y.E\| | \|....... | \|Q.S......Y | \|Q............... |
| IGHV3- | | | | .......V.......\| | | ...Q..A\| | .P.A...\| | ....G..........K\| |
| 80*01 | | | | | | | | |
| Dog_ | 1 | 84.0 | 89.0 | \|.........D..K.. | \|..D.S\| | \|....S.. | \|V.NN.....Y | \|................ |
| IGHV3- | | | | .T.....V.......\| | | ...Q..A\| | ...A...\| | ...............K\| |
| 38*01 | | | | | | | | |
| Dog_ | 1 | 84.0 | 89.0 | \|.........D.EK.. | \|..G.S\| | \|....... | \|L.RV.....R | \|................ |
| IGHV3- | | | | .......V.......\| | | ...QG..\| | ...A...\| | ............S..K\| |
| 23*01 | | | | | | | | |
| Dog_ | 1 | 84.0 | 88.0 | \|.........D.MK.. | \|.NY.N\| | \|....... | \|Y.S....... | \|................ |
| IGHV3- | | | | .......V.....I.\| | | ...Q..G\| | ...A...\| | ..............VK\| |
| 54*01 | | | | | | | | |
| Dog_ | 1 | 84.0 | 88.0 | \|.........D..K.. | \|..A.S\| | \|....... | \|Y...G....Y | \|.........R...... |
| IGHV3- | | | | .......V.......\| | | ...Q..A\| | ...A...\| | .....S........PK\| |
| 69*01 | | | | | | | | |
| Dog_ | 1 | 84.0 | 88.0 | \|.........D..K.. | \|....S\| | \|C...... | \|Y...G....W | \|................ |
| IGHV3- | | | | .......VT......\| | | .E.Q..A\| | .T.A...\| | ..N.............\| |
| 44*01 | | | | | | | | |
| Dog_ | 1 | 83.0 | 89.0 | \|........ND..K.. | \|..Y.D\| | \|....... | \|G.S......Y | \|................ |
| IGHV3- | | | | .......V.......\| | | .R.Q..A\| | .PQA...\| | ........S......M\| |
| 81*01 | | | | | | | | |
| Dog_ | 1 | 83.0 | 88.0 | \|.........D..K.E | \|..D.S\| | \|....... | \|Y.S...R... | \|................ |
| IGHV3- | | | | .......VV......\| | | ...Q..A\| | .T.A...\| | .....T.........K\| |
| 18*01 | | | | | | | | |
| Dog_ | 1 | 82.0 | 86.0 | \|.........D..K.. | \|..Y.F\| | \|.I..... | \|Y..K.....Y | \|................ |
| IGHV3- | | | | .......V.......\| | | ..NQ..G\| | .P.A...\| | ....IV....I.....\| |
| 16*01 | | | | | | | | |
| Dog_ | 1 | 81.0 | 89.0 | \|.........D..K.. | \|....S\| | \|....... | \|E.SGS....N | \|..I............. |
| IGHV3- | | | | .......VG......\| | | ...Q..A\| | ...A...\| | ..........M.....\| |
| 26*01 | | | | | | | | |
| Dog_ | 1 | 81.0 | 88.0 | \|........ED..K.. | \|.SE.S\| | \|..H.... | \|W.RY....SR | \|................ |
| IGHV3- | | | | .......V.......\| | | Q..Q...\| | ...T...\| | ..........I....D\| |
| 46*01 | | | | | | | | |
| Dog_ | 1 | 81.0 | 86.0 | \|..P......E..K.E | \|...IS\| | \|....... | \|Y..K...T.Y | \|................ |
| IGHV3- | | | | ....F..V.......\| | | ...H...\| | H..A...\| | .......G.T.....H\| |
| 76*01 | | | | | | | | |
| Dog_ | 1 | 81.0 | 85.0 | \|.........D..K.A | \|..S.S\| | \|......E | \|G...G....Y | \|............V... |
| IGHV3- | | | | .......V.......\| | | ...QL.A\| | .T.A...\| | ..........M....K\| |
| 19*01 | | | | | | | | |
| Dog_ | 1 | 81.0 | 85.0 | \|.........D..K.E | \|.FY.S\| | \|.FC...R | \|E.S.S..... | \|...........M.... |
| IGHV3- | | | | .......V.......\| | | ...Q..A\| | ...I...\| | ........M.......\| |
| 24*01 | | | | | | | | |
| Dog_ | 1 | 81.0 | 84.0 | \|.........D..KS. | \|..Y..\| | \|.....T. | \|..SN.....R | \|Q.......S....... |
| IGHV3- | | | | .......V.......\| | | ...Q..T\| | ...AM..\| | ...Q....M.L.....\| |
| 82*01 | | | | | | | | |
| Dog_ | 1 | 81.0 | 82.0 | \|.M....A..D..KL. | \|....S\| | \|..G.... | \|Y...G....Y | \|................ |
| IGHV3- | | | | .....F.V.......\| | | ...Q..A\| | ...A...\| | ..C...........VG\| |
| 33*01 | | | | | | | | |
| Dog_ | 1 | 80.0 | 85.0 | \|.........D.AK.. | \|..S.S\| | \|....... | \|A.SY.....Y | \|.........R..V... |
| IGHV3- | | | | .......V...I...\| | | ...Q..T\| | .T.A...\| | ..............VD\| |
| 47*01 | | | | | | | | |
| Dog_ | 1 | 80.0 | 82.0 | \|.L.......D..K.. | \|.....\| | \|..L.... | \|I...G.G.IY | \|......E......... |
| IGHV3- | | | | .......VS......\| | | ...E..A\| | ...T...\| | ........R.MH...K\| |
| 83*01 | | | | | | | | |
| Dog_ | 1 | 80.0 | 82.0 | \|G........D..K.. | \|..S.Q\| | \|..C.... | \|Y...G..... | \|..I.....V....... |
| IGHV3- | | | | ...T...V.Y.....\| | | ..VQ..A\| | S..A...\| | ...............G\| |
| 32*01 | | | | | | | | |
| Dog_ | 1 | 79.0 | 86.0 | \|........ED..K.. | \|.SE.S\| | \|..H.... | \|W.RY...ISR | \|........V....... |
| IGHV3- | | | | .......V.......\| | | Q..Q...\| | ...T...\| | ..........I....D\| |
| 37*01 | | | | | | | | |
| Dog_ | 1 | 79.0 | 85.0 | \|.........N..K.. | \|..AIY\| | \|..HE... | \|A.TT.....Y | \|................ |
| IGHV3- | | | | .......V...L..Y\| | | ...Q..A\| | .T.A...\| | ........MP......\| |
| 61*01 | | | | | | | | |
| Dog_ | 1 | 79.0 | 84.0 | \|.........D..K.E | \|.FY.S\| | \|.FC.... | \|E.S.S.N... | \|...............R |
| IGHV3- | | | | R......V.......\| | | ...QC.A\| | ...A...\| | .H..............\| |
| 70*01 | | | | | | | | |
| Dog_ | 1 | 78.0 | 81.0 | \|.GAAGGVW.D..K.. | \|..Y.S\| | \|....... | \|Y...G....Y | \|................ |
| IGHV3- | | | | .......V.......\| | | ...Q..G\| | ...A...\| | ..............GK\| |
| 12*01 | | | | | | | | |
| Dog_ | 1 | 78.0 | 80.0 | \|......TE.D..K.. | \|..D.D\| | \|..V.... | \|V.SNG....R | \|Q........R...... |
| IGHV3- | | | | .......V.......\| | | ...Q..T\| | ...A...\| | .....DK.M.....VS\| |
| 10*01 | | | | | | | | |
| Dog_ | 1 | 75.0 | 81.0 | \|.E....F..HM.N.. | \|..G.S\| | \|.....QK | \|H.SY.....Y | \|........T..N.... |
| IGHV3- | | | | ...G...Q.......\| | | ...Q..G\| | ...TLRD\| | ..............N.\| |
| 7*01 | | | | | | | | |
| Dog_ | 1 | 74.0 | 76.0 | \|......P..E..K.. | \|..N.G\| | \|.AH.P.. | \|GF..G...... | \|E.......V....... |
| IGHV3- | | | | .......V.......\| | | ..MQ..A\| | T.A...\| | .....S........VK\| |
| 25*01 | | | | | | | | |
| Dog_ | 1 | 71.0 | 83.0 | \|...........GEAW | \|..A..\| | \|....... | \|V.SR.....N. | \|...........M.... |
| IGHV3- | | | | .......VT......\| | | ...Q..A\| | ..A...\| | ..........M....K\| |
| 51*01 | | | | | | | | |
| Dog_ | 1 | 71.0 | 80.0 | \|............K.. | \|....Y\| | \|..H.... | \|E.S.T....N. | \|P............... |
| IGHV3- | | | | .......VQP.....\| | | M..Q.LA\| | ..A.R.\| | V.............VS\| |
| 53*01 | | | | | | | | |
| Dog_ | 1 | 70.0 | 70.0 | \|..AAGGVW.N..K.. | \|.....\| | \|..C.... | \|I...G.G..Y. | \|Q...F......M.... |
| IGHV3- | | | | E......V.......\| | | ...G..A\| | ..T...\| | ......Q.MTA...VS\| |
| 55*01 | | | | | | | | |
| Dog_ | 1 | 64.0 | 76.0 | \|.........D..SL. | \|G..KY\| | \|.IM.... | \|W.SN....K.. | \|Q....K....Y..... |
| IGHV3- | | | | .......V..E.I..\| | ----- | ...Q..T\| | ..A...\| | ........M.....NM\| |
| 4*01 | | | | --------------- | | ------- | ----------- | ---------------- |
| | | | | --------------- | | ------- | ------ | ---------------- |

--- IGHV3-23D- (7) ---

| ACCES- | | % | % | -------------- | | ------ | ------CDR2 | --------------- |
|---|---|---|---|---|---|---|---|---|
| SIONS | Count | VGENE | FWs | FW1------------- | CDR1- | FW2----- | ------- | FW3-------------- |
| IGHV3- | 1 | | | \|EVQLLESGGGLVQPG | \|SYAMS\| | \|WVRQAPG | \|AISGSGGSTY | \|RFTISRDNSKNTLVLQ |
| 23D | | | | GSLRLSCAASGFTFS\| | | KGLEWVS\| | YADSVKG\| | MNSLRAEDTAVYYCAK\| |
| Dog_ | 1 | 85.0 | 88.0 | \|....V....E..K.. | \|..Y..\| | \|.I..... | \|D..D...... | \|........V..S.... |
| IGHV3- | | | | .......V.......\| | | ...Q..A\| | .T.A...\| | ................\| |
| 31*01 | | | | | | | | |
| Dog_ | 1 | 84.0 | 90.0 | \|....V....D..K.. | \|.H.K.\| | \|....... | \|V..S..S..G | \|........A....... |
| IGHV3- | | | | .......V.......\| | | ...K..A\| | S..T...\| | ................\| |
| 58*01 | | | | | | | | |
| Dog_ | 1 | 84.0 | 88.0 | \|....V....D..K.. | \|..Y..\| | \|.I..... | \|D..D.....G | \|......E.A..K.... |
| IGHV3- | | | | .......V.......\| | | ...Q..A\| | ...A...\| | ................\| |
| 39*01 | | | | | | | | |
| Dog_ | 1 | 83.0 | 88.0 | \|....V....D..K.. | \|M.D..\| | \|....... | \|...VD.G... | \|........AR..V... |
| IGHV3- | | | | .......V.......\| | | ...Q..A\| | .T.A...\| | ................\| |
| 41*01 | | | | | | | | |
| Dog_ | 1 | 82.0 | 84.0 | \|.L..V.L..D..K.. | \|.....\| | \|..C.... | \|...S..S... | \|........A...V... |
| IGHV3- | | | | .......V.......\| | | ...Q..A\| | WV.A...\| | ...............D\| |
| 75*01 | | | | | | | | |
| Dog_ | 1 | 78.0 | 83.0 | \|....V....D.MK.. | \|..G..\| | \|..C..S. | \|...VD.R... | \|........G....... |
| IGHV3- | | | | .......V......R\| | | ...Q..A\| | .T.T...\| | ...............S\| |
| 43*01 | | | | | | | | |
| Dog_ | 1 | 73.0 | 74.0 | \|..SWWSL.ED..K.. | \|R....\| | \|..G.... | \|...D..D... | \|.....I..A..MV... |
| IGHV3- | | | | .......VT......\| | ----- | ...Q..A\| | .V.A...\| | ........I.....G.\| |
| 42*01 | | | | --------------- | | ------- | ---------- | ---------------- |
| | | | | --------------- | | ------- | ------- | ---------------- |

--- IGHV3-21- (4) ---

| ACCES- | | % | % | -------------- | | ------ | ------CDR2 | --------------- |
|---|---|---|---|---|---|---|---|---|
| SIONS | Count | VGENE | FWs | FW1------------- | CDR1- | FW2----- | ------- | FW3-------------- |
| IGHV3- | 1 | | | \|EVQLVESGGGLVKPG | \|SYSMN\| | \|WVRQAPG | \|STSSSSSVTY | \|RFTISRDNAKNSLYLQ |
| 21 | | | | GSLRLSCAASGFTFS\| | | KGLEWVS\| | VADSVKG\| | MNSLRAEDTAVYYCAK\| |
| Dog_ | 1 | 82.0 | 93.0 | \|.........D..... | \|..D.D\| | \|....... | \|E....G.ST. | \|...........T.... |
| IGHV3- | | | | .......V.......\| | | ...Q.L.\| | ...A...\| | ................\| |
| 9*01 | | | | | | | | |
| Dog_ | 1 | 84.0 | 89.0 | \|.........D....V | \|..D..\| | \|....... | \|Y...GG.ST. | \|...........T.... |
| IGHV3- | | | | .......V.......\| | | ...Q..A\| | ...A...\| | ..........M....G\| |
| 35*01 | | | | | | | | |
| Dog_ | 1 | 82.0 | 92.0 | \|.........D..... | \|N.E.V\| | \|....... | \|R.YE.G.TT. | \|...........MA... |
| IGHV3- | | | | .......V.......\| | | ......A\| | ..EA...\| | ...............S\| |
| 8*01 | | | | | | | | |
| Dog_ | 1 | 74.0 | 76.0 | \|....I....DR.... | \|....H\| | \|CL..S.. | \|Y...NG.ST. | \|.......K...M.... |
| IGHV3- | | | | ..Q....V..R....\| | ----- | N..Q..T\| | ...A...\| | ......Q.T.L....D\| |
| 45*01 | | | | --------------- | | ------- | ---------- | ---------------- |
| | | | | --------------- | | ------- | ------- | ---------------- |

--- IGHV3-7- (1) ---

| ACCES- | | % | % | -------------- | | ------ | ------CDR2 | --------------- |
|---|---|---|---|---|---|---|---|---|
| SIONS | Count | VGENE | FWs | FW1------------- | CDR1- | FW2----- | ------- | FW3-------------- |
| IGHV3- | 1 | | | \|EVQLVESGGGLVQPG | \|SYMNS\| | \|WVRQAPG | \|NIKQDGSEKY | \|RFTISRDNAKNSLYLQ |
| 7 | | | | GSLRLSCAASSFTFS\| | | KGLEWVA\| | YVDSVKG\| | MNSLRAEDTAVYYCAR\| |
| Dog_ | 1 | 75.0 | 81.0 | \|.E....F..HR.N.. | \|..G..\| | \|.....QW | \|M.SY...ST. | \|........T..M.... |
| IGHV3- | | | | ...G...Q.......\| | ----- | ...Q..S\| | .T.T.HD\| | ..............M.\| |
| 13*01 | | | | --------------- | | ------- | ---------- | ---------------- |
| | | | | --------------- | | ------- | ------- | ---------------- |

--- IGHV3-64D- (1) ---

| ACCES- | | % | % | -------------- | | ------ | ------CDR2 | --------------- |
|---|---|---|---|---|---|---|---|---|
| SIONS | Count | VGENE | FWs | FW1------------- | CDR1- | FW2----- | ------- | FW3-------------- |
| IGHV3- | 1 | | | \|EVQLVESGGGLVQPG | \|SYAMH\| | \|WVRQAPG | \|AISSNGGSTY | |
| 64D | | | | GSLRLSCSASSFTFS\| | | KGLEYVS\| | YADSVKG\| | |
| Dog_ | 1 | 58.0 | 76.0 | \|............... | \|N.S.I\| | \|......R | \|T..NS.S..N | \|........T....... |
| IGHV3- | | | | ..SPV..V.......\| | ----- | ...QWIT\| | M..T...\| | ....G.D...L....R\| |
| 29*01 | | | | --------------- | | ------- | ---------- | ---------------- |
| | | | | --------------- | | ------- | ------- | ---------------- |

--- IGHV1-46- (1) ---

| ACCES- | | % | % | -------------- | | ------ | ------CDR2 | --------------- |
|---|---|---|---|---|---|---|---|---|
| SIONS | Count | VGENE | FWs | FW1------------- | CDR1- | FW2----- | ------- | FW3-------------- |
| IGHV1- | 1 | | | \|QVQLVQSGAEVKKPG | \|SYYMH\| | \|WVRQAPG | \|TINPSGGSTS | \|RVTNTRDTSTSTVYME |
| 46 | | | | ASVKVSCKASGYTFT\| | | QGLEWNG\| | YAQKFQG\| | LSSLRSEDTAVYYCAR\| |
| Dog_ | 1 | 81.0 | 85.0 | \|E.............. | \|N...I\| | \|....... | \|Q.D.SD.A.. | \|...L.A......A... |
| IGHV1- | | | | ........T.....I\| | ----- | A..D...\| | .......\| | .....AG.I.......\| |
| 30*01 | | | | --------------- | | ------- | ---------- | ---------------- |
| | | | | --------------- | | ------- | ------- | ---------------- |

TABLE 4
Sequence alignments for VL domains (kappa) between human VGene and dog (canis
familiaris) VGene
--- IGKV20-29-(12) ---

| ACCES- | | % | % | ---------- | ------CDR1 | ------ | | --------------- |
|---|---|---|---|---|---|---|---|---|
| SIONS | Count | VGENE | FWs | FW1---------- | ------ | FW2------ | -CDR2-- | FW3-------------- |
| IGKV20- | 1 | | | \|DIVNTQTPLSLS | \|KSSQSLLHSD | \|WYLQKPGQ | \|EVSNRFS\| | \|GVPDRFSGSGSGTDFT |
| 29 | | | | VTPGQPASISC\| | GKTYLY\| | PPQLLIY\| | | LKISRVEAEDVGVYYC\| |
| Dog_ | 1 | 83.0 | 87.0 | \|............ | \|.A.......N | \|.FR..... | \|K....DP\| | \|................ |
| IGKV2- | | | | .S..E......\| | .N....\| | S..R...\| | | .R......D.A.....\| |
| 5*02 | | | | | | | | |
| Dog_ | 1 | 83.0 | 85.0 | \|............ | \|RA.......N | \|.FR..... | \|L......\| | \|W............... |
| IGKV2 | | | | .S..E......\| | .N....\| | S..G...\| | | .R......D.A.....\| |
| 513*01 | | | | | | | | |
| Dog_ | 1 | 83.0 | 85.0 | \|........P... | \|.A........ | \|........ | \|L.....T\| | \|..S............. |
| IGKV2- | | | | .S.RET.....\| | .N...D\| | S......\| | | .R......N.T.....\| |
| 4*02 | | | | | | | | |
| Dog_ | 1 | 82.0 | 85.0 | \|............ | \|.A........ | \|.FR..... | \|K....DP\| | \|................ |
| IGKV2- | | | | .S..ET.....\| | .N...N\| | S..R...\| | | .R......D.T.....\| |
| 9*01 | | | | | | | | |
| Dog_ | 1 | 81.0 | 87.0 | \|EV..I....... | \|RA....R..N | \|........ | \|L...N.T\| | \|..S............. |
| IGKV2- | | | | .S..E......\| | .N....\| | S.....D\| | | .R..............\| |
| 20*02 | | | | | | | | |
| Dog_ | 1 | 81.0 | 85.0 | \|EA.........A | \|RA.....R.. | \|........ | \|.A.K...\| | \|..S............. |
| IGKV2- | | | | ....EL.T...\| | ..S..N\| | T.RP...\| | | ................\| |
| 16*01 | | | | | | | | |
| Dog_ | 1 | 88.0 | 84.0 | \|........P... | \|.A.......N | \|.FR..... | \|H....ST\| | \|..S............. |
| IGKV2- | | | | .S..E......\| | .N...N\| | S..G...\| | | .R......D.A.....\| |
| 8*01 | | | | | | | | |
| Dog_ | 1 | 80.0 | 84.0 | \|.......N.... | \|.A.......N | \|.FR..... | \|K....DP\| | \|................ |
| IGKV2- | | | | .S..ET.....\| | .N...N\| | S..G...\| | | .R......D.A.....\| |
| 7*01 | | | | | | | | |
| Dog_ | 1 | 79.0 | 82.0 | \|..........V. | \|RA........ | \|.FR..... | \|R...C.T\| | \|..S............. |
| IGKV2- | | | | .S..ET.....\| | .N...D\| | I.KD...\| | | .R......DNA.....\| |
| 13*02 | | | | | | | | |
| Dog_ | 1 | 79.0 | 82.0 | \|............ | \|.A.......N | \|.FR..... | \|L....DP\| | \|...H............ |
| IGKV2- | | | | .S..ET.....\| | .N...F\| | S..R..N\| | ------- | .R......D.A.....\| |
| 11*02 | | | | ----------- | ----------- | -------- | | ---------------- |
| | | | | ------------ | ----- | ------- | | ---------------- |

--- IGKV2-28-(1) ---

| ACCES- | | % | % | ---------- | ------CDR1 | ------ | | --------------- |
|---|---|---|---|---|---|---|---|---|
| SIONS | Count | VGENE | FWs | FW1---------- | ------ | FW2------ | -CDR2-- | FW3-------------- |
| IGKV2- | 1 | | | \|DIVNTQSPLSLP | \|RSSQSLLHSN | \|WYLQKPGQ | \|LGSNRAS\| | \|GVPDRFSGSGSGTDFT |
| 28 | | | | VTPGEPASISC\| | GYNYLD\| | SPQLLIY\| | | LKISRVEAEDVGVYYC\| |
| Dog_ | 1 | 80.0 | 87.0 | \|......T....S | \|.AN....... | \|..N...... | \|HV..HFT\| | \|..S............. |
| IGKV2- | | | | .S...T.....\| | .NI...\| | ..G...\| | ------- | ........D.A.....\| |
| 12*02 | | | | ------------ | ---------- | --------- | | ---------------- |
| | | | | ----------- | ------ | ------ | | ---------------- |

--- IGKV4-1(1) ---

| ACCES- | | % | % | ---------- | ------CDR1 | ------ | | --------------- |
|---|---|---|---|---|---|---|---|---|
| SIONS | Count | VGENE | FWs | FW1---------- | ------- | FW2------ | -CDR2-- | FW3-------------- |
| IGKV4- | 1 | | | \|DIVNTQSPDSLA | \|KSSQSVLYSS | \|WYQQKPGQ | \|WASTRES\| | \|GVPDRFSGSGSGTDFT |
| 1 | | | | VSLGERATINC\| | NNKNYLA\| | PPKLLIY\| | | LTISSLQAEDVAVYYC\| |
| Dog_ | 1 | 76.0 | 78.0 | \|E.......G... | \|.....L...F | \|.......E | \|L..SWA.\| | \|...A...S........ |
| IGKV4- | | | | G.A..SVS...\| | .Q.....\| | R......\| | ------- | ...NN......GD...\| |
| 15*01 | | | | ------------ | ----------- | -------- | | ---------------- |
| | | | | ----------- | ------ | ------- | | ---------------- |

--- IGKV3-11-(1) ---

| ACCES- | | % | % | --------- | ---CDR1 | ------ | | --------------- |
|---|---|---|---|---|---|---|---|---|
| SIONS | Count | VGENE | FWs | FW1----------- | ---- | FW2------ | -CDR2-- | FW3-------------- |
| IGKV3- | 1 | | | \|EIVLTQSPATLSL | \|RASQSVS | \|WYQQKPGQ | \|DASNRAT\| | \|GIPARFSGSGSGTDFT |
| 11 | | | | SPGERATLSC\| | SYLA\| | APRLLIY\| | | LTISSLEPEDFAVYYC\| |
| Dog_ | 1 | 81.0 | 80.0 | \|...N.....S... | \|....... | \|........ | \|GT.....\| | \|.V.S...........S |
| IGKV3- | | | | .QE.KV.IT.\| | ....\| | ..K....\| | ------- | F.........V.....\| |
| 18*02 | | | | ------------ | ------- | -------- | | ----------------- |
| | | | | ----------- | ---- | ------- | | --------------- |

TABLE 5
Sequence alignments for VL domains (lambda) between human VGene and dog (canis
familiaris) VGene
--- IGLV1-44-(28) ---

| ACCES- | | % | % | ---------- | ----CDR1 | ------ | | --------------- |
|---|---|---|---|---|---|---|---|---|
| SIONS | Count | VGENE | FWs | FW1--------- | ----- | FW2------ | -CDR2-- | FW3-------------- |
| IGLV1- | 1 | | | \|QSVLTQPPSAS | \|SGSSSNIG | \|WYQQLPGT | \|SNNQRPS\| | \|GVPDRFSGSKSGTSAS |
| 44 | | | | ETPGQRVTISC\| | SNTVN\| | APKLLTY\| | | LATSGLQSEDEADYYC\| |
| Dog_ | 1 | 77.0 | 72.0 | \|.......A.V. | \|I....... | \|........ | \|D.SN...\| | \|............ST.Y |
| IGLV1- | | | | .SL........\| | RGY.G\| | G.RT...\| | | .Y.....A........\| |
| 157*01 | | | | | | | | |
| Dog_ | 1 | 77.0 | 72.0 | \|.......A.V. | \|Y....... | \|........ | \|G.SN...\| | \|............ST.Y |
| IGLV1- | | | | .SL........\| | RGY.G\| | R.RT...\| | | .Y.....A........\| |
| 46*01 | | | | | | | | |
| Dog_ | 1 | 76.0 | 82.0 | \|.........V. | \|T....... | \|........ | \|GLSN...\| | \|.........R..ST.Y |
| IGLV1- | | | | .FL........\| | RGY.H\| | G.RT...\| | | .Y.....A........\| |
| 94*01 | | | | | | | | |
| Dog_ | 1 | 76.0 | 81.0 | \|.......A.V. | \|I....... | \|........ | \|G.SN...\| | \|.........R..ST.Y |
| IGLV1- | | | | .SL........\| | RGV.G\| | G.RT...\| | | .Y.....A........\| |
| 125*01 | | | | | | | | |
| Dog_ | 1 | 76.0 | 78.0 | \|.......A.V. | \|I....... | \|.......R | \|.T.S...\| | \|............ST.Y |
| IGLV1- | | | | .SL...I....\| | G.N.G\| | G.RTV..\| | | .Y.....A........\| |
| 55*01 | | | | | | | | |
| Dog_ | 1 | 75.0 | 84.0 | \|.........V. | \|...TN..S | \|.......K | \|.DGD...\| | \|............K..Y |
| IGLV1- | | | | .SL........\| | IVGAS\| | .....VD\| | | .Y.Y...A........\| |
| 69*01 | | | | | | | | |
| Dog_ | 1 | 75.0 | 82.0 | \|.......T.V. | \|...TN... | \|.......K | \|.YGD...\| | \|.........N..N..Y |
| IGLV1- | | | | .SL........\| | IVGAS\| | .....V.\| | | .Y.Y...A........\| |
| 141*01 | | | | | | | | |
| Dog_ | 1 | 75.0 | 82.0 | \|.......T.V. | \|...TN... | \|.......K | \|.DGD...\| | \|............N..Y |
| IGLV1- | | | | .SL........\| | IVGAS\| | .....VD\| | | .Y.Y...A........\| |
| 108*81 | | | | | | | | |
| Dog_ | 1 | 75.0 | 76.0 | \|..M....A.V. | \|T....... | \|.......T | \|G..Y...\| | \|............S..Y |
| IGLV1- | | | | .SL..A.....\| | G.Y.G\| | G.RTV..\| | | .Y.....A....E...\| |
| 48*01 | | | | | | | | |
| Dog_ | 1 | 75.0 | 70.0 | \|.......A.V. | \|T....... | \|........ | \|DSSS...\| | \|.........R..ST.Y |
| IGLV1- | | | | .SL........\| | RGY.G\| | G.RT...\| | | .Y.....A........\| |
| 136*01 | | | | | | | | |
| Dog_ | 1 | 74.0 | 81.0 | \|.......T.V. | \|...TN... | \|.......K | \|.DGD...\| | \|.........N..N.DT |
| IGLV1- | | | | .SL........\| | TVGAS\| | .....V.\| | | .T.T...A........\| |
| 162*01 | | | | | | | | |
| Dog_ | 1 | 74.0 | 78.0 | \|.......A.V. | \|I....... | \|.F.....K | \|.SSN...\| | \|............SY.Y |
| IGLV1- | | | | .SL........\| | RYS.G\| | G.RTV..\| | | .Y.....A........\| |
| 58*01 | | | | | | | | |
| Dog_ | 1 | 74.0 | 76.0 | \|.P.......V. | \|T......L | \|.....T.K | \|VD.N...\| | \|....Q.......N..Y |
| IGLV1- | | | | .FL........\| | G.S..\| | G.RTV..\| | | .Y.....A...T....\| |
| 118*01 | | | | | | | | |
| Dog_ | 1 | 71.0 | 78.0 | \|.........V. | \|...TNS.. | \|...L.S.K | \|GTGN...\| | \|............N.GY |
| IGLV1- | | | | .SV...Y....\| | YIG..\| | .....VD\| | | .Y.Y...D........\| |
| 86*01 | | | | | | | | |
| Dog_ | 1 | 71.0 | 78.0 | \|....N....V. | \|...TND.. | \|.......N | \|GTGN...\| | \|....Q.......N.GY |
| IGLV1- | | | | .SL...I....\| | MLG..\| | .....VD\| | | .Y.Y...A........\| |
| 73*01 | | | | | | | | |
| Dog_ | 1 | 71.0 | 75.0 | \|.......A... | \|.S.TN... | \|....I..K | \|D.EK...\| | \|...N........DLST |
| IGLV1- | | | | .SL.....V..\| | IIG.K\| | .M.....\| | | .Y.N...G........\| |
| 84-1*01 | | | | | | | | |
| Dog_ | 1 | 71.0 | 75.0 | \|.......A.VT | \|T....... | \|.F...... | \|.SSN...\| | \|.........R..ST.Y |
| IGLV1- | | | | .SL........\| | GYN.G\| | G.RTV..\| | | .Y.....A....E...\| |
| 75*01 | | | | | | | | |
| Dog_ | 1 | 71.0 | 79.0 | \|.......A.V. | \|Y....... | \|.H..V.E. | \|AD.Y.A.\| | \|............ST.Y |
| IGLV1- | | | | .VL........\| | G.Y.S\| | G.RNI..\| | | .Y..V..A........\| |
| 67*01 | | | | | | | | |
| Dog_ | 1 | 89.0 | 79.0 | \|.......T.V. | \|.R.TN... | \|.......K | \|.DGDQL.\| | \|............N..N |
| IGLV1- | | | | WSL........\| | TVGAS\| | .....VD\| | | .Y.Y...A..K.....\| |
| 80*01 | | | | | | | | |
| Dog_ | 1 | 89.0 | 75.0 | \|....N.L..VL | \|...TND.. | \|...E...K | \|GTGN...\| | \|............N.GY |
| IGLV1- | | | | .SL...I....\| | MLG..\| | .....VD\| | | .Y.Y...A........\| |
| 155*01 | | | | | | | | |
| Dog_ | 1 | 67.0 | 75.0 | \|.......A.V. | \|Y....S.. | \|........ | \|GTSNL.P\| | \|...N.....R..ST.Y |
| IGLV1- | | | | .SL....I...\| | RGY.G\| | G.RT...\| | | .Y.AE..A........\| |
| 195*01 | | | | | | | | |
| Dog_ | 1 | 67.0 | 75.0 | \|.........VF | \|Y...C.V. | \|.....L.. | \|GSSNQ..\| | \|...N.....R..ST.Y |
| IGLV1- | | | | RSL........\| | RGY.Y\| | R.RT...\| | | .Y...F.A........\| |
| 158*01 | | | | | | | | |
| Dog_ | 1 | 86.0 | 72.0 | \|.........VF | \|T.....V. | \|.....L.. | \|GSSNQ..\| | \|...NQ....R..STDY |
| IGLV1- | | | | RSL........\| | RGY.I\| | R.RT...\| | | .Y...F.A........\| |
| 97-4*01 | | | | | | | | |
| Dog_ | 1 | 58.0 | 67.0 | \|............ | \|I..CF... | \|.L...... | \|YDRS...\| | \|............ST.Y |
| IGLV1- | | | | ...PE..V..\| | RVS.S\| | G.RTT..\| | ------- | .Y.....A........\| |
| 127*01 | | | | ------------ | -------- | -------- | | ---------------- |
| | | | | ---------- | ----- | ------- | | ---------------- |

--- IGLV8-61-(17) ---

| ACCES- | | % | % | ---------- | ----- | ------ | | --------------- |
|---|---|---|---|---|---|---|---|---|
| SIONS | Count | VGENE | FWs | FW1--------- | CDR1----- | FW2------ | -CDR2-- | FW3-------------- |
| IGLV8- | 1 | | | \|QTVVTQEPSFSV | \|GLFSGSVS | \|WYQQTPGQ | \|STNTRSS\| | \|GVPDRFSGSILGNKAA |
| 61 | | | | SPGQTVTLIC\| | TSVYPS\| | APRTLIV\| | | LIIYGAQADDESDYYC\| |
| Dog_ | 1 | 83.0 | 86.0 | \|.........L.. | \|........ | \|.....L.R | \|R.SS.P.\| | \|...N......S..... |
| IGLV8- | | | | ..........\| | ..N..G\| | ....I..\| | | .......PE..A....\| |
| 128*01 | | | | | | | | |
| Dog_ | 1 | 83.0 | 86.0 | \|.........L.. | \|........ | \|.....Q.R | \|N.SS.P.\| | \|...N......S..... |
| IGLV8- | | | | ..........\| | ..N..G\| | ....I..\| | | .......PE..A....\| |
| 93*01 | | | | | | | | |
| Dog_ | 1 | 83.0 | 86.0 | \|.........L.. | \|........ | \|.S...... | \|N..S.P.\| | \|...N..T...S..... |
| IGLV8- | | | | .L........\| | ..N..N\| | ....T..\| | | .......PE..A....\| |
| 36*01 | | | | | | | | |
| Dog_ | 1 | 81.0 | 82.0 | \|.........L.. | \|........ | \|.....Q.K | \|N..N.P.\| | \|.I.NK.....S....S |
| IGLV8- | | | | ..........\| | ..NH..\| | ...M...\| | | .......PE..Y....\| |
| 39*01 | | | | | | | | |
| Dog_ | 1 | 80.0 | 81.0 | \|......K..L.. | \|........ | \|.....Q.R | \|..SS.P.\| | \|...N..T...S..... |
| IGLV8- | | | | ........I.\| | ..N..G\| | .S..I..\| | | .......PE..A....\| |
| 147*01 | | | | | | | | |
| Dog_ | 1 | 78.0 | 81.0 | \|......K..L.. | \|.F...... | \|.....Q.R | \|..SS.P.\| | \|...N..P...S..... |
| IGLV8- | | | | ........I.\| | ..N..G\| | .S..I..\| | | .......PE..A....\| |
| 99*01 | | | | | | | | |
| Dog_ | 1 | 78.0 | 79.0 | \|......K..L.. | \|........ | \|.....Q.R | \|..SS.P.\| | \|...N..P...S..... |
| IGLV8- | | | | ........T.\| | ..N..G\| | .S..I..\| | | ..T....PE..A....\| |
| 161*01 | | | | | | | | |
| Dog_ | 1 | 78.0 | 74.0 | \|.I.......L.. | \|.......I | \|.......R | \|N..N.P.\| | \|...NM.....S..... |
| IGLV8- | | | | ..........\| | I.N..D\| | S..M...\| | | .......PE..AV...\| |
| 74-1*01 | | | | | | | | |
| Dog_ | 1 | 77.0 | 82.0 | \|.........L.. | \|........ | \|...M.... | \|D.SS.P.\| | \|...N......SE.... |
| IGLV8- | | | | ..........\| | .GNK.G\| | ...RI..\| | | ....E..PE..AA.H.\| |
| 110*01 | | | | | | | | |
| Dog_ | 1 | 75.0 | 81.0 | \|.......S.V.. | \|......LY | \|.....Q.. | \|D.SSCP.\| | \|E...R.....S..... |
| IGLV8- | | | | ..V.......\| | ..N.Y.\| | S..N.V.\| | | .......PE..A....\| |
| 121*01 | | | | | | | | |
| Dog_ | 1 | 72.0 | 72.0 | \|.......QEP.L | \|.......T | \|.......R | \|R..SMP.\| | \|..........S..... |
| IGLV8- | | | | ......L...\| | ..N.S.\| | ....T..\| | | .......PE..A....\| |
| 102*01 | | | | | | | | |
| Dog_ | 1 | 72.0 | 71.0 | \|......D..L.. | \|........ | \|.....QVR | \|...SVP.\| | \|...NC.T...S.K... |
| IGLV8- | | | | ..........\| | ..N..G\| | ..CM...\| | | .....D.PE..TIIVH\| |
| 76-1*01 | | | | | | | | |
| Dog_ | 1 | 72.0 | 69.0 | \|...I..DT.L.. | \|........ | \|.....Q.R | \|...SMP.\| | \|...NC.TR..S.K... |
| IGLV8- | | | | ..........\| | ..N...\| | D..M...\| | | .......PE..TIIVH\| |
| 68*01 | | | | | | | | |
| Dog_ | 1 | 68.0 | 81.0 | \|............ | \|........ | \|.....Q.R | \|N..N.P.\| | \|..........S..... |
| IGLV8- | | | | ..........\| | ..N..N\| | ..MRV..\| | | ....A..PE..A....\| |
| 43*01 | | | | | | | | |
| Dog_ | 1 | 66.0 | 89.0 | \|...GG..ALL.. | \|.......T | \|.P.....R | \|..KDCP.\| | \|....C..R..S..... |
| IGLV8- | | | | ....R.....\| | ..N..N\| | ..G.T..\| | | .......SE..AITVL\| |
| 89-3*01 | | | | | | | | |
| Dog_ | 1 | 64.0 | 67.0 | \|....G..ALL.. | \|.......T | \|.P.....R | \|..KDCP.\| | \|....C..R..S..... |
| IGLV8- | | | | .S..R.....\| | ..N..N\| | ..G.IN.\| | ------- | .......SE..VITVL\| |
| 118-3*01 | | | | ------------ | -------- | -------- | | ---------------- |
| | | | | ---------- | ------ | ------- | | ---------------- |

--- IGLV1-40-(11) ---

| ACCES- | | % | % | ---------- | ----- | ------ | | --------------- |
|---|---|---|---|---|---|---|---|---|
| SIONS | Count | VGENE | FWs | FW1--------- | CDR1----- | FW2------ | -CDR2-- | FW3------------- |
| IGLV1- | 1 | | | \|QSVLTQPPSVS | \|TGSSSNIG | \|WYQQLPGT | \|GNSNRPS\| | \|GVPDRFSGSKSGTSAS |
| 40 | | | | GAPGQRVTISC\| | ASYDVW\| | APKLLTY\| | | LAITGLQAEDEADYY\| |
| Dog_ | 1 | 84.0 | 84.0 | \|.A......... | \|....Y... | \|.......K | \|.......\| | \|............ST.. |
| IGLV1- | | | | AVL........\| | S....Q\| | S..YK..\| | | .Y.............\| |
| 40*01 | | | | | | | | |
| Dog_ | 1 | 81.0 | 81.0 | \|.A......... | \|...NY... | \|.......K | \|.......\| | \|...V........ST.Y |
| IGLV1- | | | | A.L........\| | S....Q\| | S..YK..\| | | .Y...E.........\| |
| 103*01 | | | | | | | | |
| Dog_ | 1 | 80.0 | 85.0 | \|.......A... | \|...TN... | \|.......K | \|.DD..E.\| | \|...E........S..Y |
| IGLV1- | | | | .SL........\| | GDNY..\| | ..S....\| | | .Y.............\| |
| 87*01 | | | | | | | | |
| Dog_ | 1 | 78.0 | 72.0 | \|.A.....A... | \|......V. | \|........ | \|.S.Y...\| | \|.........S..S..Y |
| IGLV1- | | | | .SL........\| | Y.NY.G\| | G.RT...\| | | .Y.S...........\| |
| 84*01 | | | | | | | | |
| Dog_ | 1 | 77.0 | 72.0 | \|.......A... | \|......V. | \|........ | \|RS.S...\| | \|.........R..ST.Y |
| IGLV1- | | | | .SL........\| | Y.NY.G\| | G.RT...\| | | .Y.S...........\| |
| 149*01 | | | | | | | | |
| Dog_ | 1 | 77.0 | 72.0 | \|.......A... | \|......V. | \|........ | DS.S....\| | \|.........R..ST.Y |
| IGLV1- | | | | .SL........\| | Y.NY.G\| | G.RT...\| | | .Y.S...........\| |
| 138*01 | | | | | | | | |
| Dog_ | 1 | 76.0 | 82.0 | \|.......A... | \|.R....V. | \|........ | \|NTNY...\| | \|............ST.Y |
| IGLV1- | | | | .SL........\| | Y.N..G\| | G.RTK..\| | | .Y.S...........\| |
| 70*01 | | | | | | | | |
| Dog_ | 1 | 74.0 | 82.0 | \|.......A... | \|......V. | \|........ | \|YTNT..Y\| | \|............N..Y |
| IGLV1- | | | | QSL........\| | YNSY.S\| | V.RTK..\| | | .Y.A...........\| |
| 112*01 | | | | | | | | |
| Dog_ | 1 | 74.0 | 76.0 | \|.A...PL.... | \|..N.YQ.. | \|.......K | \|.D.....\| | \|........FS..N..Y |
| IGLV1- | | | | A.L..T.....\| | S..A.Q\| | S.ETK..\| | | .......D.......\| |
| 150*01 | | | | | | | | |
| Dog_ | 1 | 73.0 | 76.0 | \|.A...PL.... | \|..N.YQ.A | \|.......K | \|.D.....\| | \|........FS..N..Y |
| IGLV1- | | | | A.L..T.....\| | S..A.Q\| | S.ETK..\| | ------- | .......D.......\| |
| 57*01 | | | | ----------- | -------- | -------- | | ---------------- |
| | | | | ----------- | ------ | ------- | | --------------- |

--- IGLV2-18-(8) ---

| ACCES- | | % | % | ---------- | ----- | ------ | | --------------- |
|---|---|---|---|---|---|---|---|---|
| SIONS | Count | VGENE | FWs | FW1--------- | CDR1----- | FW2------ | -CDR2-- | FW3------------- |
| IGLV2- | 1 | | | \|QSALTQPPSVS | \|TGTSSDVG | \|WYQQPPGT | \|EVSNRPS\| | \|GVPDRFSGSKSGNTASL |
| 18 | | | | GSPGQSVTISC\| | SYNRVS\| | APKLNTY\| | | YTSGLQAEDEADYY\| |
| Dog_ | 1 | 77.0 | 78.0 | \|.......S... | \|D.S..NI. | \|....F... | \|YTN....\| | \|.I.A............. |
| IGLV1- | | | | .TL..T.....\| | .S.VTE\| | S...L..\| | | ..............\| |
| 31*01 | | | | | | | | |
| Dog_ | 1 | 76.0 | 84.0 | \|..V....A... | \|..S..N.. | \|....F... | \|YD.S...\| | \|............S..Y. |
| IGLV1- | | | | ..L..R.....\| | YSSS.G\| | G.RTK..\| | | ..............\| |
| 41*01 | | | | | | | | |
| Dog_ | 1 | 73.0 | 79.0 | \|..V....A... | \|..S..N.. | \|....L... | \|YTNT...\| | \|.....Y......S..Y. |
| IGLV1- | | | | ..L..R.....\| | YG.Y.G\| | G.RTE.C\| | | ..........T...\| |
| 92*01 | | | | | | | | |
| Dog_ | 1 | 71.0 | 79.0 | \|......TA.N. | \|..S..N.. | \|....L... | \|NYNT...\| | \|.........I..S..Y. |
| IGLV1- | | | | ..L..R..V..\| | YRSY.G\| | G.RTE..\| | | ..A...........\| |
| 151*01 | | | | | | | | |
| Dog_ | 1 | 68.0 | 64.0 | \|.........N. | \|..SG..I. | \|...EL.S. | \|GTN...L\| | \|EE.A...........PM |
| IGLV1- | | | | .TL..TII...\| | R.SY..\| | E.T.L..\| | | ...........N..\| |
| 32*01 | | | | | | | | |
| Dog_ | 1 | 62.0 | 82.0 | \|........... | \|.RS..N.. | \|....L... | \|NYNY...\| | \|............R..Y. |
| IGLV1- | | | | .G..PE.....\| | YSSY.G\| | G..TI..\| | | ..A...........\| |
| 83*01 | | | | | | | | |
| Dog_ | 1 | 62.0 | 82.0 | \|........... | \|.RS..N.. | \|....L... | \|NTNT...\| | \|............S..Y. |
| IGLV1- | | | | .G..PE.....\| | YSSY.G\| | G..TI..\| | ------- | ..A...........\| |
| 70*01 | | | | ----------- | -------- | -------- | | ---------------- |
| | | | | ----------- | ------ | ------- | | --------------- |

--- IGLV3-9-(8) ---

| ACCES- | | % | % | ---------- | --- CDR1 | ------ | | --------------- |
|---|---|---|---|---|---|---|---|---|
| SIONS | Count | VGENE | FWs | FW1--------- | ---- | FW2------ | -CDR2-- | FW3-------------- |
| IGLV3- | 1 | | | \|SYELTQPLSVS | \|GGNNIGSK | \|WVQQKPGQ | \|RDSNRPS\| | \|GIPERFSGSNSGNTAT |
| 9 | | | | VALGQTARTTC\| | NVH\| | APVLVIY\| | | LTISRAQAEDEADYYC\| |
| Dog_ | 1 | 81.0 | 84.0 | \|..V...LP... | \|..DS.... | \|.....L.. | \|......T\| | \|........A....... |
| IGLV3- | | | | .T.R.......\| | S.Y\| | ....I..\| | | ....G.L.E.......\| |
| 8*01 | | | | | | | | |
| Dog_ | 1 | 81.0 | 84.0 | \|..V...LP... | \|..DS.... | \|.....L.. | \|D..S...\| | \|........A....... |
| IGLV3- | | | | .T.R.......\| | ..Y\| | ....I..\| | | ....G.L.E.......\| |
| 24*01 | | | | | | | | |
| Dog_ | 1 | 78.0 | 78.0 | \|..V...LP..N | \|..DS.... | \|.....L.. | \|G......\| | \|........D....... |
| IGLV3- | | | | .T.R.P.H...\| | S..\| | ....I..\| | | ....G.L.E...Y...\| |
| 26*01 | | | | | | | | |
| Dog_ | 1 | 77.0 | 82.0 | \|..V...SP... | \|R..S..R. | \|........ | \|N.NSQ..\| | \|........T...S... |
| IGLV3- | | | | .T.....S...\| | D..\| | ..L.I..\| | | ....G..TN.......\| |
| 2*01 | | | | | | | | |
| Dog_ | 1 | 77.0 | 81.0 | \|..V...LP.M. | \|R.DS.... | \|.....L.. | \|D..S...\| | \|........A....... |
| IGLV3- | | | | .T.R.......\| | S.Y\| | V...I..\| | | ....G.L.E.......\| |
| 14*01 | | | | | | | | |
| Dog_ | 1 | 74.0 | 80.0 | \|....S..P.AT | \|..DS.... | \|........ | \|G..S...\| | \|........A....... |
| IGLV3- | | | | .T.R....L..\| | S.E\| | P...I..\| | | ....G.L.E.......\| |
| 11*01 | | | | | | | | |
| Dog_ | 1 | 68.0 | 83.0 | \|........... | \|..D..... | \|.....L.. | \|V..S..T\| | \|........A....... |
| IGLV3- | | | | ...R.P.H...\| | S..\| | ....I..\| | | ....G.L.E.......\| |
| 3*01 | | | | | | | | |
| Dog_ | 1 | 58.0 | 62.0 | \|ANK...S.FN. | \|.RD.S.R. | \|......S. | \|D.CFQ..\| | \|.FS.Q...Y....... |
| IGLV3- | | | | .....M.....\| | SA.\| | ...NL.D\| | ------- | ....GPP.HYRLLLCH\| |
| 1*01 | | | | ----------- | -------- | -------- | | ---------------- |
| | | | | ----------- | --- | ------- | | ---------------- |

--- IGLV3-25-(3) ---

| ACCES- | | % | % | ---------- | --- | ------ | | --------------- |
|---|---|---|---|---|---|---|---|---|
| SIONS | Count | VGENE | FWs | FW1--------- | CDR1---- | FW2------ | -CDR2-- | FW3-------------- |
| IGLV3- | 1 | | | \|SYELMQPPSVS | \|SGDALPK | \|WYQQKPGQ | \|KDSERPS\| | \|GIPERFSGSSSGTTVT |
| 25 | | | | VSPGQTARTTC\| | QVAY\| | APVLVIY\| | | LTISGVQAEDEADYYC\| |
| Dog_ | 1 | 74.0 | 79.0 | \|.SV.T...... | \|..ES.SR | \|........ | \|G.R....\| | \|...D...S...EN.H. |
| IGLV3- | | | | ..L....T...\| | Y..Q\| | ..MT...\| | | .....A......E...\| |
| 29*01 | | | | | | | | |
| Dog_ | 1 | 70.0 | 72.0 | \|.PG.N.....L | \|...V.G. | \|...M..S. | \|.NN.LG.\| | \|...D.....N..N.A. |
| IGLV3- | | | | .AL..N.TN..\| | R...\| | ....L.N\| | | .....AR.........\| |
| 19*01 | | | | | | | | |
| Dog_ | 1 | 64.0 | 69.0 | \|....N.A...L | \|.R.V.G. | \|H..M..S. | \|.NN.QD.\| | \|...D.....N..N.A. |
| IGLV3- | | | | .AL..NET...\| | R...\| | ....L.N\| | ------- | .....AR.....E...\| |
| 15*01 | | | | ----------- | ------- | -------- | | ---------------- |
| | | | | ----------- | ---- | ------- | | ---------------- |

--- IGLV3-27-(2) ---

| ACCES- | | % | % | ----------- | --- | ------ | | --------------- |
|---|---|---|---|---|---|---|---|---|
| SIONS | Count | VGENE | FWs | FW1-------- | CDR1---- | FW2------ | -CDR2-- | FW3-------------- |
| IGLV3- | 1 | | | \|SYELTQPSSVS | \|SGDVLAK | \|WYQQKPGQ | \|KDSERPS\| | \|GIPERFSGSSSGTTYT |
| 27 | | | | VSPGQTARTTC\| | KYAR\| | APVLYIY\| | | LTISGAQVEDEADYYC\| |
| Dog_ | 1 | 78.0 | 81.0 | \|.SV....P... | \|..ES.S. | \|.....A.. | \|..T....\| | \|...D........N.H. |
| IGLV3- | | | | ..L....T.S.\| | Y..Q\| | V......\| | | ......RA........\| |
| 27*01 | | | | | | | | |
| Dog_ | 1 | 60.0 | 60.0 | \|..........L | \|.R...G. | \|R.H..S.. | \|.NN.QD.\| | \|...D.....N..N.A. |
| IGLV3- | | | | .AL..MET...\| | R..Y\| | ....L.N\| | ------- | ......RA....E...\| |
| 25*01 | | | | ----------- | ------- | -------- | | ---------------- |
| | | | | ----------- | ---- | ------- | | ---------------- |

--- IGLV3-37-(2) ---

| ACCES- | | % | % | ---------- | ----- | ------ | --- | --------------- |
|---|---|---|---|---|---|---|---|---|
| SIONS | Count | VGENE | FWs | FW1--------- | CDR1----- | FW2------ | CDR2---- | FW3-------------- |
| IGLV3- | 1 | | | \|QPVLTQPPSSS | \|TLPSDTNV | \|WYQQKPGS | \|YYSDSDK | \|GVPSRFSGSKDASANT |
| 37 | | | | ASPGESARLTC\| | SGYNIY\| | PPRYLLY\| | GQGS\| | GTLLISGLQSEDEADY\| |
| Dog_ | 1 | 77.0 | 88.0 | \|.........L. | \|..S.GFS. | \|........ | \|.....Y. | \|..SKS......Y...A |
| IGLV3- | | | | ..L.YY.....\| | .D.DM.\| | ...D...\| | N...\| | .L.......P......\| |
| 131*01 | | | | | | | | |
| Dog_ | 1 | 78.0 | 78.0 | \|.L.V.....L. | \|..S.GFS. | \|.F...... | \|.H..... | \|R..........T...A |
| IGLV3- | | | | ..L.S......\| | ...SVT\| | .LW....\| | H...\| | .L.......P......\| |
| 64*01 | | | | ----------- | -------- | -------- | ------- | ---------------- |
| | | | | ----------- | ------ | ------- | ---- | ---------------- |

--- IGLV3-21-(1) ---

| ACCES- | | % | % | ---------- | ---CDR1 | ------ | | --------------- |
|---|---|---|---|---|---|---|---|---|
| SIONS | Count | VGENE | FWs | FW1--------- | ---- | FW2------ | -CDR2-- | FW3-------------- |
| IGLV3- | 1 | | | \|SYVLTQPPSVS | \|GGNNIGS | \|WYQQKPGQ | \|YDSDRPS\| | \|GIPERFSGSNEGNTATL |
| 21 | | | | VAPGKTARITC\| | KSVM\| | APVLVIY\| | | TISRVEAGDEADYYC\| |
| Dog_ | 1 | 81.0 | 80.0 | \|......L.... | \|E.....D | \|........ | \|E..K...\| | \|................. |
| IGLV3- | | | | .NL....S...\| | .YAV\| | .....T.\| | ------- | ...GAR.E.......\| |
| 28*01 | | | | ----------- | ------- | -------- | | ----------------- |
| | | | | ----------- | ---- | ------- | | --------------- |

--- IGLV3-61-(1) ---

| ACCES- | | % | % | --------- | ---CDR1 | ------ | | --------------- |
|---|---|---|---|---|---|---|---|---|
| SIONS | Count | VGENE | FWs | FW1---------- | ---- | FW2------ | -CDR2-- | FW3-------------- |
| IGLV3- | 1 | | | \|SYELTQPPSVVS | \|SGEALPK | \|WYQQKPGQ | \|KDSERPS\| | \|SEPTRFSGSSSGTIVT |
| 61 | | | | VLGQNARITC\| | KYAY\| | FPVLVIY\| | | LTESEVQAEDEADYYC\| |
| Dog_ | 1 | 63.0 | 70.0 | \|.........ML. | \|..DI.G. | \|...H..S. | \|.NN..A.\| | \|...HM....N..HSA. |
| IGLV3- | | | | A....ET...\| | R...\| | A...L.N\| | ------- | .....AR.........\| |
| 4*01 | | | | ------------ | ------- | -------- | | ---------------- |
| | | | | ---------- | ---- | ------- | | ---------------- |

--- IGLV1-51-(1) ---

| ACCES- | | % | % | ---------- | ----CDR1 | ------ | | --------------- |
|---|---|---|---|---|---|---|---|---|
| SIONS | Count | VGENE | FWs | FW1-------- | ----- | FW2------ | -CDR2-- | FW3-------------- |
| IGLV1- | 1 | | | \|QSVLTQPPSVS | \|SGSSSNTG | \|WYQQLPGT | \|DNNKRPS\| | \|GTPDRFSGSKSGTSAT |
| 51 | | | | AAPGQKVTISC\| | NNYVS\| | APKLLIV\| | | LGTTGLQTGDEADYYC\| |
| Dog_ | 1 | 70.0 | 78.0 | \|.A..N..A... | \|...TND.D | \|.......K | \|SDGD...\| | \|.V.......S..N.G. |
| IGLV1- | | | | G.L........\| | YTG..\| | .....VD\| | ------- | .Y.....AE.......\| |
| 44*01 | | | | ----------- | -------- | -------- | | ---------------- |
| | | | | ----------- | ----- | ------- | | ---------------- |

--- IGLV3-39-(1) ---

| ACCES- | | % | % | ---------- | ----- | ------ | --- | --------------- |
|---|---|---|---|---|---|---|---|---|
| SIONS | Count | VGENE | FWs | FW1--------- | CDR1----- | FW2------ | CDR2---- | FW3-------------- |
| IGLV3- | 1 | | | \|QPVLTQPTSLS | \|TLRSGINV | \|WYQQKPGS | \|YKSDSDK | \|GVPSRFSGSKDASTNA |
| 39 | | | | ASPGASARFTC\| | GTYRIY\| | LPKYLLR\| | QQGS\| | GLLLISGLQSEDEADY\| |
| Dog_ | 1 | 74.0 | 72.0 | \|.......P.... | \|..S..FS. | \|.F...SE. | \|FY...NE | \|...........Y..Y. |
| IGLV3- | | | | .L.ST..P..\| | .S.H.L\| | P......\| | H..R\| | .........P......\| |
| 85*01 | | | | ------------ | -------- | -------- | ------- | ---------------- |
| | | | | ---------- | ------ | ------- | ---- | ---------------- |

TABLE 6
Sequence alignments for VH domains between human VGene and dingo (canis lupus
dingo) VGene
--- IGHV3-74-(10) ---

| ACCES- | | % | % | -------------- | | ------ | ------- | --------------- |
|---|---|---|---|---|---|---|---|---|
| SIONS | Count | VGENE | FWs | FW1------------- | CDR1- | FW2----- | CDR2------ | FW3-------------- |
| IGHV3- | 1 | | | \|EVQLVESGGGLVQPG | \|SYWMH\| | \|WVKQAPG | \|RINSDGSST | \|RFTISRDNASNTLYLQ |
| 74 | | | | GSLRLSCAASGFTFS\| | | KGLVWVS\| | SYADSVKG\| | NNSLRAEDTAVYYCAH\| |
| Dingo-1 | 1 | 87.0 | 90.0 | \|.........D..K.A | \|..G.S\| | \|....... | \|G...G.... | \|................ |
| 680000- | | | | .......V.......\| | | ...Q..A\| | ....A...\| | ...............K\| |
| 1681500 | | | | | | | | |
| Dingo- | 1 | 87.0 | 90.0 | \|.........DM.K.. | \|..Y.Y\| | \|.A..... | \|H..K..... | \|................ |
| 1338000- | | | | .......V.......\| | | ...Q...\| | ....A...\| | ...............K\| |
| 1339500 | | | | | | | | |
| Dingo- | 1 | 86.0 | 90.0 | \|....A....D..K.. | \|.NY.S\| | \|.T..... | \|Q.S...... | \|................ |
| 1320000- | | | | .......V.......\| | | ...Q...\| | ....A...\| | .....D..........\| |
| 1321500 | | | | | | | | |
| Dingo- | 1 | 85.0 | 89.0 | \|.........D..K.. | \|....S\| | \|C...... | \|Y...G.... | \|................ |
| 1970000- | | | | .......VT......\| | | .E.Q..A\| | W.T.A...\| | ................\| |
| 1971500 | | | | | | | | |
| Dingo- | 1 | 83.0 | 88.0 | \|.........D..K.. | \|.SD.S\| | \|.I....E | \|Y.SH..... | \|................ |
| 1428000- | | | | .......V.......\| | | ...Q..A\| | ....A...\| | .S.............D\| |
| 1427500 | | | | | | | | |
| Dingo- | 1 | 82.0 | 88.0 | \|.........D..K.. | \|..Y.F\| | \|.I..... | \|Y..K..... | \|................ |
| 1619000- | | | | .......V.......\| | | ..NQ..G\| | Y.P.A...\| | ....TV...L......\| |
| 1620500 | | | | | | | | |
| Dingo- | 1 | 81.0 | 84.0 | \|.......R.D..K.. | \|..S.Q\| | \|C...... | \|Y...G.... | \|..T......V...... |
| 1815000- | | | | .......V.Y.....\| | | ..VQ..A\| | .S..A...\| | ...............G\| |
| 1816500 | | | | | | | | |
| Dingo- | 1 | 77.0 | 81.0 | \|....L....D..K.. | \|G..KY\| | \|.IH.... | \|N.SH....K | \|......K......... |
| 1351000- | | | | .......V..E.E..\| | | ...Q..T\| | ....A...\| | ........M.....MM\| |
| 1507500 | | | | | | | | |
| Dingo- | 1 | 75.0 | 78.0 | \|......TE.D..K.. | \|..D.D\| | \|..C.... | \|Y.SNG.... | \|Q.........R..... |
| 1506000- | | | | .......V....E..\| | | ...Q..T\| | R...A...\| | ..N..D..M.....VS\| |
| 1507500 | | | | | | | | |
| Dingo- | 1 | 74.0 | 76.0 | \|......P..E..K.. | \|..N.G\| | \|.AH.P.. | \|GF..G.... | \|E.......V....... |
| 1726000- | | | | A......VVP.....\| | ----- | ..MQ..A\| | ..T.A...\| | .....S........VK\| |
| 1727500 | | | | --------------- | | ------- | --------- | ---------------- |
| | | | | --------------- | | ------- | -------- | ---------------- |

--- IGHV3-230-(5) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLLESGGGLVQPG | \|SYAMS\| | \|WVRQAPGK | \|AISGSGGSTY | \|RFTISRDNSKNTLYLQ |
| 230 | | | | GSLRLSCAASGFTFS\| | | GLEWVS\| | YADSVKG\| | WNSLRAEDTAVYYCAK\| |
| Dingo- | 1 | 86.0 | 89.0 | \|....V....D..K.. | \|..D..\| | \|....S... | \|..MR..S... | \|........A....... |
| 1879000- | | | | .......V.......\| | | ..Q..A\| | ...A...\| | ................\| |
| 1888500 | | | | | | | | |
| Dingo- | 1 | 85.0 | 88.0 | \|....V....D..K.. | \|..Y..\| | \|.I...... | \|D..D.....G | \|......E.A....... |
| 1895000- | | | | .......V.......\| | | ..Q..A\| | ...A...\| | ................\| |
| 1896500 | | | | | | | | |
| Dingo- | 1 | 85.0 | 88.0 | \|....V....E..K.. | \|..Y..\| | \|.I...... | \|D..D...... | \|........V..S.... |
| 1805000- | | | | .......V.......\| | | ..Q..A\| | .T.A...\| | ................\| |
| 1806500 | | | | | | | | |
| Dingo- | 1 | 83.0 | 88.0 | \|....V....D..K.. | \|N.D..\| | \|........ | \|...YD.S... | \|........AR..V... |
| 1926000- | | | | .......V.......\| | | ..Q..A\| | .T.A...\| | ................\| |
| 1927500 | | | | | | | | |
| Dingo- | 1 | 72.0 | 75.0 | \|....V....D..K.V | \|N....\| | \|..H..... | \|E..H..R..G | \|....T...A..MP..N |
| 1644000- | | | | A......V.......\| | ----- | ..Q..A\| | .T.AM..\| | ....T.K.M.M....R\| |
| 1645500 | | | | --------------- | | -------- | ---------- | ---------------- |
| | | | | --------------- | | ------ | ------- | ---------------- |

--- IGHV3-21-(4) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLVESGGGLVKPG | \|SYSMN\| | \|WVRQAPG | \|STSSSSSYTY | \|RFTISRDNAKNSLYLQ |
| 21 | | | | GSLRLSCAASGFTFS\| | | KGLEWVS\| | YADSVKG\| | MNSLRAEDTAVYYCAK\| |
| Dingo- | 1 | 87.0 | 93.0 | \|.........D..... | \|..D.D\| | \|....... | \|E....G.ST. | \|...........Y.... |
| 1496000- | | | | .......V.......\| | | ...Q.L.\| | ...A...\| | ................\| |
| 1497500 | | | | | | | | |
| Dingo- | 1 | 84.0 | 89.0 | \|.........D....V | \|..D..\| | \|....... | \|Y...GG.ST. | \|...........Y.... |
| 1835000- | | | | .......V.......\| | | ...Q..A\| | ...A...\| | ..........M....G\| |
| 1836500 | | | | | | | | |
| Dingo- | 1 | 76.0 | 84.0 | \|.........D..... | \|N.E.Y\| | \|....... | \|R.YE.G.TT. | \|...........MV... |
| 1448000- | | | | .......V.......\| | | ......A\| | ..EA...\| | .........VKGPQCE\| |
| 1449500 | | | | | | | | |
| Dingo- | 1 | 76.0 | 77.0 | \|....L....DR.... | \|....H\| | \|.L..S.. | \|Y....G.ST. | \|.......K...M.... |
| 1975000- | | | | ..Q....V..R....\| | ----- | N..Q..T\| | ...A...\| | ......Q.T.L....D\| |
| 1976500 | | | | --------------- | | ------- | ---------- | ---------------- |
| | | | | --------------- | | ------- | ------- | ---------------- |

--- IGHV3-7-(1) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLVESGGGLVQPG | \|SYWMS\| | \|WVRQAPG | \|NTKQDGSEKY | \|RFTISRDNAKNSLYLQ |
| 7 | | | | GSLRLSCAASGFTFS\| | | KGLEWVA\| | YVDSVKG\| | MNSLRAEDTAVYYCAK\| |
| Dingo- | 1 | 75.0 | 81.0 | \|.E....R..HN.N.. | \|..G..\| | \|.....QK | \|H.SY...ST. | \|........T..N.... |
| 1442000- | | | | ...G...Q.......\| | ----- | ...Q..G\| | .T.T.RD\| | ..............N.\| |
| 1443500 | | | | --------------- | | ------- | ---------- | ---------------- |
| | | | | --------------- | | ------- | ------- | ---------------- |

TABLE 7
Sequence alignments for VH domains between human VGene and fox (canis
vulpes) VGene
--- IGHV3-74-(21) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLVESGGGLVQPG | \|SYWMH\| | \|WVRQAPG | \|RIMSDGSSTS | \|RFTISRDNAKNTLYLQ |
| 74 | | | | GSLRLSCAASGFTFS\| | | KGLVWVS\| | YADSVKG\| | MNSLRAEDTAVYYCAR\| |
| Fox_1- | 1 | 87.0 | 92.0 | \|.........A..K.. | \|..A.S\| | \|....... | \|Y.S......Y | \|................ |
| 7000- | | | | .......V.......\| | | ...Q..G\| | ...A...\| | .....V..........\| |
| 8500 | | | | | | | | |
| Fox_7- | 1 | 87.0 | 93.0 | \|.........D..K.. | \|..D..\| | \|....... | \|A.SYG....N | \|................ |
| 5000- | | | | .......V.......\| | | ...Q..A\| | ...A...\| | ................\| |
| 6500 | | | | | | | | |
| Fox_5- | 1 | 87.0 | 92.0 | \|.........D..K.. | \|..Y.S\| | \|.I..... | \|Y.S......R | \|................ |
| 37000- | | | | .......V.......\| | | ...Q..A\| | ...A...\| | ................\| |
| 38500 | | | | | | | | |
| Fox_8- | 1 | 86.0 | 89.0 | \|.........D..K.. | \|..Y..\| | \|....... | \|..S......R | \|Q.......P....... |
| 1645000- | | | | .......V.......\| | | ...K..A\| | ...AL..\| | ..........L.....\| |
| 1646500 | | | | | | | | |
| Fox_8- | 1 | 86.0 | 93.0 | \|.........D..K.. | \|.DN.Q\| | \|....... | \|Y.S.G....N | \|................ |
| 836000- | | | | .......V.......\| | | ...Q..A\| | ...A...\| | ................\| |
| 837500 | | | | | | | | |
| Fox_8- | 1 | 86.0 | 90.0 | \|.........D..K.. | \|..Y.S\| | \|.I..... | \|Y...G....N | \|................ |
| 1100000- | | | | .......V.......\| | | ...Q..A\| | ...A...\| | ...............G\| |
| 1101500 | | | | | | | | |
| Fox_8- | 1 | 86.0 | 90.0 | \|.........D..K.. | \|D.A.D\| | \|....... | \|Y...G..... | \|................ |
| 784000- | | | | .......V.......\| | | ...Q..A\| | ...T...\| | ....K..........K\| |
| 785500 | | | | | | | | |
| Fox_8- | 1 | 86.0 | 90.0 | \|.........D..K.. | \|..Y..\| | \|....... | \|Q.SN.....G | \|........S....... |
| 4000- | | | | .......V.......\| | | ...Q..A\| | ...A...\| | ..........L.....\| |
| 5500 | | | | | | | | |
| Fox_8- | 1 | 25.0 | 90.0 | \|.........D..K.. | \|....Y\| | \|....... | \|E.SGS....N | \|...L............ |
| 1381000- | | | | .......VG......\| | | ...Q..A\| | ...A...\| | ................\| |
| 1382500 | | | | | | | | |
| Fox_8- | 1 | 84.0 | 89.0 | \|.........D..K.. | \|..N.Q\| | \|..P.... | \|Y.STG..... | \|...........K.... |
| 1149000- | | | | .......VV......\| | | ...Q..A\| | ...A...\| | ................\| |
| 1150500 | | | | | | | | |
| Fox_8- | 1 | 83.0 | 89.0 | \|.........D..K.. | \|D.Y.S\| | \|..C.... | \|S.IV...... | \|................ |
| 1504000- | | | | ...K...V.......\| | | ...Q..A\| | .T.A...\| | ...............S\| |
| 1505500 | | | | | | | | |
| Fox_8- | 1 | 82.0 | 86.0 | \|.........D..K.. | \|..Y.D\| | \|..P.... | \|G.S......Y | \|................ |
| 1634000- | | | | .......V...V...\| | | .R.Q..A\| | .P.A...\| | .........S.....M\| |
| 1655500 | | | | | | | | |
| Fox_8- | 1 | 82.0 | 86.0 | \|Q........D..KI. | \|.C...\| | \|....... | \|E.SGS....N | \|Q............... |
| 758000- | | | | .......V.......\| | | ...Q..A\| | ...A...\| | .......K......V.\| |
| 759500 | | | | | | | | |
| Fox_8- | 1 | 82.0 | 84.0 | \|....G....D..K.. | \|..M.S\| | \|..M.... | \|..I......Y | \|Q.........K..... |
| 937000- | | | | R......V.......\| | | ...Q...\| | ...A...\| | ......T.......GK\| |
| 938500 | | | | | | | | |
| Fox_8- | 1 | 82.0 | 92.0 | \|.........D..K.. | \|N.KTY\| | \|....... | \|..YES..T.N | \|................ |
| 17000- | | | | .......V.......\| | | ...E..A\| | ..RA...\| | ...............S\| |
| 18500 | | | | | | | | |
| Fox_8- | 1 | 82.0 | 85.0 | \|.......T.D..K.. | \|..Y.D\| | \|..C.... | \|Y.S....... | \|Q............... |
| 1620000- | | | | .......V......N\| | | ...Q..A\| | .T.A...\| | ..............TS\| |
| 1621500 | | | | | | | | |
| Fox_8- | 1 | 81.0 | 84.0 | \|....L....N..K.R | \|..T.Q\| | \|..P.... | \|Y...G..... | \|P.I.........P... |
| 873000- | | | | .......V.......\| | | ...Q..T\| | S..T...\| | ...............D\| |
| 972500 | | | | | | | | |
| Fox_8- | 1 | 80.0 | 85.0 | \|.........D..K.. | \|..D..\| | \|....... | \|W.YAS...P. | \|...........VMVH. |
| 1462000- | | | | ...G...V.......\| | | ...Q..A\| | .S.A...\| | ...............S\| |
| 1463500 | | | | | | | | |
| Fox_8- | 1 | 80.0 | 86.0 | \|.........D..K.E | \|.SY.N\| | \|....D.R | \|A.SN..R..Y | \|.............V.. |
| 1422000- | | | | .......V.......\| | | ...Q..A\| | ...A...\| | ...........M....\| |
| 1423500 | | | | | | | | |
| Fox_5- | 1 | 76.0 | 81.0 | \|.E....F..HM.N.. | \|..G.S\| | \|.....QK | \|H.SY.....Y | \|.........T..M... |
| 22000- | | | | ...G...Q.......\| | | ...Q..G\| | ...T.HD\| | ..............M.\| |
| 25500 | | | | | | | | |
| Fox_8- | 1 | 69.0 | 68.0 | \|CAAGGVM.RPD.W.V | \|R.A.S\| | \|..L.... | \|Y.S....... | \|Q.A.L.A......... |
| 1487000- | | | | .......V.......\| | ----- | .R.Q..A\| | .T.A...\| | ................\| |
| 1488500 | | | | --------------- | | ------- | ---------- | ---------------- |
| | | | | --------------- | | ------- | ------- | ---------------- |

--- IGHV3-7-(4) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLVESGGGLVQPG | \|SYWMS\| | \|WVRQAPG | \|NIKQDGSEKY | \|RFTISRDNAKNSLVLQ |
| 7 | | | | GSLRLSCAASGFTFS\| | | KGLEWVA\| | YVDSVKG\| | MNSLRAEDTAVYYCAR\| |
| Fox_8-6- | 1 | 85.0 | 92.0 | \|.........D..K.. | \|..G..\| | \|....... | \|Y.SV...ST. | \|...........T.... |
| 0-1500 | | | | .......V.......\| | | ...Q...\| | .T.A...\| | ...............K\| |
| Fox_8- | 1 | 82.0 | 88.0 | \|.........D..K.. | \|.NE..\| | \|....... | \|A.SY...ST. | \|...T.......T.... |
| 845000- | | | | .......VV...A..\| | | ...Q...\| | ...A...\| | ...............D\| |
| 846500 | | | | | | | | |
| Fox_8- | 1 | 82.0 | 89.0 | \|.........D..K.. | \|..GV.\| | \|....... | \|A.WY..T.T. | \|.....S.....T.... |
| 1447000- | | | | .......V.......\| | | ...Q...\| | .A.A...\| | ..............VK\| |
| 1448500 | | | | | | | | |
| Fox_8- | 1 | 81.0 | 86.0 | \|.........D..K.. | \|..G..\| | \|....... | \|A.WY...RT. | \|...........T.... |
| 1182000- | | | | .......V.......\| | ----- | ...QL..\| | .A.A...\| | ....K.....N.F..K\| |
| 1183500 | | | | --------------- | | ------- | ---------- | ---------------- |
| | | | | --------------- | | ------- | ------- | ---------------- |

---IGHV3-21-(3) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLVESGGGLVKPG | \|SYSMN\| | \|WVRQAPG | \|SISSSSSYIY | \|RFTISRDNAKNSLYLQ |
| 21 | | | | GSLRLSCAASGFTFS\| | | KGLEWVS\| | YADSVKG\| | MNSLRAEDTAVYYCAR\| |
| Fox_8- | 1 | 83.0 | 85.0 | \|........RD..... | \|....S\| | \|C.C.... | \|E....G.ST. | \|...........T.... |
| 1189000- | | | | .......V..E....\| | | ...Q..A\| | .T.A...\| | ........M......D\| |
| 1190500 | | | | | | | | |
| Fox_8- | 1 | 72.0 | 72.0 | \|...QL....DR...R | \|....N\| | \|.LH.S.. | \|Y....G.ST. | \|.......KD..M.... |
| 1143000- | | | | ..Q....V..R....\| | | ...Q.TT\| | ...A...\| | ......Q.T.L....D\| |
| 1144500 | | | | | | | | |
| Fox_8- | 1 | 69.0 | 89.0 | \|...QL....DR...R | \|....N\| | \|.LH.S.. | \|Y....E.ST. | \|..A....KD..M.... |
| 1040000- | | | | ..Q....V..R....\| | ----- | ...Q.TT\| | .V.A...\| | ....K.Q.T.L....D\| |
| 1041500 | | | | --------------- | | ------- | ---------- | ---------------- |
| | | | | --------------- | | ------- | ------- | ---------------- |

--- IGHV3-230-(3) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|EVQLLESGGGLVQPG | \|SYAMS\| | \|WVRQAPG | \|AISGSGGSTY | \|RFTISRDNSKNTLYLQ |
| 230 | | | | GSLRLSCAASGFTFS\| | | KGLEWVS\| | YADSVKG\| | MNSLRAEDTAVYYCAR\| |
| Fox_8- | 1 | 86.0 | 89.0 | \|....V....D..K.. | \|.....\| | \|....... | \|G..N..S..G | \|........A....... |
| 1864000- | | | | .......V.......\| | | ...Q..A\| | ...A...\| | ....K...........\| |
| 1865500 | | | | | | | | |
| Fox_8- | 1 | 84.0 | 88.0 | \|....V....D..K.. | \|.NW..\| | \|....... | \|G..N..S... | \|........A....... |
| 951000- | | | | .......V.......\| | | ...Q..A\| | ...A...\| | .....T....M.....\| |
| 932500 | | | | | | | | |
| Fox_8- | 1 | 79.0 | 82.0 | \|....V....D..K.. | \|.....\| | \|..MK..V | \|R.YA..SR.S | \|........V....... |
| 988000- | | | | .F.....V.......\| | ----- | ...Q..T\| | ...A...\| | ...........M..L.\| |
| 981500 | | | | --------------- | | ------- | ---------- | ---------------- |
| | | | | --------------- | | ------- | ------- | ---------------- |

--- IGHV1-46-(2) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV1- | 1 | | | \|QVQLVLSGAEVKKPG | \|SYYMH\| | \|WYRQAPG | \|IINPSGGSTS | \|RVTMTRDTSYSTVYMEL |
| 46 | | | | ASVKVSCKASGYTFT\| | | QGLEWNG\| | YAQKFQG\| | SSLRSEDTAVYYCAR\| |
| Fox_8- | 1 | 82.0 | 85.0 | \|E.............. | \|N....\| | \|....... | \|R.D.ED.A.N | \|...L.A......A.... |
| 1416000- | | | | ........T.....I\| | | A..D...\| | .......\| | R...A..........\| |
| 1417500 | | | | | | | | |
| Fox_8- | 1 | 79.0 | 85.0 | \|E.............. | \|N...Y\| | \|....... | \|L.D.ED.A.R | \|...L.A......A.... |
| 1549000- | | | | ........T.....I\| | ----- | A.FD...\| | ....I..\| | N...A..........\| |
| 1550500 | | | | --------------- | | ------- | ---------- | ----------------- |
| | | | | --------------- | | ------- | ------- | --------------- |

--- IGHV3-30-5-(1) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|QVQLVESGGGVVQPG | \|SYSNH\| | \|WVRQAPG | \|VISYDGSNKV | \|RFTISRDNSKNTLVLQ |
| 30-5 | | | | RSLRLSCAASGFTFS\| | | KGLEWVA\| | YADSVKG\| | MNSLRAEDTAVYYCAR\| |
| Fox_8- | 1 | 85.0 | 89.0 | \|E........DL.K.. | \|....N\| | \|....... | \|..W....RT. | \|........A....... |
| 1081000- | | | | G......V.......\| | ----- | ...Q...\| | .N.A...\| | ................\| |
| 1082500 | | | | --------------- | | ------- | ---------- | ---------------- |
| | | | | --------------- | | ------- | ------- | ---------------- |

--- IGHV3-66-(1) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------ | FW3-------------- |
| IGHV3- | 1 | | | \|EVQLVESGGGLVQPG | \|SNYMS\| | \|WVRQAPG | \|VIYSGGSTY | \|RFTISRDNSKNTLYLQ |
| 66 | | | | GSLRLSCAASGFTVS\| | | KGLEWVS\| | YADSVKG\| | MNSLRAEDTAVYYCAR\| |
| Fox_4- | 1 | 84.0 | 89.0 | \|.........D..K.. | \|.YA..\| | \|....... | \|A.SY..... | \|........A....... |
| 3000- | | | | .......V.....F.\| | ----- | ...Q..A\| | .T.A...\| | ...............K\| |
| 4500 | | | | --------------- | | ------- | --------- | ---------------- |
| | | | | --------------- | | ------- | ------- | ---------------- |

--- IGHV3-11-(1) ---

| ACCES- | | % | % | -------------- | | ------ | ------ | --------------- |
|---|---|---|---|---|---|---|---|---|
| SIONS | Count | VGENE | FWs | FW1------------- | CDR1- | FW2----- | CDR2------- | FW3-------------- |
| IGHV3- | 1 | | | \|QVQLVESGGGLVKPG | \|DYYMS\| | \|WIRQAPG | \|YISSSGSTTY | \|RFTISRDNAKNSLYLQ |
| 11 | | | | GSLRLSCAASGFTFS\| | | KGLEWVS\| | YADSVKG\| | MNSLRAEDTAVYYCAR\| |
| Fox_8- | 1 | 84.0 | 86.0 | \|E........D..... | \|..G..\| | \|.V..... | \|E......ST. | \|...........T.... |
| 1301000- | | | | V......Y.......\| | ----- | ...Q..G\| | ...A...\| | ........M......T\| |
| 1302500 | | | | --------------- | | ------- | ---------- | ---------------- |
| | | | | --------------- | | ------- | ------- | ---------------- |

TABLE 8
Sequence alignments for VH domains between human VGene and wolf (canis lupus)

| | | | | | | | | |
|---|---|---|---|---|---|---|---|---|
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | |
| [symbol] | [symbol] | [symbol] | [symbol] | [symbol] | [symbol] | [symbol] | [symbol] | |
| | | | | [symbol] | [symbol] | [symbol] | [symbol] | |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |

[symbol]

indicates data missing or illegible when filed

TABLE 9
Sequence alignment for anti-SARS antibodies (VH domains) between human and dog VGenes (canis familiaris)

| | | | | | | | | |
|---|---|---|---|---|---|---|---|---|
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |

[symbol]

indicates data missing or illegible when filed

TABLE 10
Sequence alignment for naive human antibodies (VH domains) between human and dog
VGenes (canis familiaris)

| | | | | | | | | |
|---|---|---|---|---|---|---|---|---|
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |
| [symbol] | | | | | | | | |
| [symbol] | | | | | | | | |
| [symbol] | [symbol] | [symbol] | [symbol] | [symbol] | [symbol] | [symbol] | [symbol] | [symbol] |
| [symbol] | [symbol] | | | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | | | [symbol] | [symbol] | [symbol] | [symbol] | [symbol] |

[symbol]

indicates data missing or illegible when filed

TABLE 11
Sequences of the VH and VL kappa domains of clone G1 (CD96) and clone C2
(TIGIT) scFvs identified in the naive canin scFv-phage display library.
The underlined sequences indicate the CDR regions in each domain.

| | |
|---|---|
| VH | EVQLVESGGDLVKPAGSLRLSCVASGFPFSDSDMNWVRQAPGKGLQWVAGIDEGATDTGYADAVKG |
| clone | RFTISRDDAKNTLYLQMNSLRADDTAMYYCAAGGNFAYWGQGTLVTVSS |
| G1 | |
| (CD96) | |
| VKappa | DIVMTQTPLSLSVSPGEPASISCKASQSLLHSNGNTYLYWFRQKPGQSPQGLIYLVSKRFSWVPDR |
| clone | FSGSGSGTDFTLRISRVEADDAGLYYCGQGIQYPYTFSQGTKLEIK |
| G1 | |
| (CD96) | |
| VH | GVQLVESGGDLVKPAGSLRLSCVASGFTFSNYGMSWVRQAPGKGLQWVASIASGGSATAYADAVKG |
| clone | RFTLSRDDAKNTLYLQMNNLRTEDTSMYYCVAPGFQYWGQGTLVTVSS |
| C2 | |
| (TIGIT) | |
| VKappa | DIVMTQTPLSLSVSPGETASISCKASQSLLHSNGNTYLFWFRQKSGQSPQRLIYKVSNRDPGVPDR |
| clone | LSGSGSGTDFTLRISRVEADDAGVYYCGQGIQIPRTFGQGTKLEIK |
| C2 | |
| (TIGIT) | |

TABLE 12
In vitro binding of the canine antibodies (anti-96 clone
G1 and ant-TIGIT clone C2) towards their human targets (CD96
and TIGIT respectively) as compared to control (BSA).

| | R7P1-G1 (anti- | | irrelevant | R8P1-C2 (anti- | | irrelevant |
|---|---|---|---|---|---|---|
| Phage | CD96) | | phage | TGIT) | | phage |
| pfu/ml | CD96 | BSA | CD96 | TIGIT | BSA | TIGIT |
| 1 × 10¹¹ | 2.61 | 0.06 | 0.59 | 2.05 | 0.07 | 0.24 |
| 5 × 10¹⁰ | 2.68 | 0.04 | 0.31 | 2.53 | 0.04 | 0.31 |
| 2.5 × 10¹⁰ | 2.65 | 0.03 | 0.18 | 2.66 | 0.04 | 0.25 |
| 1.2 × 10¹⁰ | 2.59 | 0.02 | 0.08 | 2.54 | 0.04 | 0.15 |
| 6 × 10⁹ | 2.28 | 0.02 | 0.08 | 2.58 | 0.03 | 0.09 |
| 3 × 10⁹ | 2.11 | 0.02 | 0.02 | 2.52 | 0.02 | 0.07 |
| 1.5 × 10⁹ | 1.73 | 0.02 | 0.02 | 2.34 | 0.02 | 0.04 |
| 7.5 × 10⁸ | 1.36 | 0.01 | 0.02 | 1.99 | 0.01 | 0.03 |
| 3.7 × 10⁸ | 0.95 | 0.02 | 0.01 | 1.61 | 0.02 | 0.03 |
| 4.8 × 10⁸ | 0.63 | 0.01 | 0.02 | 1.21 | 0.02 | 0.03 |
| 9 × 10⁷ | 0.38 | 0.03 | 0.03 | 0.79 | 0.03 | 0.04 |
| 0 | 0.05 | 0.05 | 0.05 | 0.03 | 0.04 | 0.04 |

TABLE 13
Alignment of VH and VK domains of the clone G1 (CD96) with the top 10 closest canine
germlines. Amino acids non-conserved in any germlines are underlined.

| | | --------- | | | ------ | ---------- | | |
|---|---|---|---|---|---|---|---|---|
| | | -----FW1-- | | ------FW2 | CDR2 | -----FW3--- | | |
| Dog-VH | % FWs | ----------- | CDR1- | ----- | ------- | ----------- | -CDR3- | ----FW4---- |
| CD96-VH- | | \|EVQLVESGGD | \|DSDMN\| | \|WVRQAPGKG | \|GIDEGATD | \|RFTISRDDAK | \|GGNFAY\| | \|WGQGTLVTVSS\| |
| R7P1-G1 | | LVKPAGSLRLS | | LQWVA\| | TGYADAVKG\| | NTLYLQMNSLR | | |
| | | CVASGFPFS\| | | | | ADDTAMYYCAA\| | | |
| IGHV3- | 94.25 | \|.......... | \|SY...\| | \|......... | \|Y.SS.GSS. | \|.......N... | | \|...........\| |
| 35\| | | ....V...... | | .....\| | Y.......\| | ...........E | | |
| IGHJ2*01 | | ......T..\| | | | | ........G\| | | |
| IGHV3- | 94.25 | \|.......... | \|SYY.Y\| | \|......... | \|R.SSDGSS. | \|.......N... | | \|...........\| |
| 67\| | | ....G...... | | .....\| | Y.......\| | ...........E | | |
| IGHJ2*01 | | ......T..\| | | | | ........K\| | | |
| IGHV3- | 93.10 | \|.......... | \|SYH.S\| | \|......... | \|Y.NS.GSS. | \|.......N... | | \|...........\| |
| 5\| | | ....G...... | | .....\| | S.......\| | ...........E | | |
| IGHJ2*01 | | ......T..\| | | | | ...V....S\| | | |
| IGHV3- | 91.95 | \|.......... | \|S...S\| | \|.I....... | \|Y.SNDGSS. | \|.......N... | | \|...........\| |
| 6\| | | ....G...... | | .....\| | S.......\| | ...........E | | |
| IGHJ2*01 | | ......T..\| | | | | ...V....D\| | | |
| IGHV3- | 91.95 | \|.......... | \|SYS.S\| | \|......E.. | \|..NS.GSS. | \|.......N... | | \|...........\| |
| 19\| | | ........... | | ..L..\| | Y.T.....\| | .V.........E | | |
| IGHJ2*01 | | ......T..\| | | | | ........K\| | | |
| IGHV3- | 90.80 | \|.......... | \|SN..D\| | \|......... | \|R.SNDGRS. | \|.......N... | | \|...........\| |
| 50\| | | ....G...... | | ...LT\| | ........\| | ...........E | | |
| IGHJ2*01 | | ......T..\| | | | | ...V....K\| | | |
| IGHV3-3 | 90.80 | \|.......... | \|SY..S\| | \|....S.... | \|V.WNDGSS. | \|.......N... | | \|...........\| |
| 8\| | | ....G.T.... | | .....\| | Y.......\| | ...........E | | |
| IGHJ2*01 | | ......T..\| | | | | ...V....K\| | | |
| IGHV3- | 90.80 | \|.......... | \|SY..D\| | \|......... | \|E.SSSGSS. | \|.......N... | | \|...........\| |
| 9\| | | ....G...... | | ...LS\| | Y.......\| | ...........E | | |
| IGHJ2*01 | | ......T..\| | | | | ...V....R\| | | |
| IGHV3- | 90.80 | \|.......... | \|NY..S\| | \|......... | \|A.SYDGSS. | \|.......N.R. | | \|...........\| |
| 41\| | | ....G...... | | .....\| | Y.T.....\| | .V.........E | | |
| IGHJ2*01 | | ......T..\| | | | | ...V....K\| | | |
| IGHV3- | 90.80 | \|.......... | \|SY..S\| | \|......... | \|Y.SSDGRS. | \|.......N... | | \|...........\| |
| 18\| | | ....E...... | | .....\| | S.T.....\| | ..........TE | | |
| IGHJ2*01 | | ..V...T..\| | | | | ...V....K\| | | |
| | | ---------- | ------ | | | ----------- | | |
| | | FW1-------- | CDR1--- | ------FW2 | | ----FW3---- | --CDR3 | |
| Dog-VK | % FWs | -- | --- | ------ | -CDR2-- | ---------- | --- | ----FW4--- |
| CD96-VK- | | \|DIVMTQTPLS | \|KASQSL | \|WFRQKPGQS | \|LVSKRFS\| | \|WVPDRFSGSGS | \|GQGIQY | \|FSQGTKLEIK\| |
| R7P1-G1 | | LSVSPGEPASI | LHSNGNT | PQGLIY\| | | GTDFTLRISRVE | PYT\| | |
| | | SC\| | YLY\| | | | ADDAGLYYC\| | | |
| IGKV2S13 | 97.50 | \|.......... | \|R..... | \|......... | \|...N...\| | \|........... | | \|.G........\| |
| *01\| | | ........... | ....... | ......\| | | ............ | | |
| IGKJ3*01 | | ..\| | ...\| | | | .....V...\| | | |
| IGKV2-5\| | 95.00 | \|.......... | \|...... | \|......... | \|K..N.DP\| | \|G.......... | | \|.G........\| |
| IGKJ3*01 | | ........... | ....... | ..R...\| | | ............ | | |
| | | ..\| | ...\| | | | .....V...\| | | |
| IGKV2-8\| | 93.75 | \|........P. | \|...... | \|......... | \|R..N.ST\| | \|G.S........ | | \|.G........\| |
| IGKJ3*01 | | ........... | ....... | ......\| | | ............ | | |
| | | ..\| | ..N\| | | | .....V...\| | | |
| IGKV2-7\| | 93.75 | \|......N... | \|...... | \|......... | \|K..N.DP\| | \|G.......... | | \|.G........\| |
| IGKJ3*01 | | .......T... | ....... | ......\| | | ............ | | |
| | | ..\| | ..N\| | | | .....V...\| | | |
| IGKV2-9\| | 92.50 | \|.......... | \|...... | \|......... | \|K..N.DP\| | \|G.......... | | \|.G........\| |
| IGKJ3*01 | | .......T... | ...D... | ..R...\| | | ............ | | |
| | | ..\| | ..N\| | | | ...T.V...\| | | |
| IGKV2-6\| | 92.50 | \|.......... | \|...... | \|......... | \|K..N.DP\| | \|G.......... | | \|.G........\| |
| IGKJ3*01 | | .......T... | ...D... | ..R...\| | | ............ | | |
| | | ..\| | ..N\| | | | ...T.V...\| | | |
| IGKV2-10 | 92.50 | \|.......... | \|...... | \|......... | \|K..N.DP\| | \|G.......... | | \|.G........\| |
| \|IGKJ3*01 | | .......T... | ...D... | ..R...\| | | ............ | | |
| | | ..\| | ..N\| | | | ...T.V...\| | | |
| IGKV2-11\| | 91.25 | \|.......... | \|...... | \|......... | \|...N.DP\| | \|G..H....... | | \|.G........\| |
| IGKJ3*01 | | .......T... | ....... | ..R..N\| | | ............ | | |
| | | ..\| | ..F\| | | | .....V...\| | | |
| IGKV2-12\| | 90.00 | \|.......... | \|R.N... | \|.YM...... | \|R..NH.T\| | \|G.S........ | | \|.G........\| |
| IGKJ3*01 | | .......T... | ....... | ......\| | | ......K..... | | |
| | | ..\| | ..D\| | | | .....V...\| | | |
| IGKV2-4\| | 85.00 | \|........P. | \|...... | \|.YL...... | \|...N..T\| | \|G.S........ | | \|.G........\| |
| IGKJ3*01 | | .....R.T... | ...D... | ..L...\| | | ............ | | |
| | | ..\| | ..D\| | | | .N.T.V...\| | | |

TABLE 14
Alignment of VH and VK domains of the clone C2 (TIGIT) with the top 10 closest canine
germlines. Amino acids non conserved in any germline are underlined.

| | | ------------ | | | ------ | ----------- | | |
|---|---|---|---|---|---|---|---|---|
| | | --FW1------- | | ------ | CDR2---- | ----FW3---- | | |
| Dog-VH | % FWs | ------ | CDR1- | FW2----- | --- | ---------- | CDR3- | ----FW4---- |
| TIGIT- | | \|GVQLVESGGDLV | \|NYGMS\| | \|WVRQAPGK | \|SIASGGS | \|RFTLSRDDAKN | \|PGFQY\| | \|WGQGTLVTVSS\| |
| VHR8P1C2 | | KPAGSLRLSCVAS | | GLQWVA\| | ATAYADAV | TLYLQMNNLRTE | | |
| | | GFTFS\| | | | KG\| | DTSMYYCVA\| | | |
| IGHV3-35\| | 89.66 | \|E........... | \|S.D.N\| | \|........ | \|Y.S.... | \|...I...N... | | \|...........\| |
| IGHJ2*01 | | ..V.......... | | ......\| | S.Y..... | .......S..A. | | |
| | | .....\| | | | ..\| | ..A....AG\| | | |
| IGHV3-67\| | 89.66 | \|E........... | \|S.Y.Y\| | \|........ | \|R.S.D.. | \|...I...N... | | \|...........\| |
| IGHJ2*01 | | ..G.......... | | ......\| | S.Y..... | .......S..A. | | |
| | | .....\| | | | ..\| | ..A....AK\| | | |
| IGHV3-5\| | 88.51 | \|E........... | \|S.H..\| | \|........ | \|Y.N.... | \|...I...N... | | \|...........\| |
| IGHJ2*01 | | ..G.......... | | ......\| | S.S..... | .......S..A. | | |
| | | .....\| | | | ..\| | ..AV...AS\| | | |
| IGHV3-18\| | 88.51 | \|E........... | \|S.D..\| | \|........ | \|Y.S.D.R | \|...I...N... | | \|...........\| |
| IGHJ2*01 | | ..E........V. | | ......\| | S.S.T... | .......S.... | | |
| | | .....\| | | | ..\| | ..AV...AK\| | | |
| IGHV3-69\| | 87.36 | \|E........... | \|S.A..\| | \|........ | \|Y.N.... | \|...I...N.R. | | \|...........\| |
| IGHJ2*01 | | ..G.......... | | ......\| | S.Y..... | .......S..S. | | |
| | | .....\| | | | ..\| | ..AV...PK\| | | |
| IGHV3-19\| | 87.36 | \|E........... | \|S.S..\| | \|......E. | \|G.N.... | \|...I...N... | | \|...........\| |
| IGHJ2*01 | | ............. | | ...L..\| | S.Y.T... | .V.....S..A. | | |
| | | .....\| | | | ..\| | ..A....AK\| | | |
| IGHV3-6\| | 87.36 | \|E........... | \|SSD..\| | \|.I...... | \|Y.SND.. | \|...I...N... | | \|...........\| |
| IGHJ2*01 | | ..G.......... | | ......\| | S.S..... | .......S..A. | | |
| | | .....\| | | | ..\| | ..AV...AD\| | | |
| IGHV3-44\| | 86.21 | \|E........... | \|S.W..\| | \|C....... | \|Y.N.... | \|...I...N... | | \|...........\| |
| IGHJ2*01 | | ..G........T. | | E.....\| | S.W.T... | ..........A. | | |
| | | .....\| | | | ..\| | ..AV...AR\| | | |
| IGHV3-41\| | 86.21 | \|E........... | \|..D..\| | \|........ | \|A.SYD.. | \|...I...N.R. | | \|...........\| |
| IGHJ2*01 | | ..G.......... | | ......\| | S.Y.T... | .V.....S..A. | | |
| | | .....\| | | | ..\| | ..AV...AK\| | | |
| IGHV3-2\| | 86.21 | \|E........... | \|SNY..\| | \|.I...... | \|Q.S.D.. | \|...I...N... | | \|...........\| |
| IGHJ2*01 | | ..G.......... | | .....S\| | S.S..... | .......S..D. | | |
| | | .....\| | | | ..\| | ..AV...AR\| | | |
| | | ---------- | ------ | | | ----------- | | |
| | | FW1-------- | CDR1-- | ------ | | ----FW3---- | --CDR3 | |
| Dog-VK | % FWs | -- | ---- | FW2------ | -CDR2-- | ---------- | --- | ----FW4--- |
| TIGIT- | | \|DIVMTQTPLSL | \|KASQSLL | \|WFRQKSGQS | \|KVSNRDP\| | \|GVPDRLSGSGS | \|GQGIQI | \|FGQGTKLEIK\| |
| VLR8P1C2 | | SVSPGETASISC\| | HSNGNTYL | PQRLIY\| | | GTDFTLRISRVE | PRT\| | |
| | | | F\| | | | ADDAGVYYC\| | | |
| IGKV2-5\| | 96.25 | \|............ | \|....... | \|.....P... | \|.......\| | \|.....F..... | | \|..........\| |
| IGKJ3*01 | | .....P.....\| | ........ | ......\| | | ............ | | |
| | | | Y\| | | | .........\| | | |
| IGKV2-9\| | 96.25 | \|............ | \|....... | \|.....P... | \|.......\| | \|.....F..... | | \|..........\| |
| IGKJ3*01 | | ...........\| | ..D..... | ......\| | | ............ | | |
| | | | N\| | | | ...T.....\| | | |
| IGKV2-6\| | 96.25 | \|............ | \|....... | \|.....P... | \|.......\| | \|.....F..... | | \|..........\| |
| IGKJ3*01 | | ...........\| | ..D..... | ......\| | | ............ | | |
| | | | N\| | | | ...T.....\| | | |
| IGKV2-10\| | 96.25 | \|............ | \|....... | \|.....P... | \|.......\| | \|.....F..... | | \|..........\| |
| IGKJ3*01 | | ...........\| | ..D..... | ......\| | | ............ | | |
| | | | N\| | | | ...T.....\| | | |
| IGKV2-7\| | 95.00 | \|......N..... | \|....... | \|.....P... | \|.......\| | \|.....F..... | | \|..........\| |
| IGKJ3*01 | | ...........\| | ........ | ..G...\| | | ............ | | |
| | | | N\| | | | .........\| | | |
| IGKV2-11\| | 95.00 | \|............. | \|....... | \|.....P... | \|L......\| | \|...H.F..... | | \|..........\| |
| IGKJ3*01 | | ..........\| | ........ | .....N\| | | ............ | | |
| | | | .\| | | | .........\| | | |
| IGKV2S13 | 93.75 | \|............. | \|R...... | \|.....P... | \|L....FS\| | \|W....F..... | | \|..........\| |
| *01\| | | ....P.....\| | ........ | ..G...\| | | ............ | | |
| IGKJ3*01 | | | Y\| | | | .........\| | | |
| IGKV2-8\| | 92.50 | \|........P.... | \|....... | \|.....P... | \|R....ST\| | \|..S..F..... | | \|..........\| |
| IGKJ3*01 | | ....P.....\| | ........ | ..G...\| | | ............ | | |
| | | | N\| | | | .........\| | | |
| IGKV2-12\| | 91.25 | \|............. | \|R.N.... | \|.YM..P... | \|R...HFT\| | \|..S..F..... | | \|..........\| |
| IGKJ3*01 | | ..........\| | ........ | ..G...\| | | ......K..... | | |
| | | | D\| | | | .........\| | | |
| IGKV2-4\| | 87.50 | \|........P.... | \|....... | \|.YL..P... | \|L....FT\| | \|..S..F..... | | \|..........\| |
| IGKJ3*01 | | ..R.......\| | ..D..... | ..L...\| | | ............ | | |
| | | | D\| | | | .N.T.....\| | | |

TABLE 15
Summary of the selection campaign of potential positive
phages against the two targets CD96 and TIGIT.

| | Positive | Number of | VH % id. | VK % id. | Closest human | Closest human |
|---|---|---|---|---|---|---|
| Target | phages | sequence | FW | FW | VH germlines | VK germline |
| #1 | 8 clone out | 1 | 87.36 | 85 | IGHV3-21, | IGKV2D-29, |
| (CD96) | of 196 | | | | IGHV3-74, | IGKV2-28, |
| | | | | | IGHV3-7 | IGKV2D-28, |
| | | | | | IGHJ4*02 | IGKJ2*02 |
| #2 | 11 clone out | 1 | 82.76 | 85 | IGHV3-74, | IGKV2D-29, |
| (TIGIT) | of 288 | | | | IGHV3-21, | IGKV2-28, |
| | | | | | IGHV3-7 | IGKV2D-28 |
| | | | | | IGHJ4*02 | IGKJ2*02 |

TABLE 16
Sequence similarity between the VH and VL sequence of the anti-CD96 clone G1
antibody and germline sequences of rat, chicken, rabbit, mouse and human.

| | G1 VH (CD96) | | G1 VK (CD96) | |
|---|---|---|---|---|
| | % id. | Species | % id. | Species |
| | FWs | closest germline | FWs | closest germline |
| Rat | 57.47 | IGHV3-3\|IGHJ3*01 | 78.75 | IGKV1S30*01\|IGKJ4*01 |
| Chicken | 67.82 | IGHV1-38\|IGHJ*01 | N.A | N.A |
| Rabbit | 72.41 | IGHV1S7*01\|IGHJ3*02 | 66.25 | IGKV1S54*01\|IGKJ2-3 |
| Mouse | 83.91 | IGHV5-17\|IGHJ4*01 | 78.75 | IGKV1-110\|IGKJ4*01 |
| Human | 87.36 | IGHV3-21\|IGHJ4*02 | 85.00 | IGKV2D-29\|IGKJ2*02 |

TABLE 17
Sequence similarity between the VH and VL sequence of the anti-TIGIT clone C2
antibody and germline sequences of rat, chicken, rabbit, mouse and human.

| | C2 VH (TIGIT) | | C2 VK (TIGIT) | |
|---|---|---|---|---|
| | % id. | Species | % id. | Species |
| | FWs | closest germline | FWs | closest germline |
| Rat | 52.87 | IGHV8-20\|IGHJ3*01 | 81.25 | IGKV1S27*01\|IGKJ4*01 |
| Chicken | 66.67 | IGHV1-1\|IGHJ*01 | N.A | N.A |
| Rabbit | 70.11 | IGHV1S7*01\|IGHJ3*02 | 67.50 | IGKV1S54*01\|IGKJ2-3 |
| Mouse | 79.31 | IGHV5-6\|IGHJ4*01 | 80.00 | IGKV1-110\|IGKJ4*01 |
| Human | 82.76 | IGHV3-74\|IGHJ4*02 | 85.00 | IGKV2D-29\|IGKJ2*02 |

TABLE 18
Sequence alignment between the VH and VL domains of the anti-CD96 clone G1
antibody or the anti-TIGIT clone C2 antibody and germline sequences of
different organisms (human, mouse, rabbit, chicken, rat).

| | | ------------- | | | ------ | ------------ | | ---- |
|---|---|---|---|---|---|---|---|---|
| | | FW1----------- | | ------ | CDR2--- | ---FW3------- | | FW4-- |
| ACCESSIONS | % FWs | --- | CDR1- | FW2----- | ---- | ------- | -CDR3- | -- |
| CD96-VH- | | \|EVQLVESGGDLVK | \|DSDMN\| | \|WVRQAPG | \|GIDEGA | \|RFTISRDDAKNT | \|GGNFAY\| | \|WGQG |
| R7P1-G1 | | PAGSLRLSCVASGF | | KGLQWVA\| | TDTGYAD | LYLQMNSLRADDT | | TLVTV |
| | | PFS\| | | | AVKG\| | AMYYCAA\| | | SS\| |
| Human_ | 87.36 | \|.........G... | \|SYS..\| | \|....... | \|S.SSSS | \|.......N...S | | \|.... |
| IGHV3-21\| | | .G.......A.... | | ...E..S\| | SYIY... | ..........E.. | | ..... |
| IGHJ4*02 | | T..\| | | | S...\| | .V....R\| | | ..\| |
| Mouse_ | 83.91 | \|.........G... | \|.YG.H\| | \|......E | \|Y.SS.S | \|.......N.... | | \|.... |
| IGHV5-17\| | | .G...K...A.... | | ...E...\| | STIY... | .F...T...SE.. | | ..... |
| IGHJ3*02 | | T..\| | | | T...\| | ......R\| | | .A\| |
| Rabbit_ | 72.41 | \|CQ..K....G... | \|SYY.C\| | \|....... | \|C.YA.S | \|...L...N.QS. | | \|.... |
| IGHV1S7*01\| | | .G...K.C.K.... | | ...E.IG\| | GS.H..S | VC..L...T.A.. | | ..... |
| IGHJ3*02 | | T..\| | | | W.N.\| | .T.F..R\| | | ..\| |
| Chicken_ | 67.82 | \|A.T.D....G.QT | \|SNA.G\| | \|....... | \|...DDG | \|.A.....NGQS. | | \|..H. |
| IGHV1-38\| | | .G.G.S.V.K.... | | ...E...\| | SFI.L.. | VR..L.N...E.. | | .E.I. |
| IGHJ*01 | | D..\| | | | P...\| | .T....R\| | | ..\| |
| Rat_ | 57.47 | \|....Q...PG... | \|S.YWR | \|.I.KF.. | \|Y.N-S. | \|.IS.T..TS..Q | | \|.... |
| IGHV3-3\| | | .SQ..S.T.SVT.Y | N-\| | NK.E.MG\| | GS.N.NP | FF..V..VTTE.. | | ..... |
| IGHJ3*01 | | SIT\| | | | SL.S\| | .T....R\| | | ..\| |
| | | | ------ | ------ | | ------------ | | |
| | | ----------FW1 | CDR1--- | FW2---- | | ---FW3------- | -- | ---- |
| ACCESSIQNS | % FWs | ---------- | --- | -- | -CDR2-- | ------- | CDR3--- | FW4--- |
| CD96-VK- | | \|DIVMTQTPLSLSV | \|KASQSL | \|WFRQKPG | \|LVSKRFS\| | \|WVPDRFSGSGSG | \|GQGIQY | \|FSQGT |
| R7P1-G1 | | SPGEPASISC\| | LHSNGNT | QSPQGLIY\| | | TDFTLRISRVEAD | PYT\| | KLEIK\| |
| | | | YLY\| | | | DAGLYYC\| | | |
| Human_ | 85.00 | \|............. | \|.S.... | \|.YL..... | \|E..N...\| | \|G........... | | \|.G.... |
| IGKV2D-29\| | | T..Q......\| | ...D.K. | P..L...\| | | .....K......E | | ....\| |
| IGKJ2*02 | | | ...\| | | | .V.V...\| | | |
| Mouse_ | 78.75 | \|.V.........P. | \|RS.... | \|.YL..... | \|K..N...\| | \|G........... | | \|..D..R |
| IGKV1-110\| | | .L.DQ.....\| | V...... | ..KL...\| | | .....K......E | | ....\| |
| IGKJ3*02 | | | ..H\| | | | .L.V.F.\| | | |
| Rat_ | 78.75 | \|.V......V.... | \|RS.... | \|.YL..... | \|K..N...\| | \|G........... | | \|.GS.... |
| IGKV1S30*01\| | | .L.GQT....\| | V..D... | ...L...\| | | .....K.....PE | | ...\| |
| IGKJ4*01 | | | ...\| | | | .L.V...\| | | |
| Rabbit_ | 66.25 | \|.V......SPV.A | \|Q....V | \|..Q..... | \|AA.NLA.\| | \|G..S..K..... | | \|.GK.... |
| IGKV1S54*01\| | | AV.STVT...\| | ---YN.N | P.KL...\| | | .Q...T..G.QC. | | ...\| |
| IGKJ2-3 | | | N.A\| | | | ..AT...\| | | |
| | | ------------- | | | ------ | ------------ | | |
| | | -FW1---------- | | ------ | CDR2---- | ---FW3------- | | ----FW4 |
| ACCESSIONS | % FWs | --- | CDR1- | FW2----- | --- | ------- | CDR3- | ---- |
| TIGIT- | | \|GVQLVESGGDLVK | \|NYGMS\| | \|WVRQAPG | \|SIASGGS | \|RFTLSRDDAKNT | \|PGFQY\| | \|WGQGTLV |
| VHR8P1C2 | | PAGSLRLSCVASGF | | KGLQWVA\| | ATAYADAV | LYLQMNNLRTEDT | | TVSS\| |
| | | TFS\| | | | KG\| | SMYYCVA\| | | |
| Human_ | 82.76 | \|E........G..Q | \|S.W.H\| | \|....... | \|R.N.D.. | \|...I...N.... | | \|....... |
| IGHV3-74\| | | .G.......A.... | | ...V..S\| | S.S...S. | ......S..A... | | ....\| |
| IGHJ4*02 | | ...\| | | | ..\| | AV...AR\| | | |
| Mouse_ | 79.31 | \|E............ | \|S....\| | \|....T.D | \|T.S.... | \|...I...N.... | | \|....... |
| IGHV5-6\| | | .G...K...A.... | | .R.E...\| | Y.Y.P.S. | .....SS.KS... | | ...A\| |
| IGHJ3*02 | | ...\| | | | ..\| | A....AR\| | | |
| Rabbit_ | 70.11 | \|CQ..K....G... | \|S.Y.C\| | \|....... | \|C.YA.SG | \|.......N.QS. | | \|....... |
| IGHV1S7*01\| | | .G...K.C.K.... | | ...E.IG\| | S.H..SW. | VC..L.S.TAA.. | | ....\| |
| IGHJ3*02 | | ...\| | | | N.\| | AT.F.AR\| | | |
| Rat_ | 52.87 | \|Q.T.K...PGILQ | \|T...GV | \|.I..PS. | \|N.W-WDD | \|.L.I.K.TSN.Q | | \|....... |
| IGHV8-20\| | | .SQT.S.T.TF.. | G\| | ...E.L.\| | DKY.NPSL | AF.KIT.VD.A.. | | ....\| |
| IGHJ3*01 | | .SL.\| | | | .N\| | AT...AR\| | | |
| Chicken_ | 66.67 | \|A.T.D....G.QT | \|S...G\| | \|....... | \|G.G.S.. | \|.A.I...NGQS. | | \|..H..E. |
| IGHV1-1\| | | .R.A.S.V.K.... | | ...E...\| | G...GS.. | VR..L....A... | | I...\| |
| IGHJ*01 | | ...\| | | | ..\| | GT...AK\| | | |
| | | | ------ | ------ | | ------------ | | |
| | | ---------- | CDR1--- | FW2----- | | ---FW3------- | -- | -----FW4 |
| ACCESSIONS | % FWs | FW1---------- | --- | - | -CDR2-- | ------- | CDR3--- | --- |
| TIGIT- | | \|DIVMTQTPLSLS | \|KASQSL | \|WFRQKSG | \|KVSNRDP\| | \|GVPDRLSGSGSG | \|GQGIQI | \|FGQGTKL |
| VLR8P1C2 | | VSPGETASISC\| | LHSNGNT | QSPQRLI | | TDFTLRISRVEAD | PRT\| | EIK\| |
| | | | YLF\| | Y\| | | DAGVYYC\| | | |
| Human_ | 85.00 | \|............ | \|.S.... | \|.YL..P. | \|E....FS\| | \|.....F...... | | \|....... |
| IGKV2D-29\| | | .T..QP.....\| | ...D.K. | .P..L.. | | .....K......E | | ...\| |
| IGKJ2*02 | | | ..Y\| | .\| | | .V.....\| | | |
| Rat_ | 81.25 | \|.V......V... | \|RS...F | \|.YL..P. | \|.....LS\| | \|.....F...... | | \|..S.... |
| IGKV1S27*01\| | | ..L.GQV....\| | V..D... | ....L.. | | .....K.....H. | | ...\| |
| IGKJ4*01 | | | ..N\| | .\| | | .L.....\| | | |
| Mouse_ | 80.00 | \|.V.........P | \|RS.... | \|.YL..P. | \|.....FS\| | \|.....F...... | | \|..G.... |
| IGKV1-110\| | | ..L.DQ.....\| | V...... | ...KL.. | | .....K......E | | ...\| |
| IGKJ1*01 | | | ..H\| | .\| | | .L...F.\| | | |
| Rabbit_ | 67.50 | \|.V......SPV. | \|Q....V | \|..Q..P. | \|AA..LAS\| | \|...S.FK..... | | \|..K.... |
| IGKV1S54*01\| | | AAV.S.VT...\| | ---YN.N | .P.KL.. | | .Q...T..G.QC. | | ...\| |
| IGKJ2-3 | | | N.A\| | .\| | | ..AT...\| | | |

TABLE 19
Sequences of the VH and VKappa domain of the humanized antibody against
CD96 according to an embodiment.

| Ab vs | | | | |
|---|---|---|---|---|
| CD96 | VHname | VHseq | VKname | VKsequence |
| G1Hum1 | >VHG1humA | EVQLVESGGGLVQPGGSL | >VKG1hum1 | DIVMTQTPLSLSVTPGQP |
| | | RLSCAASGFTFSDSDMNW | | ASISCKASQSLLHSNGNT |
| | | VRQAPGKGLEWVAGIDEG | | YLYWYLQKPGQPPQLLIY |
| | | ATDTGYADAVKGRFTISR | | LVSKRFSGVPDRFSGSGS |
| | | DNAKNSLYLQMNSLRAED | | GTDFTLKISRVEAEDVGV |
| | | TAVYYCAAGGNFAYWGQG | | YYCGQGIQYPYTFGQGTK |
| | | TLVTVSS | | LEIK |
| G1Hum2 | >VHG1humA | EVQLVESGGGLVQPGGSL | >VKG1hum2 | DIVMTQTPLSLSVTPGQP |
| | | RLSCAASGFTFSDSDMNW | | ASISCKASQSLLHSNGNT |
| | | VRQAPGKGLEWVAGIDEG | | YLYWYLQKPGQPPQLLIY |
| | | ATDTGYADAVKGRFTISR | | LVSKRFSWVPDRFSGSGS |
| | | DNAKNSLYLQMNSLRAED | | GTDFTLKISRVEAEDVGV |
| | | TAVYYCAAGGNFAYWGQG | | YYCGQGIQYPYTFGQGTK |
| | | TLVTVSS | | LEIK |
| G1Hum3 | >VHG1humB | EVQLVESGGGLVQPGGSL | >VKG1hum1 | DIVMTQTPLSLSVTPGQP |
| | | RLSCAASGFTFSDSDMNW | | ASISCKASQSLLHSNGNT |
| | | VRQAPGKGLEWVSGIDEG | | YLYWYLQKPGQPPQLLIY |
| | | ATDTGYADAVKGRFTISR | | LVSKRFSGVPDRFSGSGS |
| | | DNAKNSLYLQMNSLRAED | | GTDFTLKISRVEAEDVGV |
| | | TAVYYCAAGGNFAYWGQG | | YYCGQGIQYPYTFGQGTK |
| | | TLVTVSS | | LEIK |
| G1Hum4 | >VHG1humB | EVQLVESGGGLVQPGGSL | >VKG1hum2 | DIVMTQTPLSLSVTPGQP |
| | | RLSCAASGFTFSDSDMNW | | ASISCKASQSLLHSNGNT |
| | | VRQAPGKGLEWVSGIDEG | | YLYWYLQKPGQPPQLLIY |
| | | ATDTGYADAVKGRFTISR | | LVSKRFSWVPDRFSGSGS |
| | | DNAKNSLYLQMNSLRAED | | GTDFTLKISRVEAEDVGV |
| | | TAVYYCAAGGNFAYWGQG | | YYCGQGIQYPYTFGQGTK |
| | | TLVTVSS | | LEIK |

TABLE 20
Sequences of the VH and VKappa domains of the humanized antibody
against TIGIT according to an embodiment.

| Ab vs | | | | |
|---|---|---|---|---|
| TIGIT | VHname | VHseq | VKname | VKsequence |
| C2hum5 | VHC2humC | EVQLVESGGGLVQPGGSL | VKC2hum3 | DIVMTQTPLSLSVTPGQP |
| | | RLSCAASGFTFSNYGMSW | | ASISCKASQSLLHSNGNT |
| | | VRQAPGKGLEWVASIASG | | YLFWYLQKPGQPPQLLIY |
| | | GSATAYADAVKGRFTISR | | KVSNRDPGVPDRFSGSGS |
| | | DNAKNSLYLQMNSLRAED | | GTDFTLKISRVEAEDVGV |
| | | TAVYYCVAPGFQYWGQGT | | YYCGQGIQIPRTFGQGTK |
| | | LVTVSS | | LEIK |
| C2hum6 | VHC2humD | EVQLVESGGGLVKPGGSL | VKC2hum3 | DIVMTQTPLSLSVTPGQP |
| | | RLSCAASGFTFSNYGMSW | | ASISCKASQSLLHSNGNT |
| | | VRQAPGKGLEWVASIASG | | YLFWYLQKPGQPPQLLIY |
| | | GSATAYADAVKGRFTISR | | KVSNRDPGVPDRFSGSGS |
| | | DNAKNSLYLQMNSLRAED | | GTDFTLKISRVEAEDVGV |
| | | TAVYYCVAPGFQYWGQGT | | YYCGQGIQIPRTFGQGTK |
| | | LVTVSS | | LEIK |

TABLE 21
Sequence alignment between VL and VH domains of humanized antigen binding
polypeptides directed to CD96 (VHG1huma1, VHG1humB, VKG1hum1, VKG1hum2)
or TIGIT (VHC2humC, VHC2humD, VHC2hum3) and sequences of human VH or VL
domains (IGHV3-7, IGHV3-48, IGKV2D-29) or against sequences of dog VH or
VL domains (VHG1, VKG1, VHC2, VKC2).

| | | | | ----- | ------------ | | ----- |
|---|---|---|---|---|---|---|---|
| | -------------- | | ------FW2 | CDR2--- | ---FW3------- | | FW4-- |
| | FW1------------- | CDR1- | ----- | ---- | ------- | -CDR3- | -- |
| IGHV3-7\| | \|EVQLVESGGGLVQPG | \|SYWMS\| | \|WVRQAPGKG | \|NIKQDGSE | \|RFTISRDNAKNS | | \|WGQG |
| IGHJ4*02 | GSLRLSCAASGFTFS\| | | LEWVA\| | KYYVDSVKG\| | LYLQMNSLRAEDT | | TLVTV |
| | | | | | AVYYCAR\| | | SS\| |
| VHG1humA | \|............... | \|DSD.N\| | \|......... | \|G.DEGATDT | \|............ | \|GGNFAY\| | \|.... |
| | ...............\| | | .....\| | G.A.A...\| | ............. | | ..... |
| | | | | | ......A\| | | ..\| |
| IGHV3-48\| | \|............... | \|..S.N\| | \|......... | \|Y.SSSS.TI | \|............ | | \|.... |
| IGHJ4*02 | ...............\| | | ....S\| | ..A.....\| | ............. | | ..... |
| | | | | | .......\| | | ..\| |
| VHG1humB | \|............... | \|DSD.N\| | \|......... | \|G.DEGATDT | \|............ | \|GGNFAY\| | \|.... |
| | ...............\| | | ....S\| | G.A.A...\| | ............. | | ..... |
| | | | | | ......A\| | | ..\| |
| VHG1 | \|.........D..K.A | \|DSD.N\| | \|......... | \|G.DEGATDT | \|.......D...T | \|GGNFAY\| | \|.... |
| (CD96) | .......V....P..\| | | .Q...\| | G.A.A...\| | ..........D.. | | ..... |
| | | | | | .M....A\| | | ..\| |
| | | ------ | | | ------------ | | ---- |
| | ----------FW1-- | CDR1--- | ------FW2 | | ---FW3------- | | FW4-- |
| | -------- | --- | ------ | -CDR2-- | ------- | --CDR3--- | - |
| IGKV2D-29\| | \|DIVMTQTPLSLSVTP | \|KSSQSLL | \|WYLQKPGQP | \|EVSNRFS\| | \|GVPDRFSGSGSG | \| \| | \|FGQG |
| IGKJ2*02 | GQPASISC\| | HSDGKTYL | PQLLIY\| | | TDFTLKISRVEAE | | TKLEI |
| | | Y\| | | | DVGVYYC\| | | K\| |
| VKG1hum1 | \|............... | \|.A..... | \|......... | \|L..K...\| | \|............ | \|GQGIQYPYT\| | \|.... |
| | ........\| | ..N.N... | ......\| | | ............. | | ..... |
| | | .\| | | | .......\| | | .\| |
| VKG1hum2 | \|............... | \|.A..... | \|......... | \|L..K...\| | \|W........... | \|GQGIQYPYT\| | \|.... |
| | ........\| | ..N.N... | ......\| | | ............. | | ..... |
| | | .\| | | | .......\| | | .\| |
| VKG1 | \|.............S. | \|.A..... | \|.FR.....S | \|L..K...\| | \|W........... | \|GQGIQYPYT\| | \|.S.. |
| (CD96) | .E......\| | ..N.N... | ..G...\| | | .....R......D | | ..... |
| | | .\| | | | .A.L...\| | | .\| |
| | | | | ------ | ------------ | | ---- |
| | -------------- | | ------FW2 | CDR2--- | ---FW3------- | | FW4-- |
| | FW1------------- | CDR1- | ----- | ---- | ------- | CDR3- | -- |
| IGHV3-7\| | \|EVQLVESGGGLVQPG | \|SYWMS\| | \|WVRQAPGKGL | \|NIKQDGSE | \|RFTISRDNAKNS | \| \| | \|WGQG |
| IGHJ4*02 | GSLRLSCAASGFTFS\| | | EWVA\| | KYYVDSVKG\| | LYLQMNSLRAEDT | | TLVTV |
| | | | | | AVYYCAR\| | | SS\| |
| VHC2humC | \|............... | \|N.G..\| | \|.......... | \|S.ASG..AT | \|............ | \|PGFQY\| | \|.... |
| | ...............\| | | ....\| | A.A.A...\| | ............. | | ..... |
| | | | | | .....VA\| | | ..\| |
| IGHV3-21\| | \|............K.. | \|..S.N\| | \|.......... | \|S.SSSS.YI | \|............ | \| \| | \|.... |
| IGHJ4*02 | ...............\| | | ...S\| | ..A.....\| | ............. | | ..... |
| | | | | | .......\| | | ..\| |
| VHC2humD | \|............K.. | \|N.G..\| | \|.......... | \|S.ASG..AT | \|............ | \|PGFQY\| | \|.... |
| | ...............\| | | ....\| | A.A.A...\| | ............. | | ..... |
| | | | | | .....VA\| | | ..\| |
| VHC2 | \|G........D..K.A | \|N.G..\| | \|.......... | \|S.ASG..AT | \|...L...D...T | \|PGFQY\| | \|.... |
| (TIGIT) | .......V.......\| | | Q...\| | A.A.A...\| | ......N..T... | | ...... |
| | | | | | SM...VA\| | | .\| |
| | | ------ | | | ------------ | | ---- |
| | ----------FW1-- | CDR1---- | ------FW2 | | ---FW3------- | | FW4-- |
| | -------- | -- | ------ | -CDR2-- | ------- | --CDR3--- | - |
| IGKV2D-29\| | \|DIVMTQTPLSLSVTP | \|KSSQSLL | \|WYLQKPGQPP | \|EVSNRFS\| | \|GVPDRFSGSGSG | \| \| | \|FGQG |
| IGKJ2*02 | GQPASISC\| | HSDGKTYL | QLLIY\| | | TDFTLKISRVEAE | | TKLEI |
| | | Y\| | | | DVGVYYC\| | | K\| |
| VKC2hum3 | \|............... | \|.A..... | \|.......... | \|K....DP\| | \|............ | \|GQGIQIPRT\| | \|.... |
| | ........\| | ..N.N... | .....\| | | ............. | | ..... |
| | | F\| | | | .......\| | | .\| |
| VKC2 | \|.............S. | \|.A..... | \|.FR..S..S. | \|K....DP\| | \|.....L...... | \|GQGIQIPRT\| | \|.... |
| (TIGIT) | .ET.....\| | ..N.N... | .R...\| | | .....R......D | | ..... |
| | | F\| | | | .A.....\| | | .\| |

TABLE 22
Cross-references to sequence identifiers

| SEQ | | |
|---|---|---|
| ID | Sequence | |
| NO | Name | SEQUENCE |
| 1 | IGHV3-74 | EVQLVESGGGLVQPGGSLRLSCAASGFTFSSYWMHWVRQAPGKGLVWVSRINSDGSSTSYADSVKGRFTISR |
| | | DNAKNTLYLQMNSLRAEDTAVYYCAR |
| 2 | Dog_ | EVQLVESGGDLVKPGGSLRLSCVASGFTFSSYHMSWVRQAPGKGLQWVAYINSGGSSTSYADAVKGRFTISR |
| | IGHV3- | DNAKNTLYLQMNSLRAEDTAVYYCAS |
| | 5*01 | |
| 3 | Dog_ | EVQLVESGGDLVKPGGSLRLSCVASGFTFSSNYMSWIRQAPGKGLQWVSQISSDGSSTSYADAVKGRFTISR |
| | IGHV3- | DNAKNTLYLQMNSLRDEDTAVYYCAR |
| | 2*01 | |
| 4 | Dog_ | EVQLVESGGDMVKPGGSLRLSCVASGFTFSSYYMYWARQAPGKGLQWVSHINKDGSSTSYADAVKGRFTISR |
| | IGHV3- | DNAKNTLYLQMNSLRAEDTAVYYCA |
| | 3*01 | |
| 5 | Dog_ | EVQLVESGGDLVKPGGSLRLSCVASGFTFSSYYMYWVRQAPGKGLQWVARISSDGSSTYYADAVKGRFTISR |
| | IGHV3- | DNAKNTLYLQMNSLRAEDTAMYYCAK |
| | 67*01 | |
| 6 | Dog_ | EVQLVESGGDLVKPGGSLRLSCVASGFTFSSSDMSWIRQAPGKGLQWVAYISNDGSSTSYADAVKGRFTISR |
| | IGHV3- | DNAKNTLYLQMNSLRAEDTAVYYCAD |
| | 6*01 | |
| 7 | Dog_ | EVQLVESGGDLVKPGGSLRLSCVASGFTFSSNDMDWVRQAPGKGLQWLTRISNDGRSTGYADAVKGRFTISR |
| | IGHV3- | DNAKNTLYLQMNSLRAEDTAVYYCAK |
| | 50*01 | |
| 8 | Dog_ | EVQLVESGGDLVKPGGSLRLSCVASGFTFSSYYMEWVRQAPGKGLQWVAQISSDGSSTYYPDAVKGQFTISR |
| | IGHV3- | DNAKNTLYLQMNSLGAEDTAVYYCAK |
| | 80*01 | |
| 9 | Dog_ | EVQLVESGGDLVKPGGTLRLSCVASGFTFSSYDMSWVRQSPGKGLQWVAVIWNDGSSTYYADAVKGRFTISR |
| | IGHV3- | DNAKNTLYLQMNSLRAEDTAVYYCAK |
| | 38*01 | |
| 10 | Dog_ | EVQLVESGGDLEKPGGSLRLSCVASGFTFSSYGMSWVRQAPGKGLQGVSLIRYDGSSTRYADAVKGRFTISR |
| | IGHV3- | DNAKNTLYLQMNSLRAEDTAVYSCAK |
| | 23*01 | |
| 11 | Dog_ | EVQLVESGGDLMKPGGSLRLSCVASGFTISSNYMNWVRQAPGKGLQWVGYISSDGSSTSYADAVKGRFTISR |
| | IGHV3- | DNAKNTLYLQMNSLRAEDTAVYYCVK |
| | 54*01 | |
| 12 | Dog_ | EVQLVESGGDLVKPGGSLRLSCVASGFTFSSYAMSWVRQAPGKGLQWVAYINSGGSSTYYADAVKGRFTISR |
| | IGHV3- | DNARNTLYLQMNSLRSEDTAVYYCPK |
| | 69*01 | |
| 13 | Dog_ | EVQLVESGGDLVKPGGSLRLSCVTSGFTFSSYWMSCVRQAPGKELQWVAYINSGGSSTWYTDAVKGRFTISR |
| | IGHV3- | DNAKNTLYLQMNNLRAEDTAVYYCAR |
| | 44*01 | |
| 14 | Dog_ | EVQLVESGGNLVKPGGSLRLSCVASGFTFSSYYMDWVRQAPGKRLQWVAGISSDGSSTYYPQAVKGRFTISR |
| | IGHV3- | DNAKNTLYLQMNSLRAEDSAVYYCAM |
| | 81*01 | |
| 15 | Dog_ | EVQLVESGGDLVKPEGSLRLSCVVSGFTFSSYDMSWVRQAPGKGLQWVAYISSDGRSTSYTDAVKGRFTISR |
| | IGHV3- | DNAKNTLYLQMNSLRTEDTAVYYCAK |
| | 18*01 | |
| 16 | Dog_ | EVQLVESGGDLVKPGGSLRLSCVASGFTFSSYYMFWIRQAPGKGNQWVGYINKDGSSTYYPDAVKGRFTISR |
| | IGHV3- | DNAKNTLYLQMNSLTVEDTALYYCAR |
| | 16*01 | |
| 17 | Dog_ | ELTLQESGPGLVKPSQTLSLTCVVSGGSVTSSYYWNWIRQRPGRGLEWMGYWTGSTNYNPAFQGRISITADT |
| | IGHV3- | AKNQFSLQLSSMTTEDTAVYYCAR |
| | 26*01 | |
| 18 | Dog_ | EVQLVESGEDLVKPGGSLRLSCVASGFTFSSSEMSWVHQAPGQGLQWVSWIRYDGSSSRYADTVKGRFTISR |
| | IGHV3- | DNAKNTLYLQMNSLRAEDTAIYYCAD |
| | 46*01 | |
| 19 | Dog_ | EVPLVESGGELVKPEGSLRFSCVASGFTFSSYWISWVRQAPGKGLHWVSVINKDGSTTYHADAVKGRFTISR |
| | IGHV3- | DNAKNTLYLQMNSLRAEGTTVYYCAH |
| | 76*01 | |
| 20 | Dog_ | EVQLVESGGDLVKPAGSLRLSCVASGFTFSSYSMSWVRQAPEKGLQLVAGINSGGSSTYYTDAVKGRFTISR |
| | IGHV3- | DNAKNTVYLQMNSLRAEDTAMYYCAK |
| | 19*01 | |
| 21 | Dog_ | EVQLVESGGDLVKPEGSLRLSCVASGFTFSSFYMSWFCQAPRKGLQWVAEISSSGSSTSYADIVKGRFTISR |
| | IGHV3- | DNAKNMLYLQMNSLRAEDMAVYYCAR |
| | 24*01 | |
| 22 | Dog_ | EVQLVESGGDLVKSGGSLRLSCVASGFTFSSYYMHWVRQATGKGLQWVTRISNDGSSTRYADAMKGQFTISR |
| | IGHV3- | DNSKNTLYLQMNSQRAEDMALYYCAR |
| | 82*01 | |
| 23 | Dog_ | EMQLVEAGGDLVKLGGSLRLFCVASGFTFSSYWMSWVGQAPGKGLQWVAYINSGGSSTYYADAVKGRFTISR |
| | IGHV3- | DNAKNTLYLQMNCLRAEDTAVYYCVG |
| | 33*01 | |
| 24 | Dog_ | EVQLVESGGDLAKPGGSLRLSCVASGLTFSSYSMSWVRQAPGKGLQWVTAISYDGSSTYYTDAVKGRFTISR |
| | IGHV3- | DNARNTVYLQMNSLRAEDTAVYYCVD |
| | 47*01 | |
| 25 | Dog_ | ELQLVESGGDLVKPGGSLRLSCVSSGFTFSSYWMHWVLQAPGKGLEWVAIINSGGGSIYYADTVKGRFTISR |
| | IGHV3- | ENAKNTLYLQMNSLRAEDRAMHYCAK |
| | 83*01 | |
| 26 | Dog_ | GVQLVESGGDLVKPGGSLTLSCVAYGFTFSSYSMQWVCQAPGKGVQWVAYINSGGSSTSSADAVKGRFIISR |
| | IGHV3- | DNVKNTLYLQMNSLRAEDTAVYYCAG |
| | 32*01 | |
| 27 | Dog_ | EVQLVESGEDLVKPGGSLRLSCVASGFTFSSSEMSWVHQAPGQGLQWVSWIRYDGSISRYADTVKGRFTISR |
| | IGHV3- | DNVKNTLYLQMNSLRAEDTAIYYCAD |
| | 37*01 | |
| 28 | Dog_ | EVQLVESGGNLVKPGGSLRLSCVASGLTFYSYAIYWVHEAPGKGLQWVAAITTDGSSTYYTDAVKGRFTISR |
| | IGHV3- | DNAKNTLYLQMNSLRAEDMPVYYCAR |
| | 61*01 | |
| 29 | Dog_ | EVQLVESGGDLVKPERSLRLSCVASGFTFSSFYMSWFCQAPGKGLQCVAEISSSGNSTSYADAVKGRFTISR |
| | IGHV3- | DNAKNTLYLRMHSLRAEDTAVYYCAR |
| | 70*01 | |
| 30 | Dog_ | GAAGGVWGDLVKPGGSLRLSCVASGFTFSSYYMSWVRQAPGKGLQWVGYINSGGSSTYYADAVKGRFTISRD |
| | IGHV3- | NAKNTLYLQMNSLRAEDTAVYYCGK |
| | 12*01 | |
| 31 | Dog_ | EVQLVETEGDLVKPGGSLRLSCVASGFTFSSYDMDWVYQAPGKGLQWVTYISNGGSSTRYADAVKGQFTISR |
| | IGHV3- | DNARNTLYLQMNSLRDKDMAVYYCVS |
| | 10*01 | |
| 32 | Dog_ | EEQLVEFGGHMVNPGGSLGLSCQASGFTFSSYGMSWVRQAQKKGLQWVGHISYDGSSTYYADTLRDRFTISR |
| | IGHV3- | DNTKNMLYLQMNSLRAEDTAVYYCMR |
| | 7*01 | |
| 33 | Dog_ | EVQLVEPGGELVKPGASLRLSCVVPGFTFSSYNMGWAHQPPGKGMQWVAGFNSGGSSTSYTDAVKGEFTISR |
| | IGHV3- | DNVKNTLYLQMNSLRSEDTAVYYCVK |
| | 25*01 | |
| 34 | Dog_ | GEAWGSLRLSCVTSGFTFSSYAMHWVRQAPGKGLQWVAVISRDGSSTNYADAVKGRFTISRDNAKNMLYLQM |
| | IGHV3- | NSLRAEDTAMYYCAK |
| | 51*01 | |
| 35 | Dog_ | GGLVKPGGSLRLSCVDPGFTFSSYWMYWVHQAPGMGLQWLAEISSTGSSTNYADAVRGPFTISRDNAKNTLY |
| | IGHV3- | LQVNSLRAEDTAVYYCVS |
| | 53*01 | |
| 36 | Dog_ | AAGGVWGNLVKPGESLRLSCVASGFTFSSYWMHWVCQAPGKGLGWVAIINSGGGSTYYADTVKGQFTIFRDN |
| | IGHV3- | AKNMLYLQMNSLRAQDMTAYYCVS |
| | 55*01 | |
| 37 | Dog_ | SLGGSLRLSCVASEFIFSGYWKYWIHQAPGKGLQWVTWISNDGSSKSYADAVKGQFTISKDNAKYTLYLQMN |
| | IGHV3- | SLRAEDMAVYYCMM |
| | 4*01 | |
| 38 | IGHV2- | EVQLLESGGGLVQPGGSLRLSCAASGFTFSSYAMSWVRQAPGKGLEWVSAISGSGGSTYYADSVKGRFTISR |
| | 23D | DNSKNTLYLQMNSLRAEDTAVYYCAK |
| 39 | Dog_ | EVQLVESGGELVKPGGSLRLSCVASGFTFSSYYMSWIRQAPGKGLQWVADISDSGGSTYYTDAVKGRFTISR |
| | IGHV3- | DNVKNSLYLQMNSLRAEDTAVYYCAK |
| | 31*01 | |
| 40 | Dog_ | EVQLVESGGDLVKPGGSLRLSCVASGFTFSSHAKSWVRQAPGKGLKWVAVISSSGSSTGSADTVKGRFTISR |
| | IGHV3- | DNAKNTLYLQMNSLRAEDTAVYYCAK |
| | 58*01 | |
| 41 | Dog_ | EVQLVESGGDLVKPGGSLRLSCVASGFTFSSYYMSWIRQAPGKGLQWVADISDSGGSTGYADAVKGRFTISR |
| | IGHV3- | ENAKNKLYLQMNSLRAEDTAVYYCAK |
| | 39*01 | |
| 42 | Dog_ | EVQLVESGGDLVKPGGSLRLSCVASGFTFSNYDMSWVRQAPGKGLQWVAAISYDGSSTYYTDAVKGRFTISR |
| | IGHV3- | DNARNTVYLQMNSLRAEDTAVYYCAK |
| | 41*01 | |
| 43 | Dog_ | ELQLVELGGDLVKPGGSLRLSCVASGFTFSSYAMSWVCQAPGKGLQWVAAISSSGSSTYHVDAVKGRFTISR |
| | IGHV3- | DNAKNTVYLQMNSLRAEDTAVYYCAD |
| | 75*01 | |
| 44 | Dog_ | LVESGGDLMKPGGSLRLSCVASGFTFRSYGMSWVCQASGKGLQWVAAISYDGRSTYYTDTVKGRFTISRDNG |
| | IGHV3- | KNTLYLQMNSLRAEDTAVYYCAS |
| | 43*01 | |
| 45 | Dog_ | SWWSLGEDLVKPGGSLRLSCVTSGFTFSRYAMSWVGQAPGKGLQWVAAISSSGSSTYYVDAVKGRFTISIDN |
| | IGHV3- | AKNMVYLQMNSLRAEDIAVYYCGK |
| | 42*01 | |
| 46 | IGHV3- | EVQLVESGGGLVKPGGSLRLSCAASGFTFSSYSMNWVRQAPGKGLEWVSSISSSSSYIYYADSVKGRFTISR |
| | 21 | DNAKNSLYLQMNSLRAEDTAVYYCAR |
| 47 | Dog_ | EVQLVESGGDLVKPGGSLRLSCVASGFTFSSYDMDWVRQAPGKGLQWLSEISSSGSSTYYADAVKGRFTISR |
| | IGHV3- | DNAKNTLYLQMNSLRAEDTAVYYCAR |
| | 9*01 | |
| 48 | Dog_ | EVQLVESGGDLVKPVGSLRLSCVASGFTFSSYDMNWVRQAPGKGLQWVAYISSGGSSTYYADAVKGRFTISR |
| | IGHV3- | DNAKNTLYLQMNSLRAEDTAMYYCAG |
| | 35*01 | |
| 49 | Dog_ | EVQLVESGGDLVKPGGSLRLSCVASGFTFSNYEMYWVRQAPGKGLEWVARIYESGSTTYYAEAVKGRFTISR |
| | IGHV3- | DNAKNMAYLQMNSLRAEDTAVYYCAS |
| | 8*01 | |
| 50 | Dog_ | EVQLLESGGDRVKPGGSQRLSCVASRFTFSSYSMHCLRQSPGMGLQWVTYISSNGSSTYYADAVKGRFTISR |
| | IGHV3- | DKAKNMLYLQMNSLRAQDIALYYCAD |
| | 45*01 | |
| 51 | IGHV3-7 | EVQLVESGGGLVQPGGSLRLSCAASGFTFSSYWMSWVRQAPGKGLEWVANIKQDGSEKYYVDSVKGRFTISR |
| | | DNAKNSLYLQMNSLRAEDTAVYYCAR |
| 52 | Dog_ | EEQLVEFGGHMVNPGGSLGLSCQASGFTFSSYGMSWVRQAQKKGLQWVGHISYDGSSTYYTDTVRDRFTISR |
| | IGHV3- | DNTKNMLYLQMNSLRAEDTAVYYCMR |
| | 13*01 | |
| 53 | IGHV3- | EVQLVESGGGLVQPGGSLRLSCSASGFTFSSYAMHWVRQAPGKGLEYVSAISSNGGSTYYADSVKGRFTISR |
| | 64D | DNSKNTLYLQMSSLRAEDTAVYYCVK |
| 54 | Dog_ | SPVSCVASGFTFSNYSMIWVRQAPRKGLQWITTISNSGSSTNHADTVKGRFTISRDNTKNTLYLQMSSLGAD |
| | IGHV3- | DTALYYCVR |
| | 29*01 | |
| 55 | IGHV1- | QVQLVQSGAEVKKPGASVKVSCKASGYTFTSYYMHWVRQAPGQGLEWMGIINPSGGSTSYAQKFQGRVTMTR |
| | 46 | DTSTSTVYMELSSLRSEDTAVYYCAR |
| 56 | Dog_ | EVQLVQSGAEVKKPGASVKVSCKTSGYTFINYYMIWVRQAPGAGLDWMGQIDPEDGATSYAQKFQGRVTLTA |
| | IGHV1- | DTSTSTAYMELSSLRAGDIAVYYCAR |
| | 30*01 | |
| 57 | IGKV2D- | DIVMTQTPLSLSVTPGQPASISCKSSQSLLHSDGKTYLYWYLQKPGQPPQLLIYEVSNRFSGVPDRFSGSGS |
| | 29 | GTDFTLKISRVEAEDVGVYYC |
| 58 | Dog_ | DIVMTQTPLSLSVSPGEPASISCKASQSLLHSNGNTYLYWFRQKPGQSPQRLIYKVSNRDPGVPDRFSGSGS |
| | IgKV2- | GTDFTLRISRVEADDAGVYYC |
| | 5*02 | |
| 59 | Dog_ | DIVMTQTPLSLSVSPGEPASISCRASQSLLHSNGNTYLYWFRQKPGQSPQGLIYLVSNRFSWVPDRFSGSGS |
| | IgKV | GTDFTLRISRVEADDAGVYYC |
| | 2S13*01 | |
| 60 | Dog_ | DIVMTQTPPSLSVSPRETASISCKASQSLLHSDGNTYLDWYLQKPGQSPQLLIYLVSNRFTGVSDRFSGSGS |
| | IgKV2- | GTDFTLRISRVEANDTGVYYC |
| | 4*02 | |
| 61 | Dog_ | DIVMTQTPLSLSVSPGETASISCKASQSLLHSDGNTYLNWFRQKPGQSPQRLIYKVSNRDPGVPDRFSGSGS |
| | IgKV2- | GTDFTLRISRVEADDTGVYYC |
| | 9*01 | |
| 62 | Dog_ | EVVMIQTPLSLSVSPGEPASISCRASQSLRHSNGNTYLYWYLQKPGQSPQLLIDLVSNHFTGVSDRFSGSGS |
| | IgKV2- | GTDFTLRISRVEAEDVGVYYC |
| | 20*02 | |
| 63 | Dog_ | EAVMTQTPLSLAVTPGELATISCRASQSLLRSDGKSYLNWYLQKPGQTPRPLIYEASKRFSGVSDRFSGSGS |
| | IgKV2- | GTDFTLKISRVEAEDVGVYYC |
| | 16*01 | |
| 64 | Dog_ | DIVMTQTPPSLSVSPGEPASISCKASQSLLHSNGNTYLNWFRQKPGQSPQGLIYRVSNRSTGVSDRFSGSGS |
| | IgKV2- | GTDFTLRISRVEADDAGVYYC |
| | 8*01 | |
| 65 | Dog_ | DIVMTQNPLSLSVSPGETASISCKASQSLLHSNGNTYLNWFRQKPGQSPQGLIYKVSNRDPGVPDRFSGSGS |
| | IgKV2- | GTDFTLRISRVEADDAGVYYC |
| | 7*01 | |
| 66 | Dog_ | DIVMTQTPLSVSVSPGETASISCRASQSLLHSDGNTYLDWYLQKPGQIPKDLIYRVSNCFTGVSDRFSGSGS |
| | IgKV2- | GTDFTLRISRVEADNAGVYYC |
| | 13*02 | |
| 67 | Dog_ | DIVMTQTPLSLSVSPGETASISCKASQSLLHSNGNTYLFWFRQKPGQSPQRLINLVSNRDPGVPHRFSGSGS |
| | IgKV2- | GTDFTLRISRVEADDAGVYYC |
| | 11*02 | |
| 68 | IGKV2-28 | DIVMTQSPLSLPVTPGEPASISCRSSQSLLHSNGYNYLDWYLQKPGQSPQLLIYLGSNRASGVPDRFSGSGS |
| | | GTDFTLKISRVEAEDVGVYYC |
| 69 | Dog_ | DIVMTQTPLSLSVSPGETASISCRANQSLLHSNGNTYLDWYMQKPGQSPQGLIYRVSNHFTGVSDRFSGSGS |
| | IgKV2- | GTDFTLKISRVEADDAGVYYC |
| | 12*02 | |
| 70 | IGKV4-1 | DIVMTQSPDSLAVSLGERATINCKSSQSVLYSSNNKNYLAWYQQKPGQPPKLLIYWASTRESGVPDRFSGSG |
| | | SGTDFTLTISSLQAEDVAVYYC |
| 71 | Dog_ | EIVMTQSPGSLAGSAGESVSINCKSSQSLLYSFNQKNYLAWYQQKPGERPKLLIYLASSWASGVPARFSSSG |
| | IgKV4- | SGTDFTLTINNLQAEDVGDYYC |
| | 15*01 | |
| 72 | IGKV3-11 | EIVLTQSPATLSLSPGERATLSCRASQSVSSYLAWYQQKPGQAPRLLIYDASNRATGIPARFSGSGSGTDFT |
| | | LTISSLEPEDFAVYYC |
| 73 | Dog_ | EIVMTQSPASLSLSQEEKVTITCRASQSVSSYLAWYQQKPGQAPKLLIYGTSNRATGVPSRFSGSGSGTDFS |
| | IgKV3- | FTISSLEPEDVAVYYC |
| | 18*02 | |
| 74 | IGLV1-44 | QSVLTQPPSASGTPGQRVTISCSGSSSNIGSNTVNWYQQLPGTAPKLLIYSNNQRPSGVPDRFSGSKSGTSA |
| | | SLAISGLQSEDEADYYC |
| 75 | Dog_ | QSVLTQPASVSGSLGQRVTISCTGSSSNIGRGYVGWYQQLPGTGPRTLIYDNSNRPSGVPDRFSGSKSGSTA |
| | IGLV1- | TLTISGLQAEDEADYYC |
| | 157*01 | |
| 76 | Dog_ | QSVLTQPASVSGSLGQRVTISCTGSSSNIGRDYVGWYQQLPGTRPRTLIYGNSNRPSGVPDRFSGSKSGSTA |
| | IGLV1- | TLTISGLQAEDEADYYC |
| | 46*01 | |
| 77 | Dog_ | QSVLTQPPSVSGFLGQRVTISCTGSSSNIGRGYVHWYQQLPGTGPRTLIYGISNRPSGVPDRFSGSRSGSTA |
| | IGLV1- | TLTISGLQAEDEADYYC |
| | 94*01 | |
| 78 | Dog_ | QSVLTQPASVSGSLGQRVTISCTGSSSNIGRGYVGWYQQLPGTGPRTLIYGNSNRPSGVPDRFSGSRSGSTA |
| | IGLV1- | TLTISGLQAEDEADYYC |
| | 125*01 | |
| 79 | Dog_ | QSVLTQPASVSGSLGQRITISCTGSSSNIGGNNVGWYQQLPGRGPRTVIYSTNSRPSGVPDRFSGSKSGSTA |
| | IGLV1- | TLTISGLQAEDEADYYC |
| | 55*01 | |
| 80 | Dog_ | QSVLTQPPSVSGSLGQRVTISCSGSTNNISIVGASWYQQLPGKAPKLLVDSDGDRPSGVPDRFSGSKSGKSA |
| | IGLV1- | TLTITGLQAEDEADYYC |
| | 69*01 | |
| 81 | Dog_ | QSVLTQPTSVSGSLGQRVTISCSGSTNNIGIVGASWYQQLPGKAPKLLVYSVGDRPSGVPDRFSGSNSGNSA |
| | IGLV1- | TLTITGLQAEDEADYYC |
| | 141*01 | |
| 82 | Dog_ | QSVLTQPTSVSGSLGQRVTISCSGSTNNIGIVGASWYQQLPGKAPKLLVDSDGDRPSGVPDRFSGSKSGNSA |
| | IGLV1- | TLTITGLQAEDEADYYC |
| | 100*01 | |
| 83 | Dog_ | QSMLTQPASVSGSLGQKVTISCTGSSSNIGGNYVGWYQQLPGIGPRTVIYGNNYRPSGVPDRFSGSKSGSSA |
| | IGLV1- | TLTISGLQAEDEAEYYC |
| | 48*01 | |
| 84 | Dog_ | QSVLTQPASVSGSLGQRVTISCTGSSSNIGRGYVGWYQQLPGTGPRTLIYDSSSRPSGVPDRFSGSRSGSTA |
| | IGLV1- | TLTISGLQAEDEADYYC |
| | 136*01 | |
| 85 | Dog_ | QSVLTQPTSVSGSLGQRVTISCSGSTNNIGIVGASWYQQLPGKAPKLLVYSDGDRPSGVPDRFSGSNSGNSD |
| | IGLV1- | TLTITGLQAEDEADYYC |
| | 162*01 | |
| 86 | Dog_ | QSVLTQPASVSGSLGQRVTISCTGSSSNIGRYSVGWFQQLPGKGPRTVIYSSSNRPSGVPDRFSGSKSGSTA |
| | IGLV1- | TLTISGLQAEDEADYYC |
| | 58*01 | |
| 87 | Dog_ | QPVLTQPPSVSGFLGQRVTISCTGSSSNILGNSVNWYQQLTGRGPRTVIYYDNNRPSGVPDQFSGSKSGNSA |
| | IGLV1- | TLTISGLQAEDETDYYC |
| | 116*01 | |
| 88 | Dog_ | QSVLTQPPSVSGSVGQRITISCSGSTNSIGILGVNWYQLLSGKAPKLLVDGTGNRPSGVPDRFSGSKSGNSG |
| | IGLV1- | TLTITGLQPEDEADYYC |
| | 66*01 | |
| 89 | Dog_ | QSVLNQPPSVSGSLGQRITISCSGSTNDIGMLGVNWYQQLPGNAPKLLVDGTGNRPSGVPDQFSGSKSGNSG |
| | IGLV1- | TLTITGLQAEDEADYYC |
| | 73*01 | |
| 90 | Dog_ | QSVLTQPASASGSLGQRVTVSCSSSTNNIGIIGVKWYQQIPGKAHKLLIYDNEKRPSGVPNRFSGSKSGDLS |
| | IGLV1- | TLTINGLQGEDEADYYC |
| | 84-1*01 | |
| 91 | Dog_ | QSVLTQPASVTGSLGQRVTISCTGSSSNIGGYNVGWFQQLPGTGPRTVIYSSSNRPSGVPDRFSGSRSGSTA |
| | IGLV1- | TLTISGLQAEDEAEYYC |
| | 75*01 | |
| 92 | Dog_ | QSVLTQPASVSGVLGQRVTISCTGSSSNIGGNYVSWHQQVPETGPRNIIYADNYRASGVPDRFSGSKSGSTA |
| | IGLV1- | TLTISVLQAEDEADYYC |
| | 67*01 | |
| 93 | Dog_ | QSVLTQPTSVSWSLGQRVTISCSRSTNNIGIVGASWYQQLPGKAPKLLVDSDGDQLSGVPDRFSGSKSGNSA |
| | IGLV1- | NLTITGLQAEDKADYYC |
| | 80*01 | |
| 94 | Dog_ | QSVLNQLPSVLGSLGQRITISCSGSTNDIGMLGVNWYQELPGKAPKLLVDGTGNRPSGVPDRFSGSKSGNSG |
| | IGLV1- | TLTITGLQAEDEADYYC |
| | 158*01 | |
| 95 | Dog_ | QSVLTQPASVSGSLGQRVIISCTGSSSSIGRGYVGWYQQLPGTGPRTLIYGISNLPPGVPNRFSGSRSGSTA |
| | IGLV1- | TLTIAELQAEDEADYYC |
| | 106*01 | |
| 96 | Dog_ | QSVLTQPPSVFRSLGQRVTISCTGSSCNVGRGYVIWYQQLLGTRPRTLIYGSSNQPSGVPNRFSGSRSGSTA |
| | IGLV1- | TLTISGFQAEDEADYYC |
| | 159*01 | |
| 97 | Dog_ | QSVLTQPPSVFRSLGQRVTISCTGSSSNVGRGYVIWYQQLLGTRPRTLIYGSSNQPSGVPNQFSGSRSGSTD |
| | IGLV1- | TLTISGFQAEDEADYYC |
| | 97-4*01 | |
| 98 | Dog_ | PGPEVTVSCTGSCFNIGRYSVSWLQQLPGTGPRTIIYYDRSRPSGVPDRFSGSKSGSTATLTISGLQAEDEA |
| | IGLV1- | DYYC |
| | 127*01 | |
| 99 | IGLV8-61 | QTVVTQEPSFSVSPGGTVTLTCGLSSGSVSTSYYPSWYQQTPGQAPRTLIYSTNTRSSGVPDRFSGSILGNK |
| | | AALTITGAQADDESDYYC |
| 100 | Dog_ | QTVVTQEPSLSVSPGGTVTLTCGLSSGSVSTSNYPGWYQQTLGRAPRTIIYRTSSRPSGVPNRFSGSISGNK |
| | IGLV8- | AALTITGAQPEDEADYYC |
| | 128*01 | |
| 101 | Dog_ | QTVVTQEPSLSVSPGGTVTLTCGLSSGSVSTSNYPGWYQQTQGRAPRTIIYNTSSRPSGVPNRFSGSISGNK |
| | IGLV8- | AALTITGAQPEDEADYYC |
| | 93*01 | |
| 102 | Dog_ | QTVVTQEPSLSVSLGGTVTLTCGLSSGSVSTSNYPNWSQQTPGQAPRTIIYNTNSRPSGVPNRFTGSISGNK |
| | IGLV8- | AALTITGAQPEDEADYYC |
| | 36*01 | |
| 103 | Dog_ | QTVVTQEPSLSVSPGGTVTLTCGLSSGSVSTSNHPSWYQQTQGKAPRMLIYNTNNRPSGIPNCFSGSISGNK |
| | IGLV8- | ASLTITGAQPEDETDYYC |
| | 39*01 | |
| 104 | Dog_ | QTVVTQKPSLSVSPGGTVTLICGLSSGSVSTSNYPGWYQQTQGRASRTIIYSTSSRPSGVPNRFTGSISGNK |
| | IGLV8- | AALTITGAQPEDEADYYC |
| | 142*01 | |
| 105 | Dog_ | QTVVTQKPSLSVSPGGTVTLICGFSSGSVSTSNYPGWYQQTQGRASRTIIYSTSSRPSGVPNRFPGSISGNK |
| | IGLV8- | AALTITGAQPEDEADYYC |
| | 99*01 | |
| 106 | Dog_ | QTVVTQKPSLSVSPGGTVTLICGLSSGSVSTSNYPGWYQQTQGRASRTIIYSTSSRPSGVPNRFPGSISGNK |
| | IGLV8- | AALIITGAQPEDEADYYC |
| | 161*01 | |
| 107 | Dog_ | QIVVTQEPSLSVSPGGTVTLTCGLSSGSVTISNYPDWYQQTPGRSPRMLIYNTNNRPSGVPNHFSGSISGNK |
| | IGLV8- | AALTITGAQPEDEAYYYC |
| | 74-1*01 | |
| 108 | Dog_ | QTVVTQEPSLSVSPGGTVTLTCGLSSGSVSTGNKPGWYQHTPGQAPRRIIYDTSSRPSGVPDRFSGSISENK |
| | IGLV8- | AALTITEAQPEDEAAYHC |
| | 119*01 | |
| 109 | Dog_ | QTVVTQESSVSVSPVGTVTLTCGLSSGSLTTSNYTSWYQQTQGQSPRMLVYDTSSCPSEVPDHFSGSISGNK |
| | IGLV8- | AALTITGAQPEDEADYYC |
| | 121*01 | |
| 110 | Dog_ | QEPSLSPGGTVLLTCGLSSGSVTTSNYSSWYQQTPGRAPRTIIYNTNSHPSGVPDRFSGSISGNKAALTITG |
| | IGLV8- | AQPEDEADYYC |
| | 102*01 | |
| 111 | Dog_ | QTVVTQDPSLSVSPGGTVTLTCGLSSGSVSTSNYPGWYQQTQVKAPCMLIYSTNSYPSGVPNCFTGSISGKK |
| | IGLV8- | AALTITGDQPEDETIIVH |
| | 76-1*01 | |
| 112 | Dog_ | QTVITQDTSLSVSPGGTVTLTCGLSSGSVSTSNYPSWYQQTQGRDPRMLIYSTNSHPSGVPNCFTRSISGKK |
| | IGLV8- | AALTITGAQPEDETIIVH |
| | 60*01 | |
| 113 | Dog_ | GTVTLTCGLSSGSVSTSNYPNWYQQTQGRAPHRVIYNTNNRPSGVPDRFSGSISGNKAALTITAAQPEDEAD |
| | IGLV8- | YYC |
| | 43*01 | |
| 114 | Dog_ | GGQEALLSVSPGGRVTLTCGLSSGSVTTSNYPNWFQQTPGRAPGTIIYSTKDCPSGVPDCFSRSISGNKAAL |
| | IGLV8- | TITGAQSEDEAITVL |
| | 89-3*01 | |
| 115 | Dog_ | GQEALLSVSSGGRVTLTCGLSSGSVTTSNYPNWFQQTPGRAPGTIMYSTKDCPSGVPDCFSRSISGNKAALT |
| | IGLV8- | ITGAQSEDEVITVL |
| | 118-3*01 | |
| 116 | IGLV1-40 | QSVLTQPPSVSGAPGQRVTISCTGSSSNIGAGYDVHWYQQLPGTAPKLLIYGNSNRPSGVPDRFSGSKSGTS |
| | | ASLAITGLQAEDEADYYC |
| 117 | Dog_ | QAVLTQPPSVSAVLGQRVTISCTGSSTNIGSGYDVQWYQQLPGKSPKTIIYGNSNRPSGVPDRFSGSKSGST |
| | IGLV1- | ASLTITGLQAEDEADYYC |
| | 49*01 | |
| 118 | Dog_ | QAVLTQPPSVSAALGQRVTISCTGSNTNIGSGYDVQWYQQLPGKSPKTIIYGNSNRPSGVPVRFSGSKSGST |
| | IGLV1- | ATLTITGIQAEDEADYYC |
| | 103*01 | |
| 119 | Dog_ | QSVLTQPASVSGSLGQRVTISCTGSTNNIGGDNYVHWYQQLPGKAPSLLIYGDDNRESGVPERFSGSKSGSS |
| | IGLV1- | ATLTITGLQAEDEADYYC |
| | 87*01 | |
| 120 | Dog_ | QAVLTQPASVSGSLGQRVTISCTGSSSNVGYGNYVGWYQQLPGTGPRTLIYGSSYRPSGVPDRFSGSSSGSS |
| | IGLV1- | ATLTISGLQAEDEADYYC |
| | 84*01 | |
| 121 | Dog_ | QSVLTQPASVSGSLGQRVTISCTGSSSNVGYGNYVGWYQQLPGTGPRTLIYRSSSRPSGVPDRFSGSRSGST |
| | IGLV1- | ATLTISGLQAEDEADYYC |
| | 149*01 | |
| 122 | Dog_ | QSVLTQPASVSGSLGQRVTISCTGSSSNVGYGNYVGWYQQLPGTSPRTLIYDSSSRPSGVPDRFSGSRSGST |
| | IGLV1- | ATLTISGLQAEDEADYYC |
| | 138*01 | |
| 123 | Dog_ | QSVLTQPASVSGSLGQRVTISCTRSSSNVGYGNDVGWYQQLPGTGPRTIIYNTNTRPSGVPDRFSGSKSGST |
| | IGLV1- | ATLTISGLQAEDEADYYC |
| | 70*01 | |
| 124 | Dog_ | QSVLTQPASVSQSLGQRVTISCTGSSSNVGYNSYVSWYQQLPGTVPRTIIYYTNTRPYGVPDRFSGSKSGNS |
| | IGLV1- | ATLTIAGLQAEDEADYYC |
| | 112*01 | |
| 125 | Dog_ | QAVLTPLPSVSAALGQTVTISCTGNSTQIGSGYAVQWYQQLPGKSPETIIYGDSNRPSGVPDRFSGFSSGNS |
| | IGLV1- | ATLAITGLQDEDEADYYC |
| | 150*01 | |
| 126 | Dog_ | QAVLTPLPSVSAALGQTVTISCTGNSTQISSGYAVQWYQQLPGKSPETIIYGDSNRPSGVPDRFSGFSSGNS |
| | IGLV1- | ATLAITGLQDEDEADYYC |
| | 57*01 | |
| 127 | IGLV2-18 | QSALTQPPSVSGSPGQSVTISCTGTSSDVGSYNRVSWYQQPPGTAPKLMIYEVSNRPSGVPDRFSGSKSGNT |
| | | ASLTISGLQAEDEADYYC |
| 128 | Dog_ | QSALTQPSSVSGTLGQTVTISCDGSSSNIGSSNYIEWYQQFPGTSPKLLIYYTNNRPSGIPARFSGSKSGNT |
| | IGLV2- | ASLTISGLQAEDEADYYC |
| | 31*01 | |
| 129 | Dog_ | QSVLTQPASVSGSLGQRVTISCTGSSSNVGYSSSVGWYQQFPGTGPRTIIYYDSSRPSGVPDRFSGSKSGST |
| | IGLV1- | ATLTISGLQAEDEADYYC |
| | 41*01 | |
| 130 | Dog_ | QSVLTQPASVSGSLGQRVTISCTGSSSNVGYGNYVGWYQQLPGTGPRTIICYTNTRPSGVPDRYSGSKSGST |
| | IGLV1- | ATLTISGLQAEDETDYYC |
| | 92*01 | |
| 131 | Dog_ | QSALTQTASMSGSLGQRVTVSCTGSSSNVGYRSYVGWYQQLPGTGPRTIIYNTNTRPSGVPDRFSGSISGST |
| | IGLV1- | ATLTIAGLQAEDEADYYC |
| | 151*01 | |
| 132 | Dog_ | QSALTQPPSMSGTLGQTIIISCTGSGSDIGRYSYVSWYQELPSTSPTLLIYGTNNRPLEIPARFSGSKSGNT |
| | IGLV2- | APMTISGLQAEDEANYYC |
| | 32*01 | |
| 133 | Dog_ | GPGPEVTISCTRSSSNVGYSSYVGWYQQLPGTGPKTIIYNTNTRPSGVPDRFSGSKSGRTATLTIAGLQAED |
| | IGLV1- | EADYYC |
| | 83*01 | |
| 134 | Dog_ | GPGPEVTISCTRSSSNVGYSSYVGWYQQLPGTGPKTIIYNTNTRPSGVPDRFSGSKSGSTATLTIAGLQAED |
| | IGLV1- | EADYYC |
| | 79*01 | |
| 135 | IGLV3-9 | SYELTQPLSVSVALGQTARITCGGNNIGSKNVHWYQQKPGQAPVLVIYRDSNRPSGIPERFSGSNSGNTATL |
| | | TISRAQAGDEADYYC |
| 136 | Dog_ | SYVLTQLPSVSVTLRQTARITCGGDSIGSKSVYWYQQKLGQAPVLIIYRDSNRPTGIPERFSGANSGNTATL |
| | IGLV3- | TISGALAEDEADYYC |
| | 8*01 | |
| 137 | Dog_ | SYVLTQLPSVSVTLRQTARITCGGDSIGSKNVYWYQQKLGQAPVLIIYDDSSRPSGIPERFSGANSGNTATL |
| | IGLV3- | TISGALAEDEADYYC |
| | 24*01 | |
| 138 | Dog_ | SYVLTQLPSVNVTLRQPAHITCGGDSIGSKSVHWYQQKLGQAPVLIIYGDSNRPSGIPERFSGDNSGNTATL |
| | IGLV3- | TISGALAEDEAYYYC |
| | 26*01 | |
| 139 | Dog_ | SYVLTQSPSVSVTLGQTASITCRGNSIGRKDVHWYQQKPGQAPLLIIYNDNSQPSGIPERFSGTNSGSTATL |
| | IGLV3- | TISEAQTNDEADYYC |
| | 2*01 | |
| 140 | Dog_ | SYVLTQLPSMSVTLRQTARITCEGDSIGSKRVYWYQQKLGQVPVLIIYDDSSRPSGIPERFSGANSGNTATL |
| | IGLV3- | TISGALAEDEADYYC |
| | 14*01 | |
| 141 | Dog_ | LSQPPSATVTLRQTARLTCGGDSIGSKSVEWYQQKPGQPPVLIIYGDSSRPSGIPERFSGANSGNTATLTIS |
| | IGLV3- | GALAEDEADYYC |
| | 11*01 | |
| 142 | Dog_ | LKQPAHITCGGDNIGSKSVHWYQQKLGQAPVLIIYYDSSRPTGIPERFSGANSGNTATLTISGALAEDEADY |
| | IGLV3- | YC |
| | 3*01 | |
| 143 | Dog_ | ANKLTQSLFMSVALGQMARITCGRDNSGRKSAHWYQQKPSQAPVMLIDDDCFQPSGFSEQFSGTNSGNTATL |
| | IGLV3- | TISGPPARTRLLLCH |
| | 1*01 | |
| 144 | IGLV3-25 | SYELMQPPSVSVSPGQTARITCSGDALPKQYAYWYQQKPGQAPVLVIYKDSERPSGIPERFSGSSSGTTVTL |
| | | TISGVQAEDEADYYC |
| 145 | Dog_ | SSVLTQPPSVSVSLGQTATITCSGESLSRYYAQWYQQKPGQAPMTVIYGDRERPSGIPDRFSSSSSENTHTL |
| | IGLV3- | TISGAQAEDEAEYYC |
| | 29*01 | |
| 146 | Dog_ | SPGLNQPPSVLVALGQMATNTCSGDVLGKRYAYWYQHKPSQAPVLLINKNNELGSGIPDRFSGSNSGNTATL |
| | IGLV3- | TISGARAEDEADYYC |
| | 19*01 | |
| 147 | Dog_ | LNQAPSVLVALGQMETITCSRDVLGKRYAYRYQHKPSQAPVLLINKNNEQDSGIPDRFSGSNSGNTATLTIS |
| | IGLV3- | GARAEDEAEYYC |
| | 15*01 | |
| 148 | IGLV3-27 | SYELTQPSSVSVSPGQTARITCSGDVLAKKYARWFQQKPGQAPVLVIYKDSERPSGIPERFSGSSSGTTVTL |
| | | TISGAQVEDEADYYC |
| 149 | Dog_ | SSVLTQPPSVSVSLGQTATISCSGESLSKYYAQWFQQKAGQVPVLVIYKDTERPSGIPDRFSGSSSGNTHTL |
| | IGLV3- | TISGARAEDEADYYC |
| | 27*01 | |
| 150 | Dog_ | SSVLVALGQMETITCSRDVLGKRYAYRYQHKPSQAPVLLINKNNEQDSGIPDRFSGSNSGNTATLTISGARA |
| | IGLV3- | EDEAEYYC |
| | 25*01 | |
| 151 | IGLV5-37 | QPVLTQPPSSSASPGESARLTCTLPSDINVGSYNIYWYQQKPGSPPRYLLYYYSDSDKGQGSGVPSRFSGSK |
| | | DASANTGILLISGLQSEDEADY |
| 152 | Dog_ | QPVLTQPPSLSASLGTTARLTCTLSSGFSVGDYDMYWYQQKPGSPPRDLLYYYSDSYKNQGSGVSKSFSGSK |
| | IGLV5- | DTSANAGLLLISGLQPEDEADYYC |
| | 131*01 | |
| 153 | Dog_ | QLVVTQPPSLSASLGSSARLTCTLSSGFSVGSYSVTWFQQKPGSPLWYLLYYHSDSDKHQGSRVPSRFSGSK |
| | IGLV5- | DTSANAGLLLISGLQPEDEADYYC |
| | 64*01 | |
| 154 | IGLV3-21 | SYVLTQPPSVSVAPGKTARITCGGNNIGSKSVHWYQQKPGQAPVLVIYYDSDRPSGIPERFSGSNSGNTATL |
| | | TISRVEAGDEADYYC |
| 155 | Dog_ | SYVLTQLPSVSVNLGKTASITCEGNNIGDKYAYWYQQKPGQAPVLIIYEDSKRPSGIPERFSGSNSGNTATL |
| | IGLV3- | TISGARAEDEADYYC |
| | 28*01 | |
| 156 | IGLV3-16 | SYELTQPPSVSVSLGQMARITCSGEALPKKYAYWYQQKPGQFPVLVIYKDSERPSGIPERFSGSSSGTIVTL |
| | | TISGVQAEDEADYYC |
| 157 | Dog_ | PSMLVALGQMETITCSGDILGKRYAYWYQHKPSQAPVLLINKNNERASGIPHWFSGSNSGNMATLTISGARA |
| | IGLV3- | EDEADYYC |
| | 4*01 | |
| 158 | IGLV1-51 | QSVLTQPPSVSAAPGQKVTISCSGSSSNIGNNYVSWYQQLPGTAPKLLIYDNNKRPSGIPDRFSGSKSGTS |
| | | ATLGITGLQTGDEADYYC |
| 159 | Dog_ | QAVLNQPASVSGALGQKVTISCSGSTNDIDIFGVSWYQQLPGKAPKLLVDSDGDRPSGVPDRFSGSSSGNS |
| | IGLV1- | GTLTITGLQAEDEADYYC |
| | 44*01 | |
| 160 | IGLV5-39 | QPVLTQPTSLSASPGASARFTCTLRSGINVGTYRIYWYQQKPGSLPRYLLRYKSDSDKQQGSGVPSRFSGS |
| | | KDASTNAGLLLISGLQSEDEADY |
| 161 | Dog_ | QPVLTQPPSLSASLGSTARPTCTLSSGFSVGSYHILWFQQKSESPPRYLLRFYSDSNEHQGPGVPSRFSGS |
| | IGLV5- | KDTSTYAGLLLISGLQPEDEADYYC |
| | 85*01 | |
| 1 | IGHV3-74 | EVQLVESGGGLVQPGGSLRLSCAASGFTFSSYWMHWVRQAPGKGLVWVSRINSDGSSTSYADSVKGRFTIS |
| | | RDNAKNTLYLQMNSLRAEDTAVYYCAR |
| 162 | Dingo- | EVQLVESGGDLVKPAGSLRLSCVASGFTFSSYGMSWVRQAPGKGLQWVAGINSGGSSTSYADAVKGRFTIS |
| | 1680000- | RDNAKNTLYLQMNSLRAEDTAVYYCAK |
| | 1681500 | |
| 163 | Dingo- | EVQLVESGGDMVKPGGSLRLSCVASGFTFSSYYMYWARQAPGKGLQWVSHINKDGSSTSYADAVKGRFTIS |
| | 1338000- | RDNAKNTLYLQMNSLRAEDTAVYYCAK |
| | 1339500 | |
| 164 | Dingo- | EVQLAESGGDLVKPGGSLRLSCVASGFTFSSNYMSWIRQAPGKGLQWVSQISSDGSSTSYADAVKGRFTIS |
| | 1320000- | RDNAKNTLYLQMNSLRDEDTAVYYCAR |
| | 1321500 | |
| 165 | Dingo- | EVQLVESGGDLVKPGGSLRLSCVTSGFTFSSYWMSCVRQAPGKELQWVAYINSGGSSTWYTDAVKGRFTIS |
| | 1970000- | RDNAKNTLYLQMNSLRAEDTAVYYCAR |
| | 1971500 | |
| 166 | Dingo- | EVQLVESGGDLVKPGGSLRLSCVASGFTFSSSDMSWIRQAPEKGLQWVAYISNDGSSTSYADAVKGRFTIS |
| | 1426000- | RDNAKNTLYLQMSSLRAEDTAVYYCAD |
| | 1427500 | |
| 167 | Dingo- | EVQLVESGGDLVKPGGSLRLSCVASGFTFSSYYMFWIRQAPGKGNQWVGYINKDGSSTYYPDAVKGRFTIS |
| | 1619000- | RDNAKNTLYLQMNSLTVEDTALYYCAR |
| | 1620500 | |
| 168 | Dingo- | EVQLVESRGDLVKPGGSLRLSCVAYGFTFSSYSMQCVRQAPGKGVQWVAYINSGGSSTSSADAVKGRFIIS |
| | 1815000- | RDNVKNTLYLQMNSLRAEDTAVYYCAG |
| | 1816500 | |
| 169 | Dingo- | EVQLLESGGDLVKPGGSLRLSCVASEFIFSGYWKYWIHQAPGKGLQWVTWISNDGSSKSYADAVKGRFTIS |
| | 1351000- | KDNAKNTLYLQMNSLRAEDMAVYYCMM |
| | 1352500 | |
| 170 | Dingo- | EVQLVETEGDLVKPGGSLRLSCVASGFIFSSYDMDWVCQAPGKGLQWVTYISNGGSSTRYADAVKGQFTIS |
| | 1506000- | RDNARNTLYLQMNNLRDEDMAVYYCVS |
| | 1507500 | |
| 171 | Dingo- | EVQLVEPGGELVKPGASLRLSCVVPGFTFSSYNMGWAHQPPGKGMQWVAGFNSGGSSTSYTDAVKGEFTIS |
| | 1726000- | RDNVKNTLYLQMNSLRSEDTAVYYCVK |
| | 1727500 | |
| 38 | IGHV3- | EVQLLESGGGLVQPGGSLRLSCAASGFTFSSYAMSWVRQAPGKGLEWVSAISGSGGSTYYADSVKGRFTIS |
| | 23D | RDNSKNTLYLQMNSLRAEDTAVYYCAK |
| 172 | Dingo- | EVQLVESGGDLVKPGGSLRLSCVASGFTFSSYDMSWVRQSPGKGLQWVAAIMRSGSSTYYADAVKGRFTIS |
| | 1879000- | RDNAKNTLYLQMNSLRAEDTAVYYCAK |
| | 1880500 | |
| 173 | Dingo- | EVQLVESGGDLVKPGGSLRLSCVASGFTFSSYYMSWIRQAPGKGLQWVADISDSGGSTGYADAVKGRFTIS |
| | 1895000- | RENAKNTLYLQMNSLRAEDTAVYYCAK |
| | 1896500 | |
| 174 | Dingo- | EVQLVESGGELVKPGGSLRLSCVASGFTFSSYYMSWIRQAPGKGLQWVADISDSGGSTYYTDAVKGRFTIS |
| | 1805000- | RDNVKNSLYLQMNSLRAEDTAVYYCAK |
| | 1806500 | |
| 175 | Dingo- | EVQLVESGGDLVKPGGSLRLSCVASGFTFSNYDMSWVRQAPGKGLQWVAAISYDGSSTYYTDAVKGRFTIS |
| | 1926000- | RDNARNTVYLQMNSLRAEDTAVYYCAK |
| | 1927500 | |
| 176 | Dingo- | EVQLVESGGDLVKPVASLRLSCVASGFTFSNYAMSWVHQAPGKGLQWVAEISHSGRSTGYTDAMKGRFTIT |
| | 1644000- | RDNAKNMPYLKMNSLTAKDMAMYYCAR |
| | 1645500 | |
| 46 | IGHV3-21 | EVQLVESGGGLVKPGGSLRLSCAASGFTFSSYSMNWVRQAPGKGLEWVSSISSSSSYIYYADSVKGRFTIS |
| | | RDNAKNSLYLQMNSLRAEDTAVYYCAR |
| 177 | Dingo- | EVQLVESGGDLVKPGGSLRLSCVASGFTFSSYDMDWVRQAPGKGLQWLSEISSSGSSTYYADAVKGRFTIS |
| | 1496000- | RDNAKNTLYLQMNSLRAEDTAVYYCAR |
| | 1497500 | |
| 178 | Dingo- | EVQLVESGGDLVKPVGSLRLSCVASGFTFSSYDMNWVRQAPGKGLQWVAYISSGGSSTYYADAVKGRFTIS |
| | 1835000- | RDNAKNTLYLQMNSLRAEDTAMYYCAG |
| | 1836500 | |
| 179 | Dingo- | EVQLVESGGDLVKPGGSLRLSCVASGFTFSNYEMYWVRQAPGKGLEWVARIYESGSTTYYAEAVKGRFTIS |
| | 1448000- | RDNAKNMVYLQMNSLRAEDTVRGPQCE |
| | 1449500 | |
| 180 | Dingo- | EVQLLESGGDRVKPGGSQRLSCVASRFTFSSYSMHWLRQSPGMGLQWVTYISSSGSSTYYADAVKGRFTIS |
| | 1975000- | RDKAKNMLYLQMNSLRAQDIALYYCAD |
| | 1976500 | |
| 51 | IGHV3-7 | EVQLVESGGGLVQPGGSLRLSCAASGFTFSSYWMSWVRQAPGKGLEWVANIKQDGSEKYYVDSVKGRFTIS |
| | | RDNAKNSLYLQMNSLRAEDTAVYYCAR |
| 181 | Dingo- | EEQLVEFGGHMVNPGGSLGLSCQASGFTFSSYGMSWVRQAQKKGLQWVGHISYDGSSTYYTDTVRDRFTIS |
| | 1442000- | RDNTKNMLYLQMNSLRAEDTAVYYCMR |
| | 1443500 | |
| 1 | IGHV3-74 | EVQLVESGGGLVQPGGSLRLSCAASGFTFSSYWMHWVRQAPGKGLVWVSRINSDGSSTSYADSVKGRFTIS |
| | | RDNAKNTLYLQMNSLRAEDTAVYYCAR |
| 182 | Fox_1- | EVQLVESGGALVKPGGSLRLSCVASGFTFSSYAMSWVRQAPGKGLQWVGYISSDGSSTYYADAVKGRFTIS |
| | 7000- | RDNAKNTLYLQMNSLRVEDTAVYYCAR |
| | 8500 | |
| 183 | Fox_7- | EVQLVESGGDLVKPGGSLRLSCVASGFTFSSYDMHWVRQAPGKGLQWVAAISYGGSSTNYADAVKGRFTIS |
| | 5000- | RDNAKNTLYLQMNSLRAEDTAVYYCAR |
| | 6500 | |
| 184 | Fox_5- | EVQLVESGGDLVKPGGSLRLSCVASGFTFSSYYMSWIRQAPGKGLQWVAYISSDGSSTRYADAVKGRFTIS |
| | 37000- | RDNAKNTLYLQMNSLRAEDTAVYYCAR |
| | 38500 | |
| 185 | Fox_8- | EVQLVESGGDLVKPGGSLRLSCVASGFTFSSYYMHWVRQAPGKGLKWVARISSDGSSTRYADALKGQFTIS |
| | 1645000- | RDNPKNTLYLQMNSLRAEDTALYYCAR |
| | 1646500 | |
| 186 | Fox_8- | EVQLVESGGDLVKPGGSLRLSCVASGFTFSSDNMQWVRQAPGKGLQWVAYISSGGSSTNYADAVKGRFTIS |
| | 836000- | RDNAKNTLYLQMNSLRAEDTAVYYCAR |
| | 837500 | |
| 187 | Fox_8- | EVQLVESGGDLVKPGGSLRLSCVASGFTFSSYYMSWIRQAPGKGLQWVAYINSGGSSTNYADAVKGRFTIS |
| | 1100000- | RDNAKNTLYLQMNSLRAEDTAVYYCAG |
| | 1101500 | |
| 188 | Fox_8- | EVQLVESGGDLVKPGGSLRLSCVASGFTFSDYAMDWVRQAPGKGLQWVAYINSGGSSTSYADTVKGRFTIS |
| | 784000- | RDNAKNTLYLQMNSLKAEDTAVYYCAK |
| | 785500 | |
| 189 | Fox_3- | EVQLVESGGDLVKPGGSLRLSCVASGFTFSSYYMHWVRQAPGKGLQWVAQISNDGSSTGYADAVKGRFTIS |
| | 4000- | RDNSKNTLYLQMNSLRAEDTALYYCAR |
| | 5500 | |
| 190 | Fox_8- | EVQLVESGGDLVKPGGSLRLSCVGSGFTFSSYWMYWVRQAPGKGLQWVAEISGSGSSTNYADAVKGRFTLS |
| | 1381000- | RDNAKNTLYLQMNSLRAEDTAVYYCAR |
| | 1382500 | |
| 191 | Fox_8- | EVQLVESGGDLVKPGGSLRLSCVVSGFTFSSYNMQWVPQAPGKGLQWVAYISTGGSSTSYADAVKGRFTIS |
| | 1149000- | RDNAKNKLYLQMNSLRAEDTAVYYCAR |
| | 1150500 | |
| 192 | Fox_8- | EVQLVESGGDLVKPGGSLKLSCVASGFTFSDYYMSWVCQAPGKGLQWVASIIYDGSSTSYTDAVKGRFTIS |
| | 1504000- | RDNAKNTLYLQMNSLRAEDTAVYYCAS |
| | 1505500 | |
| 193 | Fox_8- | EVQLVESGGDLVKPGGSLRLSCVASGVTFSSYYMDWVFQAPGKRLQWVAGISSDGSSTYYPDAVKGRFTIS |
| | 1634000- | RDNAKNTLYLQMNSLRAEDSAVYYCAM |
| | 1635500 | |
| 194 | Fox_8- | QVQLVESGGDLVKLGGSLRLSCVASGFTFSSCWMHWVRQAPGKGLQWVAEISGSGSSTNYADAVKGQFTIS |
| | 758000- | RDNAKNTLYLQMNSLRAKDTAVYYCVR |
| | 759500 | |
| 195 | Fox_8- | EVQLGESGGDLVKPGRSLRLSCVASGFTFSSYHMSWVHQAPGKGLQWVSRIISDGSSTYYADAVKGQFTIS |
| | 937000- | RDNAKKTLYLQMNSLRTEDTAVYYCGK |
| | 938500 | |
| 196 | Fox_5- | EVQLVESGGDLVKPGGSLRLSCVASGFTFSNYKIYWVRQAPGKGLEWVARIYESGSTTNYAEAVKGRFTIS |
| | 17000- | RDNAKNTLYLQMNSLRAEDTAVYYCAS |
| | 18500 | |
| 197 | Fox_8- | EVQLVETGGDLVKPGGSLRLSCVASGFTFNSYYMDWVCQAPGKGLQWVAYISSDGSSTSYTDAVKGQFTIS |
| | 1620000- | RDNAKNTLYLQMNSLRAEDTAVYYCTS |
| | 1621500 | |
| 198 | Fox_8- | EVQLLESGGNLVKPRGSLRLSCVASGFTFSSYTMQWVPQAPGKGLQWVTYINSGGSSTSSADTVKGPFIIS |
| | 971000- | RDNAKNPLYLQMNSLRAEDTAVYYCAD |
| | 972500 | |
| 199 | Fox_8- | EVQLVESGGDLVKPGGSLGLSCVASGFTFSSYDMHWVRQAPGKGLQWVAWIYASGSSPSYSDAVKGRFTIS |
| | 1462000- | RDNAKYMVHLQMNSLRAEDTAVYYCAS |
| | 1463500 | |
| 200 | Fox_8- | EVQLVESGGDLVKPEGSLRLSCVASGFTFSSSYMNWVRQDPRKGLQWVAAISNDGRSTYYADAVKGRFTIS |
| | 1422000- | RDNAKNTVYLQMNSLRAEDTAMYYCAR |
| | 1423500 | |
| 201 | Fox_5- | EEQLVEFGGHMVNPGGSLGLSCQASGFTFSSYGMSWVRQAQKKGLQWVGHISYDGSSTYYADTVRDRFTIS |
| | 22000- | RDNTKNMLYLQMNSLRAEDTAVYYCMR |
| | 23500 | |
| 202 | Fox_8- | CAAGGVWGRPGDAWGVSLRLSCVASGFTFSRYAMSWVLQAPGKELQWVAYISSDGSSTSYTDAVKGQFAIL |
| | 1487000- | RANAKNTLYLQMNSLRAEDTAVYYCAR |
| | 1488500 | |
| 51 | IGHV3-7 | EVQLVESGGGLVQPGGSLRLSCAASGFTFSSYWMSWVRQAPGKGLEWVANIKQDGSEKYYVDSVKGRFTIS |
| | | RDNAKNSLYLQMNSLRAEDTAVYYCAR |
| 203 | Fox_6-0- | EVQLVESGGDLVKPGGSLRLSCVASGFTFSSYGMSWVRQAPGKGLQWVAYISYDGSSTYYTDAVKGRFTIS |
| | 1500 | RDNAKNTLYLQMNSLRAEDTAVYYCAK |
| 204 | Fox_8- | EVQLVESGGDLVKPGGSLRLSCVVSGFAFSSNEMSWVRQAPGKGLQWVAAISYDGSSTYYVDAVKGRFTTS |
| | 845000- | RDNAKNTLYLQMNSLRAEDTAVYYCAD |
| | 846500 | |
| 205 | Fox_8- | EVQLVESGGDLVKPGGSLRLSCVASGFTFSSYGVSWVRQAPGKGLQWVAAIWYDGTETYYADAVKGRFTIS |
| | 1447000- | SDNAKNTLYLQMNSLRAEDTAVYYCVK |
| | 1448500 | |
| 206 | Fox_8- | EVQLVESGGDLVKPGGSLRLSCVASGFTFSSYGMSWVRQAPGKGLQLVAAIWYDGSRTYYADAVKGRFTIS |
| | 1182000- | RDNAKNTLYLQMNSLKAEDTAMYFCAK |
| | 1183500 | |
| 46 | IGHV3-21 | EVQLVESGGGLVKPGGSLRLSCAASGFTFSSYSMNWVRQAPGKGLEWVSSISSSSSYIYYADSVKGRFTIS |
| | | RDNAKNSLYLQMNSLRAEDTAVYYCAR |
| 207 | Fox_8- | EVQLVESGRDLVKPGGSLRLSCVASEFTFSSYSMSCVCQAPGKGLQWVAEISSSGSSTYYTDAVKGRFTIS |
| | 1189000- | RDNAKNTLYLQMNSLRAEDMAVYYCAD |
| | 1190500 | |
| 208 | Fox_8- | EVQQLESGGDRVKPRGSQRLSCVASRFTFSSYSMHWLHQSPGKGLQWITYISSSGSSTYYADAVKGRFTIS |
| | 1143000- | RDKDKNMLYLQMNSLRAQDIALYYCAD |
| | 1144500 | |
| 209 | Fox_8- | EVQQLESGGDRVKPRGSQRLSCVASRFTFSSYSMHWLHQSPGKGLQWITYISSSESSTYYVDAVKGRFAIS |
| | 1040000- | RDKDKNMLYLQMNSLKAQDIALYYCAD |
| | 1041500 | |
| 38 | IGHV3- | EVQLLESGGGLVQPGGSLRLSCAASGFTFSSYAMSWVRQAPGKGLEWVSAISGSGGSTYYADSVKGRFTIS |
| | 23D | RDNSKNTLYLQMNSLRAEDTAVYYCAK |
| 210 | Fox_8- | EVQLVESGGDLVKPGGSLRLSCVASGFTFSSYAMSWVRQAPGKGLQWVAGISNSGSSTGYADAVKGRFTIS |
| | 1064000- | RDNAKNTLYLQMNSLKAEDTAVYYCAK |
| | 1065500 | |
| 211 | Fox_8- | EVQLVESGGDLVKPGGSLRLSCVASGFTFSSNWMSWVRQAPGKGLQWVAGISNSGSSTYYADAVKGRFTIS |
| | 951000- | RDNAKNTLYLQMNSLRTEDTAMYYCAK |
| | 952500 | |
| 212 | Fox_8- | EVQLVESGGDLVKPGGFLRLSCVASGFTFSSYAMSWVHKAPVKGLQWVTRIYASGSRTSYADAVKGRFTIS |
| | 980000- | RDNVKNTLYLQMNSLRAEDTAVHYCLK |
| | 981500 | |
| 55 | IGHV1-46 | QVQLVQSGAEVKKPGASVKVSCKASGYTFTSYYMHWVRQAPGQGLEWMGIINPSGGSTSYAQKFQGRVTMT |
| | | RDTSTSTVYMELSSLRSEDTAVYYCAR |
| 213 | Fox_8- | EVQLVQSGAEVKKPGASVKVSCKTSGYTFINYYMHWVRQAPGAGLDWMGRIDPEDGATNYAQKFQGRVTLT |
| | 1416000- | ADTSTSTAYMELRSLRAEDTAVYYCAR |
| | 1417500 | |
| 214 | Fox_8- | EVQLVQSGAEVKKPGASVKVSCKTSGYTFINYYMYWVRQAPGAGFDWMGLIDPEDGATRYAQKIQGRVTLT |
| | 1549000- | ADTSTSTAYMELNSLRAEDTAVYYCAR |
| | 1550500 | |
| 215 | IGHV3- | QVQLVESGGGVVQPGRSLRLSCAASGFTFSSYGMHWVRQAPGKGLEWVAVISYDGSNKYYADSVKGRFTIS |
| | 30-5 | RDNSKNTLYLQMNSLRAEDTAVYYCAK |
| 216 | Fox_8- | EVQLVESGGDLVKPGGSLRLSCVASGFTFSSYGMNWVRQAPGKGLQWVAVIWYDGSRTYYNDAVKGRFTIS |
| | 1081000- | RDNAKNTLYLQMNSLRAEDTAVYYCAK |
| | 1082500 | |
| 217 | IGHV3- | EVQLVESGGGLVQPGGSLRLSCAASGFTVSSNYMSWVRQAPGKGLEWVSVIYSGGSTYYADSVKGRFTISR |
| | 66 | DNSKNTLYLQMNSLRAEDTAVYYCAR |
| 218 | Fox_4- | EVQLVESGGDLVKPGGSLRLSCVASGFTFSSYAMSWVRQAPGKGLQWVAAISYGGSTYYTDAVKGRFTISR |
| | 3000- | DNAKNTLYLQMNSLRAEDTAVYYCAK |
| | 4500 | |
| 219 | IGHV3- | QVQLVESGGGLVKPGGSLRLSCAASGFTFSDYYMSWIRQAPGKGLEWVSYISSSGSTIYYADSVKGRFTIS |
| | 11 | RDNAKNSLYLQMNSLRAEDTAVYYCAR |
| 220 | Fox_8- | EVQLVESGGDLVKPGVSLRLSCVASGFTFSDYGMSWVRQAPGKGLQWVGEISSSGSSTYYADAVKGRFTIS |
| | 1301000- | RDNAKNTLYLQMNSLRAEDMAVYYCAT |
| | 1302500 | |
| 1 | IGHV3-74 | EVQLVESGGGLVQPGGSLRLSCAASGFTFSSYWMHWVRQAPGKGLVWVSRINSDGSSTSYADSVKGRFTIS |
| | | RDNAKNTLYLQMNSLRAEDTAVYYCAR |
| 221 | WolfHG_ | EVQLVESGGDLVKPGGSLRLSCVASGFTFSSNYMSWIRQAPGKGLQWVSQISSDGSSTSYADAVKGRFTIS |
| | 994390.1: | RDNAKNTLYLQMNSLRDEDTAVYYCAR |
| | 1000000- | |
| | 3000000- | |
| | 1540000- | |
| | 1541500 | |
| 222 | Wolf_HG | EVQLVESGGDLVKPGGSLRLSCVASGFTFSSYWMYWVRQAPGKGLQWVAGINSGGSSTSYADAVKGRFTIS |
| | 994390.1: | RDNAKNTVYLQMNSLRAEDTAMYYCAK |
| | 1000000- | |
| | 3000000- | |
| | 57000- | |
| | 58500 | |
| 223 | Wolf_HG | EVQLVESGGDLVKPAGSLRLSCVASGFTFSSYSMSWVRQAPGKGLQWVAGINSGGSSTSYADAVKGRFTIS |
| | 994390.1: | RDNAKNTLYLQMNSLRAEDTAVYYCAD |
| | 1000000- | |
| | 3000000- | |
| | 193000- | |
| | 194500 | |
| 224 | Wolf_HG | EVQLVESGGDLVKPGGSLRLSCVASGFTFSSYHMSWVRQAPGKGLQWVAYINSGGSSTYYADAVKGRFTIS |
| | 994390.1: | RDNAKNTLYLQMNSLRAEDTAVYYCAD |
| | 1000000- | |
| | 3000000- | |
| | 594000- | |
| | 595500 | |
| 225 | Wolf_HG | EVQLVESGGDVVKPGGSLRLSCVASGFTFSSYYMYWARQAPGKGLQWVSHINKDGSSTSYADAVKGRFTIS |
| | 994390.1: | RDNAKNTLYLQMNSLRAEDTAVYYCAK |
| | 1000000- | |
| | 3000000- | |
| | 1522000- | |
| | 1523500 | |
| 226 | Wolf_HG | EVQLVESGGDLVKPGGSLRLSCVASGFTFSSSDMSWIRQAPGKGLQWVAYISNDGSSTSYADAVKGRFTIS |
| | 994390.1: | RDNAKNTLYLQMNSLRAEDTAVYYCAD |
| | 1000000- | |
| | 3000000- | |
| | 1429000- | |
| | 1430500 | |
| 227 | Wolf_HG | EVQLVESGGDLVKPGGSLRLSCVTSGFTFSSYWMSCVRQAPGKELQWVAYINSGGSSTWYTDAVKGRFTIS |
| | 994390.1: | RDNAKNTLYLQMNSLRAEDTAVYYCAR |
| | 1000000- | |
| | 3000000- | |
| | 964000- | |
| | 965500 | |
| 228 | Wolf_HG | EVQLVESGGELVKPAGSLRLSCVTSGFTFSSYGMSWVRQAPGKGLQWVAGINSGGSSTSYADAVKGRFTIS |
| | 994390.1: | RDNAKNTVYLQMNSLRAEDTAVYYCAK |
| | 1000000- | |
| | 3000000- | |
| | 718000- | |
| | 719500 | |
| 229 | Wolf_HG | EVLLVESGGDLVKPGGSLRLSCVASGFTFSSYDMGWVRQAPGKGLQWVAYISNGGSSTRYADAVKGRFTIS |
| | 994390.1: | RDNAKNTLYLQMNSLRAEDTAVYYCAS |
| | 1000000- | |
| | 3000000- | |
| | 1463000- | |
| | 1464500 | |
| 230 | Wolf_HG | EVQLVESGGDLMKPGGSLRLSCVASGFTISSNYMNWVRQAPGKGLQWVGYISSDGSSTSYADAVKGRFTIS |
| | 994390.1: | RDNAKNTLYLQMNSLRAEDTAVYYCVK |
| | 1000000- | |
| | 3000000- | |
| | 314000- | |
| | 315500 | |
| 231 | Wolf_HG | EVQLVESGGDLVKPGGSLRLSCVASGFTFSSNDMDWVRQAPGKGLQWLTRISNDGRSTGYADAVKGRFTIS |
| | 994390.1: | RDNAKNTLYLQMNSLRAEDTAVYYCAK |
| | 1000000- | |
| | 3000000- | |
| | 365000- | |
| | 366500 | |
| 232 | Wolf_HG | EVQLVESGGDLVKPEGSLRLSCVVSGFTFSSYDMSWVRQAPGKGLQWVAYISSDGRSTSYTDAVKGRFTIS |
| | 994390.1: | RDNAKNTLYLQMNSLRTEDTAVYYCAK |
| | 1000000- | |
| | 3000000- | |
| | 1165000- | |
| | 1166500 | |
| 233 | Wolf_HG | EVQLVESGGALVKSGGSLRLSCVGSGFTFSSYWMYWDRQAPGKGLQWVAWIRYDGSSTSYAEAVKGRFTVS |
| | 994390.1: | RDNAKNTLYLQMNSLRAEDTAVYYCVR |
| | 1000000- | |
| | 3000000- | |
| | 1040000- | |
| | 1041500 | |
| 234 | Wolf_HG | EEQLVESGEDPVKPGGSLRLSCVASGFTFSSYAVSWVRQAPGKGLQWVRYISSDGSSTYYADAVKGRFTIS |
| | 994390.1: | RDNAKNTLYLPMNSLRAEDTAVYYCAR |
| | 1000000- | |
| | 3000000- | |
| | 545000- | |
| | 546500 | |
| 235 | Wolf_HG | EEQLMESEGDLVKPGGSLRLSCVASGFTFSSYDMSWVRQAPGKGLQWVGYINSGGSSTYYADAVKGRFTIS |
| | 994390.1: | RDNAKNTLYLQMNSLRAEDTAVYYCVK |
| | 1000000- | |
| | 3000000- | |
| | 1121000- | |
| | 1122500 | |
| 236 | Wolf_HG | EEQLMESEGDLVKPGGSLRLFCVASGFTFSSYDMSWVRQAPGKGLQWVGYINSGGSSTYYADAVKGRFTIS |
| | 994390.1: | RDNAKNTLYLQMNSLRAEDTAVYYCVK |
| | 1000000- | |
| | 3000000- | |
| | 536000- | |
| | 537500 | |
| 237 | Wolf_HG | EVQLVESGGDLVKPGGSLRLSCVASGFTFSSYYMFWIRQAPGKGNQWVGYINKDGSSTYYPDAVKGRFTIS |
| | 994390.1: | RDNAKNTLYLQMNSLTVEDTALYYCAR |
| | 1000000- | |
| | 3000000- | |
| | 1228000- | |
| | 1229500 | |
| 238 | Wolf_HG | EEQLVESGEDPVKPGGSLRLSCVASGFTFSSYAVSWVRQAPGKGLQWVRYISSDGSSTYYADAVKGRFTIS |
| | 994390.1: | RDNAKNTLYLPMNSLRAEDTAVYYCAR |
| | 1000000- | |
| | 3000000- | |
| | 1143000- | |
| | 1144500 | |
| 239 | Wolf_HG | EMQLVEAWGDMVEPGGSLRISCVASGFTFSSYWMSWVCQAPGKGLQWVAYINSGGSSTYYADAVKGRFTIS |
| | 994390.1: | RDNAKNTLYLQMNSLRAEDMAVYYCVR |
| | 1000000- | |
| | 3000000- | |
| | 610000- | |
| | 611500 | |
| 240 | Wolf_HG | EVQLVESGGDLAKPGGSLRLSCVASGFTFSSYEMNWVCQAPGKGLQWVAYINCGESSTYYADAVKGRFTIS |
| | 994390.1: | RDNARNTLYLQMNSLRTEDTAMYYCAK |
| | 1000000- | |
| | 3000000- | |
| | 910000- | |
| | 911500 | |
| 241 | Wolf_HG | EVQLVESGGDLAKPGGSLRLSCVASGLTFSSYSMSWVRQAPGKGLQWVTAISYDGSSTYYTDAVKGRFTIS |
| | 994390.1: | RDNARNTVYLQMNSLRAEDTAVYYCVD |
| | 1000000- | |
| | 3000000- | |
| | 443000- | |
| | 444500 | |
| 242 | Wolf_HG | EVQLVESGGDLVKPGLPLRLSCVASGFTFSSYWISWVHQAPGKGLQWVSVINKYGSTTYHADAVKGQFTIF |
| | 994390.1: | RDNAKNTLYLQMNSLRAEDTAVYYCAH |
| | 1000000- | |
| | 3000000- | |
| | 210000- | |
| | 211500 | |
| 243 | Wolf_HG | EVQLVESGGDLVKPGGSLRLSCVASGFTFNSYDMSWVHQVRGKRLQWVAYFSSDGNSTSSADAVKGRFTIS |
| | 994390.1: | RDNAKNTLYLQMNSLRAEDTAVYYCAK |
| | 1000000- | |
| | 3000000- | |
| | 989000- | |
| | 990500 | |
| 244 | Wolf_HG | EVQLVESGGDLAKPGGSRRLSCVASGFTFSSYEINWVCQAPGKGLQWVAYINCGGSSTYYADAVKGWFTIS |
| | 994390.1: | RDNARNTLYLQMNSLRAEDTVVYYCAK |
| | 1000000- | |
| | 3000000- | |
| | 575000- | |
| | 576500 | |
| 245 | Wolf_HG | EVQLVETEGDLVKPGGSLRLSCVASGFTFSSYDMDWVCQAPGKGLQWVTYISNGGSSTRYADAVKGPFTIS |
| | 994390.1: | RDNARNTLYLQMNSLRAEDTAVYYCVS |
| | 1000000- | |
| | 3000000- | |
| | 1347000- | |
| | 1348500 | |
| 246 | Wolf_HG | EVQLVESGGDLVKPGLPLRLSCVASGFTFSSYWISWVHQTPGKGLQWVSVMNKYGSTTYHADAVKGQFTIF |
| | 994390.1: | RDNAKNTLYLQMNSLRSEDMAVYYCAH |
| | 1000000- | |
| | 3000000- | |
| | 513000- | |
| | 514500 | |
| 247 | Wolf_HG | EVQLVESGGDLVKPGLPLRLSCVASGFTFSSYWISWVHQAPGKGLQWVSVMNKYGSTTYHADAVKGQFTIF |
| | 994390.1: | RDNAKNTLYLQMNSLRAEDMDVYYCVH |
| | 1000000- | |
| | 3000000- | |
| | 745000- | |
| | 746500 | |
| 248 | Wolf_HG | EEQLVEFGGHMVNPGGSLGLSCQASGFTFSSYGMSWVRQAQKKGLQWVGHISYDGSSTYYADTLRDRFTIS |
| | 994390.1: | RDNTKNMLYLQMNSLRAEDTAVYYCMR |
| | 1000000- | |
| | 3000000- | |
| | 1413000- | |
| | 1414500 | |
| 46 | IGHV3-21 | EVQLVESGGGLVKPGGSLRLSCAASGFTFSSYSMNWVRQAPGKGLEWVSSISSSSSYIYYADSVKGRFTIS |
| | | RDNAKNSLYLQMNSLRAEDTAVYYCAR |
| 249 | Wolf_HG | EVQLVESGGDLVKPGGSLRLSCVASGFTFSSYDMDWVRQAPGKGLQWLSEISSSGSSTYYADAVKGRFTIS |
| | 994390.1: | RDNAKNTLYLQMNSLRAEDTAVYYCAR |
| | 1000000- | |
| | 3000000- | |
| | 1357000- | |
| | 1358500 | |
| 250 | Wolf_HG | EVQLVESGGDLVKPGGSLRLSCVASGFTFSSYDMNWVRQAPGKGLQWVAYISSGGSSTYYADAVKGRFTIS |
| | 994390.1: | RDNAKNTLYLQMNSLRAEDTAMYYCAG |
| | 1000000- | |
| | 3000000- | |
| | 985000- | |
| | 986500 | |
| 251 | Wolf_HG | EVQLVESGGDLVKPGRSLRLSCVASGFTFSSNEMNWVRQAPGKGLQWVAAISSNGSSTYYADAVKGRFTIS |
| | 994390.1: | RDNAKNTLYLQMNSLRAEDTAVYYCAD |
| | 1000000- | |
| | 3000000- | |
| | 467000- | |
| | 468500 | |
| 252 | Wolf_HG | EVQLVESGGDLVKPGGSLRLSCVASGFTFSNYEMYWVRQAPGKGLEWVARIYESGSTTYYAEAVKGRFTIS |
| | 994390.1: | RDNAKNTLYLQMNSLRAEDTAVYYCAS |
| | 1000000- | |
| | 3000000- | |
| | 1407000- | |
| | 1408500 | |
| 253 | Wolf_HG | EVQLVESGGDLVKPGGSLRLSCVTSGFTFSSNEMNWVRQDPGKGLQWVAAISSSGSSTYYADAVKGRFTIS |
| | 994390.1: | RDNAKNTLYLQMNSLRAEDMAVYYCAD |
| | 1000000- | |
| | 3000000- | |
| | 681000- | |
| | 682500 | |
| 254 | Wolf_HG | EVQLLESGGDRVKPGGSQRLSCVASRFTFSSYSMHWLRQSPGMGLQWVTYISSSGSSTYFADAVKGRFTIS |
| | 994390.1: | RDKAKNMLYLQMNSLRAQDIALYYCAD |
| | 1000000- | |
| | 3000000- | |
| | 958000- | |
| | 959500 | |
| 38 | IGHV3-23D | EVQLLESGGGLVQPGGSLRLSCAASGFTFSSYAMSWVRQAPGKGLEWVSAISGSGGSTYYADSVKGRFTIS |
| | | RDNSKNTLYLQMNSLRAEDTAVYYCAK |
| 255 | Wolf_HG | EVQLVESGGDLVKPGGSLRLSCVASGFTFSSYGMSWVRQSPGKGLQWVAAISSSGSSTYYADAVKGRFTIS |
| | 994390.1: | RDNAKNTLYLQMNSLRAEDTAVYYCAK |
| | 1000000- | |
| | 3000000- | |
| | 929000- | |
| | 930500 | |
| 256 | Wolf_HG | EVQLVESGGDLVKPGGSLRLSCVASGFTFSSYYMSWIRQAPGKGLKWVADISDSGGSTYYTDAVKGRFTIS |
| | 994390.1: | RDNAKNTLYLQMNSLRAEDTAVYYCAK |
| | 1000000- | |
| | 3000000- | |
| | 1014000- | |
| | 1015500 | |
| 257 | Wolf_HG | KVQLVKSGGDLVKPAGSLRLSCVASGFTFSSYYMSWIRQAPEKGLQWVADINDSGGSTGYADAVKGRFTIS |
| | 994390.1: | RDNAKNTLYLQMNSLRAEDTAVYYCAK |
| | 1000000- | |
| | 3000000- | |
| | 629000- | |
| | 630500 | |
| 258 | Wolf_HG | EVQLVESGGDLVKPVASLRLSCVASGFTFSNYAMSWVRQAPGKGLQWVAEISHSGRSTGYTDAVKGRFTIT |
| | 994390.1: | RDNAKNMLYLKMNSLTAKDMAMYYCAR |
| | 1000000- | |
| | 3000000- | |
| | 1203000- | |
| | 1204500 | |
| 51 | IGHV3-7 | EVQLVESGGGLVQPGGSLRLSCAASGFTFSSYWMSWVRQAPGKGLEWVANIKQDGSEKYYVDSVKGRFTIS |
| | | RDNAKNSLYLQMNSLRAEDTAVYYCAR |
| 259 | Wolf_HG | EVQLVESGGALVKPGASLRLSCVASGFTFSSYDMSWVRQAPGKGLQWVAFISYDGSSTYYADAVRGRFTIS |
| | 994390.1: | RDNAKNSLYLQMNSLRAEDTAVYYCGK |
| | 1000000- | |
| | 3000000- | |
| | 1449000- | |
| | 1450500 | |
| 260 | Wolf_HG | EEQLVEFGGHMVNPGGSLGLSCQASGFTFSSYGMSWVRQAQKKGLQWVGHISYDGSSTYYTDTVRDRFTIS |
| | 994390.1: | RDNTKNMLYLQMNSLRAEDTAVYYCMR |
| | 1000000- | |
| | 3000000- | |
| | 1277000- | |
| | 1278500 | |
| 217 | IGHV3-66 | EVQLVESGGGLVQPGGSLRLSCAASGFTVSSNYMSWVRQAPGKGLEWVSVIYSGGSTYYADSVKGRFTIS |
| | | RDNSKNTLYLQMNSLRAEDTAVYYCAR |
| 261 | Wolf_HG | EVQLVESGGDLVKPGGSLRLSCVASGFTFSSYGMSWVRQSPGKGLQWVADISSSGSTYYADAVKGRFTIS |
| | 994390.1: | RDNAKNTLYLQMNSLRAEDTAVYYCAK |
| | 1000000- | |
| | 3000000- | |
| | 645000- | |
| | 646500 | |
| 262 | Wolf_HG | EVQLVESGGDLVKPAGSLRLSCVASGFTFSSYGMSWVRQAPGKGLQWVAGINSGGNTGYADAVKGRFTIS |
| | 994390.1: | RDNAKNTVYLQMNSLRAEDTAVYYCAK |
| | 1000000- | |
| | 3000000- | |
| | 728000- | |
| | 729500 | |
| 263 | IGHV1-69- | EVQLVQSGAEVKKPGATVKISCKVSGYTFTDYYMHWVQQAPGKGLEWMGLVDPEDGETIYAEKFQGRVTI |
| | 2 | TADTSTDTAYMELSSLRSEDTAVYYCAT |
| 264 | Wolf_HG | EVQLVQSAAEVKKPGASVKVSCKTSGYTFTDYYMHWVQQAPGAGLDWMGWIDPEDGATSYAQKFQGRVTL |
| | 994390.1: | TADTSTSTAYMELSSLRAEDTAVYYCAR |
| | 1000000- | |
| | 3000000- | |
| | 1022000- | |
| | 1023500 | |
| 265 | IGHV3-9 | EVQLVESGGDLVKPGGSLRLSCVASGFTFSSYDMDWVRQAPGKGLQWLSEISSSGSSTYYADAVKGRFTI |
| | | SRDNAKNTLYLQMNSLRAEDTAVYYCAR |
| 266 | QKY | EVQLVESGGGLVKPGGSLRLSCAASGFTFSSYSMNWVRQAPGKGLEWVSCISSSSSFIYYADSVKGRFTI |
| | 76336.1 | SRDNAKNSLYLQMNSLRAEDTAVYYCARDPVWVDGELLSGGIPFDYWGQGTLVTVSS |
| 267 | QKY | EVQLVESGGGLVKPGGSLRLSCAASGFTFSGYSMNWVRQAPGKGLEWVSSISSSSSYIYYADSVKGRFTI |
| | 76475.1 | SRDNAKNSLYLQMNSLRAEDTAVYYCARWLQLRSDYYYFGMDVWGQGTTVTVSS |
| 268 | QKY | EVQLVESGGGLVQPGGSLRLSCAASGFTFSSYEMNWVRQAPGKGLEWVSYISSSGSAIYYADSVKGRFTI |
| | 76492.1 | SRDNAKNSLYLQMNSLRVEDTAVYYCAREARSRYFDWLPSYYFDYWGQGTLVTVSS |
| 269 | QKY | EVQLVESGGGLVKPGGSLRLSCAASGFTFSSYSMNWVRQAPGKGLEWVSSISNSNSFIYYADSMKGRFTI |
| | 76474.1 | SRDNAKNSLYLQMNSLRAEDTAVYYCARVNGNSNWNFGSYYYYYMDVWGKGTTVTVSS |
| 270 | QKY | EVQLVESGGGLVQPGGSLRLSCEASGFTFSSSEINWVRQAPGKGLEWVSHISSSGSIIYYADSVKGRFTI |
| | 76481.1 | SRDNAKNSLYLQMNSLRAEDTAVYYCARRSYRSSWYYYYGMDVWGQGTTVTVSS |
| 271 | IGHV3-5 | EVQLVESGGDLVKPGGSLRLSCVASGFTFSSYHMSWVRQAPGKGLQWVAYINSGGSSTSYADAVKGRFTI |
| | | SRDNAKNTLYLQMNSLRAEDTAVYYCAS |
| 272 | QKY | EVQLVESGGGLVQPGGSLRLSCAVSGFTFSSYWMHWVRQAPGKGLVWVSRINSDGSSTSYADSVKGRFTI |
| | 76515.1 | SRDNAKNTLYLQMNSLRAEDTAVYYCAREVEQLAHMVDYWGQGTLVTVSS |
| 273 | QKY | EVQLVESGGGLVQPGGSLRLSCAASGFTFSSYWMHWVRQVPGKGLVWVSRINSDGSSTSYADSVKGRFTI |
| | 76404.1 | SRDNAKNTLYLEMNSLRAQDTAVYYCAGSPWLRGDIDYWGQGTLVTVSS |
| 274 | QKY | EVQLVESGGGLVQPGGSLRLSCAASGFTFSSYWMSWVRQAPGKGLEWVANIKEDGSEKYYVDSVKGRFTI |
| | 76616.1 | SRDNAKNSLYLQMNSLRAEDTAVYYCARDVGGYSGYDLGFDYYYYMDVWGKGTTVTVSS |
| 275 | QKY | EVQLVESGGGLVQPGGSLRLSCVASEFTFSSYWMSWFRQAPGKGLEWVSGINWNGGSTGYADSVKGRFTI |
| | 76368.1 | SRDNAKNSLYLQMNSLKTEDTAVYYCASVITFGGVIVRSYWGQGTLVTVSS |
| 276 | QKY7 | EVQLVESGGGLVQPGGSLRLSCAASGFTFSIYWMSWVRQAPGKGLQWVANIKQDASEKYYVDSVKGRFTI |
| | 6619.1 | SRDNAKNSLYLQMNSLRAEDTAVYYCARLGGSSWHFDYWGQGTLVTVSS |
| 277 | IGHV1-30 | EVQLVQSGAEVKKPGASVKVSCKTSGYTFINYYMIWVRQAPGAGLDWMGQIDPEDGATSYAQKFQGRVTL |
| | | TADTSTSTAYMELSSLRAGDIAVYYCAR |
| 278 | QKY | QVQLVQSGAEVKKPGASVKVSCKASGYTFTSYYMHWVRQAPGQGLEWMGIINPSAGSTSYAQKFQGRVTM |
| | 76391.1 | TRDTSTSTVYMELSSLRSEDTAVYYCARGTLIPAHRGAFDIWGQGTMVTVSS |
| 279 | QKY | QVQLVQSGAEVKKPGASVKVSCKASGYTFTSHYMHWVRQAPGQGLEWMGIINPSGGSTSYAQKFQGRVTM |
| | 76514.1 | TGDTSTSTVYMELSSLRSEDTAVYYCARDLAGVPAALGCWFDPWGQGTLVTVSS |
| 280 | QKY | QVQLVQAGAEVKKPGASVKVSCKASGYTFTSYYMHWVRQAPGQGLEWMGIINPSGGSTSYAQKFQGRVTM |
| | 76380.1 | TRDTSTSTVYMELSSLRSEDTAVYYCARGAAVPAAGEFDYWGQGTLVTVSS |
| 281 | QKY | QVQLVQSGAEVKKPGASVKVSCKASGYTFTSYYMHWVRQAPGQGLEWMGIINPGGGSTTYAQKFQGRVTM |
| | 76347.1 | TSDTSTSTVYMELSSLRSEDTAMYYCARGAIPPNSRAEIDYWGQGTLVTVSS |
| 282 | IGHV3-67 | EVQLVESGGDLVKPGGSLRLSCVASGFTFSSYYMYWVRQAPGKGLQWVARISSDGSSTYYADAVKGRFTI |
| | | SRDNAKNTLYLQMNSLRAEDTAMYYCAK |
| 283 | QKY | QVQLVESGGGVVQPGRSLRLSCAASGFTFSTYGMHWVRQAPGKGLEWVAVISYDGSNKYYADSVKGRFTI |
| | 76668.1 | SRDNSKNTLYLQMNSLRAEDTAMYYCAKDGSIAAADYWGQGTLVTVSS |
| 284 | QKY | QVQLVESGGGVVQPGRSLRLSCAASGFTFSSYGMHWVRQAPGKGLEWVAVISYDGSNKYYADSVKGRFTI |
| | 76590.1 | SRDNSKNTLYLQMNSLRAEDTAVYYCAKDLTIVVIPAAPNFDYWGQGTLVTVSS |
| 285 | QKY | QVQLVESGGGVVQPGRSLRLSCVASGFTFSSYGMHWVRQAPGKGLEWVAVISFDGSNKYYADSVKGRFTI |
| | 76437.1 | SRDNSKNTLYLQMNSLRAEDTAVFYCAKDLPPYASGWYEGGFDYWGRGTQVTVSS |
| 286 | QKY | QVQLVESGGGVVQPGRSLRLSCAASGFTFSSYGMHWVRQAPGKGLEWVAVISSDGSNKYYAGSVKGRFTI |
| | 76588.1 | SRDNSKNTLYLQMNSLRAEDTAVYYCAKDMVEPLFSHYYYYGMDVWGQGTTVTVSS |
| 287 | IGHV3-38 | EVQLVESGGDLVKPGGTLRLSCVASGFTFSSYDMSWVRQSPGKGLQWVAVIWNDGSSTYYADAVKGRFTI |
| | | SRDNAKNTLYLQMNSLRAEDTAVYYCAK |
| 288 | QKY | QVQLVESGGGVVQPGRSLRLSCAASGFTFSSYGMHWVRQAPGKGLEWVAVIWYDGSNKYYAESVKGRFTI |
| | 76393.1 | SRDNSKNTLYLQMNSLRAEDTAVYYCARDLHQDWVVVVAANVYGMDVWGQGTTVTVSS |
| 289 | QKY | QVQLVESGGGVVQPGRSLRLSCAASGFTFSSYGMHWVRQAPGKGLEWVAVIWFDGSNKHYADSVKGRFTI |
| | 76534.1 | SRDNSKNTLYLQMNSLRAEDTAVYYCAREGDFWSGYYTGWFDPWGQGTLVTVSS |
| 290 | QKY | QVQLVESGGGVVQPGRSLRLSCAASGFTFSSYGMHWVRQAPGKGLEWVAVIWYDGSKKDYADSVKGRFTI |
| | 76663.1 | SRDNSKNTLYLQMNSLRAEDTAVYYCARDQSQGAYILTGYRGYGMDVWGQGTTVTVSS |
| 291 | QKY | QVQLVESGGGVVQPGRSLRLSCAASGFTFSSYGMHWVRQAPDKGLEWVAVIWYDGSNKYYADSVKGRFTI |
| | 76548.1 | SRDNSKNTLYLQMNSLRAEDTAVYYCAREGQWPNQAFDIWGQGTMVTVSS |
| 282 | IGHV3-67 | EVQLVESGGDLVKPGGSLRLSCVASGFTFSSYYMYWVRQAPGKGLQWVARISSDGSSTYYADAVKGRFTI |
| | | SRDNAKNTLYLQMNSLRAEDTAMYYCAK |
| 292 | QU | QVQLVESGGGVVQPGRSLRLSCAASGFTFSSYGMHWVRQAPGKGLEWVAVISYDGSNKYYADSVKGRFTI |
| | 596105.1 | SRDNSKNTLYLQMNSLRAEDTAVYYCAR |
| 293 | QU | EVQLVESGGGLVQPGRSLRLSCAASGFTFDDYAMHWVRQAPGKGLEWVSGISWNSGSIGYADSVKGRFTI |
| | 596108.1 | SRDNAKNSLYLQMNSLRAEDTALYHCAK |
| 277 | IGHV1-30 | EVQLVQSGAEVKKPGASVKVSCKTSGYTFINYYMIWVRQAPGAGLDWMGQIDPEDGATSYAQKFQGRVTL |
| | | TADTSTSTAYMELSSLRAGDIAVYYCAR |
| 294 | QU | QVQLVQSGAEVKKPGSSVKVSCKASGGTFSSYAISWVRQAPGQGLEWMGRIIPIFGIANYAQKFQGRVTI |
| | 596106.1 | TADKSTSTAYMELSSLRSEDTAVYYCAR |
| 295 | IGHV3-38 | EVQLVESGGDLVKPGGTLRLSCVASGFTFSSYDMSWVRQSPGKGLQWVAVIWNDGSSTYYADAVKGRFTI |
| | | SRDNAKNTLYLQMNSLRAEDTAVYYCAK |
| 296 | QU | QVQLVESGGGVVQPGRSLRLSCAASGFTFSSYGMHWVRQAPGKGLEWVAVIWYDGSNKYYADSVKGRFTI |
| | 596104.1 | SRDNSKNTLYLQMNSLRAEDTAVYYCAR |
| 265 | IGHV3-9 | EVQLVESGGDLVKPGGSLRLSCVASGFTFSSYDMDWVRQAPGKGLQWLSEISSSGSSTYYADAVKGRFTI |
| | | SRDNAKNTLYLQMNSLRAEDTAVYYCAR |
| 297 | QU | EVQLVESGGGLVKPGGSLRLSCAASGFTFSSYSMNWVRQAPGKGLEWVSSISSSGSTIYYADSVKGRFTI |
| | 596107.1 | SRDNAKNSLYLQMNSLRAEDTAVYYCAR |
| 298 | Heavy | EVQLVQSGAEVKKPGESLRISCKGSGYSFTSYWISWVRQMPGKGLEWMGRIDPSDSYTNYSPSFQGHVTI |
| | V Gene | SADKSISTAYLQWSSLKASDTAMYYCAR |
| | Human | |
| 299 | Heavy | EVQLVESGGDLVKPVGSLRLSCVASGFTFSSYDMNWVRQAPGKGLQWVAYISSGGSSTYYADAVKGRFTI |
| | V Gene | SRDNAKNTLYLQMNSLRAEDTAMYYCAG |
| | Dog C1 | |
| 300 | Lambda | QSVLTQPPSVSVSPGQSVTITCTGSSSDVGGSNYVSWYQQKPGQAPKLLIYEKSDGIDSNRPSGVPDRFS |
| | V Gene | GSKSGNTASLTISGLQAEDEADYYC |
| | Human | |
| 301 | Lambda | QSVLTQPPSVSGSLGQRVTISCTGSSSNVIGSNYVGWYQQLPGTAPRTLIYGYSDSTSNRPSGVPDRFSG |
| | V Gene | SKSGNTATLTISGLQAEDEADYYC |
| | Dog C2 | |
| 302 | Kappa | DIVMTQSPSSLSASPGDRVTISCRASQSLLHSDGISSYLAWYQQKPGKAPKLLIYAASSLQSGVPSRFSG |
| | V Gene | SGSGTDFTLTISSLQPEDFATYYC |
| | Human | |
| 303 | Kappa | DIVMTQTPLSLSVSPGETASISCKASQSLLHSFNGNTYLNWFRQKPGQSPQRLIYKVSNRDPGVPDRFSG |
| | V Gene | SGSGTDFTLRISRVEADDAGVYYC |
| | Dog C3 | |
| 304 | VH clone | EVQLVESGGDLVKPAGSLRLSCVASGFPFSDSDMNWVRQAPGKGLQWVAGIDEGATDTGYADAVKGRFTI |
| | G1 CD96 | SRDDAKNTLYLQMNSLRADDTAMYYCAAGGNFAYWGQGTLVTVSS |
| 305 | Vkappa | DIVMTQTPLSLSVSPGEPASISCKASQSLLHSNGNTYLYWFRQKPGQSPQGLIYLVSKRFSWVPDRFSGS |
| | clone | GSGTDFTLRISRVEADDAGLYYCGQGIQYPYTFSQGTKLEIK |
| | G1 CD96 | |
| 306 | VH clone | GVQLVESGGDLVKPAGSLRLSCVASGFTFSNYGMSWVRQAPGKGLQWVASIASGGSATAYADAVKGRFTL |
| | C2 TIGIT | SRDDAKNTLYLQMNNLRTEDTSMYYCVAPGFQYWGQGTLVTVSS |
| 307 | Vkappa | DIVMTQTPLSLSVSPGETASISC KASQSLLHSNGNTYLFWFRQKSGQSPQRLIY KVSNRDP GVPDRL |
| | Clone | SGSGSGTDFTLRISRVEADDAGVYYCGQGIQIPRTFGQGTKLEIK |
| | C2 TIGIT | |
| 308 | IGKV2-6 | DIVMTQTPLSLSVSPGETASISCKASQSLLHSDGNTYLNWFRQKPGQSPQRLIYKVSNRDPGVPDRFSGS |
| | | GSGTDFTLRISRVEADDTGVYYC |
| 309 | IGKV2-1 | DIVMTQTPLSLSVSPGETASISCKASQSLLHSDGNTYLNWFRQKPGQSPQRLIYKVSNRDPGVPDRFSGS |
| | | GSGTDFTLRISRVEADDTGVYYC |
| 310 | IGHV3-3 | EVQLVESGGGLVKPGGSLRLSCAASGFTFSSYSMNWVRQAPGKGLEWVSSISSSSSYIYYADSVKGRFTI |
| | | SRDNAKNSLYLQMNSLRAEDTAVYYCARWGQGTLVTVSS |
| 311 | Mouse_ | EVQLVESGGGLVKPGGSLKLSCAASGFTFSDYGMHWVRQAPEKGLEWVAYISSGSSTIYYADTVKGRFTI |
| | IGHV5- | SRDNAKNTLFLQMTSLRSEDTAMYYCARWGQGTSVTVSS |
| | 17/ | |
| | IGHJ3*02 | |
| 312 | Rabbit_ | CQQLKESGGGLVKPGGSLKLCCKASGFTFSSYYMCWVRQAPGKGLEWIGCIYAGSGSTHYASWVNGRFTL |
| | IGHV1S7 | SRDNAQSTVCLQLNSLTAADTATYFCARWGQGTLVTVSS |
| | *01/ | |
| | IGHJ3*02 | |
| 313 | Rat_ | EVQLQESGPGLVKPSQSLSLTCSVTGYSITSSYRWN WIRKFPGNKLEWMGYINSAGSTNYNPSLKSRIS |
| | IGHV3-3/ | ITRDTSKNQFFLQVNSVTTEDTATYYCARWGQGTLVTVSS |
| | IGHJ3*01 | |
| 314 | Chicken_ | AVTLDESGGGLQTPGGGLSLVCKASGFDFSSNAMGWVRQAPGKGLEWVAGIDDDGSFTLYAPAVKGRATI |
| | IGHV1- | SRDNGQSTVRLQLNNLRAEDTATYYCARWGHGTEVIVSS |
| | 38/ | |
| | IGHJ*01 | |
| 315 | Human_ | DIVMTQTPLSLSVTPGQPASISCKSSQSLLHSDGKTYLYWYLQKPGQPPQLLIYEVSNRFSGVPDRFSGS |
| | IGKV2D- | GSGTDFTLKISRVEAEDVGVYYCFGQGTKLEIK |
| | 29/ | |
| | IGKJ2*02 | |
| 316 | Mouse_ | DVVMTQTPLSLPVSLGDQASISCRSSQSLVHSNGNTYLH WYLQKPGQSPKLLIYKVSNRFSGVPDRFSG |
| | IGKV1- | SGSGTDFTLKISRVEAEDLGVYFCFGSGTKLEIK |
| | 110/ | |
| | IGKJ3*02 | |
| 317 | Rabbit_ | DVVMTQTPSPVSAAVGSTVTISCQASQSVYNNNNLAWFQQKPGQPPKLLIYAASNLASGVPSRFKGSGSG |
| | IGKV1S54 | TQFTLTISGVQCDDAATYYCFGKGTKLEIK |
| | *01/ | |
| | IGKJ2-3 | |
| 318 | Rat_ | DVVMTQTPVSLSVSLGGQTSISCRSSQSLVHSDGNTYLYWYLQKPGQSPQLLIYKVSNRFSGVPDRFSGS |
| | IGKV1S30 | GSGTDFTLKISRVEPEDLGVYYCFGSGTKLEIK |
| | *01/ | |
| | IGKJ4*01 | |
| 319 | Mouse_ | EVQLVESGGDLVKPGGSLKLSCAASGFTFSSYGMSWVRQATPGKGLEWIGCIYAGSGSTHYASWVNGRFT |
| | IGHV5-6/ | ISRDNAQSTVCLQLNSLTAADTATYFCARWGQGTLVTVSS |
| | IGHJ4*02 | |
| 320 | Rat_ | QVTLKESGPGILQPSQTLSLTCTFSGFSLSTYGMGVGWIRQPSGKGLEWLANIWWDDDKYYNPSLKNRLT |
| | IGHV8-20/ | ISKDTSNNQAFLKITNVDTADTATYYCARWGQGTLVTVSS |
| | IGHJ3*01 | |
| 321 | Chicken_ | AVTLDESGGGLQTPRGALSLVCKASGFTFSSYGMGWVRQAPGKGLEWVAGIGSSGSGTAYGSAVKGRATI |
| | IGHV1-1/ | SRDNGQSTVRLQLNNLRAEDTGTYYCAKWGHGTEVIVSS |
| | IGHJ*01 | |
| 322 | Rat_ | DVVMTQTPVSLSVSLGGQVSISCRSSQSFVHSDGNTYLNWYLQKPGQSPQLLIYKVSNRLSGVPDRFSGS |
| | IGKV1S27 | GSGTDFTLKISRVEHDDLGVYYCFGSGTKLEIK |
| | *01/ | |
| | IGKJ4*01 | |
| 323 | VHG1 | EVQLVESGGGLVQPGGSLRLSCAASGFTFSDSDMNWVRQAPGKGLEWVAGIDEGATDTGYADAVKGRFTI |
| | hum A | SRDNAKNSLYLQMNSLRAEDTAVYYCAAGGNFAYWGQGTLVTVSS |
| 324 | VHG1 | EVQLVESGGGLVQPGGSLRLSCAASGFTFSDSDMNWVRQAPGKGLEWVSGIDEGATDTGYADAVKGRFTI |
| | hum B | SRDNAKNSLYLQMNSLRAEDTAVYYCAAGGNFAYWGQGTLVTVSS |
| 325 | VKG1 | DIVMTQTPLSLSVTPGQPASISCKASQSLLHSNGNTYLYWYLQKPGQPPQLLIYLVSKRFSGVPDRFSGS |
| | hum 1 | GSGTDFTLKISRVEAEDVGVYYCGQGIQYPYTFGQGTKLEIK |
| 326 | VKG1 | DIVMTQTPLSLSVTPGQPASISCKASQSLLHSNGNTYLYWYLQKPGQPPQLLIYLVSKRFSWVPDRFSGS |
| | hum 2 | GSGTDFTLKISRVEAEDVGVYYCGQGIQYPYTFGQGTKLEIK |
| 327 | VHC2 | EVQLVESGGGLVQPGGSLRLSCAASGFTFSNYGMSWVRQAPGKGLEWVASIASGGSATAYADAVKGRFTI |
| | hum C | SRDNAKNSLYLQMNSLRAEDTAVYYCVAPGFQYWGQGTLVTVSS |
| 328 | VH C2 | EVQLVESGGGLVKPGGSLRLSCAASGFTFSNYGMSWVRQAPGKGLEWVASIASGGSATAYADAVKGRFTI |
| | hum D | SRDNAKNSLYLQMNSLRAEDTAVYYCVAPGFQYWGQGTLVTVSS |
| 329 | VKC2 | DIVMTQTPLSLSVTPGQPASISCKASQSLLHSNGNTYLFWYLQKPGQPPQLLIYKVSNRDPGVPDRFSGS |
| | hum 3 | GSGTDFTLKISRVEAEDVGVYYCGQGIQIPRTFGQGTKLEIK |
| 330 | IGHV3-48 | EVQLVESGGGLVQPGGSLRLSCAASGFTFSSYSMNWVRQAPGKGLEWVSYISSSSSTIYYADSVKGRFTI |
| | | SRDNAKNSLYLQMNSLRAEDTAVYYCARWGQGTLVTVSS |
| 331 | TIGIT | MRWCLLLIWAQGLRQAPLASGMMTGTIETTGNISAEKGGSIILQCHLSSTTAQVTQVNWEQQDQLLAICN |
| | | ADLGWHISPSFKDRVAPGPGLGLTLQSLTVNDTGEYFCIYHTYPDGTYTGRIFLEVLESSVAEHGARFQI |
| | | PLLGAMAATLVVICTAVIVVVALTRKKKALRIHSVEGDLRRKSAGQEEWSPSAPSPPGSCVQAEAAPAGL |
| | | CGEQRGEDCAELHDYFNVLSYRSLGNCSFFTETG |
| 332 | CD96 | MEKKWKYCAVYYIIQIHFVKGVWEKTVNTEENVYATLGSDVNLTCQTQTVGFFVQMQWSKVTNKIDLIAV |
| | | YHPQYGFYCAYGRPCESLVTFTETPENGSKWTLHLRNMSCSVSGRYECMLVLYPEGIQTKIYNLLIQTHV |
| | | TADEWNSNHTIEIEINQTLEIPCFQNSSSKISSEFTYAWSVENSSTDSWVLLSKGIKEDNGTQETLISQN |
| | | HLISNSTLLKDRVKLGTDYRLHLSPVQIFDDGRKFSCHIRVGPNKILRSSTTVKVFAKPEIPVIVENNST |
| | | DVLVERRFTCLLKNVFPKANITWFIDGSFLHDEKEGIYITNEERKGKDGFLELKSVLTRVHSNKPAQSDN |
| | | LTIWCMALSPVPGNKVWNISSEKITFLLGSEISSTDPPLSVTESTLDTQPSPASSVSPARYPATSSVTLV |
| | | DVSALRPNTTPQPSNSSMTTRGFNYPWTSSGTDTKKSVSRIPSETYSSSPSGAGSTLHDNVFTSTARAFS |
| | | EVPTTANGSTKTNHVHITGIVVNKPKDGMSWPVIVAALLFCCMILFGLGVRKWCQYQKEIMERPPPFKPP |
| | | PPPIKYTCIQEPNESDLPYHEMETL |
| 333 | anti- | GAGGTACAGCTGGTGGAATCTGGGGGAGACCTGGTGAAGCCTGCAGGGTCCCTGAGACTGTCCTGTGTGG |
| | CD96 | CCTCTGGATTCCCCTTCAGTGACTCTGACATGAACTGGGTCCGCCAGGCTCCTGGGAAGGGGCTACAGTG |
| | R7P1-G1 | GGTCGCAGGTATTGACGAGGGTGCAACTGACACAGGCTACGCAGATGCAGTGAAGGGCCGATTCACCATC |
| | clone | TCCAGAGACGACGCCAAGAACACACTTTATCTACAGATGAACAGCCTGAGAGCCGACGACACGGCCATGT |
| | VH-DNA | ACTATTGTGCCGCGGGGGGTAATTTCGCCTACTGGGGCCAGGGAACCCTGGTCACCGTCTCGAGT |
| 334 | anti- | GATATCGTCATGACGCAGACCCCACTGTCCCTGTCCGTCAGCCCTGGAGAGCCGGCCTCCATCTCCTGCA |
| | CD96 | AGGCCAGTCAGAGCCTCCTGCACAGTAACGGGAACACCTATTTGTACTGGTTTCGACAGAAGCCAGGCCA |
| | R7P1-G1 | GTCTCCACAGGGCCTGATCTACTTGGTTTCCAAACGTTTCTCTTGGGTCCCAGACAGGTTCAGTGGCAGC |
| | clone | GGGTCAGGGACGGATTTCACCCTGAGAATCAGCAGAGTGGAGGCTGATGATGCTGGACTTTATTACTGCG |
| | VL-DNA | GGCAAGGTATACAATATCCTTATACTTTCAGCCAGGGAACCAAGCTGGAGATAAAA |
| 335 | Anti- | GGGGTGCAGCTGGTGGAGTCTGGGGGAGACCTGGTGAAGCCTGCGGGGTCCCTGAGACTGTCCTGTGTGG |
| | TIGIT | CCTCTGGATTCACCTTCAGTAACTATGGCATGAGCTGGGTCCGCCAGGCTCCTGGGAAGGGGCTGCATGG |
| | R8P1-C2 | GGTCGCGTCTATTGCCAGTGGTGGAAGTGCCACTGCTTACGCAGACGCTGTGAAGGGCCGATTCACCCTC |
| | clone | TCCAGAGACGACGCCAAGAACACACTGTATCTGCAGATGAACAACCTGAGAACCGAGGACACGTCCATGT |
| | VH-DNA | ATTACTGTGTGGCCCCGGGATTTCAGTACTGGGGCCAGGGCACCCTGGTCACTGTCTCGAGT |
| 336 | Anti- | GATATCGTCATGACACAGACCCCACTGTCCCTGTCCGTCAGCCCTGGAGAGACTGCCTCCATCTCCTGCA |
| | TIGIT | AGGCCAGTCAGAGCCTCCTGCACAGTAACGGGAACACCTATTTGTTTTGGTTTCGACAGAAGTCAGGCCA |
| | R8P1-C2 | GTCTCCACAGCGTTTGATCTATAAGGTCTCCAACAGAGACCCTGGGGTCCCAGACAGGCTCAGTGGCAGC |
| | clone | GGGTCAGGGACAGATTTCACGCTGAGAATCAGCAGAGTGGAGGCTGACGATGCTGGAGTTTATTACTGCG |
| | VL-DNA | GGCAAGGTATACAGATTCCTCGCACTTTTGGCCAAGGGACCAAACTGGAGATCAAA |

1.-31. (canceled)

32. A method for generating a polynucleotide sequence encoding for an antigen binding polypeptide or an antigen binding polypeptide-based fragment immunoreactive with a target antigen, the method comprising:
a) determining one or more nucleotide sequences encoding at least one hypervariable loop or complementary determining region (CDR) of the VH and/or the VL domain of a Canidae conventional antibody immunoreactive with said target antigen:
b) designing a polynucleotide sequence comprising:
the one or more nucleotide sequences determined in step a),
one or more nucleotide sequences encoding one or more framework regions (FR) of the VH and/or VL domain of a human antibody,
optionally one or more nucleotide sequences encoding constant domains of a human antibody,
wherein said polynucleotide sequence encodes an antigen binding polypeptide or antigen binding polypeptide-based fragment comprising a VH domain and/or a VL domain that is immunoreactive with said target antigen.

33. The method according to claim 32, wherein
in step a) nucleotide sequences encoding at least one hypervariable loop or CDR of the VH and the VL domain of a Canidae conventional antibody are determined and/or
the polynucleotide sequence designed in step b) comprises nucleotide sequences encoding one or more FRs of the VH and the VL domain of a human antibody.

34. The method according to claim 32, wherein
the nucleotide sequences in step a) encode a CDR1, a CDR2 and a CDR3 of the VH and/or the VL domain of a Canidae conventional antibody immunoreactive with said target antigen; and/or
the polynucleotide sequence designed in step b) comprises nucleotide sequences encoding the framework regions FR1, FR2, FR3 and FR4 of a human antibody.

35. The method according to claim 32, wherein step a) further comprises expressing polypeptides comprising VH and VL domains and screening said polypeptides for immunoreactivity with said target antigen so as to identify antigen binding polypeptides comprising said VH and VL domains.

36. The method according to claim 32, wherein prior to step a) a step aa) is performed, said step aa) comprising immunizing an animal of a species of the family Canidae, thereby raising said conventional Canidae antibodies against the target antigen, and collecting the antibodies reactive with the target antigen.

37. The method according to claim 32, further comprising a step c) wherein the step c) comprises generating an antigen binding polypeptide encoded by the polynucleotide sequence designed in step b) and optionally screening the polypeptide for homology with human antibodies in the VH and/or VL domain and further optionally increasing the affinity of said VH and VL domains for said target antigen by modification of one or more amino acid residues in one or more CDRs of the polypeptide and/or introducing one or more amino acid substitutions in one or more of the framework regions of the VH and/or VL domain of the human antibody.

38. The method according to claim 32 comprising:
a) determining one or more nucleotide sequences encoding at least one hypervariable loop or complementary determining region (CDR) of the VH and the VL domain of a Canidae conventional antibody immunoreactive with said target antigen, expressing polypeptides comprising said VH and VL domains and screening said polypeptides for immunoreactivity with said target antigen so as to identify antigen-binding polypeptides comprising said VH and VL domains:
b) designing a polynucleotide sequence comprising:
the one or more nucleotide sequences determined in step a);
one or more nucleotide sequences encoding one or more framework regions of the VH and VL domain of a human antibody;
optionally, one or more nucleotide sequences encoding constant domains of a human antibody:
wherein said polynucleotide sequence encodes an antigen binding polypeptide or antigen binding polypeptide-based fragment comprising a VH domain and a VL domain that is immunoreactive with said target antigen:
c) generating an antigen binding polypeptide encoded by the polynucleotide sequence designed in step b):
d) optionally, screening the antigen binding polypeptides for homology with human antibodies in the VH and VL domain:
e) optionally, increasing the affinity of said VH and VL domains for said target antigen by modification of one or more amino acid residues in one or more CDRs and/or FRs of the polypeptide.
thereby producing an antigen binding polypeptide or antigen-binding polypeptide-based fragment comprising a VH domain and a VL domain that is immunoreactive with said target antigen.

39. The method according to claim 32, wherein the species in the family Canidae is selected from domestic dogs, wolves, coyotes, foxes, jackals and dingoes.

40. The method according to claim 32, wherein the method comprises the steps of:
I. preparing an expression vector encoding an antigen binding polypeptide immunoreactive with a target antigen by:
aa) optionally, actively immunizing an animal of a species in the family Canidae, thereby raising conventional Canidae antibodies against said target antigen:
a) determining one or more nucleotide sequences encoding at least one hypervariable loop or complementary determining region (CDR) of the VH and/or the VL domain of a Canidae conventional antibody immunoreactive with said target antigen, expressing polypeptides comprising VH and VL domains and screening said polypeptides for immunoreactivity with said target antigen so as to identify antigen-binding polypeptides comprising said VH and VL domains:
b) designing a polynucleotide sequence comprising:
the one or more nucleotide sequences determined in step a),
one or more nucleotide sequences encoding one or more framework regions (FR) in the VH and/or VL domain of a human antibody;
optionally, one or more nucleotide sequences encoding constant domains of a human antibody:
wherein said polynucleotide sequence encodes an antigen binding polypeptide or antigen binding polypeptide-based fragment comprising a VH domain and a VL domain that is immunoreactive with said target antigen:
c) generating an antigen binding polypeptide encoded by the polynucleotide sequence designed in step b) by
c1) preparing cDNA or genomic DNA from a sample comprising lymphoid tissue (e.g. circulating B cells) from said immunized animals of a species of the family Canidae;
c2) amplifying regions of said cDNA or genomic DNA to obtain amplified gene segments, each gene segment comprising a sequence of nucleotides encoding a hypervariable loop or CDR of a VH domain and/or a sequence of nucleotides encoding a hypervariable loop or CDR of a VL domain of a Canidae conventional antibody.
c3) cloning the gene segments obtained in c2) into expression vectors, such that each expression vector contains a gene segment encoding one or more hypervariable loops or CDRs of a VH domain and/or a gene segment encoding one or more hypervariable loops or CDRs of a VL domain and directs expression of an antigen binding polypeptide comprising said one or more hypervariable loops or CDRs of a VH domain and/or said one or more hypervariable loops or CDRs of a VL domain, thereby producing a library of expression vectors;
c4) screening antigen binding polypeptides encoded by the library obtained in step c3) for immunoreactivity with said target antigen, and thereby selecting an expression vector encoding an antigen binding polypeptide immunoreactive with said target antigen;
c5) cloning the gene segment encoding the one or more hypervariable loops or CDRs of a VH domain of the vector selected in part c4) and the gene segment encoding the one or more hypervariable loops or CDRs of a VL domain of the vector selected in part c4) into a further expression vector, in operably linkage with a sequence of nucleotides encoding at least one or more framework regions (FR) in the VH and/or VL domain of a human antibody, and optionally, one or more constant domains of a human antibody, thereby producing an expression vector encoding an antigen binding polypeptide:
II. introducing said expression vector into a host cell or a cell-free expression system under conditions which permit expression of the encoded antigen binding polypeptide; and
III. recovering the expressed antigen binding polypeptide.

41. The method according to claim 40, wherein
the nucleotide sequences in step a) encode a CDR1, a CDR2 and a CDR3 of the VH and/or the VL domain of a Canidae conventional antibody immunoreactive with said target antigen; and/or
the polynucleotide sequence designed in step b) comprises nucleotide sequences encoding the framework regions FR1, FR2, FR3, and FR4 of the VH and/or VL domain of a human antibody.

42. The method according to claim 40, wherein said method further comprises, one or more of
between step c4) and step c5), the step of performing a light chain shuffling step and/or a heavy chain shuffling step to select an expression vector encoding a potency-optimized antigen binding polypeptide immunoreactive with said target antigen
after step c5) the step of subjecting the gene segment encoding the VH domain of the vector to germlining and/or codon optimization.

43. The method according to claim 40 wherein the antigen binding polypeptide or antigen binding polypeptide-based fragment comprises a VH domain and a VL domain and, optionally, one or more constant domains, wherein each of the hypervariable loop or CDRs in said VH domain and VL domain is obtained from a VH or VL domain identified in step a) and wherein the framework regions FR1, FR2, FR3 and FR4 are from a human antibody, and optionally wherein the one or more constant domains is human.

44. An antigen binding polypeptide comprising a VH domain and a VL domain and optionally further comprising one or more constant domains, wherein at least one of the hypervariable loops or CDRs in the VH and VL domain is obtained from a VH or VL domain of a species in the family Canidae and wherein one or more of the framework regions in the VH domain and VL domain are from a human antibody, and optionally wherein the one or more constant domains are from a human antibody.

45. The antigen binding polypeptide claim 44, wherein
at least the CDR3 in the VH domain and the VL domain is obtained from a species in the family Canidae and optionally the CDR1, CDR2 and CDR3 in the VH domain and the VL domain are obtained from a species in the family Canidae.

46. The antigen binding polypeptide according to claim 44, wherein all framework regions FR1, FR2, FR3 and FR4 in the VH and VL domain are from a human antibody.

47. The antigen binding polypeptide according to claim 45, wherein one or more amino acid substitutions in the one or more CDRs are present as compared to the amino acid sequence of the CDRs of the species in the family Canidae.

48. The antigen binding polypeptide according to claim 45, wherein one or more amino acid substitutions in the one or more FRs of the VH and VL domain are present as compared to the amino acid sequence of the FRs of the human antibody.

49. The antigen binding polypeptide according to claim 45 which is a Fab, Fab1, F(ab′)2, bi-specific Fab′, Fv fragment, diabody, linear antibody, a single chain variable fragment (scFv) or multispecific antibody formed form antibody fragments.

50. A pharmaceutical composition comprising the antigen binding polypeptide according to claim 45.

51. A polynucleotide molecule encoding an antigen binding polypeptide according to claim 45 or encoding a fragment of said antigen binding polypeptide, wherein the polypeptide or the fragment comprises at least one hypervariable loop or CDR obtained from a VH or VL domain of a species in the family Canidae, wherein one or more of the framework regions in the VH and VL domain are from a human antibody, and optionally wherein one or more constant domains is human.

52. An expression vector comprising the polynucleotide molecule of claim 51 operably linked to regulatory sequences which permit expression of the antigen binding polypeptide in a host cell or cell-free expression system.

53. A host cell or cell-free expression system comprising the expression vector of claim 52.
